(19) United States
(12) Patent Application Publication
(10) Pub. No.: US 2014/0371188 A1
(43) Pub. Date: Dec. 18, 2014

(54) COMPOSITIONS AND METHODS FOR INHIBITING MUSCLE ATROPHY AND INDUCING MUSCLE HYPERTROPHY

(71) Applicant: UNIVERSITY OF IOWA RESEARCH FOUNDATION, Iowa City, IA (US)

(72) Inventors: Christopher M. Adams, Iowa City, IA (US); Steven D. Kunkel, Iowa City, IA (US); Michael Welsh, Riverside, IA (US); Scott Ebert, Iowa City, IA (US)

(73) Assignee: UNIVERSITY OF IOWA RESEARCH FOUNDATION, Iowa City, IA (US)

(21) Appl. No.: 14/360,115

(22) PCT Filed: Nov. 21, 2012
(86) PCT No.: PCT/US2012/066341

§ 371 Date: May 22, 2014

Related U.S. Application Data
(60) Provisional application No. 61/563,288, filed on Nov. 23, 2011.

(51) Int. Cl. A61K 31/56; A61K 31/568

(52) U.S. Cl. CPC A61K 31/56; A61K 31/568
USPC 514/171

(57) ABSTRACT

In an aspect, the invention relates to compositions, methods, and kits for inhibiting or preventing skeletal muscle atrophy or inducing muscle hypertrophy by providing to an animal an effective amount of a composition comprising a Gadd45a and/or Cdkn1a inhibitor and an androgen and/or growth hormone elevator or an androgen and/or growth hormone receptor activator. This abstract is intended as a scanning tool for purposes of searching in the particular art and is not intended to be limiting of the present invention.

### CROSS-REFERENCE TO RELATED APPLICATIONS

This application claims the benefit of U.S. Provisional Application No. 61/563,288 filed on Nov. 23, 2011; which is incorporated herein by reference in its entirety.

### STATEMENT REGARDING FEDERALLY SPONSORED RESEARCH

This invention was made with government support under NIH Grant No. T32 GM073610 and NIH/NIAMS Grant No. 1R01AR059115-01, and Cardiovascular Interdisciplinary Research Fellowship No. HL007121, as well as support from the Doris Duke Charitable Foundation, the American Diabetes Association, the Department of Veterans Affairs, and the Fraternal Order of Eagles Diabetes Research Center. The U.S. government has certain rights in the invention.

### BACKGROUND

A variety of stresses, including starvation, muscle disuse, systemic illness and aging cause skeletal muscle atrophy, which is often debilitating. However, despite its broad impact, muscle atrophy remains incompletely understood. Like many complex diseases, muscle atrophy is associated with widespread positive and negative changes in gene expression (Lecker, S. H., et al. (2004) FASEB J. 18, 39-51; Sacheck, J. M., et al. (2007) FASEB J. 21, 140-155; Banduseela, V. C., et al. (2009) Physiol. Genomics 39, 141-159; Welle, S., et al. (2004) Exp. Gerontol. 39, 369-377; Welle, S., et al. (2003) Physiol. Genomics 14, 149-159; Edwards, M. G., et al. (2007) BMC Genomics 8, 80; Stevenson, E. J., et al. (2003) J. Physiol. 551, 33-48; Gonzalez de Aguilar, J. L., et al. (2008) Physiol. Genomics 32, 207-218). Some gene expression changes in atrophying muscle are known to promote atrophy, including induction of genes that promote proteolysis (Bodine, S. C., et al. (2001) Science 294, 1704-1708; Sandri, M., et al. (2004) Cell 117, 399-412; Stitt, T. N., et al. (2004) Mol. Cell 14, 395-403; Moresi, V., et al. (2010) Cell 143, 35-45; Cai, D., et al. (2004) Cell 119, 285-298; Acharyya, S., et al. (2004) J. Clin. Investig. 114, 370-378; Mammucari, C., et al. (2007) Cell Metab. 6, 458-471; Zhao, J., et al. (2007) Cell Metab. 6, 472-483; Plant, P. J., et al. (2009) J. Appl. Physiol. 107, 224-234) and repression of the gene encoding PGC-1α, a transcriptional coactivator that promotes mitochondrial biogenesis and energy production (Sandri, M., et al. (2006) Proc. Natl. Acad. Sci. U.S.A. 103, 16260-16265; Wenz, T., et al. (2009) Proc. Natl. Acad. Sci. U.S.A. 106, 20405-20410). However, most atrophy-associated gene expression changes are unstudied, and it remains unknown if these changes contribute to muscle atrophy, and if so, to what extent.

Despite these advances in the understanding the physiology and pathophysiology of muscle atrophy, there is still a scarcity of compositions that are both potent, efficacious, and selective modulators of muscle growth and also effective in the prevention and treatment of muscle atrophy, and in conditions in which the muscle atrophies or the need to increase muscle mass is involved. These needs and other needs are satisfied by the present invention.

### SUMMARY

Disclosed herein is a composition for treating or preventing skeletal muscle atrophy, the composition comprising a Gadd45a and/or Cdkn1a inhibitor and an androgen and/or growth hormone elevator. Disclosed herein is a composition for treating or preventing skeletal muscle atrophy, the composition comprising a Gadd45a and/or Cdkn1a inhibitor and androgen and/or growth hormone receptor activator. Disclosed herein is a composition for treating or preventing skeletal muscle atrophy in a mammal, the composition comprising RNAi targeting Gadd45a and/or Cdkn1a. Disclosed herein is a composition for treating or preventing skeletal muscle atrophy in a mammal, the composition comprising ursolic acid or an ursolic acid derivative.

Disclosed herein is a method for preventing or treating skeletal muscle atrophy in an animal, the method comprising administering to the animal an effective amount of a composition comprising a Gadd45a and/or Cdkn1a inhibitor and an androgen and/or growth hormone elevator. In an aspect, disclosed herein is a method for preventing or treating muscle atrophy in an animal, the method comprising administering to the animal an effective amount of a composition comprising a Gadd45a and/or Cdkn1a inhibitor and an androgen and/or growth hormone receptor activator. In an aspect, disclosed herein is a method for preventing or treating muscle atrophy in an animal, the method comprising administering to the animal an effective amount of an androgen and/or growth hormone elevator subsequent to the animal having received a Gadd45a and/or Cdkn1a inhibitor. In an aspect, disclosed herein is a method for preventing or treating muscle atrophy in an animal, the method comprising administering to the animal an effective amount of a Gadd45a and/or Cdkn1a inhibitor subsequent to the animal having received an androgen and/or growth hormone elevator. In a further aspect, disclosed herein is a method for preventing or treating muscle atrophy in an animal, the method comprising administering to the animal an effective amount of an androgen and/or growth hormone receptor activator subsequent to the animal having received a Gadd45a and/or Cdkn1a inhibitor. In a further aspect, disclosed herein is a method for preventing or treating muscle atrophy in an animal, the method comprising administering to the animal an effective amount of a Gadd45a and/or Cdkn1a inhibitor subsequent to the animal having received an androgen and/or growth hormone receptor activator.

Disclosed herein is a method for facilitating muscle hypertrophy, the method comprising the steps of (i) inhibiting expression of Gadd45a and/or Cdkn1a, and (ii) increasing cellular concentration of androgen and/or growth hormone. Further disclosed is a method for facilitating muscle hypertrophy, the method comprising the steps of (i) inhibiting expression of Gadd45a and/or Cdkn1a, and (ii) increasing activity of androgen and/or growth hormone receptor.

Disclosed herein is a method comprising the steps of inhibiting expression of Gadd45a and/or Cdkn1a and providing androgen and/or growth hormone. In a further aspect, disclosed herein is a method comprising the steps of inhibiting expression of Gadd45a and/or Cdkn1a and activating androgen and/or growth hormone receptor.

Disclosed herein is a method of treating or preventing skeletal muscle atrophy in a mammal, the method comprising administering ursolic acid or an ursolic acid derivative; and inducing expression of VEGFA and/or nNOS. Also disclosed is a method of treating or preventing skeletal muscle atrophy in a mammal, the method comprising administering ursolic acid or an ursolic acid derivative; and activating growth hormone receptor. Disclosed is a method for activating growth hormone receptor in a mammal, the method comprising administering a composition comprising ursolic acid or an ursolic acid derivative. Disclosed herein is a method for increasing skeletal muscle blood flow in a mammal, the method comprising administering a composition comprising ursolic acid or an ursolic acid derivative.

Disclosed herein is a kit comprising a Gadd45a and/or Cdkn1a inhibitor and an androgen and/or growth hormone elevator. In an aspect, disclosed herein in a kit comprising a Gadd45a and/or Cdkn1a inhibitor and instructions for administering an androgen and/or growth hormone elevator. In an aspect, disclosed herein is a kit comprising an androgen and/or growth hormone elevator and instructions for administering a Gadd45a and/or Cdkn1a inhibitor. In a further aspect, disclosed herein is a kit comprising a Gadd45a and/or Cdkn1a inhibitor and an androgen and/or growth hormone receptor activator. In an aspect, disclosed herein in a kit comprising a Gadd45a and/or Cdkn1a inhibitor and instructions for administering an androgen and/or growth hormone receptor activator. Further disclosed is a kit comprising an androgen and/or growth hormone receptor activator and instructions for administering a Gadd45a and/or Cdkn1a inhibitor.

Disclosed herein is a pharmaceutical composition comprising an androgen and/or growth hormone receptor activator, a Gadd45a and/or Cdkn1a inhibitor, and a pharmaceutically acceptable carrier. In an aspect, disclosed herein a pharmaceutical composition comprising an androgen and/or growth hormone elevator, a Gadd45a and/or Cdkn1a inhibitor, and a pharmaceutically acceptable carrier. In an aspect, disclosed herein a pharmaceutical composition comprising an inhibitor of Gadd45a and/or Cdkn1a expression and a pharmaceutically acceptable carrier. In an aspect, disclosed herein a pharmaceutical composition comprising an inhibitor of Gadd45a and/or Cdkn1a functions and a pharmaceutically acceptable carrier. In an aspect, disclosed herein a pharmaceutical composition comprising an inhibitor of Cdkn1a gene demethylation and a pharmaceutically acceptable carrier.

Disclosed herein is a screening method comprising the steps of (i) administering a candidate inhibitor to a cell, and (ii) measuring expression of Gadd45a and/or Cdkn1a in the cell, wherein decreased expression in the cell relative to a control cell identifies a potential treatment or preventative for muscle atrophy.

Also disclosed are methods for manufacturing a medicament associated with muscle atrophy or the need to increase muscle mass, comprising combining at least one disclosed composition or at least one disclosed product with a pharmaceutically acceptable carrier or diluent.

Also disclosed are uses of a disclosed composition or a disclosed product in the manufacture of a medicament for the treatment of a disorder associated with muscle atrophy or the need to increase muscle mass.

While aspects of the present invention can be described and claimed in a particular statutory class, such as the system statutory class, this is for convenience only and one of skill in the art will understand that each aspect of the present invention can be described and claimed in any statutory class.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying Figures, which are incorporated in and constitute a part of this specification, illustrate several aspects and together with the description serve to explain the principles of the invention.

FIG. 1 shows the generation and characterization of ATF4 mKO mice. (A-C) The targeting construct was transfected into ES cells derived from C57BL/6 mice, and G-418 resistant clones were analyzed for homologous recombination by Southern blotting. (A) Diagram of the targeting construct. 5′ and 3′ homology arms are indicated in red, and the conditional knockout region (1.8 Kb comprising ATF4 exons 2 and 3) is indicated in green. (B) Diagram of XbaI (X) and HindIII (H) sites and Southern blot probes. (C) Southern blots of genomic DNA from four G-418-resistant ES clones. (D-F) Flp recombinase was used to delete the Neo selectable marker gene, and standard procedures were used to generate heterozygous ATF4(L/+) mice and then homozygous ATF4(L/L) mice. (D) Diagram showing the PCR-based genotyping strategy. Sequences of primers A and B were 5′-GCAGACGTTCCTGGGTTAGATACAATAAC-3′ (SEQ ID NO:1) and 5′-GCCACTGTTTACTATCACCCCAGCC-3′(SEQ ID NO:2), respectively. (E) Genotypes of progeny from an ATF4(+/+) X ATF4(L/+) mating. Pups 1-2 and 8-9 are ATF4(+/+), whereas pups 3-7 are ATF4(L/+). (F) Genotypes from an ATF4(L/+) X ATF4(L/+) mating. Pups 2 & 6 are ATF4(+/+), pups 4 & 5 are ATF4(L/+) and pups 1 & 3 are ATF4(L/L). ATF4 mKO mice were subsequently generated by crossing ATF4(L/L) mice to MCK-Cre mice. MCK-Cre were generated on a FVB genetic background, but have been backcrossed for >10 generations into a C57BL/6 background. (G-H) Generation of conditional knockout mice lacking ATF4 in striated muscle (ATF4 mKO). (G) Diagram showing the floxed ATF4(L) allele and PCR-based strategy to detect its excision by Cre recombinase. (H) MCK-Cre excises the ATF4(L) allele in striated muscle. TA; tibialis anterior. Gastroc; gastrocnemius. (I) Loss of ATF4 does not alter the percentage or size of type I or type II fibers under basal conditions. Left: mean fiber diameter. Right: percent fiber type. Data are means±SEM from ≧150 fibers per TA, from ≧3 mice per genotype. (J) Total body and tissue weights in ATF4 mKO mice and ATF4(L/L); MCK-Cre(0/0) littermate control mice under basal and fasted conditions. Mice were allowed ad libitum access to food, or fasted by removing food but not water for 24 or 48 h. Data are means±SEM from ≧16 mice per genotype per condition. ND: not determined.

FIG. 2 shows that loss of ATF4 delays skeletal muscle atrophy induced by fasting or immobilization. (A-C) ATF4 mKO mice and littermate controls were allowed ad libitium access to food or fasted by removing food but not water for 24 h or 48 h. **P<0.01. *P<0.05. (A) Tibialis anterior (TA) muscle weights. Each data point represents the mean±SEM from ≧9 mice. (B) TA muscle fiber size in the absence and presence of a 48 h fast. Left: mean fiber diameters ±SEM. Right: fiber size distributions. Data are from ≧250 muscle fibers per TA, from ≧3 mice per condition. (C) Representative H&E stains from (B). (D-F) ATF4 mKO mice and littermate controls were subjected to unilateral TA immobilization for 0, 3 or 7 days. (D) TA muscle weights. In each mouse, weight of the immobile (atrophied) TA was normalized to the weight of the mobile (non-atrophied) TA, which was set at 1. Each data point represents the mean±SEM from ≧10 mice. *P<0.01. (E) Representative H&E stains from (D). (F) TA muscle fiber size. In each mouse, ≧350 muscle fibers were measured in each TA, and the mean fiber diameter in the immobile TA was normalized to the mean fiber diameter in the mobile TA, which was set at 1. Each data point represents the mean±SEM from ≧5 mice. *P<0.05.

FIG. 3 shows identification of Gadd45a as a transcript that is reduced in ATF4 mKO muscle and increased by ATF4 overexpression in both mouse muscle and cultured C2C12 myotubes. (A-C) ATF4, but not ATF4ΔbZIP, causes atrophy of C2C12 myotubes. Myotubes were infected with adenovirus expressing eGFP alone (Ad-GFP), eGFP+ATF4 (Ad-ATF4), or eGFP+ATF4ΔbZIP (Ad-ATF4ΔbZIP), as indicated, then harvested 48 h after infection. ATF4 constructs contained FLAG epitope tags. (A) Total cellular protein extracts were subjected to immunoblot analysis with anti-FLAG monoclonal IgG. (B) Representative images. (C) Mean myotube diameter ±SEM from 3 experiments. P-values were determined by one-way ANOVA and Dunnett's post-test. *P<0.01. (D) Affymetrix Mouse Exon 1.0 ST arrays were used to identify mRNAs that were increased by ATF4 overexpression in myotubes (Ad-ATF4 vs. Ad-ATF4ΔbZIP), decreased by loss of ATF4 in fasted mouse TA muscle (ATF4 mKO mice vs. littermate controls) and increased by ATF4 overexpression in C57BL/6 TA muscle (ATF4 plasmid vs. empty pcDNA3 plasmid). n=3 arrays per condition and statistical significance was arbitrarily defined as P≦0.01 by t-test. Numbers indicate the number of transcripts in each category. (E) qPCR confirmation that ATF4 increases Gadd45a mRNA in myotubes. Data are normalized to the level of Gadd45a mRNA in Ad-GFP-infected myotubes, and are means±SEM from 3 experiments. *P<0.05 (F) qPCR confirmation that ATF4 mKO muscles contain reduced Gadd45a mRNA. Mice were fasted for 24 h before TA muscles were harvested for qPCR analysis. mRNA levels in ATF4 mKO muscles were normalized to levels in littermate control muscles. Data are means±SEM from 10 mice per genotype. *P<0.05.

FIG. 4 shows that Gadd45a is required for skeletal muscle fiber atrophy induced by immobilization, fasting and denervation. (A-B) Gadd45a is required for immobilization-induced muscle fiber atrophy. On day 0, bilateral C57BL/6 TA muscles were transfected with either 20 μg p-miR-Control or 20 μg p-miR-Gadd45a, as indicated. All plasmids carried EmGFP as a transfection marker. On day 3, right hindlimbs were immobilized. On day 10, bilateral TA muscles were harvested for analysis. (A) Upper panel: mRNA levels were determined by qPCR and normalized to levels in mobile, p-mir-Control-transfected TA; data are means±SEM from 3 muscles per condition. Lower left: Mean fiber diameters ±SEM from 5 TAs per condition. Lower right: fiber size distributions. Statistical differences were determined using a linear mixed model with a random effect for mouse (58). Different letters are statistically different (P≦0.05). (B) Representative images. (C) Gadd45a is required for fasting-induced muscle fiber atrophy. On day 0, C57BL/6 TA muscles were transfected with either 20 μp-miR-Control (left leg) or 20 μg p-miR-Gadd45a (right leg). On day 9, mice were fasted for 24 h and then harvested for analysis. Left: Mean fiber diameters ±SEM from ≧4 TAs per condition. *P<0.01. Right: fiber size distributions. (D) Gadd45a is required for denervation-induced muscle atrophy. On day 0, C57BL/6 TA muscles were transfected bilaterally with either 20 μg p-miR-Control or 20 μg p-miR-Gadd45a. On day 3, the left sciatic nerve was transected. On day 10, bilateral TA muscles were harvested. Left: Mean fiber diameters ±SEM from ≧5 TAs per condition. Statistical differences were determined using a linear mixed model with a random effect for mouse; different letters are statistically different (P≦0.05). Right: fiber size distributions. (E) Gadd45a is required for ATF4-mediated muscle atrophy. C57BL/6 TA muscles were transfected with 10 μg p-ATF4-FLAG+ either 20 μg p-miR-Control (left TA) or 20 μg p-miR-Gadd45a (right TA), then harvested 10 days later. Left: mean fiber diameters ±SEM from 5 TAs per condition. *P=0.03. Right: fiber size distributions.

FIG. 5 shows additional data that Gadd45a is required for muscle fiber atrophy induced by immobilization, fasting and denervation. (A) Gadd45a is required for immobilization-induced muscle atrophy. On day 0, bilateral C57BL/6 TA muscles were transfected with either 20 μg p-miR-Control or 20 μg p-miR-Gadd45a #2. On day 3, right hindlimbs were immobilized, and on day 10, bilateral TA muscles were harvested for analysis. Left panel: Gadd45a mRNA levels were determined by qPCR and normalized to levels in mobile, p-mir-Control-transfected muscles, which were set at one and indicated by the dashed line. Data are means±SEM from 3 muscles per condition. Right panel: mean muscle fiber diameter. Data are means±SEM from >500 transfected fibers per TA, from 5 TAs per condition. Statistical differences were determined using a linear mixed model with a random effect for mouse; different letters are statistically different. (B) Gadd45a is required for fasting-induced muscle atrophy. C57BL/6 TA muscles were transfected with either 20 μp-miR-Control (left leg) or 20 μg p-miR-Gadd45a #2 (right leg). Nine days after transfection, mice were fasted for 24 h and then TA muscle fiber size was analyzed. Data are means±SEM from ≧350 transfected fibers per TA, from 5 TAs per condition. *P<0.01 by t-test. (C) miR-Gadd45a does not alter the percentage or size of type I or type II fibers under basal conditions. C57BL/6 TAs were transfected with 20 μg p-miR-Control, or 20 μg p-miR-Gadd45a, as indicated, then harvested 10 days later for fiber type analysis. Left: percent fiber type. Right: mean fiber diameter. Data are means±SEM from ≧125 fibers per TA, from 3 TAs per condition. (D-F) miR-Gadd45a does not alter the percentage of type I or type II fibers, but it reduces atrophy of type II fibers during immobilization-, fasting-, and denervation-induced muscle atrophy. Bilateral C57BL/6 TAs were transfected with 20 μg p-miR-Control or 20 μp-miR-Gadd45a, as indicated. (D) Three days post-transfection, right hindlimbs were immobilized. One week later, bilateral TAs were harvested for fiber type analysis. Left: percent fiber type. Right: mean fiber diameter. Data are means±SEM from ≧125 fibers per TA, from 3 TAs per condition. (E) Nine days post-transfection, mice were fasted for 24 h and then TAs were harvested for fiber type analysis. Left: percent fiber type. Right: mean fiber diameter. Data are means±SEM from ≧125 fibers per TA, from 3 TAs per condition. (F) Three days post-transfection, the left sciatic nerve was transected. One week later, bilateral TAs were harvested for fiber type analysis. Left: percent fiber type. Right: mean fiber diameter. Data are means±SEM from ≧125 fibers per TA, from 3 TAs per condition. (C-F) Statistical differences were determined by one-way ANOVA and Dunnett's post-test. *P<0.05.

FIG. 6 shows that Gadd45a overexpression induces myotube atrophy in vitro and skeletal muscle fiber atrophy in vivo. (A-C) C2C12 myotubes were infected with the indicated adenoviruses, and then measured and harvested 48 h after infection. Ad-Gadd45a is adenovirus co-expressing eGFP and Gadd45a-FLAG. (A) Protein extracts were subjected to immunoblot analysis with anti-FLAG monoclonal IgG (B) Representative images. (C) Mean myotube diameters ±SEM from 3 experiments. *P<0.01. (D-E) C57BL/6 TA muscles were transfected with 2 μg p-eGFP+ either 20 μg empty vector (pcDNA3; left TA) or 20 μp-Gadd45a-FLAG (right TA), then harvested 10 days later. (D) Muscle protein extracts were subjected to immunoblot analysis with anti-FLAG monoclonal IgG. (E) Muscle fiber size. Left: mean fiber diameters ±SEM from 3 experiments. *P<0.02. Right: fiber size distributions. (F-G) ATF4 mKO TA muscles were transfected as in (D-E) and harvested 7 days later. (F) Mean fiber diameters ±SEM from 3 experiments. *P<0.01. (G) Representative images.

FIG. 7 shows that Gadd45a overexpression induces muscle fiber atrophy in fasted ATF4 mKO mice and in type II fibers. (A) ATF4 mKO TA muscles were transfected with 2 μg pCMV-eGFP+ either 10 μg empty vector (pcDNA3; left TA) or 10 μg p-Gadd45a-FLAG (right TA). On day 6, mice were fasted for 24 h and then harvested for analysis. Data are means±SEM from ≧250 transfected fibers per TA, from 3 TA muscles per condition. *P<0.01. (B) Gadd45a overexpression induces atrophy in type II but not type I fibers. C57BL/6 TA muscles were transfected with 2 μg p-eGFP+ either 20 μg empty vector (pcDNA3; left TA) or 20 μg p-Gadd45a-FLAG (right TA), then harvested 10 days later for fiber type analysis. Data are means±SEM from ≧125 transfected fibers per TA, from 3 TA muscles per condition.

FIG. 8 shows that Gadd45a is a myonuclear protein that alters myonuclear structure and reprograms skeletal muscle gene expression. (A-B) Immunohistochemical detection of FLAG-tagged Gadd45a in myotube nuclei (A) and skeletal muscle fiber nuclei (B). In (A), myotubes were infected with Ad-Gadd45a for 48 h before immunohistochemistry. In (B), mouse muscle fibers were transfected with 2 μg p-eGFP+20 μg p-Gadd45a-FLAG and then harvested 10 days later for immunohistochemistry. (C-D) Gadd45a alters myonuclear morphology in a manner similar to muscle denervation. (C) Left-sided sciatic nerves of C57BL/6 mice were transected, and bilateral TA muscles were harvested 7 days later for transmission electron microscopy (TEM) analysis. Top: representative images. Bottom: effect of denervation on the lesser diameter of myonuclei. Data are means±SEM from >50 myonuclei per condition. *P<0.01. (D) C57BL/6 TA muscles were transfected with 20 μg pcDNA3 (left TA) or 20 μg p-Gadd45a-FLAG (right TA), then harvested 7 days later for TEM analysis. Top: representative images. Bottom: effect of Gadd45a on the lesser diameter of myonuclei. Data are means±SEM from >30 myonuclei per condition. *P<0.01. (E) Effects of denervation and Gadd45a on skeletal muscle mRNA levels. To determine effects of denervation, left-sided sciatic nerves of C57BL/6 mice were transected, and bilateral TA muscles were harvested 7 days later. Bilateral TA muscle mRNA levels were then measured with exon expression arrays, and levels in denervated muscles were normalized to levels in contralateral innervated muscles. To determine effects of Gadd45a, ATF4 mKO TA muscles were transfected and harvested as in (D). Bilateral TA muscle mRNA levels were then measured with exon expression arrays, and levels in Gadd45a-transfected muscles were normalized to levels in contralateral control muscles. n=4 arrays per condition. Statistical significance was defined as P≦0.01 by paired t-test. (E) Denervation significantly altered levels of 1674 mRNAs (out of >16,000 mRNAs measured). Pie chart shows effects of denervation and Gadd45a on these mRNAs.

FIG. 9 shows representative effects of Gadd45a on skeletal muscle mRNA levels. (A-C) mRNA levels were analyzed with qPCR (A) or exon expression arrays (B-C; n=4 arrays per condition). To determine effects of denervation, left-sided sciatic nerves of C57BL/6 mice were transected, and then bilateral TA muscles (A) or gastroc muscles (B-C) were harvested 7 d later. To determine effects of Gadd45a, TA muscles of ATF4 mKO mice were transfected with 20 μg empty vector (pcDNA3; left TA) or 20 μg p-Gadd45a-FLAG (right TA), then harvested 7 d later. (A) qPCR validation of several key mRNAs whose levels were significantly altered by Gadd45a and/or denervation, as assessed by exon expression arrays; see also Table S1. Data are mean log₂ signal changes ±SEM from ≧4 mice per condition. *P<0.05. (B) KEGG and Biocarta gene sets similarly affected by denervation and Gadd45a overexpression, as assessed by gene set enrichment analysis of the exon array data. FDR≦0.25 and P≦0.05 for all gene sets shown. (C) Denervation and Gadd45a increase Runx1 mRNA and Runx1 gene targets, with statistical significance defined as P≦0.01 by paired t-test. (D) Time course of Gadd45a overexpression. TA muscles of C57BL/6 mice were transfected with 20 μg pcDNA3 (left TA) or 20 μg p-Gadd45a-FLAG (right TA), and harvested at the indicated time post-transfection. mRNA levels were determined with qPCR. In each mouse, levels in Gadd45a-transfected muscles were normalized to levels in contralateral control muscles. Each data point represents mean log₂ signal change ±SEM from 4 mice; in some cases, error bars are too small to see. *P<0.05.

FIG. 10 shows that Gadd45a reduces PGC-1α, mitochondria, Akt activity and protein synthesis, and it increases autophagy and caspase-mediated proteolysis. (A) Gadd45a decreases PGC-1α and increases lipidated LC3 and caspase-3 protein. C57BL/6 TA muscles were transfected with 20 μg pcDNA3 (left TA) or 20 μg p-Gadd45a-FLAG (right TA), and harvested 10 days later for SDS-PAGE and immunoblot analysis with the indicated antibodies. Left: representative immunoblots. Right: quantification. In each muscle, PGC-1α, LC3-II and caspase-3 signals were normalized to the actin signal, and in each mouse, levels in the presence of Gadd45a were normalized to levels in the absence of Gadd45a. Data are means±SEM from 4 mice. *P<0.05. (B) Gadd45a reduces mitochondrial DNA. C57BL/6 TA muscles were transfected and harvested as in (A) for qPCR analysis of mitochondrial DNA (mtDNA), which was normalized to the amount of nuclear DNA (nDNA) in the same muscle. Data are means±SEM from 7 mice. *P<0.02. (C) Gadd45a reduces Akt and GSK-3β phosphorylation. C2C12 myotubes were infected with control virus (Ad-ATF4ΔbZIP) or Ad-Gadd45a, and then harvested 48 h later for SDS-PAGE and immunoblot analysis with the indicated antibodies. Left: representative immunoblots. Right: quantification. Phospho-Akt and phospho-GSK-3β signals were normalized to the actin signal from the same sample. Levels in Ad-Gadd45a-infected myotubes were then normalized to levels in control myotubes. Data are means±SEM from 4 experiments. *P<0.05. (D) Gadd45a reduces protein synthesis. C2C12 myotubes were infected with control virus (Ad-ATF4ΔbZIP) or Ad-Gadd45a. Protein synthesis was assessed 48 h later by measuring [³H]-leucine incorporation. Levels in Ad-Gadd45a-infected myotubes were then normalized to levels in control myotubes. Data are means±SEM from 5 experiments. *P<0.01. (E) Gadd45a increases proteolysis. C2C12 myotubes were incubated with [³H]-tyrosine for 20 h, washed with chase medium for 2 h, and then infected with control virus (Ad-ATF4ΔbZIP) or Ad-Gadd45a in fresh chase medium. Protein degradation was assessed 36 h later by measuring [³H]-tyrosine release. Levels in Ad-Gadd45a-infected myotubes were then normalized to levels in control myotubes. Data are means±SEM; n=8. *P<0.05. (F) Gadd45a induces autophagosome formation. C57BL/6 TA muscles were transfected as in (A), and harvested 7 days later for TEM analysis. (G) Gadd45a increases caspase-mediated proteolysis. C57BL/6 TA muscles were transfected and harvested as in (A), and then caspase-mediated proteolysis was measured. In each mouse, the level in the presence of Gadd45a was normalized to the level in the absence of Gadd45a. Data are means±SEM from 7 mice. *P<0.01.

FIG. 11 shows that Gadd45a reduces the mitochondrial protein Cox4, and increases autophagy and caspase-mediated proteolysis without causing cell death. (A) Gadd45a reduces the mitochondrial protein Cox4. C57BL/6 TA muscles were transfected with either 20 μg empty vector (pcDNA3; left TA) or 20 μp-Gadd45a-FLAG (right TA), then harvested 10 days later. Top: representative immunoblots. Below: quantification. In each sample, the Cox4 signal was normalized to the actin signal. Levels in muscles overexpressing Gadd45a were then normalized to levels in control muscles. Data are mean changes ±SEM from 7 TAs per condition. *P<0.05. (B-D) C2C12 myotubes were infected with adenovirus expressing eGFP+ATF4ΔbZIP (Ad-ATF4ΔbZIP) or eGFP+ Gadd45a (Ad-Gadd45a) then harvested 48 h later. (B) Gadd45a increases autophagy-related mRNAs, but not atrogin-1 or MuRF1 mRNAs. mRNA levels were measured by qPCR analysis. Levels in Ad-Gadd45a-infected myotubes were normalized to levels in Ad-ATF4ΔbZIP-infected myotubes, which were set at 1 and are indicated by the dashed line. Data are mean changes ±SEM from 3 experiments. *P<0.05. (C) Gadd45a increases Bnip3 protein. Top: representative immunoblots. Below: quantification. In each sample, the Bnip3 signal was normalized to the actin signal. Levels in Ad-Gadd45a-infected myotubes were then normalized to levels in Ad-ATF4ΔbZIP-infected myotubes. Data are mean changes ±SEM from 3 experiments. *P<0.05. (D) Gadd45a increases caspase-mediated proteolysis. Caspase activity in Ad-Gadd45a-infected myotubes was normalized to activity in Ad-ATF4ΔbZIP-infected myotubes. Data are means±SEM from 3 experiments. *P<0.01. (E) Gadd45a does not cause cell death. C2C12 myotubes were infected for 48 h with Ad-Gadd45a, and then stained with 0.2% trypan blue. As a positive control for cell death, myotubes were treated with 80% ethanol for 20 min before trypan blue staining

FIG. 12 shows that Gadd45a induces Cdkn1a mRNA during skeletal muscle atrophy. (A) Identification of Gadd45a and Cdkn1a as skeletal muscle transcripts that are induced by Gadd45a overexpression, denervation and fasting. Effects of Gadd45a overexpression, denervation and fasting on global mRNA levels in tibialis anterior (TA) muscles from C57BL/6 mice were evaluated with Affymetrix Mouse Exon 1.0 ST arrays as described previously (3,4). n=4 arrays per condition. Statistical significance was arbitrarily defined as a 2-fold induction and P<0.01 by t-test. Numbers indicate the number of transcripts in each category. (B) Gadd45a overexpression induces Cdkn1a mRNA. Mouse TA muscles were transfected with either 20 μg empty vector (pcDNA3; left TA) or 20 μp-Gadd45a-FLAG (right TA), then harvested 10 days later. mRNA levels were determined with qPCR. In each mouse, mRNA levels in the presence of Gadd45a overexpression were normalized to levels in the absence of Gadd45a overexpression, which were set at one. Data are means±SEM from 4 mice. *P<0.01. (C) Denervation increases Gadd45a and Cdkn1a mRNAs. The left-sided sciatic nerves of mice were transected to denervate the left TA muscle, and then bilateral TA muscles were harvested 7 days later. mRNA levels were determined with qPCR. In each mouse, mRNA levels from the denervated TA were normalized to values from the innervated TA, which were set at one. Data are means±SEM from 4 mice. *P<0.01. (D) Fasting increases Gadd45a and Cdkn1a mRNAs. Mice were allowed ad libitum access to food (fed) or fasted for 24 h before TA muscles were harvested for analysis. mRNA levels were determined with qPCR. mRNA levels from fasted TAs were normalized to values from fed TAs, which were set at one. Data are means±SEM from 4 mice. *P<0.05. (E) Immobilization increases Gadd45a and Cdkn1a mRNAs. Mice were subjected to unilateral TA immobilization, and then bilateral TA muscles were harvested 3 days later. mRNA levels were determined with qPCR. In each mouse, mRNA levels from the immobile TA were normalized to values from the mobile TA, which were set at one. Data are means±SEM from 6 mice. *P<0.01. (F) Gadd45a increases Cdkn1a protein expression in vivo. Mouse TA muscles were transfected as in (B), and harvested 10 days later for SDS-PAGE and immunoblot analysis. Top, representative immunoblots. Bottom, quantification. In each muscle, the Cdkn1a signal was normalized to the actin signal; in each mouse, levels in the presence of Gadd45a were normalized to levels in the absence of Gadd45a. Data are means±SEM from 4 mice. *P<0.05.

FIG. 13 shows that Gadd45a demethylates and activates the Cdkn1a gene promoter. (A) Gadd45a reduces Cdkn1a promoter methylation in cultured skeletal myotubes. C2C12 myotubes were infected with Ad-ATF4ΔbZIP or Ad-Gadd45a for 48 h before genomic DNA was harvested and analyzed with methylated DNA immunoprecipitation (MeDIP)-chip. Data are -log₁₀ P-values from 110 probes in the tiled region surrounding the Cdkn1a transcription start sites (TSS). Arrows indicate the two TSS and the location of the 273 by differentially methylated region that was selected for further study in (B-G); this region lies −1419 to −1146 bp upstream of Cdkn1a TSS2. (B) Gadd45a interacts with the Cdkn1a promoter. C2C12 myotubes were infected with Ad-ATF4ΔbZIP or Ad-Gadd45a for 48 h, then harvested for chromatin immunoprecipitation studies with the indicated antibodies and PCR primers directed at the 273 bp differentially methylated Cdkn1a promoter region identified in (A). (C) Fasting reduces Cdkn1a promoter methylation in mouse skeletal muscle. Mice were allowed ad libitum access to food (fed) or fasted for 24 h, and then TA muscle genomic DNA was harvested and subjected to bisulfite sequencing. Symbols represent the 4 CpG dinucleotides in the 273 bp differentially methylated Cdkn1a promoter region from (A-B). Open=unmethylated cytosine, closed=methylated cytosine. Each line represents one clone. (D) Illustration of the Cdkn1a reporter construct. The 273 bp differentially methylated Cdkn1a promoter region in (A-C) was inserted into pGL3-Basic upstream of luciferase to generate the Cdkn1a reporter. (E) In vitro methylation reduces Cdkn1a reporter activity in mouse muscle. The Cdkn1a reporter was incubated in vitro in the absence or presence of M.SssI CpG methyltransferase, as indicated. Mouse TA muscles were then transfected with 300 ng pRL-Renilla (both TAs) plus either 15 μg unmethylated Cdkn1a reporter (left TA) or 15 μg methylated Cdkn1a reporter (right TA). One week later, muscles were harvested and luciferase activity was measured. Mean firefly luciferase activity was normalized to mean Renilla luciferase activity in the same muscle, and then levels in the right TA were normalized to levels in the left TA. Data are means±SEM from 8 TAs per condition. *P<0.01. (F) Gadd45a activates the methylated Cdkn1a reporter in mouse muscle. TA muscles were transfected with 15 μg methylated Cdkn1a reporter and 300 ng pRL-Renilla (both TAs) plus either 20 μg pcDNA3 (left TA) or 20 μg p-Gadd45a-FLAG (right TA). One week later, muscles were harvested and luciferase activity was measured as in (E). (G) Gadd45a demethylates the Cdkn1a reporter in mouse muscle. TA muscles were then transfected as in (F). One week later, Cdkn1a reporter DNA was extracted and subjected to bisulfite sequencing using plasmid-specific primers.

FIG. 14 shows that Cdkn1a is required for skeletal muscle fiber atrophy induced by immobilization, denervation, fasting and Gadd45a overexpression. (A-C) Cdkn1a is required for immobilization-induced muscle fiber atrophy. On day 0, bilateral mouse TA muscles were transfected with either 20 μg p-miR-Control, or 20 μg p-miR-Cdkn1a. Both plasmids carried EmGFP as a transfection marker. On day 3, right hindlimbs were immobilized. On day 10, bilateral TA muscles were harvested for analysis. (A) Gadd45a and Cdkn1a mRNA levels in immobilized muscles were determined by qPCR. Data are means±SEM from 4 muscles per condition. *P<0.05. (B) Muscle fiber size measurements. Left, mean fiber diameters ±SEM from 5 TAs per condition. Statistical differences were determined using a linear mixed model with a random effect for mouse (57). Different letters are statistically different (P<0.05). Right, fiber size distributions. (C) Representative images from (B). (D) Cdkn1a is required for denervation-induced muscle fiber atrophy. On day 0, mouse TA muscles were transfected bilaterally with either 20 μg p-miR-Control or 20 μg p-miR-Cdkn1a. On day 3, the left sciatic nerve was transected. On day 10, bilateral TA muscles were harvested. Left, mean fiber diameters ±SEM from ≧5 TAs per condition. Statistical differences were determined using a linear mixed model with a random effect for mouse; different letters are statistically different (P<0.05). Right, fiber size distributions. (E) Cdkn1a is required for fasting-induced muscle fiber atrophy. On day 0, mouse TA muscles were transfected with either 20 μg p-miR-Control (left leg) or 20 μg p-miR-Cdkn1a (right leg). On day 9, mice were fasted for 24 h and then harvested for analysis. Left, mean fiber diameters ±SEM from ≧4 TAs per condition. *P<0.01. Right, fiber size distributions. (F) Cdkn1a is required for Gadd45a-mediated muscle fiber atrophy. Mouse TA muscles were transfected with 10 μg p-Gadd45a-FLAG plus either 20 μg p-miR-control (left TA) or 20 μg p-miR-Cdkn1a (right TA), then harvested 10 days later. Left, mean fiber diameters ±SEM from 6 TAs per condition. *P<0.01. Right, fiber size distributions.

FIG. 15 shows additional data that Cdkn1a is required for skeletal muscle fiber atrophy during immobilization and fasting. (A-B) Cdkn1a is required for immobilization-induced muscle atrophy. On day 0, bilateral C57BL/6 tibialis anterior (TA) muscles were transfected with either 20 μg p-miR-Control or 20 μg p-miR-Cdkn1a #2. On day 3, right hindlimbs were immobilized, and on day 10, bilateral TA muscles were harvested for analysis. (A) Cdkn1a mRNA levels were determined by qPCR and normalized to levels in mobile, p-mir-Control-transfected muscles, which were set at one and indicated by the dashed line. Data are means±SEM from 3 muscles per condition. (B) Mean muscle fiber diameters ±SEM from 5 TAs per condition. Statistical differences were determined using a linear mixed model with a random effect for mouse; different letters are statistically different. (C) Cdkn1a is required for fasting-induced muscle atrophy. C57BL/6 TA muscles were transfected with either 20 μg p-miR-Control (left leg) or 20 μg p-miR-Cdkn1a #2 (right leg). Nine days after transfection, mice were fasted for 24 h and then TA muscle fiber size was analyzed. Data are mean muscle fiber diameters ±SEM from 5 TAs per condition. *P<0.01 by t-test.

FIG. 16 shows that increased Cdkn1a expression induces skeletal muscle fiber atrophy in vivo and skeletal myotube atrophy in vitro. (A-C) Cdkn1a induces atrophy of mouse muscle fibers. TA muscles were transfected with 2 μg p-eGFP plus either 15 μg pcDNA3 (left TA) or 15 μg p-Cdkn1a-FLAG (right TA), then harvested 10 days later. (A) Protein extracts were subjected to immunoblot analysis with anti-FLAG monoclonal IgG. (B) Representative fluorescence microscopy images of muscle cross sections. (C) Left, mean fiber diameters ±SEM from 5 TAs per condition. *P<0.01. Right, fiber size distributions. (D) Cdkn1a reduces specific tetanic force generated by muscles ex vivo. Mouse TA and extensor digitorum longus (EDL) muscles were transfected with 2 μg p-eGFP plus either 15 μg pcDNA3 or 15 μg p-Cdkn1a-FLAG. Nine days later, EDLs were harvested for measurement of specific tetanic force. Data are means±SEM from ≧6 mice per condition. *P<0.05. (E-G) Cdkn1a induces atrophy of cultured skeletal myotubes. (E) C2C12 myotubes were infected for 48 h with Ad-tTA with and without Ad-Cdkn1a. Protein extracts were subjected to immunoblot analysis with anti-FLAG monoclonal IgG. (F-G) C2C12 myotubes were infected for 48 h with Ad-ATF4ΔbZIP or Ad-Cdkn1a plus Ad-tTA. (F) Representative fluorescence microscopy images of myotubes. (G) Mean myotube diameters ±SEM from 3 separate experiments. *P<0.05.

FIG. 17 shows that Cdkn1a does not cause myotube death. Cdkn1a does not cause cell death. C2C12 myotubes were infected for 48 h with Ad-Cdkn1a plus Ad-tTA, and then stained with 0.2% trypan blue. As a positive control for cell death, myotubes were treated with 80% ethanol for 20 min before trypan blue staining

FIG. 18 shows that Cdkn1a decreases PGC-1α, mitochondria, Akt activity and protein synthesis and increases proteolysis. (A) Mouse TA muscles were transfected with 15 μg empty vector (pcDNA3; left TA) or 15 μp-Cdkn1a-FLAG (right TA), then harvested 10 days later for qPCR analysis. In each mouse, mRNA levels in the presence of Cdkn1a overexpression were normalized to levels in the absence of Cdkn1a overexpression. Each data point represents mean log₂ signal change ±SEM from 4 mice. *P<0.05. (B-C) Cdkn1a reduces skeletal muscle PGC-1α and Cox4 protein levels. Mouse TA muscles were transfected and harvested as in (A) for SDS-PAGE and immunoblot analysis with the indicated antibodies. Top, representative immunoblots. Bottom, quantification. In each muscle, PGC-1α or Cox4 signals were normalized to the actin signal; in each mouse, levels in the presence of Cdkn1a were normalized to levels in the absence of Cdkn1a. Data are means±SEM from 4 mice. *P<0.05. (D) Cdkn1a reduces mitochondrial DNA. Mouse TA muscles were transfected as in (A) and harvested 7 days later for qPCR analysis of mitochondrial DNA (mtDNA), which was normalized to the amount of nuclear DNA (nDNA) in the same muscle. Data are means±SEM from 4 mice. *P<0.01. (E) Cdkn1a reduces Akt phosphorylation. C2C12 myotubes were infected with Ad-ATF4ΔbZIP or Ad-Cdkn1a plus Ad-tTA, and then harvested 48 h later for SDS-PAGE and immunoblot analysis with the indicated antibodies. Top, representative immunoblots. Bottom, quantification. In each sample, the phospho-Akt signal was normalized to the total Akt signal. Levels in the presence of Cdkn1a were then normalized to levels in the absence of Cdkn1a. Data are means±SEM from 3 experiments. *P<0.01. (F) Cdkn1a reduces protein synthesis. C2C12 myotubes were infected with Ad-ATF4ΔbZIP or Ad-Cdkn1a plus Ad-tTA for 48 h and then protein synthesis was assessed by measuring [³H]-leucine incorporation into total cellular protein. Levels in the presence of Cdkn1a were then normalized to levels in the absence of Cdkn1a. Data are means±SEM. n=6 samples per condition. *P<0.01. (G) Cdkn1a increases total and lipidated LC3 protein in vivo. Mouse TA muscles were transfected and harvested as in (A) for SDS-PAGE and immunoblot analysis with the indicated antibodies. Top, representative immunoblots. Bottom, quantification. In each muscle, the LC3-II signal was normalized to the actin signal, and in each mouse, levels in the presence of Cdkn1a were normalized to levels in the absence of Cdkn1a. Data are means±SEM from 4 mice. *P<0.02. (H) Cdkn1a increases proteolysis. C2C12 myotubes were incubated with [³H]-tyrosine for 20 h, washed with chase medium for 2 h, and then infected with Ad-ATF4ΔbZIP or Ad-Cdkn1a plus Ad-tTA in fresh chase medium. Protein degradation was assessed 40 h later by measuring [³H]-tyrosine release. Levels in the presence of Cdkn1a were then normalized to levels in the absence of Cdkn1a. Data are means±SEM. n=8 samples per condition. *P<0.05.

FIG. 19 shows that ursolic acid significantly reduces the induction of Gadd45a and Cdkn1a mRNAs during skeletal muscle immobilization and that ursolic acid reduces immobilization-induced skeletal muscle atrophy and enhances recovery from immobilization-induced skeletal muscle atrophy. (A-E) Beginning on day 0, 6-8 wk old male C57BL/6 mice were given i.p. injections of ursolic acid (200 mg/kg) or an equal volume of vehicle (corn oil) twice a day. On day 2, the left tibialis anterior (TA) muscle of each mouse was immobilized. During immobilization, vehicle or ursolic acid continued to be administered via i.p. injection twice daily, and the right TA remained mobile and served as an intrasubject control. (A) On day 5, mice were euthanized and bilateral TA muscles were harvested. mRNA levels were determined with qPCR. In each mouse, mRNA levels from the immobile TA were normalized to values from the mobile TA, which were set at one. Data are means±SEM from ≧6 mice per condition. * P<0.05. (B-E) On day 8, bilateral TA muscles were harvested and weighed. (B) Effect of ursolic acid on skeletal muscle weight. In each mouse, the left (immobile) TA weight was normalized to the right (mobile) TA weight. Data are means±SEM from 10 mice per condition; ***P<0.001 by unpaired t-test. (C-E) Effect of ursolic acid on skeletal muscle fiber diameter. (C) Data are mean fiber diameters ±SEM from 10 immobilized TA muscles per condition; ***P<0.0001 by unpaired t-test. (D) Representative cross-sections of muscle fibers immunostained with anti-laminin antibody. (E) Data are fiber size distributions of >3000 fibers from 10 immobilized TA muscles per condition. (F) The left TA muscles of mice were immobilized for 7 days to induce atrophy, then remobilized by removing the staple from the left TA muscle. Treatment with vehicle or ursolic acid (200 mg/kg) was then initiated. Both vehicle and ursolic acid were given via i.p. injection twice daily. Data are means±SEM from 8 mice per condition; **P<0.01 by unpaired t-test.

FIG. 20 shows that ursolic acid increases mRNAs involved in anabolic signaling (androgen receptor (AR)), inhibition of muscle atrophy (IGF-I, AR and PGC-1α), angiogenesis, vascular flow and oxygen delivery (VEGFA and NOS1), glucose utilization (HK2) and mitochondrial biogenesis and oxididative phosphorylation (PGC-1α and TEAM), and that ursolic acid activates the growth hormone receptor (GHR). (A) C57BL/6 mice were fed diets lacking or containing 0.14% ursolic acid for 6 weeks before quadriceps muscles were harvested for qPCR analysis of the indicated mRNAs. mRNA levels in ursolic acid-treated mice were normalized to mRNA levels in control mice, which were set at one. Data are means±SEM from 10 mice per condition; *P<0.05, **P<0.01. (B) Cultured C2C12 myoblasts were serum-starved for 6 hours, and then incubated for 2 minutes in the absence or presence of ursolic acid (10 μM) and/or recombinant human growth hormone (100 ng/ml), as indicated. Total cellular protein extracts were subjected to immunoprecipitation with anti-GHR antibody, followed by immunoblot analysis with anti-phospho-tyrosine or anti-GHR antibodies to assess phospho-GHR and total GHR, respectively.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. DEFINITIONS

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, “skeletal muscle atrophy” or “muscle atrophy” refers to a wasting or loss of muscle tissue. The art is familiar with the many common causes of atrophy including, but not limited to, aging, cerebrovascular accident (stroke), spinal cord injury, peripheral nerve injury (peripheral neuropathy), other injury, prolonged immobilization, osteoarthritis, rheumatoid arthritis, prolonged corticosteroid therapy, diabetes (diabetic neuropathy), burns, poliomyelitis, amyotrophic lateral sclerosis (ALS or Lou Gehrig's disease), Guillain-Barre syndrome, muscular dystrophy, myotonia, congenital myotonic dystrophy, and myopathy.

As used herein, a “Gadd45a and/or Cdkn1a inhibitor” refers to any substance, compound, composition, or agent that inhibits or reduces the expression and/or activity of Gadd45a and/or Cdkn1a. Examples of Gadd45a and/or Cdkn1a inhibitors include, but are not limited to, ursolic acid, ursolic acid derivatives, RNA interference, and antisense olignonucleotides.

As used herein, “an androgen and/or growth hormone elevator” refers to any substance, compound, composition, or agent that elevates or increases the expression and/or activity and/or concentration of androgen and/or growth hormone. Examples of an androgen and/or growth hormone elevator include, but are not limited to, androgens such as testosterone, growth hormone, ghrelin, ghrelin analogs, substances that increase the expression or activity of ghrelin, and aromatase inhibitors.

As used herein, “an androgen and/or growth hormone receptor activator” refers to any substance, compound, composition, or agent that elevates or increases the expression and/or activity and/or concentration of androgen and/or growth hormone receptors. Examples of an androgen and/or growth hormone receptor activator include, but are not limited to, androgens such as testosterone, growth hormone, selective androgen receptor modulators, and protein tyrosine phosphatase inhibitors.

As used herein, the term “analog” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds.

As used herein, “homolog” or “homologue” refers to a polypeptide or nucleic acid with homology to a specific known sequence. Specifically disclosed are variants of the nucleic acids and polypeptides herein disclosed which have at least 40, 41, 42, 43, 44, 45, 46, 47, 48, 49, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated or known sequence. Those of skill in the art readily understand how to determine the homology of two proteins or nucleic acids. For example, the homology can be calculated after aligning the two sequences so that the homology is at its highest level. It is understood that one way to define any variants, modifications, or derivatives of the disclosed genes and proteins herein is through defining the variants, modification, and derivatives in terms of homology to specific known sequences.

As used herein, “ursolic acid” refers to ursolic acid, or extracts containing ursolic acid from plants such as apples, holy basil, bilberries, cranberries, elder flower, peppermint, lavender, oregano, thyme, sage, hawthorn, bearberry or prunes.

As used herein, “ursolic acid derivatives” refers to corosolic acid, betulinic acid, hederagenin, boswellic acids, UA0713, a substituted ursolic acid analog, an ursane compound or any other pentacyclic triterpene acids that prevents muscle atrophy, reduces muscle atrophy, increases muscle mass, increases muscle strength in an animal, including in humans, increases Akt phosphorylation, increases S6K phosphorylation, or stimulates biochemical events known to precede or follow Akt phosphorylation or S6K phosphorylation. For example, and not to be limiting, biochemical events known to precede or follow Akt phosphorylation or S6K phosphorylation can be events such as insulin receptor phosphorylation, IGF-I receptor phosphorylation, insulin receptor substrate (IRS) protein phosphorylation, phosphoinositide-3 kinase phosphorylation, phosphoinositide-3 kinase activation, phosphoinositide dependent kinase 1 activation, mammalian target of rapamycin complex 2 activation, adrenergic receptor activation, heterotrimeric G protein activation, adenylate cyclase activation, increased intracellular cyclic AMP, AMP kinase activation, protein kinase A activation, protein kinase C activation, CREB activation, mitogen activated protein kinase pathway activation, mammalian target of rapamycin complex 1 activation, 4E-BP1 phosphorylation, 4E-BP1 inactivation, GSK3β phosphorylation, GSK3 β inactivation, increased protein synthesis, increased glucose uptake, Foxo transcription factor phosphorylation, Foxo transcription factor inactivation, Cdkn1a phosphorylation, Cdkn1a inactivation, reduced atrogin-1 mRNA, reduced MuRF1 mRNA, increased VEGFA mRNA, or increased IGF1 mRNA.

As used herein, “DNA demethylation” refers to the removal of a methyl group from a nucleotide in a DNA sequence. As known to the art, cytosine 5′ methylation of CpG dinucleotides within and around genes exerts a major influence on transcription in many plants and animals. DNA methylation is an epigenetic modification that is essential for gene silencing and genome stability in many organisms. DNA methylation targets the machinery necessary to assemble specialized chromatin enriched in deacetylated histones.

As used herein, “cyclin dependent kinases” or Cdks refer to family of serine/threonine protein kinases whose members are small proteins (˜34-40 kDa) composed of little more than the catalytic core shared by all protein kinases. All Cdks share the feature that their enzymatic activation requires the binding of a regulatory cyclin subunit. In most cases, full activation also requires phosphorylation of a threonine residue near the kinase active site. The art is familiar with Cdks. For example, animal cells contain at least nine Cdks, only four of which (Cdk1, 2, 4 and 6) are involved directly in cell-cycle control. Cdk7 contributes indirectly by acting as a Cdk-activating kinase (CAIS) that phosphorylates other Cdks, and Cdks are also components of the machinery that controls basal gene transcription by RNA polymerase II (Cdk7, 8 and 9) and are involved in controlling the differentiation of nerve cells (Cdk5).

As used herein, the term “subject” refers to the target of administration, e.g., an animal. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more muscle disorders prior to the administering step. In some aspects of the disclosed method, the subject has been diagnosed with a need for increasing muscle mass prior to the administering step. In some aspects of the disclosed method, the subject has been diagnosed with a need for increasing muscle mass prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a muscle atrophy disorder” means having been subjected to a physical examination by a person of skill, for example, a physician, and found to have a condition that can be diagnosed or treated by a compound or composition that can increase muscle mass. As a further example, “diagnosed with a need for increasing muscle mass” refers to having been subjected to a physical examination by a person of skill, for example, a physician, and found to have a condition characterized by muscle atrophy or other disease wherein increasing muscle mass would be beneficial to the subject. Such a diagnosis can be in reference to a disorder, such as muscle atrophy, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to muscle atrophy) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “EC₅₀,” is intended to refer to the concentration or dose of a substance (e.g., a compound or a drug) that is required for 50% enhancement or activation of a biological process, or component of a process, including a protein, subunit, organelle, ribonucleoprotein, etc. EC₅₀ also refers to the concentration or dose of a substance that is required for 50% enhancement or activation in vivo, as further defined elsewhere herein. Alternatively, EC₅₀ can refer to the concentration or dose of compound that provokes a response halfway between the baseline and maximum response. The response can be measured in an in vitro or in vivo system as is convenient and appropriate for the biological response of interest. For example, the response can be measured in vitro using cultured muscle cells or in an ex vivo organ culture system with isolated muscle fibers. Alternatively, the response can be measured in vivo using an appropriate research model such as rodent, including mice and rats. The mouse or rat can be an inbred strain with phenotypic characteristics of interest such as obesity or diabetes. As appropriate, the response can be measured in a transgenic or knockout mouse or rat wherein the gene or genes has been introduced or knocked-out, as appropriate, to replicate a disease process.

As used herein, “IC₅₀,” is intended to refer to the concentration or dose of a substance (e.g., a compound or a drug) that is required for 50% inhibition or diminution of a biological process, or component of a process, including a protein, subunit, organelle, ribonucleoprotein, etc. IC₅₀ also refers to the concentration or dose of a substance that is required for 50% inhibition or diminution in vivo, as further defined elsewhere herein. Alternatively, IC₅₀ also refers to the half maximal (50%) inhibitory concentration (IC) or inhibitory dose of a substance. The response can be measured in a in vitro or in vivo system as is convenient and appropriate for the biological response of interest. For example, the response can be measured in vitro using cultured muscle cells or in an ex vivo organ culture system with isolated muscle fibers. Alternatively, the response can be measured in vivo using an appropriate research model such as rodent, including mice and rats. The mouse or rat can be an inbred strain with phenotypic characteristics of interest such as obesity or diabetes. As appropriate, the response can be measured in a transgenic or knockout mouse or rat wherein the a gene or genes has been introduced or knocked-out, as appropriate, to replicate a disease process.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polter refers to one or more —OCH₂CH₂O— units in the polter, regardless of whether ethylene glycol was used to prepare the polter. Similarly, a sebacic acid residue in a polter refers to one or more —CO(CH₂)₈CO— moieties in the polter, regardless of whether the residue is obtained by reacting sebacic acid or an ester thereof to obtain the polter.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dode cyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polter” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an interger from 1 to 500. “Polter” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes azetidine, dioxane, furan, imidazole, isothiazole, isoxazole, morpholine, oxazole, oxazole, including, 1,2,3-oxadiazole, 1,2,5-oxadiazole and 1,3,4-oxadiazole, piperazine, piperidine, pyrazine, pyrazole, pyridazine, pyridine, pyrimidine, pyrrole, pyrrolidine, tetrahydrofuran, tetrahydropyran, tetrazine, including 1,2,4,5-tetrazine, tetrazole, including 1,2,3,4-tetrazole and 1,2,4,5-tetrazole, thiadiazole, including, 1,2,3-thiadiazole, 1,2,5-thiadiazole, and 1,3,4-thiadiazole, thiazole, thiophene, triazine, including 1,3,5-triazine and 1,2,4-triazine, triazole, including, 1,2,3-triazole, 1,3,4-triazole, and the like.

The term “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

As used herein, the term “stable” refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o}C(O)NR^{o}₂; —N(R^{o}C(S)NR^{o}₂; —(CH₂)₀₋₄N(R^{o})C(O)OR^{o}; —N(R^{o})N(R^{o}C(O)R^{o}; —N(R^{o})N(R^{o}C(O)NR^{o}₂; —N(R^{o}N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; (CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂NR^{o}₂; —N(R^{o}S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched)alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR^{*}₂, ═NNHC(O)R^{*}, ═NNHC(O)OR^{*}, ═NNHS(O)₂R^{*}, ═NR^{*}, ═NOR^{*}, —O(C(R^{*}₂))₂₋₃O—, or —S(C(R^{*}₂))₂₋₃S—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{*}₂)₂₋₃O—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{*} include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, brosylate, and halides.

The terms “hydrolysable group” and “hydrolysable moiety” refer to a functional group capable of undergoing hydrolysis, e.g., under basic or acidic conditions. Examples of hydrolysable residues include, without limitatation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

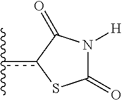

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

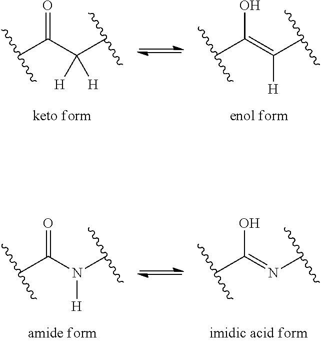 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

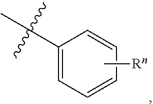

which is understood to be equivalent to a formula:

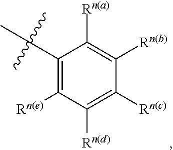

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOSITIONS

In one aspect, the invention relates to a composition for treating or preventing skeletal muscle atrophy, the composition comprising a Gadd45a and/or Cdkn1a inhibitor and an androgen and/or growth hormone elevator. In an aspect, the invention relates to a composition for treating or preventing skeletal muscle atrophy, the composition comprising a Gadd45a and/or Cdkn1a inhibitor and androgen and/or growth hormone receptor activator.

In a further aspect, the invention relates to compositions useful in methods to modulate muscle growth, methods to inhibit muscle atrophy and to increase muscle mass, methods to induce skeletal muscle hypertrophy, methods to enhance tissue growth, and pharmaceutical compositions comprising compositions used in the methods.

In one aspect, the compositions of the invention are useful in the treatment of muscle disorders. In a further aspect, the muscle disorder can be skeletal muscle atrophy secondary to malnutrition, muscle disuse (secondary to voluntary or involuntary bed rest), neurologic disease (including multiple sclerosis, amyotrophic lateral sclerosis, spinal muscular atrophy, critical illness neuropathy, spinal cord injury or peripheral nerve injury), orthopedic injury, casting, and other post-surgical forms of limb immobilization, chronic disease (including cancer, congestive heart failure, chronic pulmonary disease, chronic renal failure, chronic liver disease, diabetes mellitus, Cushing syndrome and chronic infections such as HIV/AIDS or tuberculosis), burns, sepsis, other illnesses requiring mechanical ventilation, drug-induced muscle disease (such as glucorticoid-induced myopathy and statin-induced myopathy), genetic diseases that primarily affect skeletal muscle (such as muscular dystrophy and myotonic dystrophy), autoimmune diseases that affect skeletal muscle (such as polymyositis and dermatomyositis), spaceflight, atherosclerotic vascular diseases, hypogonadism, hypopituitarism, or age-related sarcopenia.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compositions can be employed in the disclosed methods of using.

i) Gadd45a and/or Cdkn1a Inhibitor

Disclosed herein are compositions for treating or preventing skeletal muscle atrophy, the composition comprising a Gadd45a and/or Cdkn1a inhibitor. In an aspect, the composition comprises a therapeutically effective amount of a Gadd45a and/or Cdkn1a inhibitor. In an aspect, the composition comprises a prophylactically effective amount of a Gadd45a and/or Cdkn1a inhibitor. In an aspect, the amount of inhibitor in the composition is greater than 100 mg/kg. In a further aspect, the amount of inhibitor in the composition is greater than 50 mg/kg. In an aspect, the amount of inhibitor in the composition is greater than 25 mg/kg. In an even further aspect, the amount of inhibitor in the composition is greater than 10 mg/kg. In an even further aspect, the amount of inhibitor in the composition is greater than 5 mg/kg. In an even further aspect, the amount of inhibitor in the composition is greater than 1 mg/kg. In an even further aspect, the amount of inhibitor in the composition is greater than 0.5 mg/kg. In an even further aspect, the amount of inhibitor in the composition is greater than 0.1 mg/kg. In an aspect, the Gadd45a and/or Cdkn1a inhibitor acts via inhibition of Gadd45a-dependent DNA demethylation enzymes. In a further aspect, the Gadd45a and/or Cdkn1a inhibitor acts via inhibition of ATF4.

a. Ursolic Acid or Ursolic Acid Derivatives

In one aspect, the invention relates to compositions useful in methods to inhibit muscle atrophy and to increase muscle mass by providing to a subject in need thereof an effective amount of ursolic acid or a derivative thereof, and pharmaceutical compositions comprising compositions used in the methods. In an aspect, the Gadd45a and/or Cdkn1a inhibitor is uroslic acid or an ursolic acid derivative such as boswellic acid, corosolic acid, betulinic acid, or UA0713. Ursolic acid is a highly water-insoluble pentacyclic triterpene acid that possesses a wide range of biological effects, including anti-cancer, anti-oxidant, anti-inflammatory, anti-allergic, hepatoprotective, gastroprotective, hypolipidemic, hypoglycemic, lipolytic anti-obesity, anti-atherogenic and immunomodulatory effects (Liu J (1995) Journal of ethnopharmacology 49(2):57-68; Liu J (2005) Journal of ethnopharmacology 100(1-2): 92-94; Wang Z H, et al. (2010) European journal of pharmacology 628(1-3): 255-260; Jang S M, et al. (2009) Int Immunopharmacol 9(1):113-119). At the molecular level, ursolic acid inhibits the STAT3 activation pathway, reduces matrix metalloproteinase-9 expression via the glucocorticoid receptor, inhibits protein tyrosine phosphatases, acts as an insulin mimetic, activates PPARα, inhibits NF-kB transcription factors, translocates hormone-sensitive lipase to stimulate lipolysis and inhibits the hepatic polyol pathway, among many other described effects.

As medicine, ursolic acid is well tolerated and can be used topically and orally. Ursolic acid is present in many plants, including apples, basil, bilberries, cranberries, elder flower, peppermint, rosemary, lavender, oregano, thyme, hawthorn, prunes. Apple peels contain high quantity of ursolic acid and related compounds which are responsible for the anti-cancer activity of apple. Ursolic acid can also serve as a starting material for synthesis of more potent bioactive derivatives, such as anti-tumor agents.

Other names for ursolic acid include 3-β-hydroxy-urs-12-en-28-oic acid, urson, prunol, micromerol, urson, and malol. The structure is shown below:

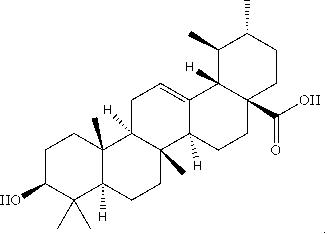 [SMILES: C[C@H]1[C@H](C)CC[C@]2(C(=O)O)CC[C@@H]3C(=CC[C@H]4[C@@]3(C)CC[C@H]3C(C)(C)[C@@H](O)CC[C@]43C)[C@H]12]

Other closely related pentacyclic triterpene acids with insulin sensitizing actions include oleanolic acid (Wang et at, 2010), corosolic acid (Sivakumar et at, 2009) and UA0713 (Zhang et at, 2006).

In one aspect, the invention relates to compounds of the formula:

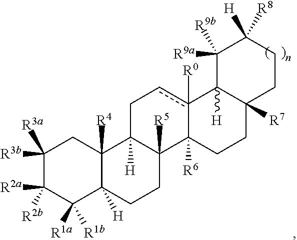

wherein each [symbol] is an optional covalent bond, and R⁰ is optionally present; wherein n is 0 or 1; wherein R⁰, when present, is hydrogen; wherein R^{1a} is selected from C1-C6 alkyl and —C(O)ZR¹⁰; wherein R^{1b} is selected from C1-C6 alkyl, or wherein R^{1a} and R^{1b} are optionally covalently bonded and, together with the intermediate carbon, comprise an optionally substituted C3-C5 cycloalkyl or C2-C5 heterocycloalkyl; wherein one of R^{2a} and R^{2b} is —OR¹¹, and the other is hydrogen, or R^{2a} and R^{2b} together comprise ═O; wherein each of R^{3a} and R^{3b} is independently selected from hydrogen, hydroxyl, C1-C6 alkyl, and C1-C6 alkoxyl, provided that R^{3a} and R^{3b} are not simultaneously hydroxyl, wherein R^{3a} and R^{3b} are optionally covalently bonded and, together with the intermediate carbon, comprise an optionally substituted C3-C5 cycloalkyl or C2-C5 heterocycloalkyl; wherein each of R⁴, R⁵, and R⁶ is independently selected from C1-C6 alkyl; wherein R⁷ is selected from C1-C6 alkyl, —CH₂OR¹² and —C(O)ZR¹²; wherein R⁸ is selected from hydrogen and C1-C6 alkyl; wherein each of R^{9a} and R^{9b} is independently selected from hydrogen and C1-C6 alkyl, provided that R^{9a} and R^{9b} are not simultaneously hydrogen; or wherein R^{9a} and R^{9b} are covalently bonded and, along with the intermediate carbon, together comprise C3-C5 cycloalkyl or C2-C5 heterocycloalkyl; wherein R¹⁰ is selected from hydrogen and C1-C6 alkyl; wherein R¹¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, wherein Z is N, R¹² and R¹³ are covalently bonded and —NR¹²R¹³ comprises a moiety of the formula:

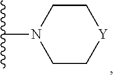

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; or a pharmaceutically acceptable salt, hydrate, solvate, or polymorph thereof.

In a further aspect, the invention relates to compounds of a formula:

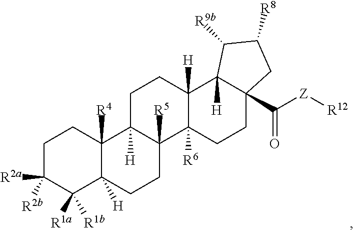

wherein each of R^{1a} and R^{1b} is C1-C6 alkyl; wherein one of R^{2a} and R^{2b} is —OR¹¹, and the other is hydrogen; wherein each of R⁴, R⁵, and R⁶ is independently C1-C6 alkyl; wherein R⁸ is selected from hydrogen and C1-C6 alkyl; wherein R^{9b} is C1-C6 alkyl; wherein R¹¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, wherein Z is N, R¹² and R¹³ are covalently bonded and —NR¹²R¹³ comprises a moiety of the formula:

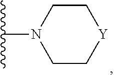

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl.

In a further aspect, the invention relates to compounds of a formula:

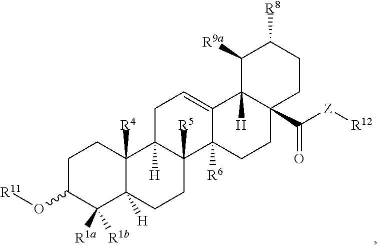

wherein R^{1a} is selected from C1-C6 alkyl and —C(O)ZR¹⁰; wherein R^{1b} is selected from C1-C6 alkyl, or R^{1a} and R^{1b} are covalently bonded and, together with the intermediate carbon, comprise an optionally substituted 3- to 7-membered spirocycloalkyl; wherein R⁸ is C1-C6 alkyl; wherein R^{9a} is C1-C6 alkyl; wherein R¹¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, wherein Z is N, R¹² and R¹³ are covalently bonded and —NR¹²R¹³ comprises a moiety of the formula:

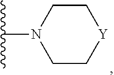

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl.

In a further aspect, the invention relates to compounds of a formula:

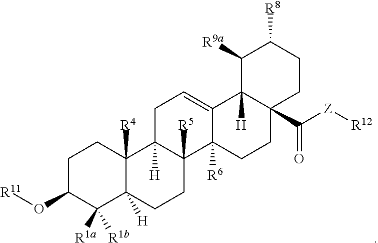

In a further aspect, the invention relates to compounds of a formula:

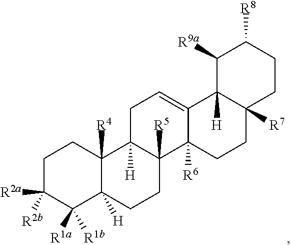

wherein R^{1a} is —C(O)ZR¹⁰; wherein R^{1b} is C1-C6 alkyl; wherein one of R^{2a} and R^{2b} is —OR¹¹, and the other is hydrogen; wherein each of R⁴, R⁵, and R⁶ is independently selected from C1-C6 alkyl; wherein R⁷ is selected from C1-C6 alkyl; wherein R⁸ is selected from hydrogen and C1-C6 alkyl; wherein R^{9a} is selected from hydrogen and C1-C6 alkyl; wherein Z is selected from —O— and —NR¹³—; wherein R¹⁰ is selected from hydrogen and C1-C6 alkyl; wherein R¹¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹³ is selected from hydrogen and C1-C4 alkyl; or, wherein Z is N, R¹² and R¹³ are covalently bonded and —NR¹²R¹³ comprises a moiety of the formula:

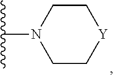

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl.

In a further aspect, the invention relates to compounds of a formula:

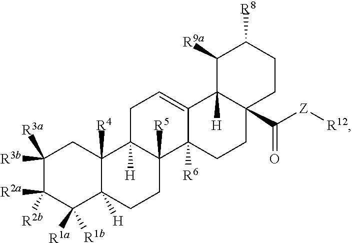

wherein each of R^{1a} and R^{1b} is independently C1-C6 alkyl; wherein one of R^{2a} and R^{2b} is OR¹¹, and the other is hydrogen; wherein one of R^{1a} and R^{3b} is —OR¹¹, and the other is hydrogen; wherein each of R⁴, R⁵, and R⁶ is independently selected from C1-C6 alkyl; wherein R⁸ is C1-C6 alkyl; wherein R^{9a} is C1-C6 alkyl; wherein each R¹¹ is independently selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, wherein Z is N, R¹² and R¹³ are covalently bonded and —NR¹²R¹³ comprises a moiety of the formula:

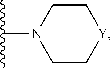

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl.

In a further aspect, the invention relates to compounds of a formula:

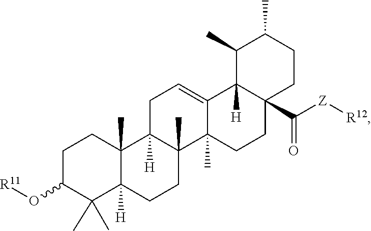

wherein R¹¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, wherein Z is N, R¹² and R¹³ are covalently bonded and —NR¹²R¹³ comprises a moiety of the formula:

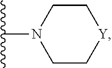

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; or a pharmaceutically acceptable salt, hydrate, solvate, or polymorph thereof.

In a further aspect, the compound is administered in an amount effective to prevent or treat muscle atrophy in the animal. In a still further aspect, the compound is administered in amount is greater than about 50 mg per day when the compound is ursolic acid, beta-boswellic acid, corosolic acid, betulinic acid, or UA0713. In a yet further aspect, the compound is administered in an amount greater than about 50 mg per day and effective to enhance muscle formation in the mammal. In a still further aspect, the compound is administered in amount is greater than about 100 mg per day when the compound is ursolic acid, beta-boswellic acid, corosolic acid, betulinic acid, or UA0713. In a yet further aspect, the compound is administered in an amount greater than about 100 mg per day and effective to enhance muscle formation in the mammal. In a still further aspect, the compound is administered in amount is greater than about 500 mg per day when the compound is ursolic acid, beta-boswellic acid, corosolic acid, betulinic acid, or UA0713. In a yet further aspect, the compound is administered in an amount greater than about 500 mg per day and effective to enhance muscle formation in the mammal. In a still further aspect, the compound is administered in amount is greater than about 1000 mg per day when the compound is ursolic acid, beta-boswellic acid, corosolic acid, betulinic acid, or UA0713. In a yet further aspect, the compound is administered in an amount greater than about 1000 mg per day and effective to enhance muscle formation in the mammal.

In a further aspect, the invention relates to compounds of a formula selected from:

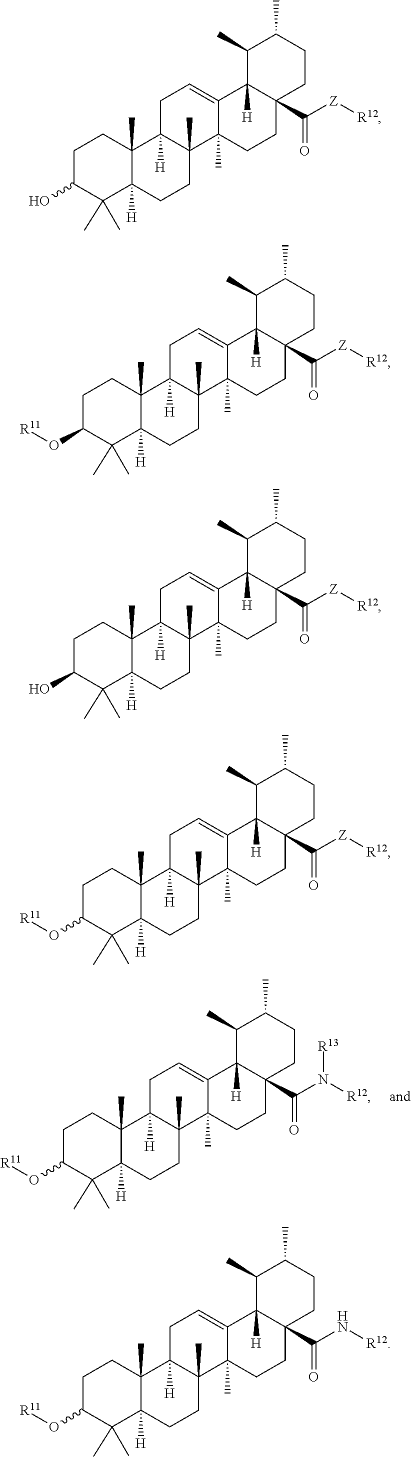

### (1) R⁰ Groups and Optional Bonds

In one aspect, an optional covalent bond can be represented by [symbol] . Thus, in certain aspects, a particular bond is present, thereby providing a single covalent bond. In further aspects, a particular bond is present, thereby providing a double covalent bond. In further aspects, a particular bond is absent, thereby providing a double covalent bond.

In one aspect, R^{o0} is optionally present. That is, in certain aspects, R⁰ is present. In further aspects, R⁰ is absent. In a further aspect, R⁰, when present, is hydrogen. It is understood that the presence and/or absence of R⁰ Groups and optional bonds serve to satisfy valence of the adjacent chemical moieties.

### (2) R¹ Groups

In one aspect, R^{1a} is selected from C1-C6 alkyl and —C(O)ZR¹⁰; wherein R^{1b} is selected from C1-C6 alkyl; or wherein R^{1a} and R^{1b} are covalently bonded and, along with the intermediate carbon, together comprise optionally substituted C3-C5 cycloalkyl or optionally substituted C2-C5 heterocycloalkyl. In a further aspect, R^{1a} is —CO₂H. In a further aspect, R^{1b} is methyl. In a further aspect, R^{1a} and R^{1b} are both methyl.

In one aspect, R^{1a} is —C(O)ZR¹⁰. In a further aspect, R^{1a} is selected from C1-C6 alkyl, for example, methyl, ethyl, propyl, butyl, pentyl, or hexyl. In a further aspect, R^{1b} is selected from C1-C6 alkyl, for example, methyl, ethyl, propyl, butyl, pentyl, or hexyl.

In a further aspect, R^{1a} and R^{1b} are covalently bonded and, along with the intermediate carbon, together comprise optionally substituted C3-C5 cycloalkyl or optionally substituted C2-C5 heterocycloalkyl.

### (3) R² Groups

In one aspect, R^{2a} and R^{2b} are independently selected from hydrogen and —OR¹¹, provided that at least one of R^{2a} and R^{2b} is —OR¹¹; or wherein R^{2a} and R^{2b} together comprise ═O. In a further aspect, R^{2a} is hydrogen, and R^{2b} is —OR¹¹. In a further aspect, R^{2a} is —OR¹¹, and R^{2b} is hydrogen. In a further aspect, R^{2a} and R^{2b} together comprise ═O.

In a further aspect, R^{2a} is hydrogen. In a further aspect, R^{2a} is —OR¹¹; wherein R¹¹ is selected from hydrogen, C1-C6 alkyl, and —C(O)R¹⁴; wherein R¹⁴ is C1-C6 alkyl. In a further aspect, R^{2b} is —OR¹¹; wherein R¹¹ is selected from hydrogen, C1-C6 alkyl, and —C(O)R¹⁴; and wherein R¹⁴ is C1-C6 alkyl. In a further aspect, R^{2b} is —OR¹¹; wherein R¹¹ is hydrogen.

In a further aspect, R^{2b} is hydrogen. In a further aspect, R^{2a} is —OR¹¹; wherein R¹¹ is selected from hydrogen, C1-C6 alkyl, and —C(O)R¹⁴; wherein R¹⁴ is C1-C6 alkyl. In a further aspect, R^{2a} is —OR¹¹; wherein R¹¹ is hydrogen.

### (4) R³ Groups

In one aspect, each of R^{3a} and R^{3b} is independently selected from hydrogen, hydroxyl, C1-C6 alkyl, and C1-C6 alkoxyl, provided that R^{3a} and R^{3b} are not simultaneously hydroxyl; or wherein R^{3a} and R^{3b} are covalently bonded and, along with the intermediate carbon, together comprise optionally substituted C3-C5 cycloalkyl or optionally substituted C2-C5 heterocycloalkyl.

In a further aspect, R^{3a} is hydrogen. In a further aspect, R^{3b} is —OR¹¹; wherein R¹¹ is selected from hydrogen, C1-C6 alkyl, and —C(O)R¹⁴; wherein R¹⁴ is C1-C6 alkyl.

### (5) R⁴ Groups

In one aspect, R⁴ is independently selected from C1-C6 alkyl, for example, methyl, ethyl, propyl, butyl, pentyl, or hexyl. In a further aspect, R⁴ is methyl. In a further aspect, R⁴, R⁵, and R⁶ are all methyl.

### (6) R⁵ Groups

In one aspect, R⁵ is independently selected from C1-C6 alkyl, for example, methyl, ethyl, propyl, butyl, pentyl, or hexyl. In a further aspect, R⁵ is methyl.

### (7) R⁶ Groups

In one aspect, R⁶ is independently selected from C1-C6 alkyl, for example, methyl, ethyl, propyl, butyl, pentyl, or hexyl. In a further aspect, R⁶ is methyl.

### (8) R⁷ Groups

In one aspect, R⁷ is selected from C1-C6 alkyl, —CH₂OR¹², and —C(O)ZR¹². In a further aspect, R⁷ is C1-C6 alkyl, for example, methyl, ethyl, propyl, butyl, pentyl, or hexyl. In a further aspect, R⁷ is —CH₂OR¹². In a further aspect, R⁷ is and —C(O)ZR¹².

### (9) R⁸ Groups

In one aspect, R⁸ is selected from hydrogen and C1-C6 alkyl. In a further aspect, R⁸ is hydrogen. In a further aspect, R⁸ is C1-C6 alkyl, for example, methyl, ethyl, propyl, butyl, pentyl, or hexyl.

### (10) R⁹ Groups

In one aspect, each of R^{9a} and R^{9b} is independently selected from hydrogen and C1-C6 alkyl, provided that R^{9a} and R^{9b} are not simultaneously hydrogen; or wherein R^{9a} and R^{9b} are covalently bonded and, along with the intermediate carbon, together comprise optionally substituted C3-C5 cycloalkyl or optionally substituted C2-C5 heterocycloalkyl.

In a further aspect, R^{9a} is hydrogen. In a further aspect, R^{9a} is C1-C6 alkyl, for example, methyl, ethyl, propyl, butyl, pentyl, or hexyl. In a further aspect, R^{9b} is hydrogen. In a further aspect, R^{9b} is C1-C6 alkyl, for example, methyl, ethyl, propyl, butyl, pentyl, or hexyl. In a further aspect, R^{9b} is selected from methyl, ethyl, vinyl, n-propyl, propen-2-yl, i-propyl, 2-propenyl, n-butyl, 1-buten-2-yl, 1-buten-3-yl, i-butyl, 1-buten-2-yl, 1-buten-3-yl, s-butyl, 2-buten-1-yl, 2-buten-2-yl, 2-buten-3-yl, and t-butyl.

In a further aspect, R^{9a} and R^{9b} are covalently bonded and, along with the intermediate carbon, together comprise optionally substituted C3-C5 cycloalkyl or optionally substituted C2-C5 heterocycloalkyl.

### (11) R¹⁰ Groups

In one aspect, R¹⁰ is selected from hydrogen and C1-C6 alkyl. In a further aspect, R¹⁰ is hydrogen. In a further aspect, R¹⁰ is C1-C6 alkyl, for example, methyl, ethyl, propyl, butyl, pentyl, or hexyl.

### (12) R¹¹ Groups

In one aspect, each R¹¹ is independently selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl.

In a further aspect, R¹¹ is hydrogen. In a further aspect, R¹¹ is selected from C1-C6 alkyl, C1-C5 heteroalkyl, C3-C6 cycloalkyl, C4-C6 heterocycloalkyl, phenyl, heteroaryl, and —C(O)R¹⁴. In a further aspect, R¹¹ is C1-C6 alkyl. In a further aspect, R¹¹ is C1-C5 heteroalkyl. In a further aspect, R¹¹ is C3-C6 cycloalkyl. In a further aspect, R¹¹ is C4-C6 heterocycloalkyl. In a further aspect, R¹¹ is phenyl. In a further aspect, R¹¹ is heteroaryl. In a further aspect, R¹¹ is —C(O)R¹⁴.

In a further aspect, R¹¹ is unsubstituted. In a further aspect, R¹¹, where permitted, is substituted with 0-2 groups. In a further aspect, R¹¹, where permitted, is substituted with 1 group. In a further aspect, R¹¹, where permitted, is substituted with 2 groups.

### (13) R¹² Groups

In one aspect, R¹² is selected from hydrogen and optionally substituted organic residue having from 1 to 20 carbons. In a further aspect, R¹² is hydrogen. In a further aspect, R¹² is optionally substituted organic residue having from 1 to 20 carbons. In a further aspect, R¹² is optionally substituted organic residue having from 3 to 12 carbons.

In a further aspect, R¹² is hydrogen. In a further aspect, R¹² is alkyl. In a further aspect, R¹² is heteroalkyl. In a further aspect, R¹² is cycloalkyl. In a further aspect, R¹² is heterocycloalkyl. In a further aspect, R¹² is aryl. In a further aspect, R¹² is heteroaryl. In a further aspect, R¹² is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl. In a further aspect, R¹² comprises a group having a formula:

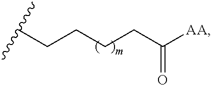

wherein m is an integer from 0 to 10 (e.g., 0, 1, 2, 3, 4, 5, 6, 7, 8, 9, or 10); and wherein AA represents an amino acid residue. In a further aspect, R¹² is AA is a phenylalanine residue. In a further aspect, R¹² comprises a group having a formula:

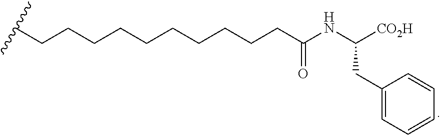 [SMILES: *CCCCCCCCCCC(=O)N[C@@H](Cc1ccccc1)C(=O)O]

### (14) R¹³ Groups

In one aspect, R¹³ is selected from hydrogen and C1-C4 alkyl; or, wherein Z is N, R¹² and R¹³ are covalently bonded and —NR¹²R¹³ comprises a moiety of the formula:

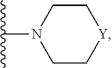

wherein Y is selected from —O—, —S—, —SO—, —SO₂—, —NH—, and —NCH₃—.

In a further aspect, R¹³ is hydrogen. In a further aspect, R¹³ is C1-C4 alkyl, for example, methyl, ethyl, propyl, or butyl. In a further aspect, Z is N, and —NR¹²R¹³ comprises a moiety of the formula:

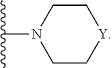

### (15) R¹⁴ Groups

In one aspect, R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl.

In a further aspect, R¹⁴ is C1-C6 alkyl, for example, methyl, ethyl, propyl, butyl, pentyl, or hexyl. In a further aspect, R¹⁴ is unsubstituted. In a further aspect, R¹⁴, where permitted, is substituted with 0-2 groups. In a further aspect, R¹⁴, where permitted, is substituted with 1 group. In a further aspect, R¹⁴, where permitted, is substituted with 2 groups.

### (16) AA Groups

In one aspect, AA represents an amino acid residue, for example, phenylalanine

### (17) Y Groups

In one aspect, Y is selected from —O—, —S—, —SO—, —SO₂—, —NH—, and —NCH₃—.

### (18) Z Groups

In one aspect, Z is selected from —O— and —NR¹³—. In a further aspect, Z is —O—. In a further aspect, Z is —NR¹³—; wherein R¹³ is hydrogen. In a further aspect, Z is —NR¹³—; wherein R¹³ is C1-C4 alkyl.

### (19) Example Compounds

In one aspect, a compound can be present as one or more of the following structures:

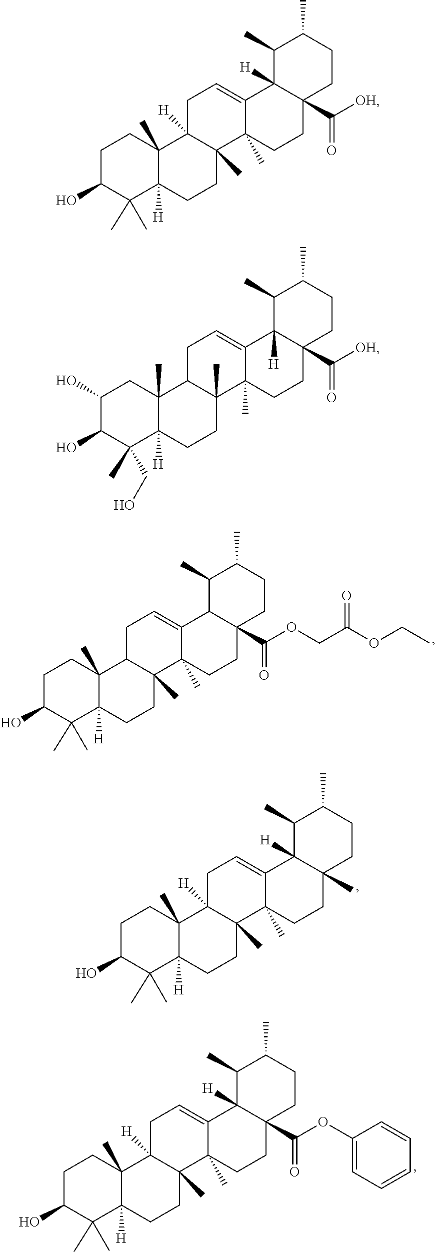

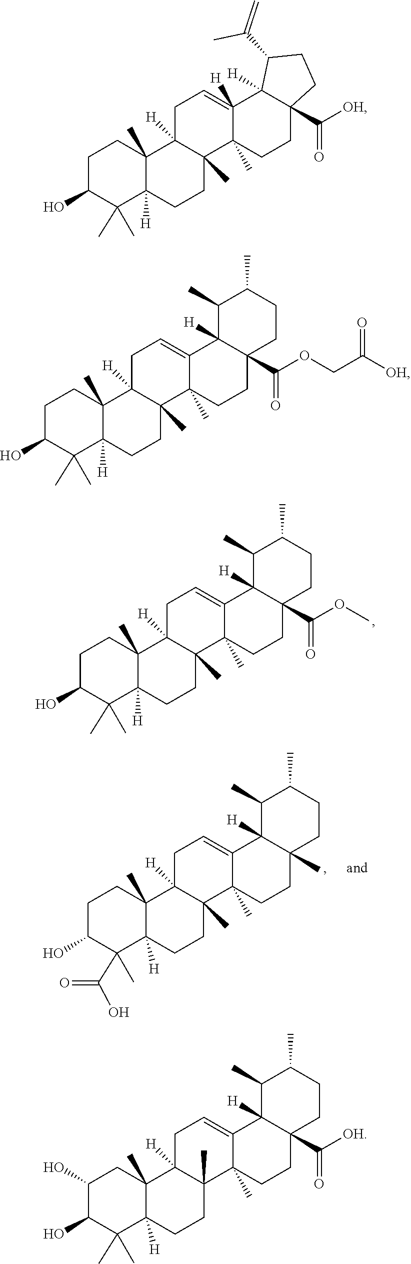

In a further aspect, a compound can be present as one or more of the following structures:

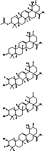

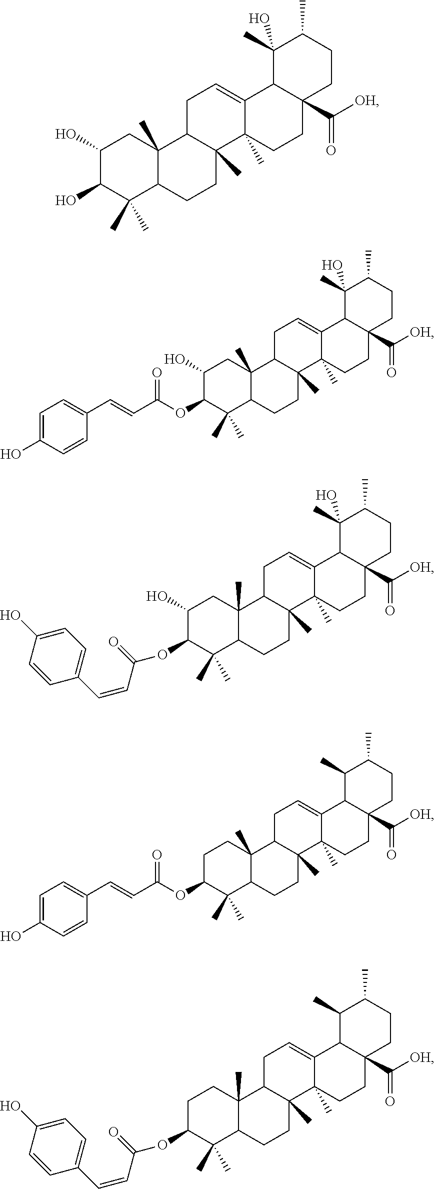

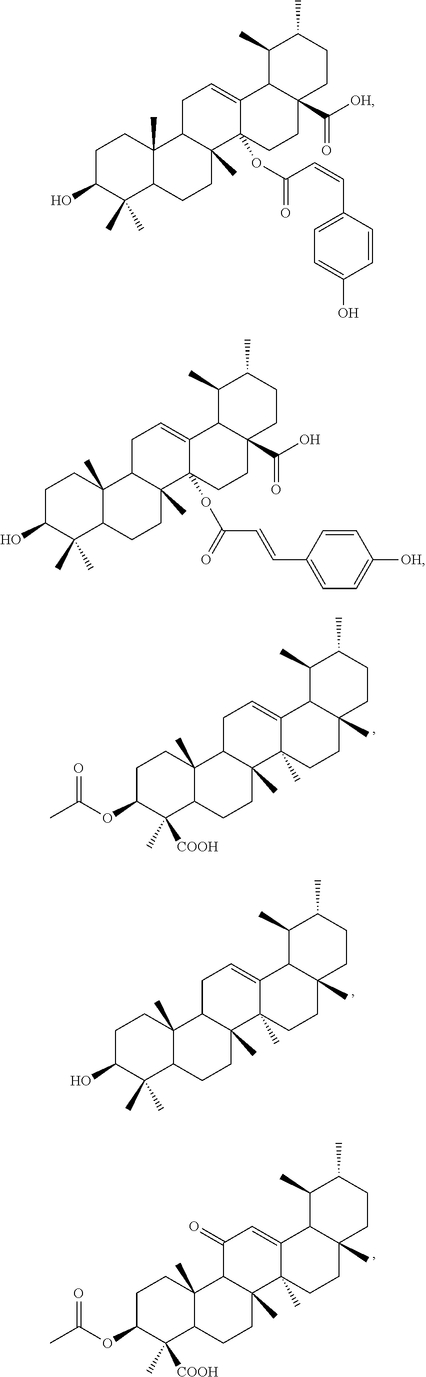

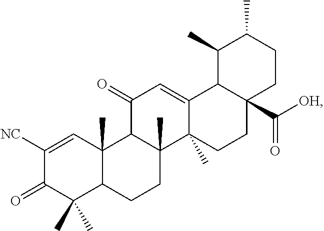

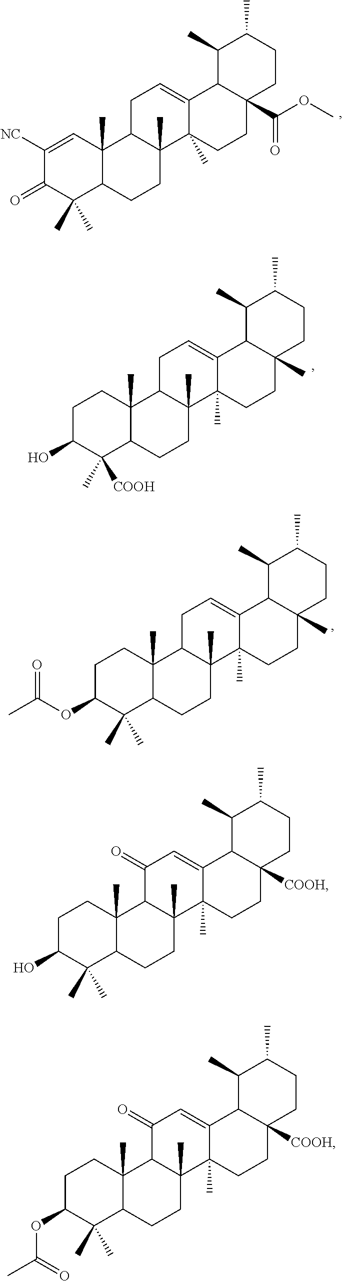

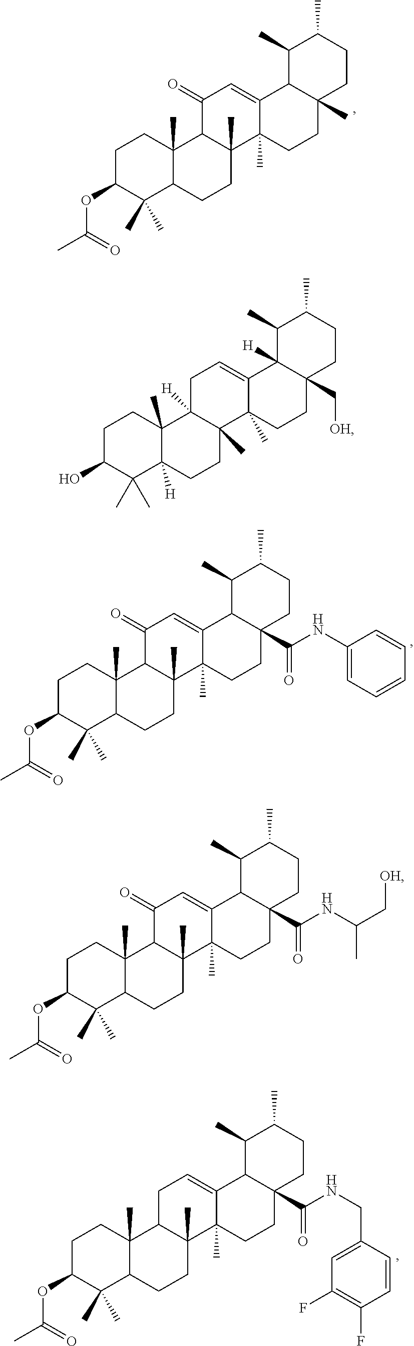

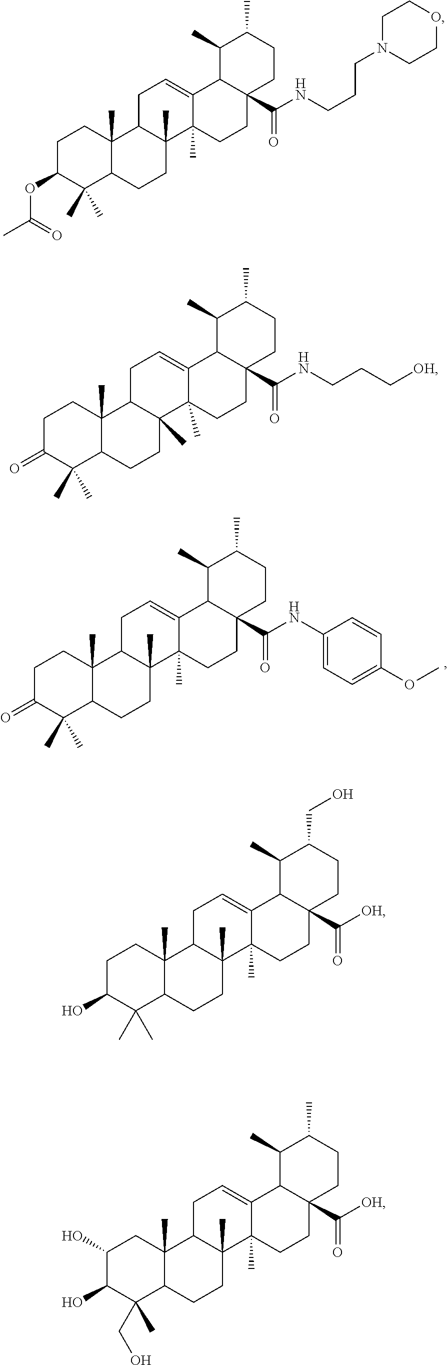

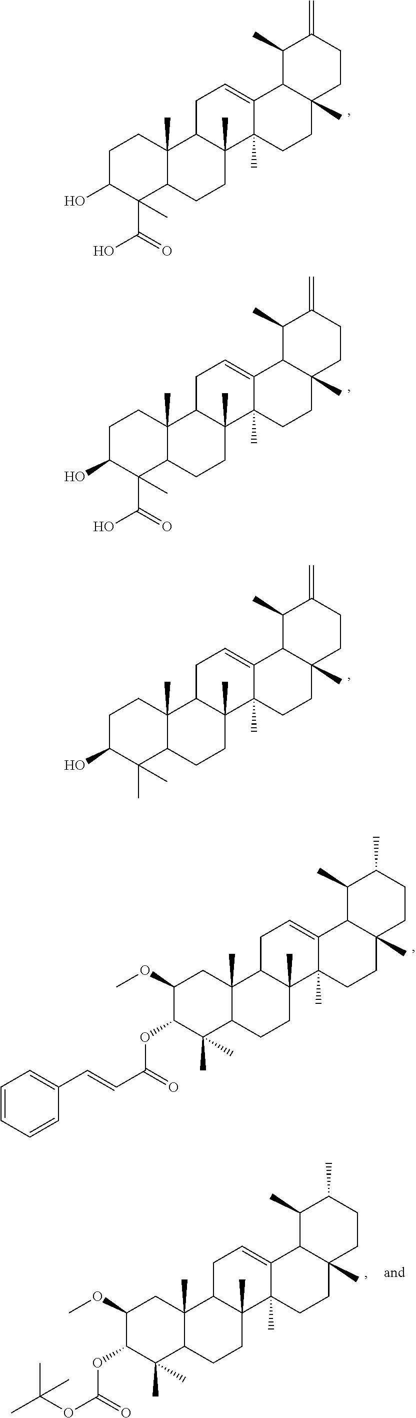

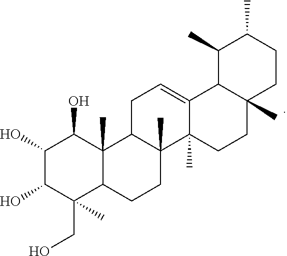

b. RNA Interference

Disclosed herein is composition for treating or preventing skeletal muscle atrophy, the composition comprising a Gadd45a and/or Cdkn1a inhibitor and an androgen and/or growth hormone elevator. Also disclosed is a composition for treating or preventing skeletal muscle atrophy, the composition comprising a Gadd45a and/or Cdkn1a inhibitor and an androgen and/or growth hormone receptor activator. In an aspect, the Gadd45a and/or Cdkn1a inhibitor is RNA interference (RNAi) targeting Gadd45a and/or Cdkn1a. In an aspect, the RNA interference is miRNA targeting Gadd45a and/or Cdkn1a. In an aspect, the RNA interference is siRNA targeting Gadd45a and/or Cdkn1a. In a further aspect, the RNA interference is shRNA targeting Gadd45a and/or Cdkn1a. In a further aspect, the RNAi (e.g., miRNA, siRNA, or shRNA) targets Cdkn1a.

RNAi relies on complementarity between the RNA and its target mRNA to bring about destruction of the target. In vivo, long stretches of dsRNA can interact with the DICER endoribonuclease to be cleaved into short (21-23 nt) dsRNA with 3′ overhangs. Then, the endogenous or synthetic short stretches of dsRNA enter the multinuclease-containing RNA-induced silencing complex (RISC) and these enzymes lead to specific cleavage of complementary targets. While short (<23 nt) segments of RNA are generally considered optimal for gene silencing it has also been shown that longer (<30 nt) sequences can lead to efficient, and perhaps even more potent, gene silencing.

The skilled person is familiar with the several different types of commonly used RNAi: short-interfering RNA (siRNA), short-hairpin RNA (shRNA), and micro RNA (miRNA), all of which can inhibit expression of the target gene product. The siRNA and shRNA (generally 20-22 nt in length, but they can be up to 30 nt) were designed to overcome issues with immune system stimulation and complete translation arrest observed when longer RNA sequences were used for RNAi, and to optimize the silencing effects.

(1) miRNA

MicroRNA (miRNA) is an RNAi-inducing agent that refers to single-stranded, non-coding RNA molecules of about 19 to about 27 base pairs that regulate gene expression in a sequence specific manner. miRNAs are post-transcriptional regulators that bind to complementary sequences on target messenger RNA transcripts (mRNAs), usually resulting in translational repression or target degradation and gene silencing.

(2) siRNA

Short interfering RNAs (siRNAs), also known as small interfering RNAs, are double-stranded RNAs that can induce sequence-specific post-transcriptional gene silencing, thereby decreasing gene expression. siRNas can be of various lengths as long as they maintain their function. In some examples, siRNA molecules are about 19-23 nucleotides in length, such as at least 21 nucleotides, and for example at least 23 nucleotides. In one example, siRNA triggers the specific degradation of homologous RNA molecules, such as mRNAs, within the region of sequence identity between both the siRNA and the target RNA. In an example, siRNAs can effect the sequence-specific degradation of target mRNAs when base-paired with 3′ overhanging ends. The direction of dsRNA processing determines whether a sense or an antisense target RNA can be cleaved by the produced siRNA endonuclease complex. Thus, siRNAs can be used to modulate transcription or translation, for example, by decreasing expression of Gadd45a or Cdkn1a. In an aspect, siRNAs can be used to modulate transcription or translation, for example, by decreasing expression of Cdkn1a. siRNAs can be generated by utilizing, for example, Invitrogen's BLOCK-IT™ RNAi Designer (https://rnaidesigner.invitrogen.com/rnaiexpress).

(3) shRNA

shRNA (short hairpin RNA) is a DNA molecule that can be cloned into expression vectors to express siRNA (typically 19-29 nt RNA duplex) for RNAi interference studies. shRNA has the following structural features: a short nucleotide sequence ranging from about 19-29 nucleotides derived from the target gene, followed by a short spacer of about 4-15 nucleotides (i.e., loop) and about a 19-29 nucleotide sequence that is the reverse complement of the initial target sequence.

c. Antisense Oligonucleotides

Disclosed herein is composition for treating or preventing skeletal muscle atrophy, the composition comprising a Gadd45a and/or Cdkn1a inhibitor and an androgen and/or growth hormone elevator. Also disclosed is a composition for treating or preventing skeletal muscle atrophy, the composition comprising a Gadd45a and/or Cdkn1a inhibitor and androgen and/or growth hormone receptor activator. In an aspect, the Gadd45a and/or Cdkn1a inhibitor is one or more antisense oligonucleotides. In a further aspect, the antisense oligonucleotides can be designed for Cdkn1a.

Generally, the term “antisense” refers to a nucleic acid molecule capable of hybridizing to a portion of an RNA sequence (such as mRNA) by virtue of some sequence complementarity. The antisense nucleic acids disclosed herein can be oligonucleotides that are double-stranded or single-stranded, RNA or DNA or a modification or derivative thereof, which can be directly administered to a cell (for example by administering the antisense molecule to the subject), or which can be produced intracellularly by transcription of exogenous, introduced sequences (for example by administering to the subject a vector that includes the antisense molecule under control of a promoter).

The art is familiar with antisense oligonucleotides. Antisense oligonucleotides or molecules are designed to interact with a target nucleic acid molecule (i.e., Gadd45a and/or Cdkn1a) through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (kd) less than or equal to 10-6, 10-8, 10-10, or 10-12.

Antisense nucleic acids are polynucleotides, for example nucleic acid molecules that are at least 6 nucleotides in length, at least 10 nucleotides, at least 15 nucleotides, at least 20 nucleotides, at least 100 nucleotides, at least 200 nucleotides, such as 6 to 100 nucleotides. However, antisense molecules can be much longer. In particular examples, the nucleotide is modified at one or more base moiety, sugar moiety, or phosphate backbone (or combinations thereof), and can include other appending groups such as peptides, or agents facilitating transport across the cell membrane or blood-brain barrier, hybridization triggered cleavage agents or intercalating agents.

In an aspect, the antisense oligonucleotide can be conjugated to another molecule, such as a peptide, hybridization triggered cross-linking agent, transport agent, or hybridization-triggered cleavage agent. Antisense oligonucleotides can include a targeting moiety that enhances uptake of the molecule by host cells. The targeting moiety can be a specific binding molecule, such as an antibody or fragment thereof that recognizes a molecule present on the surface of the host cell. Antisense molecules can be generated by utilizing the Antisense Design algorithm of Integrated DNA Technologies, Inc., available at http://www.idtdna.com/Scitools/Applications/AntiSense/Antisense.aspx/.

ii) Androgen and/or Growth Hormone Elevator

Disclosed herein is a composition for treating or preventing skeletal muscle atrophy, the composition comprising a Gadd45a and/or Cdkn1a inhibitor and an androgen and/or growth hormone elevator. In an aspect, the composition comprises a therapeutically effective amount of an androgen and/or growth hormone elevator. In an aspect, the composition comprises a prophylactically effective amount of an androgen and/or growth hormone elevator.

In an aspect, the androgen and/or growth hormone elevator is growth hormone or a growth hormone analog. Growth hormone (GH), such as human growth hormone (HGH), plays roles in metabolism, immune surveillance, heart development, and behavior, all of which are mediated by the growth hormone receptor (GHR).

In an aspect, the androgen and/or growth hormone elevator is an androgen, such as a steroid androgen. Steroid androgens are known to the art and examples of steroid androgens include, but are not limited to, testosterone, dihydrotestosterone, or androstenedione, and analogs thereof.

In an aspect, the androgen and/or growth hormone elevator is ghrelin or a ghrelin analog. Ghrelin analogs include, but are not limited to, BIM-28125 and BIM-28131. (Palus et al., 2011). In an aspect, the androgen and/or growth hormone elevator increases expression or activity of ghrelin. Ghrelin is a 28-amino acid orexigenic peptide secreted mainly from the stomach and proximal small intestine (Kojima et al., 1999). It is currently the only known circulating hormone that stimulates appetite and promotes food intake (Ariyasu et al., 2001; Date et al., 2000; Kojima et al., 1999). Ghrelin is unique in that it is the only substance that is secreted in response to a reduction in gastrointestinal contents, and it is suppressed by eating (Williams and Cummings, 2005). Active (acyl ghrelin) and inactive (des-acyl ghrelin) isoforms of ghrelin have been identified.

Activation of ghrelin is through the enzyme ghrelin O-acyltransferase (GOAT), which adds an N-octanoylated serine in position 3 to the proghrelin peptide (Gutierrez et al., 2008). This modification of ghrelin with acylation of a medium chain fatty acid is unique and is essential for ghrelin to bind to its receptor, the growth hormone secretagogue receptor (GHS-R) type 1a. The GHS-R is expressed in the hypothalamus, heart, lung, pancreas, intestine, and adipose tissue (Kojima et al., 1999). In human and animal studies, activation of the GHS-R receptor results in increased food intake (Nakazato et al., 2001; Wren et al., 2000), increased adiposity (Tschop et al., 2000), and growth hormone secretion.

Ghrelin or ghrelin analogs exert its action on appetite and food intake largely through central processes (Chen et al., 2004; Kamegai et al., 2001; Willesen et al., 1999). Signaling of circulating ghrelin is mediated by neurons of the arcuate nucleus of the hypothalamus. In particular, neurons expressing two potent orexigenic neuropeptides, neuropeptide Y (NPY) and agouti-related protein (AgRP), have been demonstrated to reduce the activity of proopiomelanocortin (POMC) neurons via ghrelin. Therefore, NPY and AgRP are mediators of the orexigenic effect of circulating ghrelin via inhibition of melanocortin signaling. It is important to note that there is also evidence that ghrelin signaling reaches the arcuate nucleus via vagal afferents. Date et al. (2002) demonstrated that subdiaphragmatic vagotomy or chemical vagal deafferentiation with capsaicin blocked the ability to peripherally administer ghrelin to stimulate food intake.

In an aspect, the androgen and/or growth hormone elevator is an aromatase inhibitor. Aromatase inhibitors decrease estrogen levels by affecting a key component of the production pathway, aromatase cytochrome P450. Aromatase inhibitors are known to the art and examples of androgens include, but are not limited to, aminoglutethimide, testolactone, anastrozole, letrozole, exemestane, vorozole, formestane, fadrozole, 4-hydroxyandrostenedione, 1,4,6-androstatrien-3,17-dione, and 4-androstene-3,6,17-trione.

iii) Androgen and/or Growth Hormone Receptor Activator

Disclosed herein is a composition for treating or preventing skeletal muscle atrophy, the composition comprising a Gadd45a and/or Cdkn1a inhibitor and an androgen and/or growth hormone receptor activator. In an aspect, the composition comprises a therapeutically effective amount of an androgen and/or growth hormone receptor activator. In an aspect, the composition comprises a prophylactically effective amount of an androgen and/or growth hormone receptor activator.

In an aspect, the androgen and/or growth hormone receptor activator is growth hormone or a growth hormone analog. Growth hormone and growth hormone homologs and analogs are known in the art.

In an aspect, androgens such as steroid androgens are known to the art and examples of steroid androgens include, but are not limited to, testosterone, dihydrotestosterone, or androstenedione, and analogs thereof

In an aspect, the androgen and/or growth hormone receptor activator is a selective androgen receptor modulator (SARMs). SARMs provide the benefits of traditional anabolic/androgenic steroids such as testosterone including increased muscle mass, fat loss, and bone density, while showing a lower tendency to produce unwanted side effects. The art is familiar with SARMs. In an aspect, the SARM can be, but is not limited to, GTx-024, BMS-564,929, LGD-4033, AC-262,356, JNJ-28330835, LGD-2226, LGD-3303, S-40503, or S-23.

In a further aspect, the androgen and/or growth hormone receptor activator is a protein tyrosine phosphatase inhibitor. As known in the art, protein tyrosine phosphatases (PTP) belong to a family of enzymes that are players in cellular signal transduction system and perturbation in their functioning is implicated in many disease-states. Protein tyrosine phosphatase inhibitors are known to the art and include, but are not limited to, protein tyrosine phosphatase, non-receptor types 1 (PTPN1), 2 (PTPN2), 3 (PTPN3), 6 (PTPN6), and 11 (PTPN11).

iv) Prevention or Treatment of Muscle Atrophy and Induction of Muscle Hypertrophy

In one aspect, the disclosed compositions treat or prevent muscle atrophy. In an aspect, the muscle atrophy can be caused by fasting. In an aspect, the muscle atrophy can be caused by immobilization. In an aspect, the muscle atrophy can be caused by denervation.

In a further aspect, the disclosed compositions increase muscle mass or muscle size. In a still further aspect, the disclosed compositions induce muscle hypertrophy. In one aspect, the disclosed compositions enhance muscle strength. In yet a further aspect, the disclosed compositions inhibit muscle atrophy and increase muscle mass. In an even further aspect, the disclosed compositions inhibit muscle atrophy and induce muscle hypertrophy. In an aspect, the disclosed compositions can increase muscle mass or size, induce muscle hypertrophy, enhance muscle strength, inhibit muscle inhibit muscle atrophy, or can effect a combination thereof.

In a further aspect, the inhibition of muscle atrophy is in an animal. In an even further aspect, the increase in muscle mass is in an animal. In a still further aspect, the animal is a mammal. In a yet further aspect, the mammal is a human. In a further aspect, the mammal is a mouse. In yet a further aspect, the mammal is a rodent.

Disclosed herein is a composition for treating or preventing skeletal muscle atrophy in a mammal, the composition comprising RNAi targeting Gadd45a and/or Cdkn1a. In an aspect, the mammal is a human. In an aspect, the disclosed composition inhibits DNA demethylation in muscle. In a further aspect, the target of DNA demethylation is the Cdkn1a gene. In an aspect, the composition stimulates anabolic signaling in muscle. In an aspect, the composition increases skeletal blood flow and oxygen delivery in muscle. In an aspect, the composition increases glucose utilization in muscle. In an aspect, the composition increases energy expenditure in muscle. In an aspect, the composition inhibits apoptosis in muscle. In an aspect, the composition decreases catabolic signaling. In an aspect, the composition restores or increases expression of genes involved in the maintenance of muscle mass and function.

Disclosed is a composition for increasing skeletal muscle blood flow in a mammal, the composition comprising ursolic acid or an ursolic acid derivative. In an aspect, the composition is prescribed for treatment of peripheral vascular disease. In an aspect, the composition induces expression of VEGFA and/or nNOS.

Disclosed is a composition for activating growth hormone receptor in a mammal, the composition comprising ursolic acid or an ursolic acid derivative. In an aspect, the mammal is a human.

Disclosed herein is a composition comprising a Gadd45a and/or Cdkn1a inhibitor and an androgen and/or growth hormone elevator, wherein the composition inhibits DNA demethylation of Cdkn1a in skeletal muscle. In an aspect, the disclosed composition stimulates anabolic signaling in skeletal muscle. In an aspect, the disclosed composition increases skeletal blood flow and oxygen delivery in muscle. In an aspect, the disclosed composition increases glucose utilization in muscle. In an aspect, the disclosed composition increases energy expenditure in muscle. In an aspect, the disclosed composition inhibits apoptosis in muscle. In an aspect, the disclosed composition decreases catabolic signaling. In an aspect, the Gadd45a and/or Cdkn1a inhibitor acts via inhibition of Gadd45a-dependent DNA demethylation enzymes. In a further aspect, the Gadd45a and/or Cdkn1a inhibitor acts via inhibition of ATF4.

Disclosed herein is a composition comprising a Gadd45a and/or Cdkn1a inhibitor and an androgen and/or growth hormone receptor activator, wherein the composition inhibits DNA demethylation of Cdkn1a in skeletal muscle. In an aspect, the disclosed composition stimulates anabolic signaling in skeletal muscle. In an aspect, the disclosed composition increases skeletal blood flow and oxygen delivery in muscle. In an aspect, the disclosed composition increases glucose utilization in muscle. In an aspect, the disclosed composition increases energy expenditure in muscle. In an aspect, the disclosed composition inhibits apoptosis in muscle. In an aspect, the disclosed composition decreases catabolic signaling.

In an aspect, the disclosed composition comprising a Gadd45a and/or Cdkn1a inhibitor and an androgen and/or growth hormone elevator restores or increases expression of genes involved in the maintenance of muscle mass and function. In an aspect, the gene is involved in insulin/IGF-1 signaling. In an aspect, the gene is involved in growth hormone signaling (e.g., growth hormone receptor or GHR). In an aspect, the gene is involved in testosterone signaling (e.g., androgen receptor or AR). In an aspect, the gene is involved in thyroid hormone signaling (e.g., thyroid hormone receptor-alpha or THRA). In an aspect, the gene is involved nitric oxide signaling (e.g., neuronal nitric oxide synthetase or nNOS or NOS1). In an aspect, the gene is involved in VEGF signaling (e.g., vascular endothelial growth factor A or VEGFA). In an aspect, the gene is involved in glucose uptake (e.g., insulin-responsive glucose transporter 4 or GLUT4, hexokinase-2 or HK2). In an aspect, the gene is involved citrate cycle signaling. In an aspect, the gene is involved in oxidative phosphorylation. In an aspect, the gene is involved in mitochondrial biogenesis (e.g., transcription factor A, mitochondrial or TFAM; peroxisome proliferator-activated receptor gamma, coactivator 1 alpha or PGC-1- or PPARGC1A).

In an aspect, the disclosed composition comprising a Gadd45a and/or Cdkn1a inhibitor and an androgen and/or growth hormone receptor activator restores or increases expression of genes involved in the maintenance of muscle mass and function. In an aspect, the gene is involved in insulin/IGF-1 signaling. In an aspect, the gene is involved in growth hormone signaling (e.g., growth hormone receptor or GHR). In an aspect, the gene is involved in testosterone signaling (e.g., androgen receptor or AR). In an aspect, the gene is involved in thyroid hormone signaling (e.g., thyroid hormone receptor-alpha or THRA). In an aspect, the gene is involved nitric oxide signaling (e.g., neuronal nitric oxidase synthetase or nNOS or NOS1). In an aspect, the gene is involved in VEGF signaling (e.g., vascular endothelial growth factor A or VEGFA). In an aspect, the gene is involved in glucose uptake (e.g., insulin-responsive glucose transporter 4 or GLUT4, hexokinase-2 or HK2). In an aspect, the gene is involved citrate cycle signaling. In an aspect, the gene is involved in oxidative phosphorylation. In an aspect, the gene is involved in mitochondrial biogenesis (e.g., transcription factor A, mitochondrial or TFAM; peroxisome proliferator-activated receptor gamma, coactivator 1 alpha or PGC-1- or PPARGC1A).

Disclosed herein is a composition for treating or preventing skeletal muscle atrophy, the composition comprising a Gadd45a and/or Cdkn1a inhibitor and an androgen and/or growth hormone elevator, wherein the inhibitor is ursolic acid and the elevator is growth hormone. In an aspect, the inhibitor is ursolic acid and the elevator is a steroid androgen. In an aspect, the inhibitor is ursolic acid and the elevator is ghrelin. In an aspect, the inhibitor is ursolic acid and the elevator is a ghrelin analog. Ghrelin analogs include, but are not limited to, BIM-28125 and BIM-28131. In an aspect, the inhibitor is ursolic acid and the elevator increases expression of activity of ghrelin. In an aspect, the inhibitor is ursolic acid and the elevator is an aromatase inhibitor.

Disclosed herein is a composition for treating or preventing skeletal muscle atrophy, the composition comprising a Gadd45a and/or Cdkn1a inhibitor and an androgen and/or growth hormone elevator, wherein the inhibitor is an ursolic acid derivative and the elevator is growth hormone. In an aspect, the inhibitor is an ursolic acid derivative and the elevator is an androgen. In an aspect, the inhibitor is an ursolic acid derivative and the elevator is ghrelin. In an aspect, the inhibitor is an uroslic acid derivative and the elevator is a ghrelin analog. Ghrelin analogs include, but are not limited to, BIM-28125 and BIM-28131. In an aspect, the inhibitor is an ursolic acid derivative and the elevator increases expression of activity of ghrelin. In an aspect, the inhibitor is an ursolic acid derivative and the elevator is an aromatase inhibitor.

Disclosed herein is a composition for treating or preventing skeletal muscle atrophy, the composition comprising a Gadd45a and/or Cdkn1a inhibitor and an androgen and/or growth hormone elevator, wherein the inhibitor is RNA interference and the elevator is growth hormone. In an aspect, the inhibitor is RNA interference and the elevator is an androgen. In an aspect, the inhibitor is RNA interference and the elevator is ghrelin. In an aspect, the inhibitor is RNA interference and the elevator is a ghrelin analog. Ghrelin analogs include, but are not limited to, BIM-28125 and BIM-28131. In an aspect, the inhibitor is RNA interference and the elevator increases expression of activity of ghrelin. In an aspect, the inhibitor is RNA interference and the elevator is an aromatase inhibitor. In an aspect, the RNA interferences targets Gadd45a and/or Cdkn1a. In a further aspect, the RNA interference is miRNA targeting Gadd45a and/or Cdkn1a. In a further aspect, the RNA interference is siRNA targeting Gadd45a and/or Cdkn1a. In yet a further aspect, the RNA interference is shRNA targeting Gadd45a and/or Cdkn1a.

Disclosed herein is a composition for treating or preventing skeletal muscle atrophy, the composition comprising a Gadd45a and/or Cdkn1a inhibitor and an androgen and/or growth hormone elevator, wherein the inhibitor is one or more antisense oligonucleotide molecules and the elevator is growth hormone. In an aspect, the inhibitor is one or more antisense oligonucleotide molecules and the elevator is an androgen. In an aspect, the inhibitor is one or more antisense oligonucleotide molecules and the elevator is ghrelin. In an aspect, the inhibitor is one or more antisense oligonucleotides and the elevator is a ghrelin analog. Ghrelin analogs include, but are not limited to, BIM-28125 and BIM-28131. In an aspect, the inhibitor is one or more antisense oligonucleotide molecules and the elevator increases expression of activity of ghrelin. In an aspect, the inhibitor is one or more antisense oligonucleotide molecules and the elevator is an aromatase inhibitor. In an aspect, the one or more antisense oligonucleotide molecules target Gadd45a and/or Cdkn1a.

Disclosed herein is a composition for treating or preventing skeletal muscle atrophy, the composition comprising a Gadd45a and/or Cdkn1a inhibitor and an androgen and/or growth hormone receptor activator, wherein the inhibitor is ursolic acid and the activator is growth hormone. In an aspect, the inhibitor is ursolic acid and the activator is an androgen. In an aspect, the inhibitor is ursolic acid and the activator is a selective androgen receptor modulator. In an aspect, the inhibitor is ursolic acid and the activator is a protein tyrosine phosphatase inhibitor.

Disclosed herein is a composition for treating or preventing skeletal muscle atrophy, the composition comprising a Gadd45a and/or Cdkn1a inhibitor and an androgen and/or growth hormone receptor activator, wherein the inhibitor is an ursolic acid derivative and the activator is growth hormone. In an aspect, the inhibitor is an ursolic acid derivative and the activator is an androgen. In an aspect, the inhibitor is an ursolic acid derivative and the activator is a selective androgen receptor modulator. In an aspect, the inhibitor is an ursolic acid derivative and the activator is a protein tyrosine phosphatase inhibitor.

Disclosed herein is a composition for treating or preventing skeletal muscle atrophy, the composition comprising a Gadd45a and/or Cdkn1a inhibitor and an androgen and/or growth hormone receptor activator, wherein the inhibitor is RNA interference and the activator is growth hormone. In an aspect, the inhibitor is RNA interference and the activator is a steroid androgen. In an aspect, the inhibitor is RNA interference and the activator is a selective androgen receptor modulator. In an aspect, the inhibitor is RNA interference and the activator is a protein tyrosine phosphatase inhibitor. In an aspect, the RNA interference targets Gadd45a and/or Cdkn1a. In a further aspect, the RNA interference is miRNA targeting Gadd45a and/or Cdkn1a. In a further aspect, the RNA interference is siRNA targeting Gadd45a and/or Cdkn1a. In yet a further aspect, the RNA interference is shRNA targeting Gadd45a and/or Cdkn1a.

Disclosed herein is a composition for treating or preventing skeletal muscle atrophy, the composition comprising a Gadd45a and/or Cdkn1a inhibitor and androgen and/or growth hormone receptor activator, wherein the inhibitor is one or more antisense oligonucleotide molecules and the activator is growth hormone. In an aspect, the inhibitor is one or more antisense oligonucleotide molecules and the activator is a steroid androgen. In an aspect, the inhibitor is antisense oligonucleotide molecules and the activator is a selective androgen receptor modulator. In an aspect, the inhibitor is antisense oligonucleotide molecules and the activator is a protein tyrosine phosphatase inhibitor. In an aspect, the one or more antisense oligonucleotide molecules target Gadd45a and/or Cdkn1a.

In an aspect, the Gadd45a and/or Cdkn1a inhibitor of the disclosed compositions acts via inhibition of Gadd45a-dependent DNA demethylation enzymes. In a further aspect, the Gadd45a and/or Cdkn1a inhibitor acts via inhibition of ATF4.

It is contemplated that one or more compositions can optionally be omitted from the disclosed invention.

### C. METHODS OF MAKING THE COMPOUNDS

In one aspect, the disclosed compounds comprise the products of the synthetic methods described herein. In a further aspect, the disclosed compounds comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one compound of any of disclosed compounds or at least one product of the disclosed methods with a pharmaceutically acceptable carrier or diluent.

In one aspect, the invention relates to methods of making functionalized ursane compounds useful in methods of inhibiting muscle atrophy and increasing muscle mass. Such compounds can be useful in the treatment of various maladies associated with muscle wasting, useful for increasing muscle mass and/or muscle strength, as well as in enhancing muscle formation and/or muscular performance. The compounds of the invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein. The following examples are provided so that the invention might be more fully understood, are illustrative only, and should not be construed as limiting.

i) Route 1: Alkyl Etherification

In one aspect, functionalized ursane compounds of the present invention can be prepared generically as shown below.

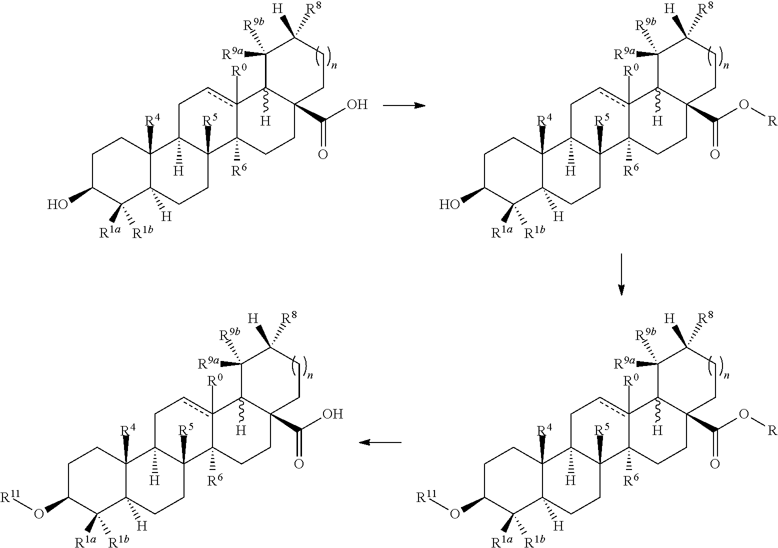

Compounds are represented in generic form, with substituents as noted in compound descriptions elsewhere herein. A more specific example is set forth below.

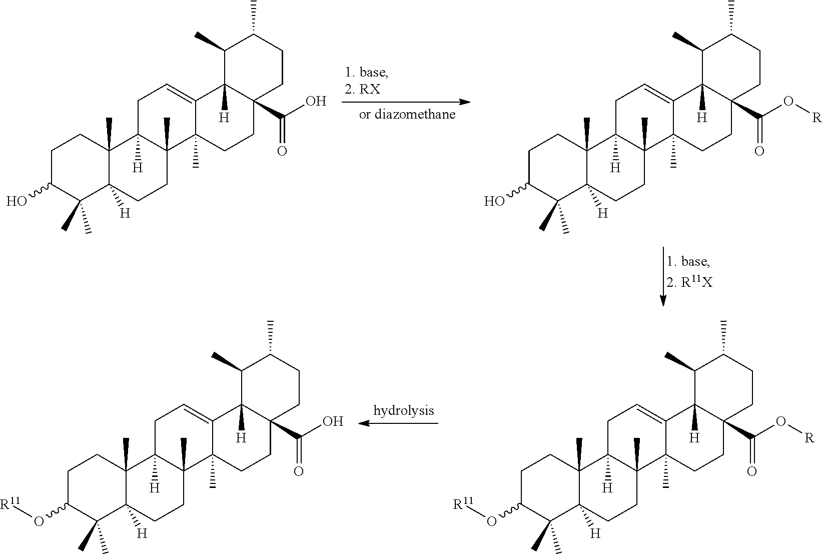

In one aspect, Route 1 step 1 begins with a free acid. In an appropriate solvent, a base (e.g., K₂CO₃, NaOH) strong enough to deprotonate the carboxylic acid, but not the alcohol, is added, and the reaction is conducted at a temperature effective and for a time effective to insure carboxylic acid deprotonation. An appropriate alkyl halide or halide equivalent is added to the reaction mixture, and the reaction is conducted at a temperature effective and for a time effective to insure alkylation of the carboxyl group. In a further aspect, an alternate Route 1 step 1 also begins with the free carboxylic acid. Diazomethane is added, and the reaction is conducted at a temperature effective and for a time effective to insure reaction.

In a further aspect, Route 1 step 2 the alkyl ester is dissolved in an appropriate dry solvent under anhydrous reaction conditions. A base is added, and the reaction is conducted at a temperature effective and for a time effective to insure deprotonation. Then, an appropriate alkyl, heteroalkyl, cycloalkyl, or heterocycloalkyl halide or halide equivalent (i.e., R¹¹X) is added to the reaction mixture. In one aspect, the reaction is conducted at a temperature effective and for a time effective to insure complete reaction.

In a further aspect, in Route 1 step 3, the O-alkylated ursane compound alkyl ester is hydrolyzed with an appropriate base, such as LiOH, in an appropriate organic-aqueous mixed solvent system at a temperature effective and for a time effective to insure reaction. Then the reaction mixture can be acidified to a suitable pH with an appropriate aqueous acid of a sufficient concentration and at a temperature effective and for a time effective to insure reaction.

ii) Route 2: Aryl Etherification

In one aspect, functionalized ursane compounds of the present invention can be prepared generically as shown below.

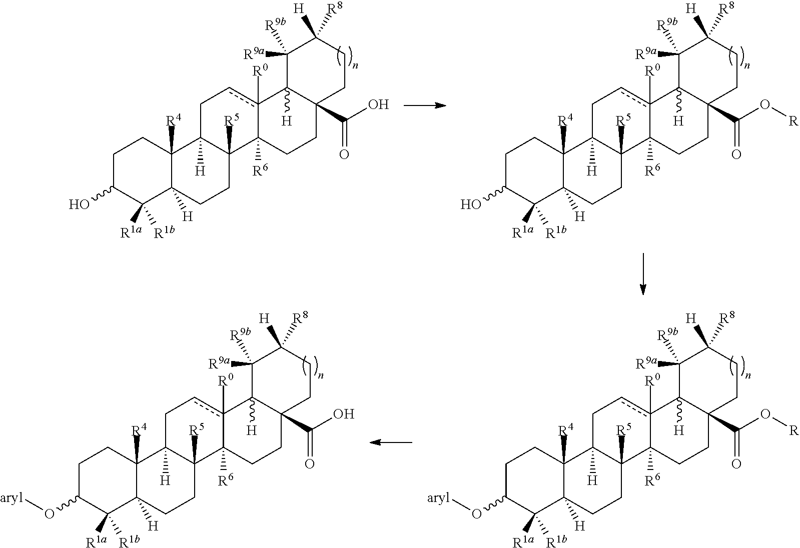

Compounds are represented in generic form, with substituents as noted in compound descriptions elsewhere herein. A more specific example is set forth below.

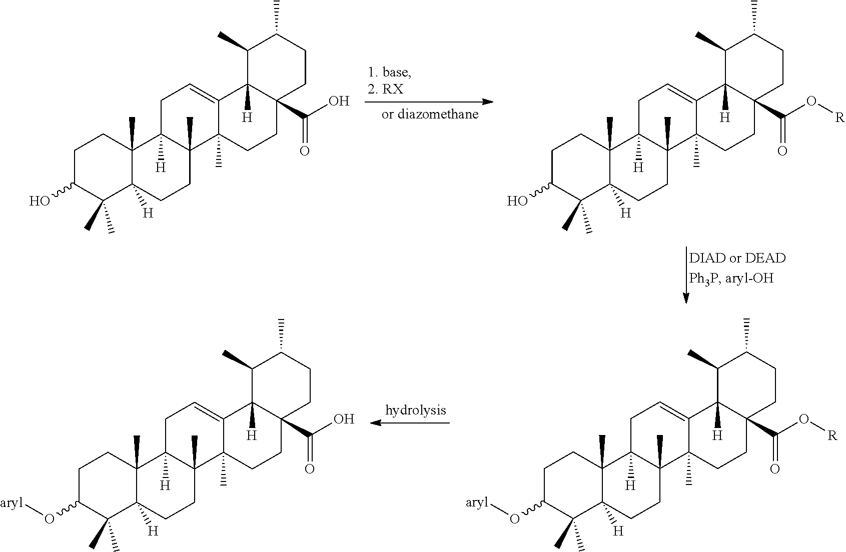

In one aspect, Route 2 step 1 begins with the ursane compound free carboxylic acid. In an appropriate solvent, a base (e.g., K₂CO₃, NaOH) strong enough to deprotonate the carboxylic acid, but not the alcohol group, is added, and the reaction is conducted at a temperature effective and for a time effective to insure deprotonation. Then, an appropriate alkyl halide or halide equivalent is added to the reaction mixture, and the reaction is conducted at a temperature effective and for a time effective to insure alkylation of the carboxyl group. In a further aspect, an alternate Route 2 step 1 begins with the ursane compound free carboxylic acid in an appropriate solvent. Diazomethane is added, and the reaction is conducted at a temperature effective and for a time effective to insure reaction.

In a further aspect, Route 2 step 2, the ursane compound alkyl ester is dissolved in an appropriate, dry solvent, along with phenol, an aryl alcohol, or appropriate heteroaryl alcohol, under anhydrous reaction conditions, followed by the addition of triphenylphosphine. The reaction is conducted at an effective temperature and for an effective time period. Then, an appropriate coupling agent, such as DIAD or DEAD, is added, and the reaction is conducted at a temperature effective and for a time effective to insure reaction. In a further aspect, in Route 2 step 3, the O-arylated or heteroarylated ursane compound alkyl ester can be treated with an appropriate base, such as LiOH, in an appropriate organic-aqueous mixed solvent system at a temperature effective and for a time effective to insure complete reaction. The reaction mixture can then be acidified to a suitable pH.

iii) Route 3: Acylation

In one aspect, functionalized ursane compounds of the present invention can be prepared generically as shown below.

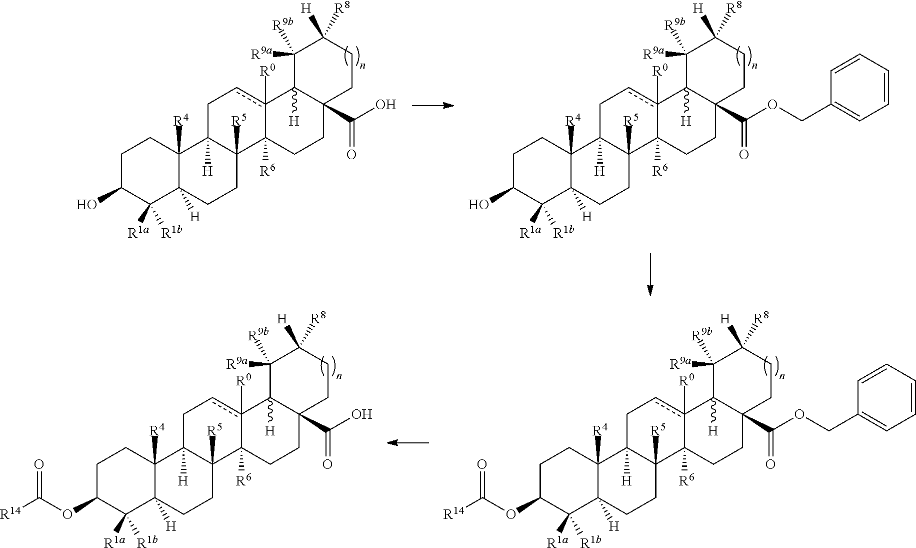

Compounds are represented in generic form, with substituents as noted in compound descriptions elsewhere herein. A more specific example is set forth below.

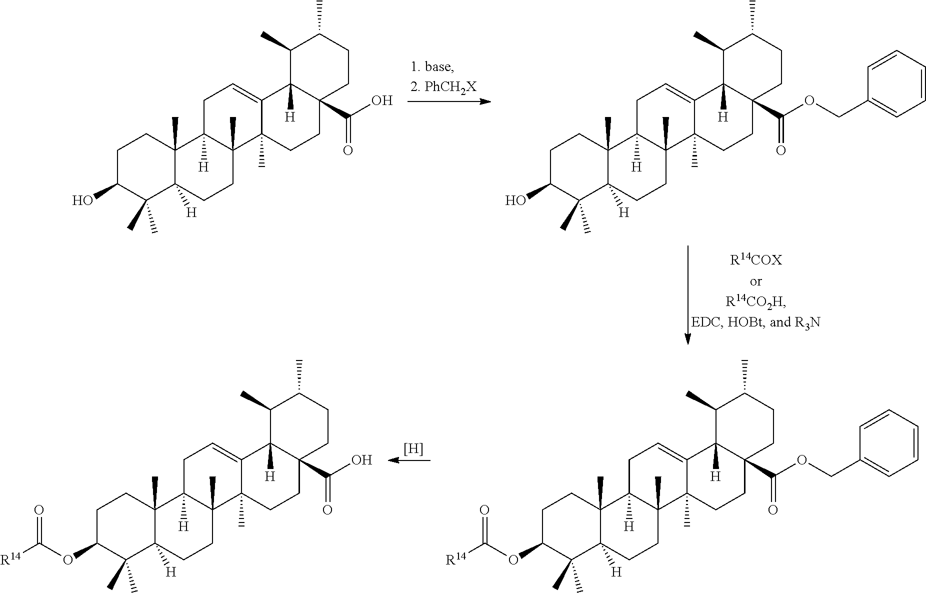

In one aspect, Route 3 step 1 begins with the ursane compound free carboxylic acid. In an appropriate solvent, a base (e.g., K₂CO₃, NaOH) strong enough to deprotonate the carboxylic acid, but not the alcohol group, is added, and the reaction is allowed to progress at a temperature effective and for a time effective to insure carboxylic acid deprotonation. Then, an appropriate benzyl halide or halide equivalent is added to the reaction mixture, and the reaction is conducted at a temperature effective and for a time effective to insure protection of the carboxyl group.

In Route 3 step 2, the ursane compound benzyl ester is dissolved in an appropriate, dry solvent under anhydrous reaction conditions, followed by the addition of an appropriate acid scavenger (weak base, e.g., K₂CO₃ or DIEA). The acyl halide (e.g., R¹⁴COX) or equivalent acylating reagent is then added. The reaction is conducted at a temperature effective and for a time effective to insure reaction. In a further aspect, in an alternate Route 3 step 2, the ursane compound benzyl ester and a suitable carboxylic acid (e.g., R¹⁴CO₂H) are dissolved in an appropriate, dry solvent under anhydrous reaction conditions. Ethyl-(N′,N′-dimethylamino)propylcarbodiimide hydrochloride (EDC), 1-hydroxybenzotriazole (HOBt), and a trialkylamine (R₃N) are then added, and the reaction is conducted at a temperature effective and for a time effective to insure reaction.

In Route 3 step 3, the acylated ursane compound benzyl ester is reduced under standard conditions (e.g., hydrogenation with hydrogen gas in the presence of a suitable palladium catalyst), thereby liberating the ursane compound free carboxlic acid.

iv) Route 4: Esterification

In one aspect, functionalized ursane compounds of the present invention can be prepared generically as shown below.

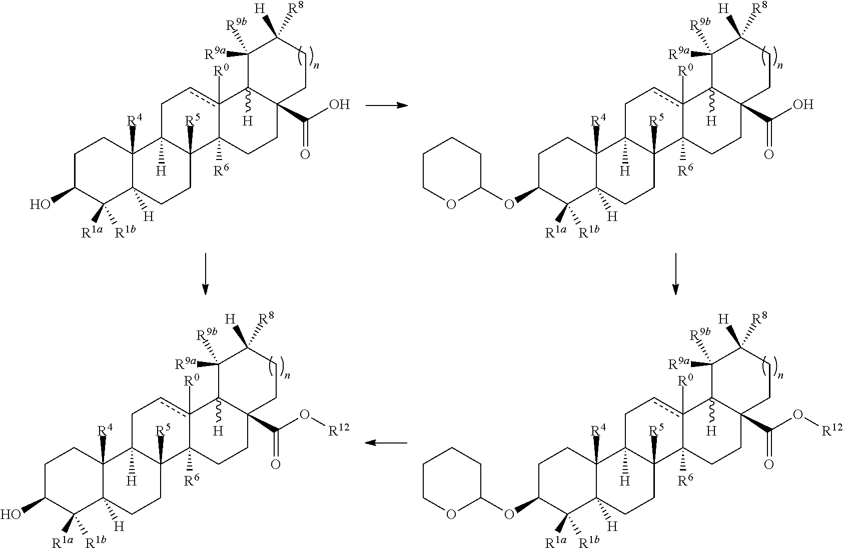

Compounds are represented in generic form, with substituents as noted in compound descriptions elsewhere herein. A more specific example is set forth below.

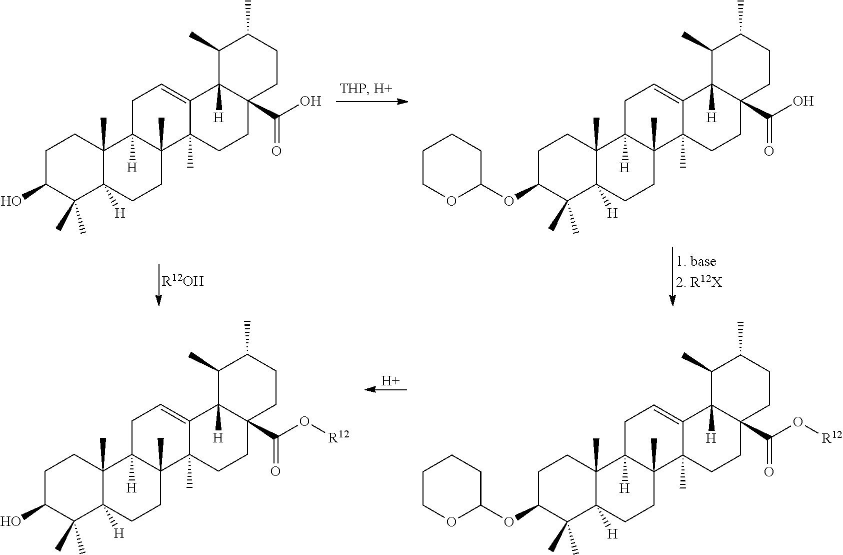

In one aspect, Route 4 step 1 begins with the ursane compound free carboxylic acid. An appropriate alcohol (e.g., R¹²OH) is added, and the reaction is conducted at a temperature effective and for a time effective to time to insure reaction.

In a further aspect in an alternate synthesis, Route 4 step 1 begins with the ursane compound free carboxylic acid in a dry solvent under dry reaction conditions. Tetrahydropyran (THP) is added, along with an acid catalyst (e.g., pTsOH). The reaction is conducted at a temperature effective and for a time effective to insure protection of the hydroxyl group. A base (e.g., NaOH or NaH) is then added to the THP-protected ursane compound free carboxylic acid, in a dry solvent under anhydrous reaction conditions. The reaction is conducted at a temperature effective and for a time effective to insure carboxylic acid deprotonation. Then, an appropriate alkyl halide (i.e., R¹²X) or equivalent is added to the reaction mixture, and the reaction is conducted at a temperature effective and for a time effective to insure alkylation of the carboxyl group. Route 4 step 3 begins with the THP-protected ursane compound alkyl ester in an alcohol solvent. An acid catalyst (e.g., pTsOH) is added, and the reaction is conducted at a temperature effective and for a time effective to insure deprotection.

v) Route 5: Amide Formation

In one aspect, functionalized ursane compounds of the present invention can be prepared generically as shown below.

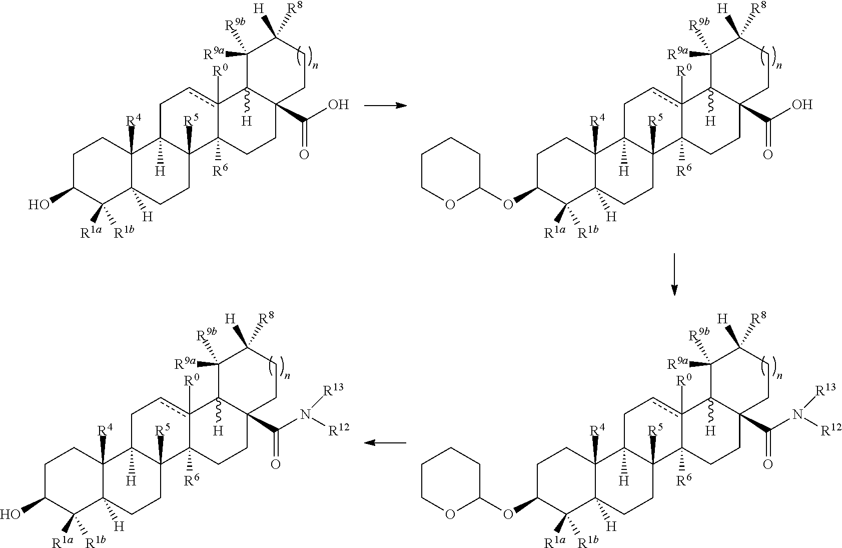

Compounds are represented in generic form, with substituents as noted in compound descriptions elsewhere herein. A more specific example is set forth below.

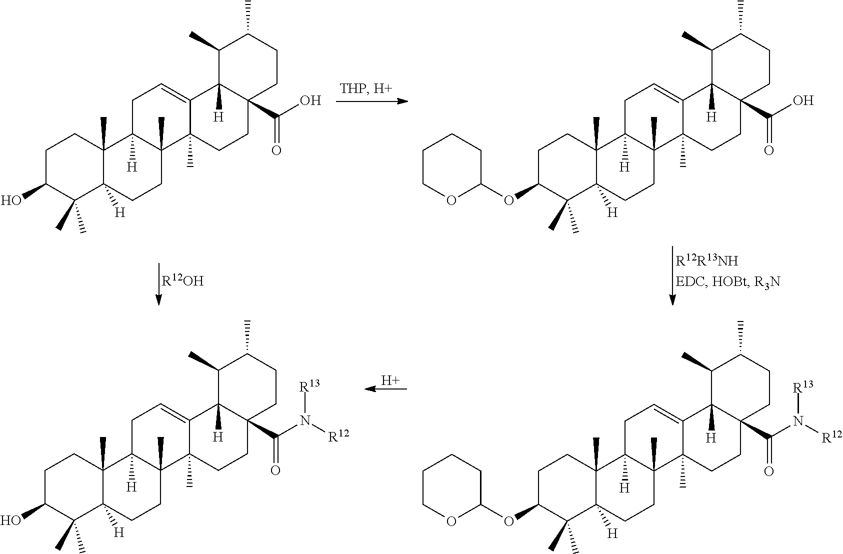

In one aspect, Route 5 step 1 begins with the ursane compound free carboxylic acid in a dry solvent. Under dry reaction conditions, tetrahydropyran (THP) and an acid catalyst (e.g., pTsOH) are added. The reaction is then conducted at a temperature effective and for a time effective to insure protection of the hydroxyl group. In Route 5 step 2, the THP-protected ursane compound free carboxylic acid is dissolved in an appropriate, dry solvent. Under anhydrous reaction conditions, a suitable amine (e.g., R¹²R¹³NH) is added, along with ethyl-(N′,N′-dimethylamino)propylcarbodiimide hydrochloride (EDC), 1-hydroxybenzotriazole (HOBt), and a trialkylamine (R₃N), and the reaction is conducted at a temperature effective and for a time effective to time to insure complete reaction. In Route 5 step 3, the THP-protected ursane compound amide can then be deprotected by addition of an acid catalyst (e.g., pTsOH), and the reaction is conducted at a temperature effective and for a time effective to insure reaction.

vi) Route 6: Reduction to Alcohol

In one aspect, functionalized ursane compounds of the present invention can be prepared generically as shown below.

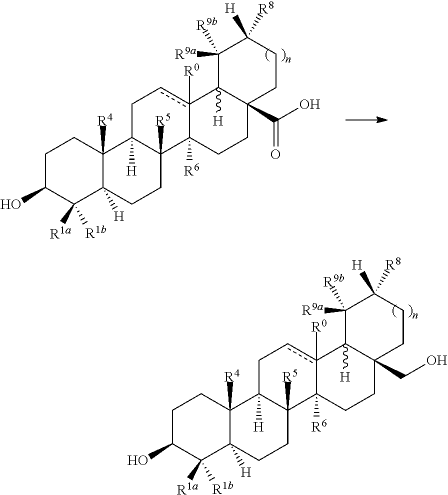

Compounds are represented in generic form, with substituents as noted in compound descriptions elsewhere herein. A more specific example is set forth below.

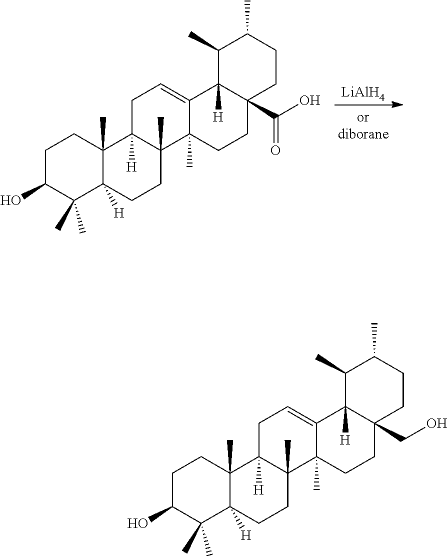

In one aspect, the ursane compound free carboxylic acid, in a dry solvent, can be reacted with lithium aluminum hydride (LiAlH₄) under dry reaction conditions to provide the corresponding primary alcohol. Alternatively, the ursane compound free carboxylic acid, in a dry solvent, can be reacted with diborane (B₂H₆) under dry reaction conditions to provide the corresponding primary alcohol. It is understood that protecting group chemistry, if needed, can also be used to protect sensitive remote functionality during these reaction steps.

vii) Route 7: Hydroxyl Inversion

In one aspect, functionalized ursane compounds of the present invention can be prepared generically as shown below.

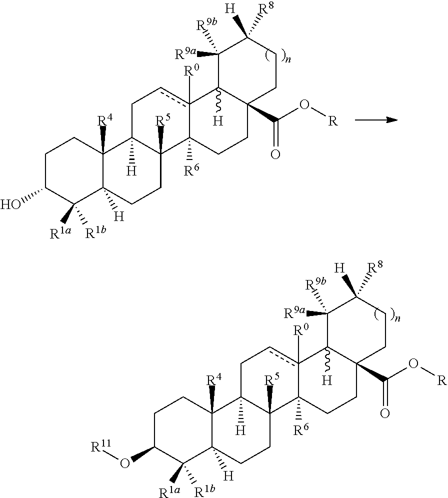

Compounds are represented in generic form, with substituents as noted in compound descriptions elsewhere herein. A more specific example is set forth below.

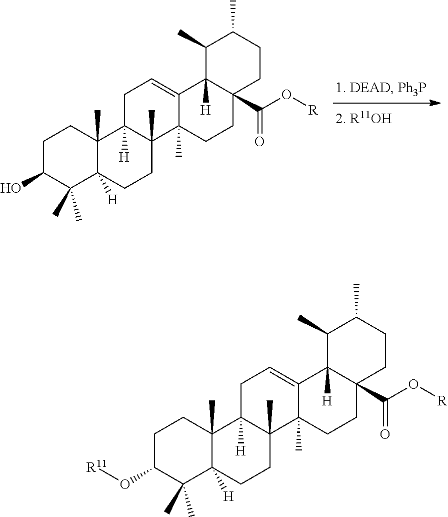

In one aspect, a hydroxyl functionality can be substituted with another group (e.g., alkoxyl, acyl, amino, etc.), while inverting the stereochemistry at the adjacent carbon, by reaction with an appropriate protic nucleophile in the presence of diethylazodicarboxylate (DEAD) and triphenylphosphine under Mitsunobu reaction conditions. While —OR¹¹ is shown, it is understood that additional moieties (e.g., acetoxyl, amino, etc.) can be substituted at that position by appropriate selection of protic nucleophile (e.g., acetic acid, ammonia, etc.).

viii) Plant Sources of Ursolic Acid Derivatives

Many pentacyclic acid triterpenes useful as synthetic precursors to the ursolic acid derivatives in the synthetic methods described above may be isolated and purified from a natural source such as plants or materials derived from plants. Alternatively, certain known synthetic precursors useful in the preparation of ursolic acid derivatives can often be obtained from commercial sources. Ursolic acid is a useful known synthetic precursor to ursolic acid derivatives that can be used as a synthetic precursor to prepare certain disclosed compounds. For example, ursolic acid can be isolated from plants such as Holy Basil (Ocimum sanctum L.), peppermint leaves (Mentha piperita L.), lavender (Lavandula augustifolia Mill.), oregano (Origanum vulgare L.), thyme (Thymus vulgaris L.), hawthorn (Crataegus laevigata (Poir) DC), cherry laurel leaves (Prunus laurocerasus L.), loquat leaves (Eriobotrya japonica L.), glossy privet leaves (Ligustrum lucidum Ait. L.), bilberry (Vacciunum myrtillus L.), Devil's Claw (Harpagophytum procumbens DC), Elder Flowers (European var.; Sambucus nigra L.), and periwinkle (Vinca minor L.).

A variety of methods that are generally applicable to purifying ursolic acid and ursolic acid derivatives. For example, Nishimura, et al. (J. Nat. Prod. 1999, 62, 1061-1064) described the identification of 2,3-dihydroxy-24-nor-urs-4(23), 12-dien-28-oic acid and 23-hydroxyursolic acid. Nishimura described procedures to isolate these. Procedures described herein demonstrate these compounds will be contained in flash chromatography fraction 3 (FCF3) as described in the examples. Similar HPLC procedures described herein can be used to further purify these compounds including using a gradient with water with 0.05% TFA and acetonitrile with 0.05% TFA, mobile phase A and B respectively, with a C18 BetaMax Neutral column (250×8 mm; 5 um). The gradient may consist of 40% β isocratic for 5 min, then from approximately 40% to 70% B in 30 min. A skilled artisan would recognize the general applicability of the methods described in Nishimura et al to efficiently isolate either the ursolic acid, ursolic acid derivatives or structurally related pentacyclic acid triterpenes from various plants.

Other illustrative methods that are generally applicable to purifying ursolic acid and ursolic acid derivatives are also known. For example, Chaturvedula, et al. (J. Nat. Prod. 2004, 67, p. 899-901) described the isolation of 3-acetoxy-2-hydroxy ursolic acid, 3-(p-coumaroyl)ursolic acid, and 2,3-diacetoxyursolic acid. Adnyana, et al. (J. Nat. Prod. 2001, 64, p. 360-363) described the isolation of 2,3,6,19-tetrahydroxyoleanolic acid, 2,3,19-trihydroxyoleanolic acid, 2,3,19,23-tetrahydroxyursolic acid, and 2,3,23-trihydroxyoleanolic acid. Ikuta, et al. (J. Nat. Prod. 2003, 66, p. 1051-1054) described the isolation of 2,3-dihydroxyurs-12-en-11-on-28-oic acid and 2,3-dihydroxy-11-methoxyurs-12-en-28-oic acid. For example, similar HPLC procedures such as those described in U.S. Pat. No. 7,612,045 can be used to further purify these compounds including using a gradient with water with 0.05% TFA and acetonitrile with 0.05% TFA, mobile phase A and B respectively, with a C18 BetaMax Neutral column (250×8 mm; 5 um). The gradient may consist of 40% β isocratic for 5 min, then from approximately 40% to 70% B in 30 min.

Finally, another source of the known synthetic precursors useful in the synthetic methods described above to prepare ursolic acid derivatives are commercial sources or vendors. Purified forms of corosolic acid, ursolic acid, oleanolic acid, madecassic acid, asiatic acid, pygenic acid (A, B or C), caulophyllogenin and echinocystic acid may be obtained from a commercial source. For example, ursolic acid and oleanolic acid may be purchased from Sigma-Aldrich Chemical Company (St. Louis, Mo., USA) and corosolic acid, asiatic acid, madecassic acid, pygenic acid (A, B, or C), caulophyllogenin and echinocystic acid may be purchased from Chromadex (Santa Ana, Calif., USA). The compounds obtained from commercial sources may be furthered separated and purified as needed using methods such as column chromatography, high pressure liquid chromatography (HPLC), and/or recrystallization described herein. Additional methods of isolation of precursors are described in U.S. Pat. No. 7,612,045, U.S. patent application Ser. No. 10/355,201, and U.S. patent application Ser. No. 10/445,943.

It is further anticipated that the compounds of the invention can be obtained by direct synthesis. Direct synthesis may include either total synthesis or semi-synthesis. Exemplary synthetic methods for obtaining these compounds are described above. Additional synthetic procedures useful in the preparation of ursolic acid derivatives are described in U.S. Pat. No. 3,903,089, U.S. Pat. No. 7,612,045, and U.S. patent application Ser. No. 10/445,943, U.S. patent application Ser. No. 10/355,201. Further synthetic methods useful in the preparation of ursolic acid derivatives are Meng, Y., et al. (2010) Molecules 15:4033-4040; Gao, Y., et al. (2010) Molecules 15:4439-4449; Sporn, M. B., et al. (2011) Journal of Natural Products 74:537-545; Chadalapaka, G., et al. (2008) Biorganic and Medicinal Chemistry Letters 18(8):2633-2639; and, Sun, H., et al. (2006) Botanical Studies 47:339-368.

It is contemplated that each disclosed methods can further comprise additional steps, manipulations, and/or components. It is also contemplated that any one or more step, manipulation, and/or component can be optionally omitted from the invention. It is understood that a disclosed methods can be used to provide the disclosed compounds. It is also understood that the products of the disclosed methods can be employed in the disclosed methods of using.

### D. PHARMACEUTICAL COMPOSITIONS

In one aspect, the invention relates to pharmaceutical compositions comprising the disclosed composition comprising a Gadd45a and/or Cdkn1a inhibitor and an androgen and/or growth hormone elevator. In a further aspect, the invention relates to pharmaceutical compositions comprising the disclosed composition comprising a Gadd45a and/or Cdkn1a inhibitor and an androgen and/or growth hormone receptor activator. In an aspect, the disclosed pharmaceutical compositions can be provided comprising a therapeutically effective amount of the inhibitor and/or the elevator, and a pharmaceutically acceptable carrier. The disclosed pharmaceutical compositions can be provided comprising a prophylactically effective amount of the inhibitor and/or the elevator, and pharmaceutically acceptable carrier. In an aspect, the disclosed pharmaceutical compositions can be provided comprising a therapeutically effective amount of the inhibitor and/or the activator, and a pharmaceutically acceptable carrier. The disclosed pharmaceutical compositions can be provided comprising a phrophylactically effective amount of the inhibitor and/or the activator, and a pharmaceutically acceptable carrier.

In one aspect, the invention relates to a pharmaceutical composition comprising a pharmaceutically acceptable carrier and an effective amount of an Gadd45a and/or Cdkn1a inhibitor having a structure represented by a formula:

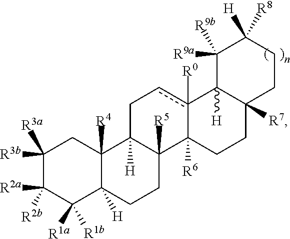

wherein each [symbol] is an optional covalent bond, and R⁰ is optionally present; wherein n is 0 or 1; wherein R⁰, when present, is hydrogen; wherein R^{1a} is selected from C1-C6 alkyl and —C(O)ZR¹⁰; wherein R^{1b} is selected from C1-C6 alkyl, or wherein R^{1a} and R^{1b} are optionally covalently bonded and, together with the intermediate carbon, comprise an optionally substituted C3-C5 cycloalkyl or C2-C5 heterocycloalkyl; wherein one of R^{2a} and R^{2b} is —OR¹¹, and the other is hydrogen, or R^{2a} and R^{2b} together comprise ═O; wherein each of R^{3a} and R^{3b} is independently selected from hydrogen, hydroxyl, C1-C6 alkyl, and C1-C6 alkoxyl, provided that R^{3a} and R^{3b} are not simultaneously hydroxyl, wherein R^{3a} and R^{3b} are optionally covalently bonded and, together with the intermediate carbon, comprise an optionally substituted C3-C5 cycloalkyl or C2-C5 heterocycloalkyl; wherein each of R⁴, R⁵, and R⁶ is independently selected from C1-C6 alkyl; wherein R⁷ is selected from C1-C6 alkyl, —CH₂OR¹² and —C(O)ZR¹²; wherein R⁸ is selected from hydrogen and C1-C6 alkyl; wherein each of R^{9a} and R^{9b} is independently selected from hydrogen and C1-C6 alkyl, provided that R^{9a} and R^{9b} are not simultaneously hydrogen; or wherein R^{9a} and R^{9b} are covalently bonded and, along with the intermediate carbon, together comprise C3-C5 cycloalkyl or C2-C5 heterocycloalkyl; wherein R¹⁰ is selected from hydrogen and C1-C6 alkyl; wherein R¹¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, wherein Z is N, R¹² and R¹³ are covalently bonded and —NR¹²R¹³ comprises a moiety of the formula:

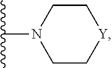

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; or a pharmaceutically acceptable salt, hydrate, solvate, or polymorph thereof, in an amount effective to prevent or treat muscle atrophy in the animal, wherein the amount is greater than about 1000 mg per day when the compound is ursolic acid, boswellic acid, corosolic acid, betulinic acid, or UA0713.

In one aspect, the animal is an animal. In a further aspect, the animal is a mammal. In a yet further aspect, the mammal is a primate. In a still further aspect, the mammal is a human. In an even further aspect, the human is a patient.

In a further aspect, the animal is a domesticated animal. In a still further aspect, the domesticated animal is a domesticated fish, domesticated crustacean, or domesticated mollusk. In a yet further aspect, the domesticated animal is poultry. In an even further aspect, the poultry is selected from chicken, turkey, duck, and goose. In a still further aspect, the domesticated animal is livestock. In a yet further aspect, the livestock animal is selected from pig, cow, horse, goat, bison, and sheep.

In a further aspect, the pharmaceutical composition is administered following identification of the mammal in need of treatment of muscle atrophy. In a still further aspect, the pharmaceutical composition is administered following identification of the mammal in need of prevention of muscle atrophy. In an even further aspect, the mammal has been diagnosed with a need for treatment of muscle atrophy prior to the administering step.

In a further aspect, the compound is not ursolic acid, beta-boswellic acid, corosolic acid, betulinic acid, or UA0713. In a yet further aspect, the compound is ursolic acid, beta-boswellic acid, corosolic acid, betulinic acid, or UA0713.

In certain aspects, the disclosed pharmaceutical compositions comprise the disclosed (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in moulds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions which require modulation of cellular function related to muscle growth an appropriate dosage level will generally be about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 miligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The present invention is further directed to a method for the manufacture of a medicament for modulating cellular activity related to muscle growth (e.g., treatment of one or more disorders associated with muscle dysfunction or atrophy) in mammals (e.g., humans) comprising combining one or more disclosed compounds, products, or compositions with a pharmaceutically acceptable carrier or diluent. Thus, in one aspect, the invention relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

### E. METHODS OF USING THE COMPOSITIONS

i) Muscle Atrophy

Muscle atrophy is defined as a decrease in the mass of the muscle; it can be a partial or complete wasting away of muscle. When a muscle atrophies, this leads to muscle weakness, since the ability to exert force is related to mass. Muscle atrophy is a co-morbidity of several common diseases, and patients who have “cachexia” in these disease settings have a poor prognosis.

Muscle atrophy can also be skeletal muscle loss or weakness secondary to malnutrition, bedrest, neurologic disease (including multiple sclerosis, amyotrophic lateral sclerosis, spinal muscular atrophy, critical illness neuropathy, spinal cord injury or peripheral nerve injury), orthopedic injury, casting, and other post-surgical forms of limb immobilization, chronic disease (including cancer, congestive heart failure, chronic pulmonary disease, chronic renal failure, chronic liver disease, diabetes mellitus, Cushing syndrome, growth hormone deficiency, IGF-I deficiency, androgen deficiency, estrogen deficiency, and chronic infections such as HIV/AIDS or tuberculosis), cancer chemotherapy, burns, sepsis, other illnesses requiring mechanical ventiliation, drug-induced muscle disease (such as glucorticoid-induced myopathy and statin-induced myopathy), genetic diseases that primarily affect skeletal muscle (such as muscular dystrophy and myotonic dystrophy), autoimmune diseases that affect skeletal muscle (such as polymyositis and dermatomyositis), spaceflight, or age-related sarcopenia.

There are many diseases and conditions which cause muscle atrophy, including malnutrition, bedrest, neurologic disease (including multiple sclerosis, amyotrophic lateral sclerosis, spinal muscular atrophy, critical illness neuropathy, spinal cord injury or peripheral nerve injury), orthopedic injury, casting, and other post-surgical forms of limb immobilization, chronic disease (including cancer, congestive heart failure, chronic pulmonary disease, chronic renal failure, chronic liver disease, diabetes mellitus, Cushing syndrome, growth hormone deficiency, IGF-I deficiency, androgen deficiency, estrogen deficiency, and chronic infections such as HIV/AIDS or tuberculosis), cancer chemotherapy, burns, sepsis, other illnesses requiring mechanical ventiliation, drug-induced muscle disease (such as glucorticoid-induced myopathy and statin-induced myopathy), genetic diseases that primarily affect skeletal muscle (such as muscular dystrophy and myotonic dystrophy), autoimmune diseases that affect skeletal muscle (such as polymyositis and dermatomyositis), spaceflight, or age-related sarcopenia.

Muscle atrophy occurs by a change in the normal balance between protein synthesis and protein degradation. During atrophy, there is a down-regulation of protein synthesis pathways, and an activation of protein breakdown pathways. Protein degradation pathways which seem to be responsible for much of the muscle loss seen in a muscle undergoing atrophy are autophagy, caspase-dependent proteolysis and the ATP-dependent, ubiquitin/proteasome pathway.

Muscle atrophy can be opposed by the signaling pathways which induce muscle hypertrophy, or an increase in muscle size. Therefore one way in which exercise induces an increase in muscle mass is to downregulate the pathways which have the opposite effect. One important rehabilitation tool for muscle atrophy includes the use of functional electrical stimulation to stimulate the muscles which has had limited success in the rehabilitation of paraplegic patients.

Ursolic acid or ursolic acid derivatives can be used as a therapy for illness- and age-related muscle atrophy. It can be useful as a monotherapy or in combination with other strategies that have been considered, such as myostatin inhibition (Zhou, X., et al. (2010) Cell 142(4): 531-543). Given its capacity to reduce adiposity, fasting blood glucose and plasma lipid levels, ursolic acid or ursolic acid derivatives can also be used as a therapy for obesity, metabolic syndrome and type 2 diabetes.

The disclosed compounds can be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

Systemic administration of ursolic acid (by parenteral injection or by oral consumption) can be used to promote muscle growth and reduce muscle atrophy in all muscles, including those of the limbs and the diaphragm. Local administration of ursolic acid (by a topical route or localized injection) can be used to promote local muscle growth, as can be required following a localized injury or surgery.

In one aspect, the subject compounds can be coadministered with agents that reduce skeletal muscle atrophy, increase skeletal muscle mass, increase skeletal muscle strength, increase skeletal muscle insulin signaling, increase skeletal muscle IGF-I signaling and/or increase skeletal muscle glucose uptake including but not limited to tomatidine, tomatidine analogs, tacrine, tacrine analogs, allantoin, allantoin analogs, connesine, connesine analogs, naringenin, naringenin analogs, hippeastrine, hippeastrine analogs, ungerine, ungerine analogs, insulin, insulin analogs, insulin-like growth factor 1, metformin, thiazoladinediones, sulfonylureas, meglitinides, leptin, dipeptidyl peptidase-4 inhibitors, glucagon-like peptide-1 agonists, tyrosine-protein phosphatase non-receptor type inhibitors, myostatin signaling inhibitors, TGF-beta signaling inhibitors, beta-2 adrenergic agents including clenbuterol, androgens, selective androgen receptor modulator (such as GTx-024, BMS-564,929, LGD-4033, AC-262,356, JNJ-28330835, LGD-2226, LGD-3303, S-40503, or S-23), aromatase inhibitors (such as anastrozole, letrozole, exemestane, vorozole, formestane, fadrozole, 4-hydroxyandrostenedione, 1,4,6-androstatrien-3,17-dione, and 4-androstene-3,6,17-trione), growth hormone, a growth hormone analog, ghrelin, a ghrelin analog. A disclosed compound or salt thereof can be administered orally, intramuscularly, intravenously or intraarterially. A disclosed compound or salt thereof can be substantially pure. A disclosed compound or salt thereof can be administered at about 10 mg/day to 10 g/day.

In another aspect, the subject compounds can be administered in combination with agents that agents that reduce skeletal muscle atrophy, increase skeletal muscle mass, increase skeletal muscle strength, increase skeletal muscle insulin signaling, increase skeletal muscle IGF-I signaling and/or increase skeletal muscle glucose uptake including but not limited to tomatidine, tomatidine analogs, tacrine, tacrine analogs, allantoin, allantoin analogs, connesine, connesine analogs, naringenin, naringenin analogs, hippeastrine, hippeastrine analogs, ungerine, ungerine analogs, insulin, insulin analogs, insulin-like growth factor 1, metformin, thiazoladinediones, sulfonylureas, meglitinides, leptin, dipeptidyl peptidase-4 inhibitors, glucagon-like peptide-1 agonists, tyrosine-protein phosphatase non-receptor type inhibitors, myostatin signaling inhibitors, TGF-beta signaling inhibitors, beta-2 adrenergic agents including clenbuterol, androgens, selective androgen receptor modulator (such as GTx-024, BMS-564,929, LGD-4033, AC-262,356, JNJ-28330835, LGD-2226, LGD-3303, S-40503, or S-23), aromatase inhibitors (such as anastrozole, letrozole, exemestane, vorozole, formestane, fadrozole, 4-hydroxyandrostenedione, 1,4,6-androstatrien-3,17-dione, and 4-androstene-3,6,17-trione), growth hormone, a growth hormone analog, ghrelin, a ghrelin analog. A disclosed compound or salt thereof can be administered orally, intramuscularly, intravenously or intraarterially. A disclosed compound or salt thereof can be substantially pure. A disclosed compound or salt thereof can be administered at about 10 mg/day to 10 g/day.

The pharmaceutical compositions and methods of the present invention can further comprise other therapeutically active as noted herein which are usually applied in the treatment of the above mentioned pathological conditions.

ii) Treatment Methods

The compounds disclosed herein are useful for treating, preventing, ameliorating, controlling or reducing the risk of a variety of muscle disorders. Examples of such muscle disorders include, but are not limited to, skeletal muscle atrophy secondary to malnutrition, bedrest, neurologic disease (including multiple sclerosis, amyotrophic lateral sclerosis, spinal muscular atrophy, critical illness neuropathy, spinal cord injury or peripheral nerve injury), orthopedic injury, casting, and other post-surgical forms of limb immobilization, chronic disease (including cancer, congestive heart failure, chronic pulmonary disease, chronic renal failure, chronic liver disease, diabetes mellitus, Cushing syndrome, growth hormone deficiency, IGF-I deficiency, androgen deficiency, estrogen deficiency, and chronic infections such as HIV/AIDS or tuberculosis), cancer chemotherapy, burns, sepsis, other illnesses requiring mechanical ventiliation, drug-induced muscle disease (such as glucorticoid-induced myopathy and statin-induced myopathy), genetic diseases that primarily affect skeletal muscle (such as muscular dystrophy and myotonic dystrophy), autoimmune diseases that affect skeletal muscle (such as polymyositis and dermatomyositis), spaceflight, or age-related sarcopenia.

The compounds disclosed herein are useful for treating, preventing, ameliorating, controlling or reducing the risk of a variety of muscle disorders, including those that occur when an animal such as a human has hypogonadism or hypopituitarism, or when the human has suffered an injury to limb or body, or when the human is wearing or has worn a cast, a splint, or a brace, or when a human will undergo surgery for an illness or injury, or when a human is or has been on mechanical ventiliation, or when the human is or has been in spaceflight, or when the human is being treated or has been treated for prostate cancer.

a. Preventing or Treating Skeletal Muscle Atrophy

Disclosed herein is a method for preventing or treating skeletal muscle atrophy in an animal, the method comprising administering to the animal an effective amount of a composition comprising a Gadd45a and/or Cdkn1a inhibitor and an androgen and/or growth hormone elevator.

In an aspect, the composition comprises a therapeutically effective amount of a Gadd45a and/or Cdkn1a inhibitor. In an aspect, the composition comprises a prophylactically effective amount of a Gadd45a and/or Cdkn1a inhibitor. In an aspect, the Gadd45a and/or Cdkn1a inhibitor is ursolic acid. In an aspect, the inhibitor is an ursolic acid derivative. In a further aspect, the inhibitor is RNA interference. In a further aspect, the inhibitor is one or more antisense oligonucleotides. In an aspect, the composition comprises a therapeutically effective amount of an androgen and/or growth hormone elevator. In an aspect, the composition comprises a prophylactically effective amount of an androgen and/or growth hormone elevator. In an aspect, the androgen and/or growth hormone elevator is androgen. In an aspect, the elevator is growth hormone. In a further aspect, the elevator is ghrelin or a ghrelin analog or something that increases the expression or activity of ghrelin. In a further aspect, the elevator is an aromatase inhibitor.

Disclosed herein is a method for preventing or treating muscle atrophy in an animal, the method comprising administering to the animal an effective amount of a composition comprising a Gadd45a and/or Cdkn1a inhibitor and an androgen and/or growth hormone elevator, further comprises inhibiting demethylation of the Cdkn1a gene in skeletal muscle. In an aspect, the disclosed method further comprises stimulating anabolic signaling in skeletal muscle. In an aspect, the disclosed method further comprises increasing skeletal blood flow and oxygen delivery in muscle. In an aspect, the disclosed method further comprises increasing glucose utilization in muscle. In an aspect, the disclosed method further comprises increasing energy expenditure in muscle. In an aspect, the disclosed method further comprises inhibiting apoptosis in muscle. In an aspect, the disclosed method further comprises decreasing catabolic signaling.

Disclosed herein is a method for preventing or treating muscle atrophy in an animal, the method comprising administering to the animal an effective amount of a composition comprising a Gadd45a and/or Cdkn1a inhibitor and an androgen and/or growth hormone receptor activator. In an aspect, the composition comprises a therapeutically effective amount of a Gadd45a and/or Cdkn1a inhibitor. In an aspect, the composition comprises a prophylactically effective amount of a Gadd45a and/or Cdkn1a inhibitor. In an aspect, the Gadd45a and/or Cdkn inhibitor is ursolic acid. In an aspect, the inhibitor is an ursolic acid derivative. In a further aspect, the inhibitor is RNA interference. In a further aspect, the inhibitor is one or more antisense oligonucleotides. In an aspect, the composition comprises a therapeutically effective amount of an androgen and/or growth hormone receptor activator. In an aspect, the composition comprises a prophylactically effective amount of an androgen and/or growth hormone receptor activator. In an aspect, the androgen and/or growth hormone receptor activator is androgen. In an aspect, the receptor activator is growth hormone. In a further aspect, the receptor activator is a selective androgen receptor modulator. In a further aspect, the receptor activator is a protein tyrosine phosphatase inhibitor.

In an aspect, the disclosed method comprising administering an effective amount of a composition comprising a Gadd45a and/or Cdkn1a inhibitor and an androgen and/or growth hormone receptor activator, further comprises inhibiting DNA demethylation of Cdkn1a in skeletal muscle. In an aspect, the disclosed method further comprises stimulating anabolic signaling in skeletal muscle. In an aspect, the disclosed method further comprises increasing skeletal blood flow and oxygen delivery in muscle. In an aspect, the disclosed method further comprises increasing glucose utilization in muscle. In an aspect, the disclosed method further comprises increasing energy expenditure in muscle. In an aspect, the disclosed method further comprises inhibiting apoptosis in muscle. In an aspect, the disclosed method further comprises decreasing catabolic signaling.

Also disclosed herein, is a method for preventing or treating skeletal muscle atrophy in an animal, the method comprising administering to the animal an effective amount of a composition comprising a one or more agents that inhibit Gadd45a expression and/or Cdkn1a expression, agents that inhibit Gadd45a function and/or Cdkn1a function, or agents that inhibit active DNA demethylation, or a combination thereof, thereby preventing or treating muscle atrophy. In one aspect, the method does comprise agents that increase androgen and/or growth hormone signaling. In an aspect, the composition comprises a therapeutically effective amount of one or more agents that inhibit Gadd45a expression and/or Cdkn1a expression, agents that inhibit Gadd45a function and/or Cdkn1a function, or agents that inhibit active DNA demethylation, or a combination thereof. In an aspect, the composition comprises a prophylactically effective amount of one or more agents that inhibit Gadd45a expression and/or Cdkn1a expression, agents that inhibit Gadd45a function and/or Cdkn1a function, or agents that inhibit active DNA demethylation, or a combination thereof. In an aspect, the one or more agents that inhibit Gadd45a expression and/or Cdkn1a expression, agents that inhibit Gadd45a function and/or Cdkn1a function, or agents that inhibit active DNA demethylation, or a combination thereof comprises ursolic acid. In an aspect, the one or more agents that inhibit Gadd45a expression and/or Cdkn1a expression, agents that inhibit Gadd45a function and/or Cdkn1a function, or agents that inhibit active DNA demethylation, or a combination thereof comprises an ursolic acid derivative. In a further aspect, the one or more agents that inhibit Gadd45a expression and/or Cdkn1a expression, agents that inhibit Gadd45a function and/or Cdkn1a function, or agents that inhibit active DNA demethylation, or a combination thereof comprises RNA interference. In a further aspect, the one or more agents that inhibit Gadd45a expression and/or Cdkn1a expression, agents that inhibit Gadd45a function and/or Cdkn1a function, or agents that inhibit active DNA demethylation, or a combination thereof comprises antisense oligonucleotides.

In the disclosed methods of preventing or treating skeletal muscle atrophy in an animal, the animal can be a human. In an aspect, the human can be in utero, or an infant, or a child, or an adolescent, or an adult. In an aspect, the human can be aged. In an aspect, the human can have one or more diseases or conditions, including but not limited to, diabetes, cancer, HIV/AIDS, heart failure, chronic obstructive pulmonary disease, cirrhosis, renal failure, Cushing syndrome, multiple sclerosis, muscular dystrophy, peripheral vascular diseases, amyotrophic lateral sclerosis, spinal muscular atrophy, and arthritis. In an aspect, the human has suffered a stroke, a brain injury, or spinal cord injury. In an aspect, the human is on bed rest. In an aspect, the human has been on bed rest. In an aspect, the human has received treatment for cancer. In an aspect, the human is receiving treated for cancer. In an aspect, the human has suffered fractures. In a further aspect, the human is receiving exogenous glucocorticoids. In an aspect, the human is malnourished.

In an aspect, the disclosed methods for preventing or treating skeletal muscle atrophy can further comprise administering the composition during and/or following a period of muscle non-use. In an aspect, the disclosed methods for preventing or treating skeletal muscle atrophy can further comprise administering the composition as a bolus and/or at regular intervals. In an aspect, the disclosed methods for preventing and treating skeletal muscle further can comprise administering the composition intravenously, intraperitoneally, intramuscularly, subcutaneously, or transdermally.

The disclosed methods for preventing and treating skeletal muscle atrophy can further comprise administering the composition in conjunction with at least one other treatment or therapy. In an aspect, the other treatment or therapy is physical therapy.

The disclosed methods for preventing and treating skeletal muscle atrophy can further comprise diagnosing the animal with muscle atrophy. In an aspect, the animal is diagnosed with muscle atrophy prior to administration of the composition. The disclosed methods for preventing and treating skeletal muscle atrophy can further comprise identifying an animal in need of treatment for muscle atrophy.

The disclosed methods for preventing and treating skeletal muscle atrophy can further comprise evaluating the efficacy of the composition. In an aspect, evaluating the efficacy of the composition comprises measuring muscle atrophy prior to administering the composition and measuring muscle atrophy after administering the composition. In an aspect, evaluating the efficacy of the composition comprises measuring muscle strength prior to administering the composition and measuring muscle strength after administering the composition. In a further aspect, evaluating the efficacy of the composition comprises measuring muscle mass prior to administering the composition and measuring muscle mass after administering the composition. In an aspect, evaluating the efficacy of the composition can occur at regular intervals.

The disclosed methods for preventing and treating skeletal muscle atrophy can further comprise optionally adjusting at least one aspect of method. In an aspect, adjusting at least one aspect of method comprises changing the dose of the composition. In an aspect, adjusting at least one aspect of method comprises changing the frequency of administration of the composition. In an aspect, adjusting at least one aspect of method comprises changing the route of administration of the composition. In an aspect, adjusting at least one aspect of method comprises one or more of the dose of the composition, the frequency of administration of the composition, or the route of administration of the composition.

Disclosed herein is a method of treating or preventing skeletal muscle atrophy in a mammal, the method comprising administering ursolic acid or an ursolic acid derivative; and inducing expression of VEGFA and/or nNOS. Also disclosed is a method for increasing skeletal muscle blood flow in a mammal, the method comprising administering a composition comprising ursolic acid or an ursolic acid derivative. In an aspect, the mammal has peripheral vascular disease. In an aspect, the composition induces expression of VEGFA and/or nNOS.

Dislcosed herein is a method of treating or preventing skeletal muscle atrophy in a mammal, the method comprising administering ursolic acid or an ursolic acid derivative; and activating growth hormone receptor.

Disclosed herein is a method for preventing or treating muscle atrophy in an animal, the method comprising administering to the animal an effective amount of an androgen and/or growth hormone elevator subsequent to the animal having received a Gadd45a and/or Cdkn1a inhibitor. Also disclosed herein is a method for preventing or treating muscle atrophy in an animal, the method comprising administering to the animal an effective amount of a Gadd45a and/or Cdkn1a inhibitor subsequent to the animal having received an androgen and/or growth hormone elevator.

In an aspect, the composition comprises a therapeutically effective amount of a Gadd45a and/or Cdkn1a inhibitor. In an aspect, the composition comprises a prophylactically effective amount of a Gadd45a and/or Cdkn1a inhibitor. In an aspect, the Gadd45a and/or Cdkn1a inhibitor is ursolic acid. In an aspect, the inhibitor is an ursolic acid derivative. In a further aspect, the inhibitor is RNA interference. In a further aspect, the inhibitor is one or more antisense oligonucleotides. In an aspect, the composition comprises a therapeutically effective amount of an androgen and/or growth hormone elevator.

In an aspect, the composition comprises a prophylactically effective amount of an androgen and/or growth hormone elevator. In an aspect, the androgen and/or growth hormone elevator is androgen. In an aspect, the elevator is growth hormone. In a further aspect, the elevator is ghrelin or a ghrelin analog or something that increases the expression or activity of ghrelin. In a further aspect, the elevator is an aromatase inhibitor.

Disclosed herein is a method for preventing or treating muscle atrophy in an animal, the method comprising administering to the animal an effective amount of an androgen and/or growth hormone receptor activator subsequent to the animal having received a Gadd45a and/or Cdkn1a inhibitor. Also disclosed herein is a method for preventing or treating muscle atrophy in an animal, the method comprising administering to the animal an effective amount of a Gadd45a and/or Cdkn1a inhibitor subsequent to the animal having received an androgen and/or growth hormone receptor activator. In an aspect, the composition comprises a therapeutically effective amount of a Gadd45a and/or Cdkn1a inhibitor. In an aspect, the composition comprises a prophylactically effective amount of a Gadd45a and/or Cdkn1a inhibitor. In an aspect, the Gadd45a and/or Cdkn1a inhibitor is ursolic acid. In an aspect, the inhibitor is an ursolic acid derivative. In a further aspect, the inhibitor is RNA interference. In a further aspect, the inhibitor is one or more antisense oligonucleotides. In an aspect, the composition comprises a therapeutically effective amount of an androgen and/or growth hormone receptor activator. In an aspect, the composition comprises a prophylactically effective amount of an androgen and/or growth hormone receptor activator. In an aspect, the androgen and/or growth hormone receptor activator is androgen. In an aspect, the receptor activator is growth hormone. In a further aspect, the receptor activator is a selective androgen receptor modulator. In a further aspect, the receptor activator is a protein tyrosine phosphatase inhibitor.

In an aspect, the disclosed method comprising administering an effective amount of a composition comprising a Gadd45a and/or Cdkn1a inhibitor to an animal subsequent to the animal having received an androgen/growth hormone elevator, or administering an effective amount of a composition comprising a Gadd45a and/or Cdkn1a inhibitor to an animal subsequent to the animal having received an androgen and/or growth hormone receptor activator, can further comprise diagnosing the animal with muscle atrophy. The disclosed methods for preventing and treating skeletal muscle atrophy can further comprise identifying an animal in need of treatment for muscle atrophy. In an aspect, the disclosed method can further comprise evaluating the efficacy of the composition. In an aspect, evaluating the efficacy of the composition comprises measuring muscle atrophy prior to administering the Gadd45a and/or Cdkn1a inhibitor and the activator or elevator and measuring muscle atrophy after administering the Gadd45a and/or Cdkn1a inhibitor and the activator or elevator. In an aspect, evaluating the efficacy of the composition comprises measuring muscle strength prior to administering the Gadd45a and/or Cdkn1a inhibitor and the activator or elevator and measuring muscle strength after administering Gadd45a and/or Cdkn1a inhibitor and the activator or elevator. In a further aspect, evaluating the efficacy of the composition comprises measuring muscle mass prior to administering Gadd45a and/or Cdkn1a inhibitor and the activator or elevator and measuring muscle mass after administering Gadd45a and/or Cdkn1a inhibitor and the activator or elevator. In an aspect, evaluating the efficacy of the composition can occur at regular intervals.

In an aspect, the disclosed method comprising administering an effective amount of a composition comprising an androgen/growth hormone elevator to an animal subsequent to the animal having received a Gadd45a and/or Cdkn1a inhibitor, or administering an effective amount of a composition comprising an androgen and/or growth hormone receptor activator subsequent to the animal having received a Gadd45a and/or Cdkn1a inhibitor, can further comprise diagnosing the animal with muscle atrophy. The disclosed methods for preventing and treating skeletal muscle atrophy can further comprise identifying an animal in need of treatment for muscle atrophy. In an aspect, the disclosed method can further comprise evaluating the efficacy of the composition comprises measuring muscle atrophy prior to administering the activator or elevator and the Gadd45a and/or Cdkn1a inhibitor and measuring muscle atrophy after administering the activator or elevator and the Gadd45a and/or Cdkn1a inhibitor. In an aspect, evaluating the efficacy of the composition comprises measuring muscle strength prior to administering the activator or elevator and the Gadd45a and/or Cdkn1a inhibitor and measuring muscle strength after administering the activator or elevator and the Gadd45a and/or Cdkn1a inhibitor. In a further aspect, evaluating the efficacy of the composition comprises measuring muscle mass prior to administering the activator or elevator and the Gadd45a and/or Cdkn1a inhibitor and measuring muscle mass after administering the activator or elevator and the Gadd45a and/or Cdkn1a inhibitor. In an aspect, evaluating the efficacy of the composition can occur at regular intervals.

In an aspect, the Gadd45a and/or Cdkn1a inhibitor of the methods disclosed above acts via inhibition of Gadd45a-dependent DNA demethylation enzymes. In a further aspect, the Gadd45a and/or Cdkn1a inhibitor acts via inhibition of ATF4.

In one aspect, the invention relates to a method for preventing or treating muscle atrophy in an animal, the method comprising administering to the animal a compound of the formula:

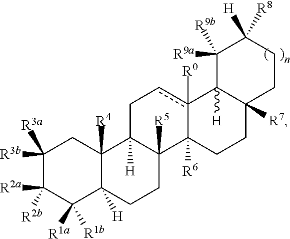

wherein each [symbol] is an optional covalent bond, and R⁰ is optionally present; wherein n is 0 or 1; wherein R⁰, when present, is hydrogen; wherein R^{1a} is selected from C1-C6 alkyl and —C(O)ZR¹⁰; wherein R^{1b} is selected from C1-C6 alkyl, or wherein R^{1a} and R^{1b} are optionally covalently bonded and, together with the intermediate carbon, comprise an optionally substituted C3-C5 cycloalkyl or C2-C5 heterocycloalkyl; wherein one of R^{2a} and R^{2b} is —OR¹¹, and the other is hydrogen, or R^{2a} and R^{2b} together comprise ═O; wherein each of R^{3a} and R^{3b} is independently selected from hydrogen, hydroxyl, C1-C6 alkyl, and C1-C6 alkoxyl, provided that R^{3a} and R^{3b} are not simultaneously hydroxyl, wherein R^{3a} and R^{3b} are optionally covalently bonded and, together with the intermediate carbon, comprise an optionally substituted C3-C5 cycloalkyl or C2-C5 heterocycloalkyl; wherein each of R⁴, R⁵, and R⁶ is independently selected from C1-C6 alkyl; wherein R⁷ is selected from C1-C6 alkyl, —CH₂OR¹² and —C(O)ZR¹²; wherein R⁸ is selected from hydrogen and C1-C6 alkyl; wherein each of R^{9a} and R^{9b} is independently selected from hydrogen and C1-C6 alkyl, provided that R^{9a} and R^{9b} are not simultaneously hydrogen; or wherein R^{9a} and R^{9b} are covalently bonded and, along with the intermediate carbon, together comprise C3-C5 cycloalkyl or C2-C5 heterocycloalkyl; wherein R¹⁰ is selected from hydrogen and C1-C6 alkyl; wherein R¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, wherein Z is N, R¹² and R¹³ are covalently bonded and —NR¹²R¹³ comprises a moiety of the formula:

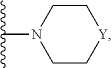

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; or a pharmaceutically acceptable salt, hydrate, solvate, or polymorph thereof, in an amount effective to prevent or treat muscle atrophy in the animal, wherein the amount is greater than about 1000 mg per day when the compound is ursolic acid, beta-boswellic acid, corosolic acid, betulinic acid, or UA0713.

In a further aspect, the compound administered is a disclosed compound or a product of a disclosed method of making a compound.

In a further aspect, the animal is a mammal. In a yet further aspect, the mammal is a primate. In a still further aspect, the mammal is a human. In an even further aspect, the human is a patient. In a further aspect, the animal is a domesticated animal. In a still further aspect, the domesticated animal is a domesticated fish, domesticated crustacean, or domesticated mollusk. In a yet further aspect, the domesticated animal is poultry. In an even further aspect, the poultry is selected from chicken, turkey, duck, and goose. In a still further aspect, the domesticated animal is livestock. In a yet further aspect, the livestock animal is selected from pig, cow, horse, goat, bison, and sheep.

In a further aspect, the effective amount is a therapeutically effective amount. In a still further aspect, the effective amount is a prophylactically effective amount. In a yet further aspect, muscle atrophy is prevented by administration of the compound. In an even further aspect, muscle atrophy is treated by administration of the compound. In a still further aspect, the method further comprises the step of identifying the mammal in need of treatment of muscle atrophy. In a yet further aspect, the method further comprises the step of identifying the mammal in a need of prevention of muscle atrophy. In an even further aspect, the mammal has been diagnosed with a need for treatment of muscle atrophy prior to the administering step.

In a further aspect, the compound is not ursolic acid, beta-boswellic acid, corosolic acid, betulinic acid, or UA0713. In a still further aspect, the compound is ursolic acid, beta-boswellic acid, corosolic acid, betulinic acid, or UA0713. In yet further aspect, the compound is not administered as a foodstuff

b. Facilitating Muscle Hypertrophy

Disclosed herein is a method for facilitating muscle hypertrophy, the method comprising the steps of (i) inhibiting expression of Gadd45a and/or Cdkn1a, and (ii) increasing cellular concentration of androgen and/or growth hormone. In an aspect, increasing cellular concentration comprises administering exogenous androgen and/or growth hormone. In a further aspect, increasing cellular concentration comprises improving the half-life of endogenous androgen and/or growth hormone. In a further aspect, increasing cellular concentration comprises increasing expression of androgen and/or growth hormone.

Also disclosed herein is a method for facilitating muscle hypertrophy, the method comprising the steps of (1) inhibiting expression of Gadd45a and/or Cdkn1a, and (ii) increasing activity of androgen and/or growth hormone receptor. In an aspect, the Gadd45a and/or Cdkn1a inhibitor acts via inhibition of Gadd45a-dependent DNA demethylation enzymes. In a further aspect, the Gadd45a and/or Cdkn inhibitor acts via inhibition of ATF4.

The methods of facilitating muscle hypertrophy disclosed herein can further comprise inhibiting DNA demethylation of Cdkn1a in skeletal muscle. In an aspect, the disclosed methods can further comprise stimulating anabolic signaling in skeletal muscle. In an aspect, the disclosed methods can further comprise increasing skeletal blood flow and oxygen delivery in muscle. In an aspect, the disclosed methods can further comprise increasing glucose utilization in muscle. In an aspect, the disclosed methods can further comprise increasing energy expenditure in muscle. In an aspect, the disclosed methods can further comprise inhibiting apoptosis in muscle. In an aspect, the disclosed methods can further comprise decreasing catabolic signaling.

In an aspect, the Gadd45a and/or Cdkn1a inhibitor of the disclosed methods of facilitating muscle hypertrophy can be ursolic acid, an ursolic acid derivative, RNA interference, or one or more antisense oligonucleotides. In an aspect, the androgen and/or growth hormone elevator of the disclosed methods of facilitating muscle hypertrophy can be an androgen, a growth hormone, ghrelin, a ghrelin analog, something that increases the expression or activity of ghrelin, or an aromatase inhibitor. In an aspect, the androgen and/or growth hormone receptor activator of the disclosed methods of facilitating muscle hypertrophy can be an androgen, a growth hormone, a selective androgen receptor modulator, or a protein tyrosine phosphatase inhibitor.

The methods for facilitating muscle hypertrophy disclosed herein can further comprise restoring or increasing expression of genes involved in the maintenance of muscle mass and function. In an aspect, the gene is involved in insulin/IGF-1 signaling (e.g., IRS2). In an aspect, the gene is involved in growth hormone signaling (e.g., growth hormone receptor or GHR). In an aspect, the gene is involved in testosterone signaling (e.g., androgen receptor or AR). In an aspect, the gene is involved in thyroid hormone signaling (thyroid hormone receptor-alpha or THRA). In an aspect, the gene is involved nitric oxide signaling (e.g., neuronal nitric oxide synthetase or nNOS or NOS1). In an aspect, the gene is involved in VEGF signaling (e.g., vascular endothelial growth factor A or VEGFA). In an aspect, the gene is involved in glucose uptake (e.g., insulin-responsive glucose transporter 4 or GLUT4, hexokinase-2 or HK2). In an aspect, the gene is involved citrate cycle signaling (e.g., succinyl CoA ligase-alpha or SUCLG1). In an aspect, the gene is in involved in oxidative phosphorylation (e.g., cytochrome C oxidase 11 or COX11). In an aspect, the gene is involved in mitochondrial biogenesis (e.g., transcription factor A, mitochondrial or TFAM; peroxisome proliferator-activated receptor gamma, coactivator 1 alpha or PGC-1α or PPARGC1A).

In one aspect, the invention relates to a method for increasing muscle mass and/or muscular strength in an animal, the method comprising administering to the animal a compound of the formula:

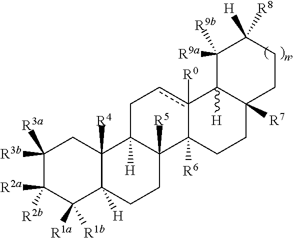

wherein each [symbol] is an optional covalent bond, and R⁰ is optionally present; wherein n is 0 or 1; wherein R⁰, when present, is hydrogen; wherein R^{1a} is selected from C1-C6 alkyl and —C(O)ZR¹⁰; wherein R^{1b} is selected from C1-C6 alkyl, or wherein R^{1a} and R^{1b} are optionally covalently bonded and, together with the intermediate carbon, comprise an optionally substituted C3-C5 cycloalkyl or C2-C5 heterocycloalkyl; wherein one of R^{2a} and R^{2b} is —OR¹¹, and the other is hydrogen, or R^{2a} and R^{2b} together comprise ═O; wherein each of R^{3a} and R^{3b} is independently selected from hydrogen, hydroxyl, C1-C6 alkyl, and C1-C6 alkoxyl, provided that R^{3a} and R^{3b} are not simultaneously hydroxyl, wherein R^{3a} and R^{3b} are optionally covalently bonded and, together with the intermediate carbon, comprise an optionally substituted C3-C5 cycloalkyl or C2-C5 heterocycloalkyl; wherein each of R⁴, R⁵, and R⁶ is independently selected from C1-C6 alkyl; wherein R⁷ is selected from C1-C6 alkyl, —CH₂OR¹² and —C(O)ZR¹²; wherein R⁸ is selected from hydrogen and C1-C6 alkyl; wherein each of R^{9a} and R^{9b} is independently selected from hydrogen and C1-C6 alkyl, provided that R^{9a} and R^{9b} are not simultaneously hydrogen; or wherein R^{9a} and R^{9b} are covalently bonded and, along with the intermediate carbon, together comprise C3-C5 cycloalkyl or C2-C5 heterocycloalkyl; wherein R¹⁰ is selected from hydrogen and C1-C6 alkyl; wherein R¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹ permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, wherein Z is N, R¹² and R¹³ are covalently bonded and —NR¹²R¹³ comprises a moiety of the formula:

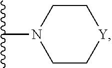

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; or a pharmaceutically acceptable salt, hydrate, solvate, or polymorph thereof, in an amount effective to prevent or treat muscle atrophy in the animal, wherein the amount is greater than about 1000 mg per day when the compound is ursolic acid, beta-boswellic acid, corosolic acid, betulinic acid, or UA0713. In a further aspect, the compound administered is a disclosed compound or a product of a disclosed method of making a compound.

In a further aspect, the compound administered is a disclosed compound or a product of a disclosed method of making a compound.

In a further aspect, the animal is a mammal. In a yet further aspect, the mammal is a primate. In a still further aspect, the mammal is a human. In an even further aspect, the human is a patient. In a further aspect, the animal is a domesticated animal. In a still further aspect, the domesticated animal is a domesticated fish, domesticated crustacean, or domesticated mollusk. In a yet further aspect, the domesticated animal is poultry. In an even further aspect, the poultry is selected from chicken, turkey, duck, and goose. In a still further aspect, the domesticated animal is livestock. In a yet further aspect, the livestock animal is selected from pig, cow, horse, goat, bison, and sheep.

In a further aspect, the effective amount is a therapeutically effective amount. In a still further aspect, the effective amount is a prophylactically effective amount. In a yet further aspect, muscle atrophy is prevented by administration of the compound. In an even further aspect, muscle atrophy is treated by administration of the compound. In a still further aspect, the method further comprises the step of identifying the mammal in need of treatment of muscle atrophy. In a yet further aspect, the method further comprises the step of identifying the mammal in need of prevention of muscle atrophy. In an even further aspect, the mammal has been diagnosed with a need for treatment of muscle atrophy prior to the administering step.

In a further aspect, the compound is not ursolic acid, beta-boswellic acid, corosolic acid, betulinic acid, or UA0713. In a still further aspect, the compound is ursolic acid, beta-boswellic acid, corosolic acid, betulinic acid, or UA0713. In yet further aspect, the compound is not administered as a foodstuff.

c. Inhibiting Expression of Gadd45a and/or Cdkn1a and Providing Androgen and/or Growth Hormone

Disclosed herein is a method comprising the steps of inhibiting expression of Gadd45a and/or Cdkn1a and providing androgen and/or growth hormone. In an aspect, inhibiting and providing steps are performed in vitro. In an aspect, inhibiting and providing steps are performed in vivo. In an aspect, inhibiting and providing steps in an animal. In an aspect, the animal is a primate. In an aspect, the animal is a mammal. In an aspect, the animal is a human.

The method comprising inhibiting expression of Gadd45a and/or Cdkn1a and providing androgen and/or growth hormone disclosed herein can further comprise inhibiting DNA demethylation of Cdkn in skeletal muscle. In an aspect, the disclosed method can further comprise stimulating anabolic signaling in skeletal muscle. In an aspect, the disclosed method can further comprise increasing skeletal blood flow and oxygen delivery in muscle. In an aspect, the disclosed method can further comprise increasing glucose utilization in muscle. In an aspect, the disclosed method can further comprise increasing energy expenditure in muscle. In an aspect, the disclosed method can further comprise inhibiting apoptosis in muscle. In an aspect, the disclosed method can further comprise decreasing catabolic signaling. In an aspect, the Gadd45a and/or Cdkn1a inhibitor acts via inhibition of Gadd45a-dependent DNA demethylation enzymes. In a further aspect, the Gadd45a and/or Cdkn1a inhibitor acts via inhibition of ATF4.

d. Inhibiting Expression of Gadd45a and/or Cdkn1a and Providing Androgen and/or Growth Hormone Receptor

Disclosed herein is a method comprising the steps of inhibiting expression of Gadd45a and/or Cdkn and activating androgen and/or growth hormone receptor. In an aspect, inhibiting and providing steps are performed in vitro. In an aspect, inhibiting and providing steps are performed in vivo. In an aspect, inhibiting and providing steps in an animal. In an aspect, the animal is a primate. In an aspect, the animal is a mammal. In an aspect, the animal is a human.

The disclosed methods comprising inhibiting expression of Gadd45a and/or Cdkn1a and providing androgen and/or growth hormone receptor can further comprise inhibiting DNA demethylation of Cdkn1a in skeletal muscle. In an aspect, the disclosed method can further comprise stimulating anabolic signaling in skeletal muscle. In an aspect, the disclosed method can further comprise increasing skeletal blood flow and oxygen delivery in muscle. In an aspect, the disclosed method can further comprise increasing glucose utilization in muscle. In an aspect, the disclosed method can further comprise increasing energy expenditure in muscle. In an aspect, the disclosed method can further comprise inhibiting apoptosis in muscle. In an aspect, the disclosed method can further comprise decreasing catabolic signaling. In an aspect, the Gadd45a and/or Cdkn1a inhibitor acts via inhibition of Gadd45a-dependent DNA demethylation enzymes. In a further aspect, the Gadd45a and/or Cdkn1a inhibitor acts via inhibition of ATF4.

e. Increasing Skeletal Muscle Glucose Uptake

Also disclosed herein is a method of increasing skeletal muscle glucose uptake comprising, administering to an animal an effective amount of a composition comprising one or more agents that inhibit Gadd45a expression and/or Cdkn1a expression, agents that inhibit Gadd45a function and/or Cdkn1a function, or agents that inhibit active DNA demethylation, or a combination thereof, thereby increasing skeletal muscle glucose uptake. In an aspect, inhibiting and providing steps are performed in vitro. In an aspect, inhibiting and providing steps are performed in vivo. In an aspect, inhibiting and providing steps in an animal. In an aspect, the animal is a primate. In an aspect, the animal is a mammal. In an aspect, the animal is a human.

In one aspect, the method does comprise agents that increase androgen and/or growth hormone signaling. In an aspect, the composition comprises a therapeutically effective amount of one or more agents that inhibit Gadd45a expression and/or Cdkn1a expression, agents that inhibit Gadd45a function and/or Cdkn1a function, or agents that inhibit active DNA demethylation, or a combination thereof. In an aspect, the composition comprises a prophylactically effective amount of one or more agents that inhibit Gadd45a expression and/or Cdkn1a expression, agents that inhibit Gadd45a function and/or Cdkn1a function, or agents that inhibit active DNA demethylation, or a combination thereof. In an aspect, the one or more agents that inhibit Gadd45a expression and/or Cdkn1a expression, agents that inhibit Gadd45a function and/or Cdkn1a function, or agents that inhibit active DNA demethylation, or a combination thereof comprises ursolic acid. In an aspect, the one or more agents that inhibit Gadd45a expression and/or Cdkn1a expression, agents that inhibit Gadd45a function and/or Cdkn1a function, or agents that inhibit active DNA demethylation, or a combination thereof comprises an ursolic acid derivative. In a further aspect, the one or more agents that inhibit Gadd45a expression and/or Cdkn1a expression, agents that inhibit Gadd45a function and/or Cdkn1a function, or agents that inhibit active DNA demethylation, or a combination thereof comprises RNA interference. In a further aspect, the one or more agents that inhibit Gadd45a expression and/or Cdkn1a expression, agents that inhibit Gadd45a function and/or Cdkn1a function, or agents that inhibit active DNA demethylation, or a combination thereof comprises antisense oligonucleotides.

f. Increasing Skeletal Muscle Oxidative Metabolism

Also disclosed herein is a method of increasing skeletal muscle oxidative metabolism comprising, administering to an animal an effective amount of a composition comprising one or more agents that inhibit Gadd45a expression and/or Cdkn1a expression, agents that inhibit Gadd45a function and/or Cdkn1a function, or agents that inhibit active DNA demethylation, or a combination thereof, thereby increasing skeletal muscle oxidative metabolism. In an aspect, inhibiting and providing steps are performed in vitro. In an aspect, inhibiting and providing steps are performed in vivo. In an aspect, inhibiting and providing steps in an animal. In an aspect, the animal is a primate. In an aspect, the animal is a mammal. In an aspect, the animal is a human.

In one aspect, the method does comprise agents that increase androgen and/or growth hormone signaling. In an aspect, the composition comprises a therapeutically effective amount of one or more agents that inhibit Gadd45a expression and/or Cdkn1a expression, agents that inhibit Gadd45a function and/or Cdkn1a function, or agents that inhibit active DNA demethylation, or a combination thereof. In an aspect, the composition comprises a prophylactically effective amount of one or more agents that inhibit Gadd45a expression and/or Cdkn1a expression, agents that inhibit Gadd45a function and/or Cdkn1a function, or agents that inhibit active DNA demethylation, or a combination thereof. In an aspect, the one or more agents that inhibit Gadd45a expression and/or Cdkn1a expression, agents that inhibit Gadd45a function and/or Cdkn1a function, or agents that inhibit active DNA demethylation, or a combination thereof comprises ursolic acid. In an aspect, the one or more agents that inhibit Gadd45a expression and/or Cdkn1a expression, agents that inhibit Gadd45a function and/or Cdkn1a function, or agents that inhibit active DNA demethylation, or a combination thereof comprises an ursolic acid derivative. In a further aspect, the one or more agents that inhibit Gadd45a expression and/or Cdkn1a expression, agents that inhibit Gadd45a function and/or Cdkn1a function, or agents that inhibit active DNA demethylation, or a combination thereof comprises RNA interference. In a further aspect, the one or more agents that inhibit Gadd45a expression and/or Cdkn1a expression, agents that inhibit Gadd45a function and/or Cdkn1a function, or agents that inhibit active DNA demethylation, or a combination thereof comprises antisense oligonucleotides.

g. Increasing Skeletal Muscle Blood Flow

Also disclosed herein is a method of increasing skeletal muscle blood flow comprising, administering to an animal an effective amount of a composition comprising one or more agents that inhibit Gadd45a expression and/or Cdkn1a expression, agents that inhibit Gadd45a function and/or Cdkn1a function, or agents that inhibit active DNA demethylation, or a combination thereof, thereby increasing skeletal muscle blood flow. In an aspect, inhibiting and providing steps are performed in vivo. In an aspect, inhibiting and providing steps in an animal. In an aspect, the animal is a primate. In an aspect, the animal is a mammal. In an aspect, the animal is a human.

In one aspect, the composition does not comprise agents that increase androgen and/or growth hormone signaling. In an aspect, the composition comprises a therapeutically effective amount of one or more agents that inhibit Gadd45a expression and/or Cdkn1a expression, agents that inhibit Gadd45a function and/or Cdkn1a function, or agents that inhibit active DNA demethylation, or a combination thereof. In an aspect, the composition comprises a prophylactically effective amount of one or more agents that inhibit Gadd45a expression and/or Cdkn1a expression, agents that inhibit Gadd45a function and/or Cdkn1a function, or agents that inhibit active DNA demethylation, or a combination thereof. In an aspect, the one or more agents that inhibit Gadd45a expression and/or Cdkn1a expression, agents that inhibit Gadd45a function and/or Cdkn1a function, or agents that inhibit active DNA demethylation, or a combination thereof comprises ursolic acid. In an aspect, the one or more agents that inhibit Gadd45a expression and/or Cdkn1a expression, agents that inhibit Gadd45a function and/or Cdkn1a function, or agents that inhibit active DNA demethylation, or a combination thereof comprises an ursolic acid derivative. In a further aspect, the one or more agents that inhibit Gadd45a expression and/or Cdkn1a expression, agents that inhibit Gadd45a function and/or Cdkn1a function, or agents that inhibit active DNA demethylation, or a combination thereof comprises RNA interference. In a further aspect, the one or more agents that inhibit Gadd45a expression and/or Cdkn1a expression, agents that inhibit Gadd45a function and/or Cdkn1a function, or agents that inhibit active DNA demethylation, or a combination thereof comprises antisense oligonucleotides.

h. Increasing Skeletal Muscle Energy Expenditure

Also disclosed herein is a method of increasing skeletal muscle energy expenditure comprising, administering to an animal an effective amount of one or more agents that inhibit Gadd45a expression and/or Cdkn1a expression, agents that inhibit Gadd45a function and/or Cdkn1a function, or agents that inhibit active DNA demethylation, or a combination thereof, thereby of increasing skeletal muscle energy expenditure. In an aspect, inhibiting and providing steps are performed in vitro. In an aspect, inhibiting and providing steps are performed in vivo. In an aspect, inhibiting and providing steps in an animal. In an aspect, the animal is a primate. In an aspect, the animal is a mammal. In an aspect, the animal is a human.

In one aspect, the method does comprise agents that increase androgen and/or growth hormone signaling. In an aspect, the composition comprises a therapeutically effective amount of one or more agents that inhibit Gadd45a expression and/or Cdkn1a expression, agents that inhibit Gadd45a function and/or Cdkn1a function, or agents that inhibit active DNA demethylation, or a combination thereof. In an aspect, the composition comprises a prophylactically effective amount of one or more agents that inhibit Gadd45a expression and/or Cdkn1a expression, agents that inhibit Gadd45a function and/or Cdkn1a function, or agents that inhibit active DNA demethylation, or a combination thereof. In an aspect, the one or more agents that inhibit Gadd45a expression and/or Cdkn1a expression, agents that inhibit Gadd45a function and/or Cdkn1a function, or agents that inhibit active DNA demethylation, or a combination thereof comprises ursolic acid. In an aspect, the one or more agents that inhibit Gadd45a expression and/or Cdkn1a expression, agents that inhibit Gadd45a function and/or Cdkn1a function, or agents that inhibit active DNA demethylation, or a combination thereof comprises an ursolic acid derivative. In a further aspect, the one or more agents that inhibit Gadd45a expression and/or Cdkn1a expression, agents that inhibit Gadd45a function and/or Cdkn1a function, or agents that inhibit active DNA demethylation, or a combination thereof comprises RNA interference. In a further aspect, the one or more agents that inhibit Gadd45a expression and/or Cdkn1a expression, agents that inhibit Gadd45a function and/or Cdkn1a function, or agents that inhibit active DNA demethylation, or a combination thereof comprises antisense oligonucleotides.

i. Enhancing Muscle Formation

In one aspect, the invention relates to a method of enhancing muscle formation in a mammal, the method comprising administering to the mammal a compound of the formula:

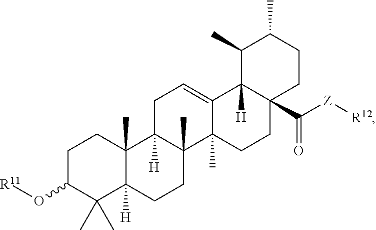

wherein R¹¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, R¹² and R¹³, when present, are covalently bonded and —NR¹²R¹³ comprises a moiety represented by the formula:

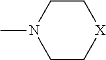

wherein X is selected from O, S, SO, SO₂, NH and NCH₃; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; or a pharmaceutically acceptable salt, hydrate, solvate, or polymorph thereof, in an amount of at least about 200 mg/kg and effective to enhance muscle formation in the mammal.

In a further aspect, the compound administered is a disclosed compound or a product of a disclosed method of making a compound.

In a further aspect, the mammal is a human. In a still further aspect, the human is a patient. In a yet further aspect, administration of the compound prevents muscle atrophy in the mammal. In an even further aspect, administration of the compound treats muscle atrophy in the mammal. In a still further aspect, administration of the compound increases muscle mass in the mammal. In a yet further aspect, administration of the compound increases muscular strength in the mammal.

In a further aspect, the compound is administered in an effective amount. In a yet further aspect, the effective amount is a therapeutically effective amount. In a still further aspect, the effective amount is a prophylactically effective amount. In a still further aspect, the method further comprises the step of identifying the mammal in need of treatment of muscle atrophy. In a yet further aspect, the method further comprises the step of identifying the mammal in need of prevention of muscle atrophy. In an even further aspect, the mammal has been diagnosed with a need for treatment of muscle atrophy prior to the administering step.

In a further aspect. the mammal is a domesticated animal. In a yet further aspect, domesticated animal is livestock. In a yet further aspect, the livestock animal is selected from pig, cow, horse, goat, bison, and sheep.

In a further aspect, the compound is not ursolic acid. In a still further aspect, the compound is ursolic acid. In a further aspect, the compound is not ursolic acid, beta-boswellic acid, corosolic acid, betulinic acid, or UA0713. In a still further aspect, the compound is ursolic acid, beta-boswellic acid, corosolic acid, betulinic acid, or UA0713. In yet further aspect, the compound is not administered as a foodstuff

iii) Enhancing Tissue Growth in Vitro

In one aspect, the invention relates to a method of enhancing tissue growth in vitro, the method comprising administering to the tissue a compound of the formula:

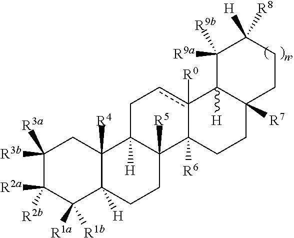

wherein each [symbol] is an optional covalent bond, and R⁰ is optionally present; wherein n is 0 or 1; wherein R⁰, when present, is hydrogen; wherein R^{1a} is selected from C1-C6 alkyl and —C(O)ZR¹⁰; wherein R^{1b} is selected from C1-C6 alkyl, or wherein R^{1a} and R^{1b} are optionally covalently bonded and, together with the intermediate carbon, comprise an optionally substituted C3-C5 cycloalkyl or C2-C5 heterocycloalkyl; wherein one of R^{2a} and R^{2b} is —OR¹¹, and the other is hydrogen, or R^{2a} and R^{2b} together comprise ═O; wherein each of R^{3a} and R^{3b} is independently selected from hydrogen, hydroxyl, C1-C6 alkyl, and C1-C6 alkoxyl, provided that R^{3a} and R^{3b} are not simultaneously hydroxyl, wherein R^{3a} and R^{3b} are optionally covalently bonded and, together with the intermediate carbon, comprise an optionally substituted C3-C5 cycloalkyl or C2-C5 heterocycloalkyl; wherein each of R⁴, R⁵, and R⁶ is independently selected from C1-C6 alkyl; wherein R⁷ is selected from C1-C6 alkyl, —CH₂OR¹² and —C(O)ZR¹²; wherein R⁸ is selected from hydrogen and C1-C6 alkyl; wherein each of R^{9a} and R^{9b} is independently selected from hydrogen and C1-C6 alkyl, provided that R^{9a} and R^{9b} are not simultaneously hydrogen; or wherein R^{9a} and R^{9b} are covalently bonded and, along with the intermediate carbon, together comprise C3-C5 cycloalkyl or C2-C5 heterocycloalkyl; wherein R¹⁰ is selected from hydrogen and C1-C6 alkyl; wherein R¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹ permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, wherein Z is N, R¹² and R¹³ are covalently bonded and —NR¹²R¹³ comprises a moiety of the formula:

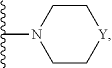

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; or a pharmaceutically acceptable salt, hydrate, solvate, or polymorph thereof, in an amount effective to enhance growth of the tissue.

In a further aspect, the compound administered is a disclosed compound or a product of a disclosed method of making a compound.

In a further aspect, the tissue comprises animal cells. In a still further aspect, the animal cells are muscle cells. In a yet further aspect, the muscle cells are myosatellite cells. In an even further aspect, the myosatellite cells are grown on a scaffold.

iv) Manufacture of a Medicament

In one aspect, the invention relates to a method for the manufacture of a medicament for inhibiting muscle atrophy and for increasing muscle mass in a mammal comprising combining a therapeutically effective amount of a disclosed compound or product of a disclosed method with a pharmaceutically acceptable carrier or diluent.

In a further aspect, the medicament is modulates muscle growth. In a still further aspect, the medicament inhibits muscle atrophy. In a yet further aspect, the medicament increases muscle mass. In an even further aspect, the medicament induces skeletal muscle hypertrophy.

v) Methods of Testing for Performance Enhancing Use

In one aspect, the invention relates to a method of testing for performance enhancing use of a ursolic acid analog in an animal, the method comprising: (a) obtaining a biological test sample from the animal; and (b) measuring the amount of a compound of formula:

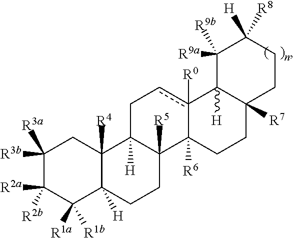

wherein each [symbol] is an optional covalent bond, and R⁰ is optionally present; wherein n is 0 or 1; wherein R⁰, when present, is hydrogen; wherein R^{1a} is selected from C1-C6 alkyl and —C(O)ZR¹⁰; wherein R^{1b} is selected from C1-C6 alkyl, or wherein R^{1a} and R^{1b} are optionally covalently bonded and, together with the intermediate carbon, comprise an optionally substituted C3-C5 cycloalkyl or C2-C5 heterocycloalkyl; wherein one of R^{2a} and R^{2b} is —OR¹¹, and the other is hydrogen, or R^{2a} and R^{2b} together comprise ═O; wherein each of R^{3a} and R^{3b} is independently selected from hydrogen, hydroxyl, C1-C6 alkyl, and C1-C6 alkoxyl, provided that R^{3a} and R^{3b} are not simultaneously hydroxyl, wherein R^{3a} and R^{3b} are optionally covalently bonded and, together with the intermediate carbon, comprise an optionally substituted C3-C5 cycloalkyl or C2-C5 heterocycloalkyl; wherein each of R⁴, R⁵, and R⁶ is independently selected from C1-C6 alkyl; wherein R⁷ is selected from C1-C6 alkyl, —CH₂OR¹² and —C(O)ZR¹²; wherein R⁸ is selected from hydrogen and C1-C6 alkyl; wherein each of R^{9a} and R^{9b} is independently selected from hydrogen and C1-C6 alkyl, provided that R^{9a} and R^{9b} are not simultaneously hydrogen; or wherein R^{9a} and R^{9b} are covalently bonded and, along with the intermediate carbon, together comprise C3-C5 cycloalkyl or C2-C5 heterocycloalkyl; wherein R¹⁰ is selected from hydrogen and C1-C6 alkyl; wherein R¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹ permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, wherein Z is N, R¹² and R¹³ are covalently bonded and —NR¹²R¹³ comprises a moiety of the formula:

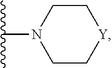

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; or a pharmaceutically acceptable salt, hydrate, solvate, or polymorph thereof, in the test sample to determine whether a superphysiological amount of the compound is present in the biological test sample; wherein the superphysiological amount of the compound in the biological test sample is indicative of performance enhancing use of the compound.

In a further aspect, the superphysiological amount is greater than the peak concentration from administration at a level of about 1000 mg per day. In a still further aspect, the superphysiological amount is the amount that results from administration of the compound at a level greater than 200 mg per day. In a still further aspect, the superphysiological amount is the amount resulting from administration of the compound at a level greater than 200 mg per day. In an even further aspect, the biological test sample is obtained about 12 hours to about 96 hours following administration of the compound.

In a further aspect, the animal is a mammal. In a yet further aspect, the animal is a domesticated animal. In a still further aspect, the mammal is a human.

In a further aspect, the biological sample is blood, urine, saliva, hair, muscle, skin, fat, or breath.

vi) Use of Compositions

In one aspect, the invention relates to the use of a composition for increasing muscle mass in a mammal, the compound having a structure represented by a formula:

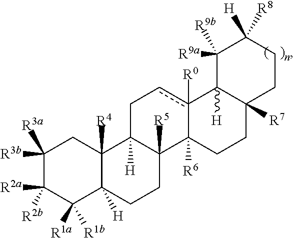

wherein each [symbol] is an optional covalent bond, and R⁰ is optionally present; wherein n is 0 or 1; wherein R⁰, when present, is hydrogen; wherein R^{1a} is selected from C1-C6 alkyl and —C(O)ZR¹⁰; wherein R^{1b} is selected from C1-C6 alkyl, or wherein R^{1a} and R^{1b} are optionally covalently bonded and, together with the intermediate carbon, comprise an optionally substituted C3-C5 cycloalkyl or C2-C5 heterocycloalkyl; wherein one of R^{2a} and R^{2b} is —OR¹¹, and the other is hydrogen, or R^{2a} and R^{2b} together comprise ═O; wherein each of Ria and R^{3b} is independently selected from hydrogen, hydroxyl, C1-C6 alkyl, and C1-C6 alkoxyl, provided that R^{3a} and R^{3b} are not simultaneously hydroxyl, wherein R^{3a} and R^{3b} are optionally covalently bonded and, together with the intermediate carbon, comprise an optionally substituted C3-C5 cycloalkyl or C2-C5 heterocycloalkyl; wherein each of R⁴, R⁵, and R⁶ is independently selected from C1-C6 alkyl; wherein R⁷ is selected from C1-C6 alkyl, —CH₂OR¹² and —C(O)ZR¹²; wherein R⁸ is selected from hydrogen and C1-C6 alkyl; wherein each of R^{9a} and R^{9b} is independently selected from hydrogen and C1-C6 alkyl, provided that R^{9a} and R^{9b} are not simultaneously hydrogen; or wherein R^{9a} and R^{9b} are covalently bonded and, along with the intermediate carbon, together comprise C3-C5 cycloalkyl or C2-C5 heterocycloalkyl; wherein R¹⁰ is selected from hydrogen and C1-C6 alkyl; wherein R¹¹ is selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, R¹² and R¹³, when present, are covalently bonded and —NR¹²R¹³ comprises a moiety represented by the formula:

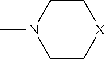

wherein X is selected from O, S, SO, SO₂, NH and NCH₃; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; or a pharmaceutically acceptable salt, hydrate, solvate, or polymorph thereof

In a further aspect, a use is the treatment of a mammal. In a yet further aspect, the mammal is a human. In a still further aspect, the human is a patient. In a yet further aspect, a use is administration of the compound to a mammal to prevent muscle atrophy. In a yet further aspect, a use is administration of the compound to increase muscular strength in the mammal. In a further aspect. the mammal is a domesticated animal. In a yet further aspect, domesticated animal is livestock. In a yet further aspect, the livestock animal is selected from pig, cow, horse, goat, bison, and sheep.

In a further aspect, a use is administration of the compound in an effective amount. In a yet further aspect, the effective amount is a therapeutically effective amount. In a still further aspect, the effective amount is a prophylactically effective amount. In a still further aspect, prior to use the mammal in need of treatment of muscle atrophy is identified. In a yet further aspect, prior to use the mammal in need of prevention of muscle atrophy is identified. In an even further aspect, the mammal has been diagnosed with a need for treatment of muscle atrophy prior to the administering step.

In a further aspect, the compound is not ursolic acid. In a still further aspect, the compound is ursolic acid. In a further aspect, the compound is not ursolic acid, beta-boswellic acid, corosolic acid, betulinic acid, or UA0713. In a still further aspect, the compound is ursolic acid, beta-boswellic acid, corosolic acid, betulinic acid, or UA0713. In yet further aspect, the compound is not used as a foodstuff. In an even further aspect, the compound is used in an amount is greater than about 1000 mg per day when the compound is ursolic acid, beta-boswellic acid, corosolic acid, betulinic acid, or UA0713.

vii) Reduction of Gadd45 and/or Cdkn1a Expression

Gadd45a expression can be reduced in several ways. First, Gadd45a gene transcription can be reduced by increasing the expression or function of a protein that decreases transcription of the Gadd45a gene (including but not limited to Myc and ZBRK1); or by decreasing the expression or function of protein that increases transcription of the Gadd45a gene (including but not limited to PERK, PKR, HRI, GCN2, ATF4, ATF2, FoxO1, FoxO3a, ATM, p53, BRCA1, WT1, Oct-1, NF-I, NF-Y, Egr-1 and C/EBPα) (Lal, A., et al. (2006) Cell cycle (Georgetown, Tex. 5, 1422-1425; Ebert, S. M., et al. (2010) Molecular Endocrinology 24, 790-799; Ebert, S. M., et al. (2012) The Journal of biological chemistry 287, 27290-273011; Tran, H., et al. (2002) Science (New York, N.Y 296, 530-534; Kamei, Y., et al. (2004) The Journal of biological chemistry 279, 41114-41123; Jiang, H. Y., Jet al. (2007) The Journal of biological chemistry 282, 3755-3765; Zhan, Q. (2005) Mutation research 569, 133-143; Reinhardt, H. C., et al. (2010) Mol Cell 40, 34-49). Disclosed herein is a method of reducing Gadd45a gene transcription comprising decreasing the expression or function of protein that increases transcription of the Gadd45a gene comprising administering to an animal an effective amount of a composition that decreases the expression or function of protein that increases transcription of the Gadd45a gene, wherein the protein comprises PERK, PKR, HRI, GCN2, ATF4, ATF2, FoxO1, FoxO3a, ATM, p53, BRCA1, WT1, Oct-1, NF-I, NF-Y, Egr-1, C/EBPα, or a combination thereof, thereby reducing Gadd45a gene transcription. In one aspect, the composition that decreases the expression or function of protein that increases transcription of the Gadd45a gene is co-administered with a compound or composition disclosed herein. Also disclosed herein is a method of reducing Gadd45a gene transcription comprising increasing the expression or function of a protein that decreases transcription of the Gadd45a gene comprising administering to an animal an effective amount of a composition that increases the expression or function of a protein that decreases transcription of the Gadd45a gene, wherein the protein comprises Myc, ZBRK1, or a combination thereof, thereby reducing Gadd45a gene transcription. In one aspect, the composition that increases the expression or function of a protein that decreases transcription of the Gadd45a gene is co-administered with a compound or composition disclosed herein.

Second, the stability of Gadd45a mRNA can be reduced by increasing the expression or function of a protein or microRNA that increases degradation of Gadd45a mRNA (including but not limited to AUF1 and PARN); or by decreasing the expression or function of a protein that stabilizes Gadd45a mRNA (including but not limited to nucleolin, p38, MK2 and hnRNPA0) (Lal, A., et al. (2006) Cell cycle (Georgetown, Tex. 5, 1422-1425; Reinhardt, H. C., et al. (2010) Mol Cell 40, 34-49). Disclosed herein is a method of reducing the stability of Gadd45a mRNA comprising increasing the expression or function of a protein or microRNA that increases degradation of Gadd45a mRNA comprising administering to an animal an effective amount of a composition that increases the expression or function of a protein or microRNA that increases degradation of Gadd45a mRNA, wherein the protein or microRNA comprises AUF1, PARN, or a combination thereof, thereby reducing the stability of Gadd45a mRNA. In one aspect, the composition that increases the expression or function of a protein or microRNA that increases degradation of Gadd45a mRNA is co-administered with a compound or composition disclosed herein.

Third, the translation of Gadd45a mRNA can be decreased by increasing the expression or function of a protein that decreases Gadd45a mRNA translation (including but not limited to TIAR) (Lal, A., et al. (2006) Cell cycle (Georgetown, Tex. 5, 1422-1425; Reinhardt, H. C., et al. (2010) Mol Cell 40, 34-49). Disclosed herein is a method of decreasing the translation of Gadd45a mRNA comprising increasing the expression or function of a protein that decreases Gadd45a mRNA translation comprising administering to an animal an effective amount of a composition that increases the expression or function of a protein that decreases Gadd45a mRNA translation, wherein the protein comprises TIAR, thereby decreasing the translation of Gadd45a mRNA. In one aspect, the composition that increases the expression or function of a protein that decreases Gadd45a mRNA translation is co-administered with a compound or composition disclosed herein.

Gadd45a function and Cdkn1a gene demethylation can be reduced by decreasing the expression or function of a protein that facilitates Gadd45a-mediated DNA demethylation (including but not limited to XPA, XPC, XPF, CSB, XPG, TAF12, AID, Apobec enxymes, Mbd4 and TDG); or by increasing the expression or function of a protein that increases methylation of the Cdkn1a gene (including but not limited to DNMT3A, DNMT3B and DNMT3L) (Chedin, F. (2011) Progress in molecular biology and translational science 101, 255-285; Niehrs, C., et al. (2012) Trends in cell biology 22, 220-227; Le May, N., et al. (2010) Mol Cell 38, 54-66; Brenner, C., et al. (2005) The EMBO journal 24, 336-346). Disclosed herein is a method of reducing Gadd45a function and Cdkn1a gene demethylation comprising decreasing the expression or function of a protein that facilitates Gadd45a-mediated DNA demethylation comprising administering to an animal an effective amount of a composition that decreases the expression or function of a protein that facilitates Gadd45a-mediated DNA demethylation, wherein the protein comprises XPA, XPC, XPF, CSB, XPG, TAF12, AID, Apobec enxymes, Mbd4, TDG, or a combination thereof, thereby reducing Gadd45a function and Cdkn1a gene demethylation. In one aspect, the composition that decreases the expression or function of a protein that facilitates Gadd45a-mediated DNA demethylation can be co-administered with a compound or composition disclosed herein. Also disclosed herein is a method of reducing Gadd45a function and Cdkn1a gene demethylation increasing the expression or function of a protein that increases methylation of the Cdkn1a gene comprising administering to an animal an effective amount of a composition that increases the expression or function of a protein that increases methylation of the Cdkn1a gene, wherein the protein comprises DNMT3A, DNMT3B, DNMT3L, or a combination thereof, thereby reducing Gadd45a function and Cdkn1a gene demethylation. In one aspect, the composition that increases the expression or function of a protein that increases methylation of the Cdkn1a gene is co-administered with a compound or composition disclosed herein.

Cdkn1a expression can be reduced by increasing the expression or function of a protein that decreases transcription of the Cdkn1a gene (including but not limited to Myc, MIZ1, DNMT3A and AP4); or by decreasing the expression or function of protein that increases transcription of the Cdkn1a gene (including but not limited to SMAD2, SMAD3, SMAD4, p53, p73, KLF4, KLF6, GAX, HOXA10, E2F1, E2F3, BRCA1, STAT1, STAT3, STAT5, C/EBPα, C/EBPβ, Sp1, Sp3, MYOD1, NEUROD1, retinoic acid receptors and the vitamin D receptor) (Abbas, T., et al. (2009) Nat Rev Cancer 9, 400-414). Disclosed herein is a method of reducing Cdkn1a expression comprising increasing the expression or function of a protein that decreases transcription of the Cdkn1a gene comprising administering to an animal an effective amount of a composition that increases the expression or function of a protein that decreases transcription of the Cdkn1a gene, wherein the protein comprises Myc, MIZ1, DNMT3A, AP4, or a combination thereof, thereby reducing Cdkn1a expression. In one aspect, the composition that increases the expression or function of a protein that decreases transcription of the Cdkn1a gene can be co-administered with a compound or composition disclosed herein. Also disclosed herein is a method of reducing Cdkn1a expression comprising decreasing the expression or function of protein that increases transcription of the Cdkn1a gene comprising administering to an animal an effective amount of a composition that decreases the expression or function of protein that increases transcription of the Cdkn1a gene, wherein the protein comprises SMAD2, SMAD3, SMAD4, p53, p73, KLF4, KLF6, GAX, HOXA10, E2F1, E2F3, BRCA1, STAT1, STAT3, STAT5, C/EBPα, C/EBPβ, Sp1, Sp3, MYOD1, NEUROD1, retinoic acid receptors, the vitamin D receptor, or a combination thereof, thereby reducing Cdkn1a expression. In one aspect, the composition that decreases the expression or function of protein that increases transcription of the Cdkn1a gene can be co-administered with a compound or composition disclosed herein.

Cdkn1a expression can also be decreased by altering the expression or function of proteins or microRNAs that regulate the stability and translation of Cdkn1a mRNA (Jung, Y. S., et al. (2010) Cell Signal 22, 1003-1012), or by increasing the activity of proteins that decrease the stability of Cdkn1a protein (including but not limited to SKP1, CUL1, SKP2 CUL4, DDB1, CDT2 and anaphase-promoting-complex-cell division cycle 20) (Abbas, T., et al. (2009) Nat Rev Cancer 9, 400-414). Disclosed herein is a method of reducing Cdkn1a expression comprising altering the expression or function of proteins or microRNAs that regulate the stability and translation of Cdkn1a mRNA comprising administering to an animal an effective amount of a composition that alters the expression or function of proteins or microRNAs that regulate the stability and translation of Cdkn1a mRNA, wherein the protein comprises SKP1, CUL1, SKP2 CUL4, DDB1, CDT2, anaphase-promoting-complex-cell division cycle 20, or a combination thereof, thereby reducing the Cdkn1a expression. In one aspect, the composition that alters the expression or function of proteins or microRNAs that regulate the stability and translation of Cdkn1a mRNA can be co-administered with a compound or composition disclosed herein. Also disclosed herein is a method of reducing Cdkn1a expression comprising increasing the activity of proteins that decrease the stability of Cdkn1a protein comprising administering to an animal an effective amount of a composition that increases the activity of proteins that decrease the stability of Cdkn1a protein, wherein the protein comprises SKP1, CUL1, SKP2 CUL4, DDB1, CDT2, anaphase-promoting-complex-cell division cycle 20, or a combination thereof, thereby reducing the Cdkn1a expression. In one aspect, the composition that increases the activity of proteins that decrease the stability of Cdkn1a protein is co-administered with a compound or composition disclosed herein.

Cdkn1a function can be reduced by decreasing the expression or function of a protein whose activity directly or indirectly requires Cdkn1a (including but not limited to CDK8 and Rb); or by increasing the expression or function of a protein whose activity is inhibited by Cdkn1a (including but not limited to CDK1, CDK2 and CDK4) (Abbas, T., et al. (2009) Nat Rev Cancer 9, 400-414; Porter, D. C., et al. (2012) Proceedings of the National Academy of Sciences of the United States of America 109, 13799-13804). Disclosed herein is a method of reducing Cdkn1a function comprising decreasing the expression or function of a protein whose activity directly or indirectly requires Cdkn1a comprising administering to an animal an effective amount of a composition that decreases the expression or function of a protein whose activity directly or indirectly requires Cdkn1a, wherein the protein comprises CDK8, Rb, or a combination thereof, thereby reducing Cdkn1a function. In one aspect, the composition that decreases the expression or function of a protein whose activity directly or indirectly requires Cdkn1a is co-administered with a compound or composition disclosed herein. Disclosed herein is a method of reducing Cdkn1a function comprising increasing the expression or function of a protein whose activity is inhibited by Cdkn1a comprising administering to an animal an effective amount of a composition that increases the expression or function of a protein whose activity is inhibited by Cdkn1a, wherein the protein comprises CDK1, CDK2, CDK4, or a combination thereof, thereby reducing Cdkn1a function. In one aspect, the composition that increases the expression or function of a protein whose activity is inhibited by Cdkn1a is co-administered with a compound or composition disclosed herein.

viii) Screening Methods

Disclosed herein is a screening method, comprising the steps of administering a candidate inhibitor to a cell, and measuring expression of Gadd45a and/or Cdkn1a in the cell, wherein decreased expression in the cell relative to a control cell identifies a potential treatment or preventative for muscle atrophy. In an aspect, the cell can be a skeletal muscle cell. In an aspect, the cell can be a muscle fiber. In a further aspect, the cell can be a myotube. In a further aspect, cell can be a myoblast. In an aspect, the cell can be a stem cell.

ix) Kits

Disclosed herein is a kit comprising a Gadd45a and/or Cdkn1a inhibitor and an androgen and/or growth hormone elevator. In an aspect, the inhibitor and the elevator are coformulated. In a further aspect, the inhibitor and the elevator are copackaged. In an aspect, the kit further comprises instructions for treatment of skeletal muscle atrophy

In an aspect, the inhibitor of the disclosed kit is ursolic acid and the elevator of the disclosed kit is growth hormone. In an aspect, the inhibitor is ursolic acid and the elevator is an androgen. In an aspect, the inhibitor is ursolic acid and the elevator is ghrelin. In an aspect, the inhibitor is ursolic acid and the elevator is a ghrelin analog. Ghrelin analogs include, but are not limited to, BIM-28125 and BIM-28131. In an aspect, the inhibitor is ursolic acid and the elevator increases expression of activity of ghrelin. In an aspect, the inhibitor is ursolic acid and the elevator is an aromatase inhibitor.

In an aspect, the inhibitor of the disclosed kit is an ursolic acid derivative and the elevator of the disclosed kit is growth hormone. In an aspect, the inhibitor is an ursolic acid derivative and the elevator is an androgen. In an aspect, the inhibitor is ursolic acid and the elevator is ghrelin. In an aspect, the inhibitor is an ursolic acid derivative and the elevator is a ghrelin analog. Ghrelin analogs include, but are not limited to, BIM-28125 and BIM-28131. In an aspect, the inhibitor is an ursolic acid derivative and the elevator increases expression of activity of ghrelin. In an aspect, the inhibitor is an ursolic acid derivative and the elevator is an aromatase inhibitor.

In an aspect, the inhibitor of the disclosed kit is RNA interference and the elevator of the disclosed kit is growth hormone. In an aspect, the inhibitor is RNA interference and the elevator is an androgen. In an aspect, the inhibitor is RNA interference and the elevator is ghrelin. In an aspect, the inhibitor is RNA inteference and the elevator is a ghrelin analog. Ghrelin analogs include, but are not limited to, BIM-28125 and BIM-28131. In an aspect, the inhibitor is RNA interference and the elevator increases expression of activity of ghrelin. In an aspect, the inhibitor is RNA interference and the elevator is an aromatase inhibitor. In an aspect, the RNA interference targets Gadd45a and/or Cdkn1a. In a further aspect, the RNA interference is miRNA targeting Gadd45a and/or Cdkn1a. In a further aspect, the RNA interference is siRNA targeting Gadd45a and/or Cdkn1a. In yet a further aspect, the RNA interference is shRNA targeting Gadd45a and/or Cdkn1a.

In an aspect, the inhibitor of the disclosed kit is one or more antisense oligonucleotide molecules and the elevator of the disclosed kit is growth hormone. In an aspect, the inhibitor is one or more antisense oligonucleotide molecules and the elevator is an androgen. In an aspect, the inhibitor is one or more antisense oligonucleotide molecules nd the elevator is ghrelin. In an aspect, the inhibitor is one or more antisense oligonucleotide molecules and the elevator is a ghrelin analog. Ghrelin analogs include, but are not limited to, BIM-28125 and BIM-28131. In an aspect, the inhibitor is one or more antisense oligonucleotide molecules and the elevator increases expression of activity of ghrelin. In an aspect, the inhibitor is one or more antisense oligonucleotide molecules and the elevator is an aromatase inhibitor. In an aspect, the one or more antisense oligonucleotide molecules target Gadd45a and/or Cdkn1a.

Disclosed herein is a kit comprising a Gadd45a and/or Cdkn1a inhibitor and instructions for administering an androgen and/or growth hormone elevator. In an aspect, the inhibitor of the disclosed kit is ursolic acid. In an aspect, the inhibitor is an ursolic acid derivative. In a further aspect, the inhibitor is RNA interference. In an aspect, the inhibitor is one or more antisense oligonucleotide molecules. The elevator can be growth hormone, an androgen, ghrelin, a ghrelin analog, something that increases the activity or expression of ghrelin, or an aromatase inhibitor.

Disclosed herein is a kit comprising an androgen and/or growth hormone elevator and instructions for administering a Gadd45a and/or Cdkn1a inhibitor. In an aspect, the elevator can be growth hormone. In an aspect, the elevator can be an androgen. In an aspect, the elevator can be ghrelin, a ghrelin analog, or something that increases the activity or expression of ghrelin. In an aspect, the elevator can be an aromatase inhibitor. The inhibitor can be ursolic acid, an ursolic acid derivative, RNA interference, or one or more antisense oligonucleotide molecules.

Disclosed herein is a kit comprising a Gadd45a and/or Cdkn1a inhibitor and an androgen and/or growth hormone receptor activator. In an aspect, the inhibitor and the activator are coformulated. In a further aspect, the inhibitor and the activator are copackaged. In an aspect, the kit further comprises instructions for treatment of skeletal muscle atrophy.

In an aspect, the inhibitor of the disclosed kit is ursolic acid and the activator of the disclosed kit growth hormone. In an aspect, the inhibitor is ursolic acid and the activator is an androgen. In an aspect, the inhibitor is ursolic acid and the activator is a selective androgen receptor modulator. In an aspect, the inhibitor is ursolic acid and the activator is a protein tyrosine phosphatase inhibitor.

In an aspect, the inhibitor of the disclosed kit is an ursolic acid derivative and the activator of the disclosed kit is growth hormone. In an aspect, the inhibitor is an ursolic acid derivative and the activator is an androgen. In an aspect, the inhibitor is an ursolic acid derivative and the activator is a selective androgen receptor modulator. In an aspect, the inhibitor is an ursolic acid derivative and the activator is a protein tyrosine phosphatase inhibitor.

In an aspect, the inhibitor of the disclosed kit is RNA interference and the activator of the disclosed kit is growth hormone. In an aspect, the inhibitor is RNA interference and the activator is an androgen. In an aspect, the inhibitor is RNA interference and the activator is a selective androgen receptor modulator. In an aspect, the inhibitor is RNA interference and the activator is a protein tyrosine phosphatase inhibitor. In an aspect, the RNA interference targets Gadd45a and/or Cdkn1a. In a further aspect, the RNA interference is miRNA targeting Gadd45a and/or Cdkn1a. In a further aspect, the RNA interference is siRNA targeting Gadd45a and/or Cdkn1a. In yet a further aspect, the RNA interference is shRNA targeting Gadd45a and/or Cdkn1a.

In an aspect, the inhibitor of the disclosed kit is one or more antisense oligonucleotide molecules and the activator of the disclosed kit is growth hormone. In an aspect, the inhibitor is one or more antisense oligonucleotide molecules and the activator is an androgen. In an aspect, the inhibitor is one or more antisense oligonucleotide molecules and the activator is a selective androgen receptor modulator. In an aspect, the inhibitor is one or more antisense oligonucleotide molecules and the activator is a protein tyrosine phosphatase inhibitor.

Disclosed herein is a kit comprising a Gadd45a and/or Cdkn1a inhibitor and instructions for administering an androgen and/or growth hormone receptor activator. In an aspect, the inhibitor of the disclosed kit is ursolic acid. In an aspect, the inhibitor is an ursolic acid derivative. In a further aspect, the inhibitor is RNA interference. In an aspect, the inhibitor is one or more antisense oligonucleotide molecules. The activator can be growth hormone, a steroid androgen, a selective androgen receptor modulator, or a protein tyrosine phosphatase inhibitor.

Disclosed herein is a kit comprising an androgen and/or growth hormone receptor activator and instructions for administering a Gadd45a and/or Cdkn1a inhibitor. In an aspect, the activator can be growth hormone. In an aspect, the activator can be an androgen. In an aspect, the activator can be a selective androgen receptor modulator. In an aspect, the activator can be a protein tyrosine phosphatase inhibitor. The inhibitor can be ursolic acid, an ursolic acid derivative, RNA interference, or one or more antisense oligonucleotide molecules.

In one aspect, the invention relates to a kit comprising at least one compound having a structure represented by a formula:

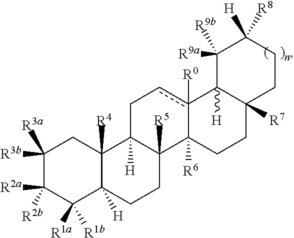

wherein each [symbol] is an optional covalent bond, and R⁰ is optionally present; wherein n is 0 or 1; wherein R⁰, when present, is hydrogen; wherein R^{1a} is selected from C1-C6 alkyl and —C(O)ZR¹⁰; wherein R^{1b} is selected from C1-C6 alkyl; or wherein R^{1a} and R^{1b} are covalently bonded and, along with the intermediate carbon, together comprise optionally substituted C3-C5 cycloalkyl or optionally substituted C2-C5 heterocycloalkyl; wherein R^{2a} and R^{2b} are independently selected from hydrogen and —OR¹¹, provided that at least one of R^{2a} and R^{2b} is —OR¹¹; or wherein R^{2a} and R^{2b} together comprise ═O; wherein each of R^{3a} and R^{3b} is independently selected from hydrogen, hydroxyl, C1-C6 alkyl, and C1-C6 alkoxyl, provided that R^{3a} and R^{3b} are not simultaneously hydroxyl; or wherein R^{3a} and R^{3b} are covalently bonded and, along with the intermediate carbon, together comprise optionally substituted C3-C5 cycloalkyl or optionally substituted C2-C5 heterocycloalkyl; wherein each of R⁴, R⁵, and R⁶ is independently selected from C1-C6 alkyl; wherein R⁷ is selected from C1-C6 alkyl, —CH₂OR¹², and —C(O)ZR¹²; wherein R⁸ is selected from hydrogen and C1-C6 alkyl; wherein each of R^{9a} and R^{9b} is independently selected from hydrogen and C1-C6 alkyl, provided that R^{9a} and R^{9b} are not simultaneously hydrogen; or wherein R^{9a} and R^{9b} are covalently bonded and, along with the intermediate carbon, together comprise optionally substituted C3-C5 cycloalkyl or optionally substituted C2-C5 heterocycloalkyl; wherein R¹⁰ is selected from hydrogen and C1-C6 alkyl; wherein each R¹ is independently selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; wherein R¹² is selected from hydrogen and optionally substituted organic residue having from 1 to 20 carbons; wherein Z is selected from —O— and —NR¹³—; wherein R¹³ is selected from hydrogen and C1-C4 alkyl; or, wherein Z is N, R¹² and R¹³ are covalently bonded and —NR¹²R¹³ comprises a moiety of the formula:

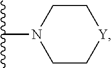

wherein Y is selected from —O—, —S—, —SO—, —SO₂—, —NH—, —NCH₃—; and wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl; or a pharmaceutically acceptable salt, hydrate, solvate, or polymorph thereof, and one or more of: (a) a protein supplement; (b) an anabolic agent; (c) a catabolic agent; (d) a dietary supplement; (e) at least one agent known to treat a disorder associated with muscle wasting; (f) instructions for treating a disorder associated with cholinergic activity; or (g) instructions for using the compound to increase muscle mass and/or muscular strength.

In a further aspect, the kit comprises a disclosed compound or a product of a disclosed method.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

x) Non-Medical Uses

Also provided are the uses of the disclosed compounds and products as pharmacological tools in the development and standardization of in vitro and in vivo test systems for the evaluation of the effects of modulators of muscle hypertrophy or inhibitors of muscle atrophy related activity in laboratory animals such as cats, dogs, rabbits, monkeys, rats and mice, as part of the search for new therapeutic agents of increase muscle mass and/or inhibit muscle hypertrophy.

### F. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

(1) General Methods

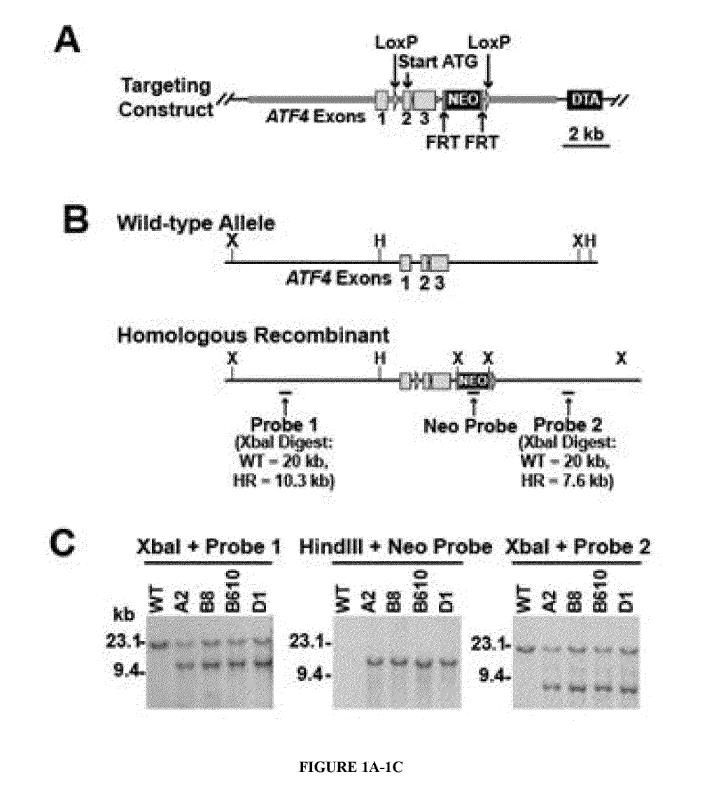

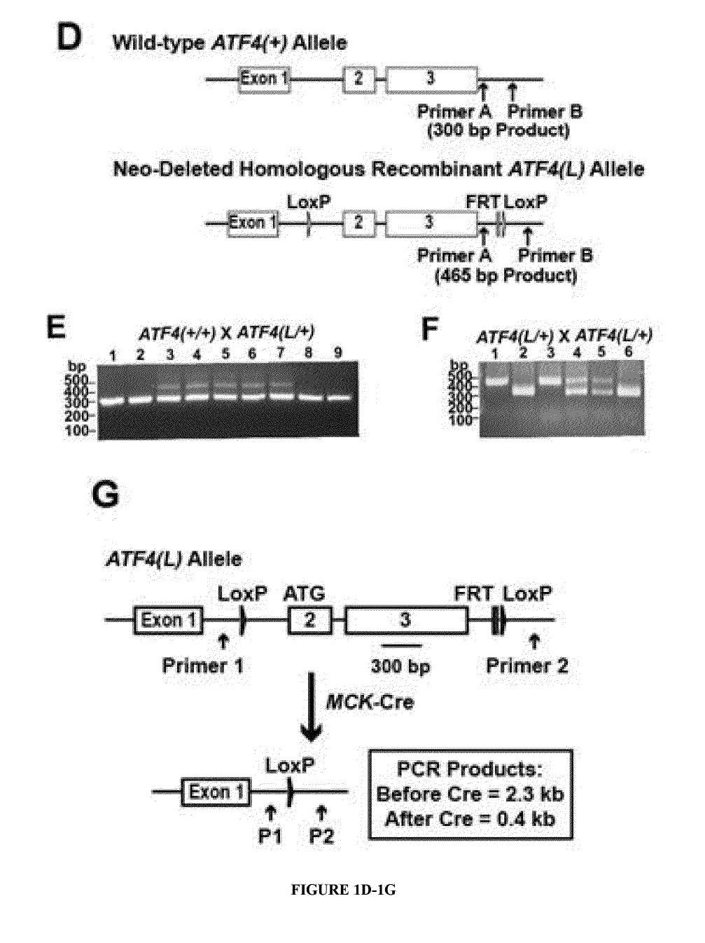

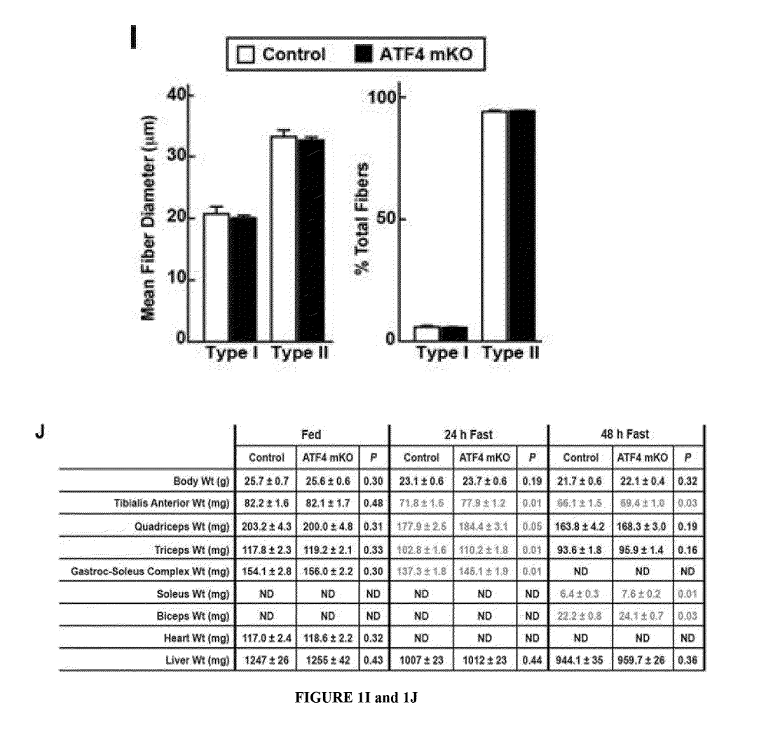

Mouse Protocols—ATF4 mKO mice were generated and genotyped as described in FIG. 1. ATF4 mKO mice were compared to ATF4(L/L); MCK-Cre(0/0) littermates, and all experiments used 9-12 week old males. C57BL/6 mice were also males, obtained from NCI at ages 6-8 weeks, and used for experiments within 3 weeks of their arrival. Fasting, unilateral hindlimb denervation and electroporation of mouse TA muscles were performed as described previously (Ebert, S. M., et al. (2010) Mol. Endocrinol. 24, 790-799; Kunkel, S. D., et al. (2011) Cell Metab. 13, 627-638). Unilateral TA muscle immobilization was performed under isoflurane anesthesia using an Autosuture Royal 35W skinstapler (Tyco Healthcare, Point Claire, Q C, Canada) as described previously (Caron, A. Z., et al. (2009) J. Appl. Physiol. 106, 2049-2059; Burks, T. N., et al. (2011) Sci. Translat. Med. 3, 82ra37). Except during fasting experiments, mice were provided ad libitum access to standard chow (Harlan Teklad formula 7013) and water. During fasting, food but not water was removed. All animal procedures were approved by the Institutional Animal Care and Use Committee of the University of Iowa.

Adenoviruses and Plasmids—Ad-ATF4 and Ad-ATF4ΔbZIP were generated by subcloning ATF4-FLAG and ATF4ΔbZIP-FLAG (21), respectively, into the pacAd5 K-N pA shuttle plasmid (Zhang, Z., et al. (2007) J. Neurosci. 27, 2693-2703), after which replication-deficient (E1, E3 deleted) recombinant adenoviruses co-expressing eGFP were generated by the University of Iowa Gene Transfer Vector Core as described previously (Anderson, R. D., et al. (2000) Gene Ther. 7, 1034-1038). Ad-GFP control virus has been described previously (Zhang, Z., et al. (2007) J. Neurosci. 27, 2693-2703). Adenovirus titers were determined by plaque assays on 293 cells. Viruses were stored in phosphate-buffered saline (PBS) with 3% sucrose at −80° C. p-miR-Gadd45a and p-miR-Gadd45a #2 were generated by ligating Mmi507625 and Mmi507626 oligonucleotide duplexes (Invitrogen), respectively, into the pcDNA6.2GW/EmGFP miR plasmid (Invitrogen), which contains a CMV promoter driving co-cistronic expression of engineered pre-miRNAs and EmGFP (Invitrogen). p-miR-Control encodes a non-targeting pre-miRNA hairpin sequence (miR-neg control; Invitrogen) in pcDNA6.2GW/EmGFP miR plasmid. p-miR-Cdkn1a and p-miR-Cdkn1a #2 were generated by ligating Mmi506257 and Mmi506259 oligonucleotide duplexes (Invitrogen), respectively, into the pcDNA6.2GW/EmGFP miR plasmid. To generate p-Gadd45a-FLAG, the coding region of mouse Gadd45a (NM_{—}007836) was amplified from mouse muscle cDNA, then cloned into p3XFLAG-CMV10 (Sigma), which placed three copies of the FLAG epitope tag at the NH₃-terminus. Ad-Gadd45a was generated by subcloning Gadd45a-FLAG into pacAd5 K-N pA and following the same protocol used for Ad-ATF4 and Ad-ATF4ΔbZIP. The Cdkn1a reporter construct was generated by amplifying a fragment of the mouse Cdkn1a promoter (−1419 to −1146 bp upstream Cdkn1a TSS #2) using genomic DNA from mouse skeletal muscle and the following primers: 5′-CTTCTGCTGGGTGTGATGGC-3′ (sense) (SEQ ID NO:3) and 5′-CCCAAGATCCAGACAGTCCAC-3′ (anti-sense) (SEQ ID NO:4). This amplified fragment was then cloned into the Kpn1 and HindIII sites in the pGL3-Basic vector (Promega). pRL-CMV-Renilla luciferase plasmid was from Promega. To generate p-Cdkn1a-FLAG, the coding region of mouse Cdkn1a (NM_{—}007669) was amplified from mouse muscle cDNA, then cloned into p3XFLAG-CMV10 (Sigma), which placed three copies of the FLAG epitope tag at the NH₃-terminus. Ad-Cdkn1a contains eGFP under control of an RSV promoter and Cdkn1a-FLAG (described above) under control of a tetracycline response element (TRE). Ad-Cdkn1a was generated by subcloning Cdkn1a-FLAG into the pAd5TRE pA shuttle plasmid, after which replication-deficient (E1, E3 deleted) recombinant adenoviruses co-expressing eGFP were generated by the University of Iowa Gene Transfer Vector Core as described previously (Gomes, M. D., et al. (2001) Proc. Natl. Acad. Sci. U.S.A. 98, 14440-14445). Ad-tTA was described previously (Laure, L., et al. (2009) FEBS J. 276, 669-684) and expresses a Tet-Off tetracycline transactivator protein. Adenovirus titers were determined by plaque assays on 293 cells. Adenoviruses were stored in phosphate-buffered saline (PBS) with 3% sucrose at −80° C.

Immunohistochemistry, H&E Staining and Light Microscopy of Mouse Muscle—For fiber type analysis, mouse tibialis anterior muscles (TAs) were harvested and fixed in 10% zinc formalin for 16 h, processed with RMC1530 parafin tissue processor, and then embedded in paraffin. A Leica RM2135 ultramicrotome was used to prepare 5 μm sections, which were then deparaffinized and subjected to epitope retrieval with Antigen Unmasking Solution (Vector Labs H-3300) and a Pelco Biowave. Nonspecific peroxidase activity was quenched with 3% H₂O₂ in methanol. Blocking and primary antibody incubation utilized the mouse on mouse (M.O.M.) kit (Vector Labs, BMK-2202) and either fast myosin heavy chain (Sigma Aldrich Company, #M4276) or slow myosin heavy chain (Sigma Aldrich Company, clone NOQ7.5.4.D, #M8421). Slides were then washed and incubated with Envision plus anti-mouse HRP (Dako K4001) antibody followed by visualization utilizing DAB (DAB peroxidase substrate Kit, 3,3′ diaminobenzidine Kit, Vector Labs SK-4100). To localize Gadd45a, TAs were fixed in 4% paraformaldehyde for 16 h, placed in 30% sucrose (wt/vol) for 24 h and then embedded in tissue freezing medium. A Microm HM 505 E cryostat was then used to prepare 8 um sections, which were rinsed 3× with PBS (pH 7.4) and then blocked with PBS containing 5% normal goat serum (NGS) for 1 h, followed by an overnight incubation with a 1:50 dilution of rabbit monoclonal anti-FLAG (Sigma, Product No. F2555) in PBS containing 5% NGS at 4° C. After incubation, muscle sections were washed 3× with PBS and then incubated with Alexa 568-conjugated secondary antibody (1:400) for 1 h at room temperature. Muscle sections were then washed 3× with PBS and then covered with Vectashield mounting medium. For H&E staining, sections were prepared using the same technique used for fiber type analysis, and then stained with H&E according to standard protocols. To analyze transfected fibers, TA sections were prepared and imaged as described previously (Ebert, S. M., et al. (2010) Mol. Endocrinol. 24, 790-799). All sections were examined and photographed on an Olympus IX-71 microscope equipped with a DP-70 camera. Image analysis was performed using ImageJ software. Muscle fiber diameters were measured using the lesser diameter technique as described previously (Dubowitz, V., Lane, R., and Sewry, C. A. (2007) Muscle Biopsy: A Practical Approach, 3rd ed., Saunders Elsevier, Philadelphia, Pa.). In each muscle, we measured the diameter of ≧300 transfected fibers, using the lesser diameter technique as described previously (Caron, A. Z., et al. (2009) J. Appl. Physiol. 106, 2049-2059).

Transmission Electron Microscopy (TEM) of mouse muscle—Mouse TA muscles were fixed in 2.5% glutaraldehyde in 0.1 M cacodylate buffer (pH 7.4) at 4° C. overnight, rinsed 3× with calcodylate 0.1 M buffer and then postfixed and stained with 1% osmium tetroxide (OsO₄) and 1.5% potassium ferrocyanide (K₄Fe(CN)₆) in cacodylate 0.2 M buffer for 1.5 h at room temperature. Skeletal muscle sections were then stained with 2.5% uranyl acetate for 30 min, dehydrated by a series of ethanol dilutions (50-100%), infiltrated with graded mixtures of propylene oxides and Epoxy Resin 12, and then embedded in 100% Epoxy Resin 12. Ultra-thin sections (≈85 nm) were cut using a Leica UC6 ultramicrotome and stained with 2% uranyl acetate and lead citrate. Sections were examined and photographed with a JEM-1230 transmission electron microscope equipped with a Gatan Ultra Scan 2 k×2 k CCD camera. Myonuclear diameter was measured with the lesser diameter method and ImageJ software.

C2C12 Myotube Culture and Infection—Mouse C2C12 myoblasts were obtained from ATCC (CRL-1772), and maintained at 37° C. and 5% CO₂ in Dulbecco's modified Eagle's medium (DMEM) (ATCC #30-2002) containing antibiotics (100 units/ml penicillin, 100 μg/ml streptomycin sulfate) and 10% (v/v) fetal bovine serum (FBS). Myoblasts were set up for experiments on day 0 in 6-well plates at a density of 2.5×10⁵ cells/well. On day 2, differentiation was induced by replacing 10% FBS with 2% horse serum. On day 7, cells were rinsed once with PBS, and then 1 ml DMEM containing adenovirus was added to each well. An MOI of 250 was used for Ad-ATF4, Ad-ATF4ΔbZIP and Ad-Gadd45a. To overexpress Cdkn1a, we used an MOI of 125 for Ad-Cdkn1a plus an MOI of 125 for Ad-tTA. Two hours later, 1 ml DMEM containing 1% horse serum plus antibiotics was added to each well. On day 8, cells were rinsed twice with PBS, and then 2 ml DMEM containing 2% horse serum and antibiotics was added to each well. Infection efficiency was >90%. All assays except protein degradation (described below) were performed on day 9, 48 h post-infection.

Myotube Protein Synthesis and Protein Degradation—[³H]-leucine (120 Ci/mmol) and [³H]-tyrosine (40 Ci/mmol) were obtained from ARC. For analysis of protein synthesis, [³H]-leucine incorporation into cultured myotubes was determined as described previously (Malmberg, S. E., and Adams, C. M. (2008) J. Biol. Chem. 283, 19229-19234). Protein degradation assays were performed according to a previously described protocol (Zhao, J., et al. (2007) FoxO3 coordinately activates protein degradation by the autophagic/lysosomal and proteasomal pathways in atrophying muscle cells. Cell Metab. 6, 472-483): myotubes were incubated with [³H]-tyrosine (4 μCi/ml) for 20 h (to label long-lived proteins) and then switched to chase medium (DMEM, antibiotics, and 2 mM unlabeled tyrosine) for 2 h. Myotubes were then rinsed with PBS, after which 1 ml chase medium containing adenovirus (MOI 250) was added to each well. Two hours later, 1 ml DMEM containing 1% horse serum plus antibiotics was added to each well. Medium samples were collected 36 h post-infection and mixed with TCA (15% final concentration) for 1 h at 4° C. Precipitated proteins were washed twice with 10% TCA and twice with 95% EtOH, and then radioactivity was measured by liquid scintillation analysis. The acid-soluble radioactivity reflects the amount of proteins degraded and was expressed relative to the total cellular radioactivity present at the time of infection.

Histological Analysis of Myotubes—All myotube imaging was performed on an Olympus IX-71 microscope equipped with a DP-70 camera and epifluorescence filters. Image analysis was performed using ImageJ software. To analyze myotube size, three width measurements were averaged per GFP-positive myofiber and measured ≧60 myotubes per sample. To localize Gadd45a, myotubes were washed two times with ice-cold PBS, fixed in 4% paraformaldehyde for 10 min, and permeabilized with PBS (7.4 pH) containing 0.5% Triton X-100 for 15 min. Permeabilized myotubes were blocked with PBS containing 1% bovine serum albumin (BSA) and 5% normal goat serum (NGS) for 1 h, followed by an overnight incubation with 1:50 dilution of rabbit monoclonal anti-FLAG (Sigma, Product No. F2555) in PBS containing 1% BSA at 4° C. After incubation, the myotubes were washed 3× with PBS and then incubated with Alexa 568-conjugated secondary antibody (1:400) for 1 h at room temperature. Myotubes were then washed 3× with PBS and then covered with Vectashield mounting medium. For trypan blue staining, myotubes were rinsed 3× with PBS, stained with 0.2% trypan blue (in PBS) for 5 minutes at room temperature, and then rinsed 2× with PBS. As a positive control for cell death, myotubes were treated with 80% ethanol for 20 minutes prior to staining with 0.2% trypan blue.

Exon Arrays and Quantitative Real-Time RT-PCR (qPCR) in Muscle and Myotubes—Extraction of skeletal muscle RNA and RNA hybridizations to Mouse Exon 1.0 ST arrays (Affymetrix) were performed as described previously (Ebert, S. M., et al. (2010) Mol. Endocrinol. 24, 790-799). Exon expression arrays examining the effects of Gadd45a overexpression, fasting and muscle denervation in TA muscles of C57BL/6 mice were described previously (Banduseela, V. C., et al. (2009) Physiol. Genomics 39, 141-159; Welle, S., et al. (2004) Exp. Gerontol. 39, 369-377) and can be found in the NCBI Gene Expression Omnibus under GEO Series accession numbers GSE39196, GSE20103 and GSE39195, respectively. Myotube RNA was extracted using TRIzol solution (Invitrogen), and then purified using the RNeasy kit and RNase Free DNase Set (Qiagen). qPCR analyses of mRNAs encoding mouse ATF4, Gadd45a, Bax, caspase-3 (Casp3), androgen receptor (Ar), thyroid hormone receptor-α (Thra), growth hormone receptor (Ghr), hexokinase 2 (Hk2), Suclg1, Coxl1, Tfam, Nos1, MuRF1 (Trim63), atrogin-1 (Fbxo32), cathepsin L (Ctsl), Bnip3, Cdkn1a, PGC-1α (Ppargc1a), GLUT4 (S1c2a4), LC3a (Mapl1c3a) and Vegfa were performed using TaqMan Gene Expression Assays (Applied Biosystems). For qPCR studies, first strand cDNA was synthesized in a 20 μl reaction that contained 2 μg of RNA, random hexamer primers and components of the High Capacity cDNA reverse transcription kit (Applied Biosystems). qPCR was carried out using a 7500 Fast Real-Time PCR System (Applied Biosystems). All qPCR reactions were performed in triplicate and the cycle threshold (Ct) values were averaged to give the final results. To analyze the data, the ΔCt method was used, with level of 36B4 mRNA serving as the invariant control.

Immunoblot Analysis of Mouse Skeletal Muscle and C2C12 Myotubes—Skeletal muscle protein extracts were prepared as described previously (Ebert, S. M., et al. (2010) Mol. Endocrinol. 24, 790-799). Myotube protein extracts were prepared by scraping PBS-washed myotubes into cold lysis buffer (10 mM Tris-HCl, pH 7.6, 100 mM NaCl, and 1% (w/v) SDS, cOmplete Mini protease inhibitor cocktail (Roche), and a 1:100 dilution of phosphatase inhibitor cocktail 3 (Sigma)), and then lysing with 10 passes through a 22-gauge needle. Aliquots of muscle and myotube protein extracts were mixed with 0.25 volume of sample buffer (250 mM Tris-HCl, pH 6.8, 10% SDS, 25% glycerol, 0.2% (w/v) bromophenol blue, and 5% (w/v) 2-mercaptoethanol) and heated for 5 min at 95° C. A separate aliquot of each extract was used to determine protein concentration by the BCA kit (Pierce), after which an equal amount of protein from each sample was subjected to SDS-PAGE, and then transferred to Hybond-C extra nitrocellulose filters (Millipore). Immunoblots were performed at 4° C. for 16 h using 1:1500 dilution of mouse anti-FLAG monoclonal antibody (Sigma, Product No. F1804), a 1:35,000 dilution of polyclonal anti-actin antiserum (Sigma, Product No. A2103), a 1:1000 dilution of polyclonal anti-PGC-1α (Abcam, Product No. ab54481), a 1:1000 dilution of polyclonal anti-Bnip3 (Cell Signaling, #3769), a 1:1500 dilution of polyclonal anti-LC3I/II (Cell Signaling, #4108), a 1:8000 dilution of polyclonal anti-Cox IV (Abcam, Product No.ab16056) or a 1:2000 dilution of antibodies detecting Caspase-3, total Akt, phospho-Akt (Ser473), total GSK-3β or phospho-GSK-3β (Ser9) (Cell Signaling Products #9662, 4691L, 4060S, 9315 and 9323, respectively).

Analysis of Caspase Activity—Mouse TA muscles were snap frozen in liquid N₂, and homogenized in 1 ml of cold lysis buffer solution containing 50 mM Tris (pH 7.4), 150 mM NaCl, 0.1% (v/v) Triton X-100, 1.5 mM MgCl₂, 1 mM sodium EDTA, 1 mM sodium EGTA, and cOmplete Mini protease inhibitor cocktail (Roche) using a polytron (Tissue Master 240, Omni International) for 1 min on setting #8. C2C12 myotube homogenates were prepared by scrapping PBS-washed myotubes into cold lysis buffer solution (above) that contained cOmplete Mini protease inhibitor cocktail (Roche) then lysed with 10 passes through a 22-gauge needle. Muscle and myotube homogenates were centrifuged at 4° C. and 10,000 g for 10 min, and caspase activity assays were set-up in white-walled 96-well plates; each assay contained 20 μg protein from the sample supernatant mixed with an equal volume of caspase reagent (Promega, Madison, Wis.). Reactions were incubated on a rocker for 30 min at room temperature, and then luminescence was measured on a SpectraMax L luminescence microplate reader (Molecular Devices, Sunnyvale, Calif.). All reactions were performed in triplicate and values were averaged to give the final results.

Analysis of Mitochondrial DNA—Mouse skeletal muscle DNA was extracted using the QIAamp DNA mini kit (Qiagen). Mitochondrial and nuclear DNA was quantified by qPCR; reactions contained, in a final volume of 20 μl, 10 ng muscle DNA, 660 nM forward and reverse primers, and 10 μl 2× Power SYBR Green Master Mix (ABI). Ndufv1 and mtDNA primer sequences were previously described (Chen, H., et al. (2010) Cell 141, 280-289; Amthor, H., et al. (2007) Proc. Natl. Acad. Sci. U.S.A. 104, 1835-1840). qPCR was carried out using a 7500 Fast Real-Time PCR System (Applied Biosystems). All qPCR reactions were performed in triplicate and the cycle threshold (Ct) values were averaged to give the final results. To analyze the data, the ΔCt method was used, with level of 36B4 mRNA serving as the invariant control.

DNA Isolation from Myotubes and Muscle—Myotubes were washed, harvested into PBS, centrifuged at 500 g at 4° C. for 5 min, resuspended in 0.5 ml buffer A (60 mM Tris (pH 8.0), 100 mM EDTA, 0.5% (w/v) SDS and 500 μg/ml proteinase K), and then incubated at 45° C. for 24 h. Skeletal muscle was minced and then incubated in buffer A at 45° C. for 24 h. DNA was extracted by three sequential phenol:chloroform extractions, precipitated in ethanol, spooled, moved to a fresh 1.5 ml tube, and then washed once with 70% EtOH. DNA was then air dried and resuspended in DNAase/RNAse-free H₂O.

Methylated DNA Immunoprecipitation (MeDIP)-Chip—Purified genomic DNA (6 μg) was digested with 24 U MseI supplemented with 100 μg/ml BSA at 37° C. for 15 h, followed by a 20 min incubation at 65° C. to inactivate MseI. Digested genomic DNA was purified using the QIAquick PCR Purification kit (Qiagen 28106). Fragment size (100 to 2000 bp) was determined using an Agilent Bioanalyzer DNA7500 chip, and DNA concentration was determined using a Nanodrop ND-1000. Digested genomic DNA (1.25 μg) was incubated with 1 μg monoclonal mouse anti-5-methyl-cytidine (Eurogentec BI-MECY-0500) at 4° C. for 16 h, and then precipitated with 48 μl protein A agarose suspension (Invitrogen 15918-014) at 4° C. for 2 h. Precipitates were washed and then treated with 70 μg proteinase K (NEB P8102S) at 55° C. for 16 h. The MeDIP was purified by phase extracting with 250 μl of phenol, followed by 250 μl chloroform:isoamyl alcohol, and then precipitated with NaCl and ethanol. After washing with 70% ethanol, the pellets were reconstituted in 10 mM Tris HCl (pH 8.5) and quantitated by Nanodrop analysis. Both input and MeDIP fractions for each sample were amplified using the Sigma GenomePlex Complete WGA 2 kit. Amplified material was purified using QIAquick spin columns. Concentration and purity was verified using Nanodrop and size distribution was verified using a Bioanalyzer DNA7500 chip. Amplified DNA was labeled and hybridized to NimbleGen mm9 2.1M Deluxe Mouse Promoter Arrays (Roche) according to the manufacturer's recommendations. Microarrays were scanned using a NimbleGen MS 200 scanner. Probe-specific P-values were determined using NimbleScan (Roche) software's default parameters for the one-sided Kolmogorov-Smirnov (KS) test.

Bisulfate Sequencing—Bisulfite treatment of purified genomic DNA (2 μg) was performed using the EpiTect Bisulfite Kit (Qiagen) according to the manufacturer's protocol. To amplify bisulfite-converted clones from mouse skeletal muscle DNA, we used the PCR primers 5′-TGTAGTTTTAATTTTAAGTAAGG-3′ (sense) (SEQ ID NO:5) and 5′-CACTAAAATAACATTAATAAAAAAC-3′ (anti-sense) (SEQ ID NO:6). To amplify bisulfite-converted clones from the Cdkn1a reporter plasmid, we used the PCR primers 5′-AGGTATTATTTTTGTTGGGTGTG-3′ (sense) (SEQ ID NO:7) and 5′-CAAAATCCAAACAATCCACTAA-3′ (anti-sense) (SEQ ID NO:8). PCR products were cloned into pCR2.1-TOPO vector using the TOPO TA Cloning Kit (Invitrogen). DNA sequencing was performed at the University of Iowa DNA Facility.

Chromatin Immunoprecipitation—Primers were designed to amplify the portion of the Cdkn1a promoter that was previously analyzed with bisulfite sequencing. Primer sequences were 5′-CTTCTGCTGGGTGTGATGGC-3′ (sense) (SEQ ID NO:3) and 5′-CCCAAGATCCAGACAGTCCAC-3′ (anti-sense) (SEQ ID NO:4). Following a 48 h infection with Ad-Gadd45a or Ad-Control, myotubes were fixed with 1% formaldehyde for 15 minutes at room temperature. Sonication was performed with a Branson Sonifier 450, and conditions were empirically determined to cleave genomic DNA into 250 bp to 800 bp fragments. The final sonication conditions were 3 rounds of 10 seconds of 0.5 second pulses on power output setting #5. Chromatin immunoprecipitation was performed using mouse anti-FLAG monoclonal antibody and the EZ-ChIP kit (Millipore) according to the manufacturer's instructions.

In Vitro Methylation of the Cdkn1a Reporter and Measurement of Its Activity—The Cdkn1a reporter construct was methylated with M.SssI CpG methyltransferase (NEB, M0226) according to manufacturer's instructions. Unmethylated reporter construct was incubated in parallel without methyltransferase. Following incubation, plasmids were precipitated and resuspended in sterile saline. Muscles were homogenized in 1 ml 1× Passive Lysis Buffer (Promega), and then homogenates were centrifuged at 4° C. and 5,000 g for 5 min. Luciferase assays were set-up in white-walled 96-well plates. Each assay initially contained 25 μg protein from the sample supernatant plus 100 μl Luciferase Assay Reagent II (Promega). After measuring firefly luciferase activity with a SpectraMax L luminescence microplate reader (Molecular Devices, Sunnyvale, Calif.), firefly luciferase activity was quenched and Renilla luciferase activity was activated by adding 100 μl of Stop & Glo Reagent (Promega). Reactions were performed in duplicate, and mean firefly luciferase activity was normalized to mean Renilla luciferase activity to give the final results.

Quantification of Mouse Skeletal Muscle Specific Tetanic Force—The lower hindlimb was removed (by transsecting the upper hindlimb mid-way through the femur), and placed in Krebs Ringer solution (NaCl 120 mM; NaHCO₃ 23.8 mM; D-glucose 10 mM; KCl 4.8 mM; CaCl₂ 2.5 mM; KH₂PO₄ 1.2 mM; MgSO₄ 1.2 mM; HEPES 5 mM; CaCl₂ 2.5 mM) aerated with 95% O₂ and 5% CO₂. The gastrocnemius, soleus and TA muscles, as well as the distal half of the tibia and fibula, were then removed leaving the extensor digitorum longus (EDL) with their origins and insertions intact. The associated nerve and vessel supplies were trimmed last to ensure optimum condition of muscles prior to entering the organ bath. A staple was placed through the knee joint with a suture attached. The mean time from euthanasia to maximal force measurements was 10 min. Isometric contractile properties of the EDL muscle were evaluated in vitro according to methods described previously (Wenz, T., (2009) Proc. Natl. Acad. Sci. U.S.A. 106, 20405-20410). Each ex vivo preparation was mounted vertically in a water jacket bath (Aurora Scientific 1200A Intact Muscle Test System, filled with aerated Krebs solution that was bubbled with 95% O2 and 5% CO2 and thermostatically maintained at 25° C. The suture was attached to a servocontrolled lever (Model 805A; Aurora Scientific) superiorly and metatarsals were clamped inferiorly. Muscles were field stimulated by supramaximal square-wave pulses (0.2 ms duration, Model 701C; Aurora Scientific), that were amplified (Model 604A; Aurora Scientific), and delivered to two platinum plate electrodes that flanked the length of the muscle to produce a maximum isometric contraction. Optimum muscle length (Lₒ) and optimum stimulation voltage were determined from micromanipulation of muscle length and a series of twitch contractions. Maximum isometric tetanic force (Pₒ) was determined from the plateau of the tetanic curve following stimulation with supramaximal voltage (40 V) at 150 Hz with 2 min rest between recordings to prevent fatigue. Contractile measurements were recorded using a digital controller (Model 600A; Aurora Scientific) operating ASI Dynamic Muscle Control acquisition software (v4.1, Aurora Scientific). Muscles were stimulated once every 2 s at optimum length, voltage and frequency, with stimulation duration of 350 ms and final forces produced during the stimulation protocol were recorded. Following force testing, muscles were removed from the bath, trimmed of their tendons and any adhering non-muscle tissue, and weighed on an analytical balance. Optimum fiber length (L_{f}) was determined by multiplying Lₒ by fiber length to muscle length ratio determined previously (0.44 for the EDL muscle (Burks, T. N., (2011) Sci. Translat. Med. 3, 82ra37)). Muscle mass, L_{f} and Pₒ were used to calculate maximum tetanic force normalized per total muscle fiber crosssectional area (kN/m²). Muscle cross sectional area was determined by dividing muscle mass (mg) by the product of L_{f} and 1.06 mg/mm³ (the density of mammalian skeletal muscle (Zhang, Z., et al. (2007) J. Neurosci. 27, 2693-2703)).

Statistical Analysis—Unless otherwise noted in the figure legends, we used paired t-tests to compare within subject samples and unpaired t-tests for all other comparisons.

(2) Introduction to FIGS. 1-11

A variety of stresses, including starvation, muscle disuse, systemic illness and aging cause skeletal muscle atrophy, which is often debilitating. However, despite its broad impact, muscle atrophy lacks an effective medical therapy and its pathogenesis remains incompletely understood. Like many complex diseases, muscle atrophy is associated with widespread positive and negative changes in gene expression (Lecker, S. H., et al. (2004) FASEB J. 18, 39-51; Sacheck, J. M., et al. (2007) FASEB J. 21, 140-155; Banduseela, V. C., et al. (2009) Physiol. Genomics 39, 141-159; Welle, S., et al. (2004) Exp. Gerontol. 39, 369-377; Welle, S., et al. (2003) Physiol. Genomics 14, 149-159; Edwards, M. G., et al. (2007) BMC Genomics 8, 80; Stevenson, E. J., et al. (2003) J. Physiol. 551, 33-48; Gonzalez de Aguilar, J. L., et al. (2008) Physiol. Genomics 32, 207-218). Some gene expression changes in atrophying muscle are known to promote atrophy, including induction of genes that promote proteolysis (Bodine, S. C., et al. (2001) Science 294, 1704-1708; Sandri, M., et al. (2004) Cell 117, 399-412; Stitt, T. N., et al. (2004) Mol. Cell 14, 395-403; Moresi, V., et al. (2010) Cell 143, 35-45; Cai, D., et al. (2004) Cell 119, 285-298; Acharyya, S., et al. (2004) J. Clin. Investig. 114, 370-378; Mammucari, C., et al. (2007) Cell Metab. 6, 458-471; Zhao, J., et al. (2007) Cell Metab. 6, 472-483; Plant, P. J., et al. (2009) J. Appl. Physiol. 107, 224-234) and repression of the gene encoding PGC-1α, a transcriptional coactivator that promotes mitochondrial biogenesis and energy production (Sandri, M., et al. (2006) Proc. Natl. Acad. Sci. U.S.A. 103, 16260-16265; Wenz, T., et al. (2009) Proc. Natl. Acad. Sci. U.S.A. 106, 20405-20410). However, most atrophy-associated gene expression changes are unstudied, and it remains unknown if these changes contribute to muscle atrophy, and if so, to what extent.

Some recent studies have investigated the potential role of activating transcription factor 4 (ATF4, also called CREB2) in muscle atrophy. ATF4 is a basic leucine zipper (bZIP) transcription factor that mediates a variety of cellular stress responses (Harding, H. P., et al. (2003) Mol. Cell 11, 619-633). Oligonucleotide microarrays showed that starvation, denervation, diabetes, cancer and renal failure increase ATF4 mRNA in skeletal muscle (Lecker, S. H., et al. (2004) FASEB J. 18, 39-51; Sacheck, J. M., et al. (2007) FASEB J. 21, 140-155). A subsequent study showed that ATF4 overexpression in mouse skeletal muscle is sufficient to induce muscle fiber atrophy (Ebert, S. M., et al. (2010) Mol. Endocrinol. 24, 790-799). Conversely, an RNA interference construct targeting ATF4 mRNA reduces muscle fiber atrophy induced by fasting (Ebert, S. M., et al. (2010) Mol. Endocrinol. 24, 790-799). Collectively, these studies indicated an important role for ATF4 in fasting-induced muscle atrophy, and raised the possibility that ATF4 might also mediate other types of muscle atrophy, such as disuse atrophy, which most commonly occurs when muscles are immobilized by limb casting or bedrest.

The mechanism by which ATF4 promotes muscle atrophy is not yet known. ATF4 does not increase atrogin-1/MAFbx or MuRF1 mRNAs (Ebert, S. M., et al. (2010) Mol. Endocrinol. 24, 790-799), the first well-characterized atrophy-associated transcripts, which are partially required for muscle atrophy (Bodine, S. C., et al. (2001) Science 294, 1704-1708; Gomes, M. D., et al. (2001) Proc. Natl. Acad. Sci. U.S.A. 98, 14440-14445). This indicates the existence of a previously unrecognized pathway that operates in parallel to, or downstream of, known atrophy pathways. A previous study used exon expression arrays to identify five mouse skeletal muscle mRNAs that are induced by both ATF4 overexpression and fasting: Gadd45a, Cdkn1a, Peg3, Ankrd1 and Csrp3 (Ebert, S. M., et al. (2010) Mol. Endocrinol. 24, 790-799). Of these, Gadd45a is particularly intriguing because other microarray studies also associated Gadd45a induction with skeletal muscle atrophy in mice, pigs and humans (Banduseela, V. C., et al. (2009) Physiol. Genomics 39, 141-159; Welle, S., et al. (2004) Exp. Gerontol. 39, 369-377; Welle, S., et al. (2003) Physiol. Genomics 14, 149-159; Edwards, M. G., et al. (2007) BMC Genomics 8, 80; Stevenson, E. J., et al. (2003) J. Physiol. 551, 33-48; Gonzalez de Aguilar, J. L., et al. (2008) Physiol. Genomics 32, 207-218). However, the role of Gadd45a in skeletal muscle is not known. Indeed, many mRNAs are induced in atrophic muscle, and at least some (including Ankrd1, atrogin-1 and MuRF1) are not sufficient to cause muscle atrophy (Sandri, M., et al. (2004) Cell 117, 399-412; Moresi, V., et al. (2010) Cell 143, 35-45; Laure, L., et al. (2009) FEBS J. 276, 669-684). Thus, it is not known if ATF4 causes atrophy by inducing Gadd45a.

The studies described herein tested whether ATF4 might play a broader role in muscle atrophy by generating and studying muscle-specific ATF4 knockout (ATF4 mKO) mice. When it became clear that ATF4 promotes both fasting- and immobilization-induced atrophy, a search for the downstream mechanism was undertaken.

(a) Loss of ATF4Delays Skeletal Muscle Atrophy Induced by Fasting or Immobilization

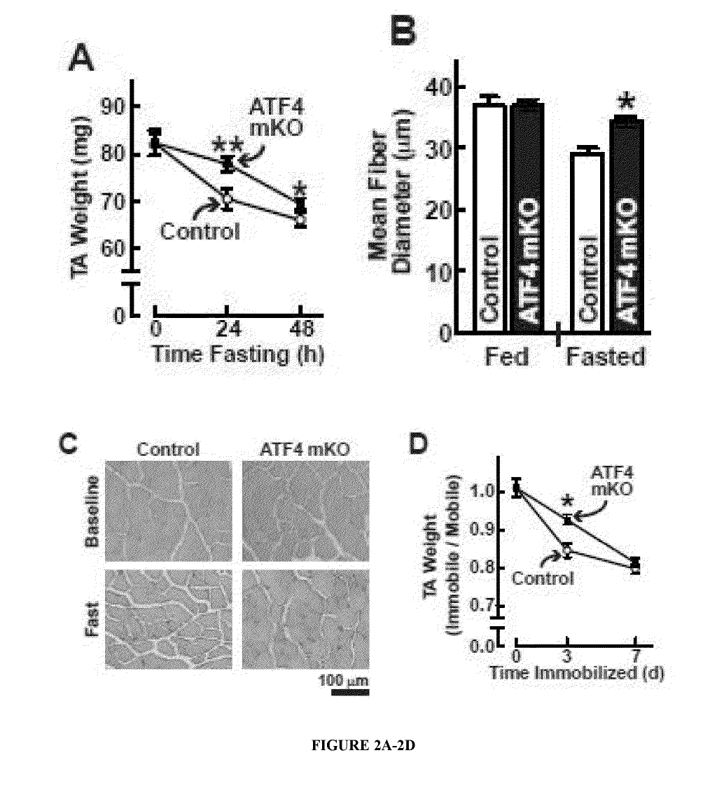

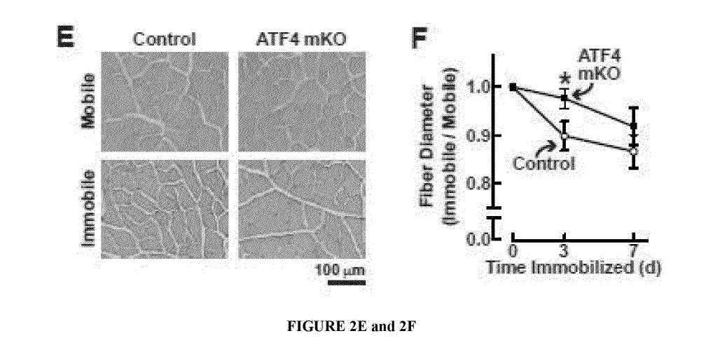

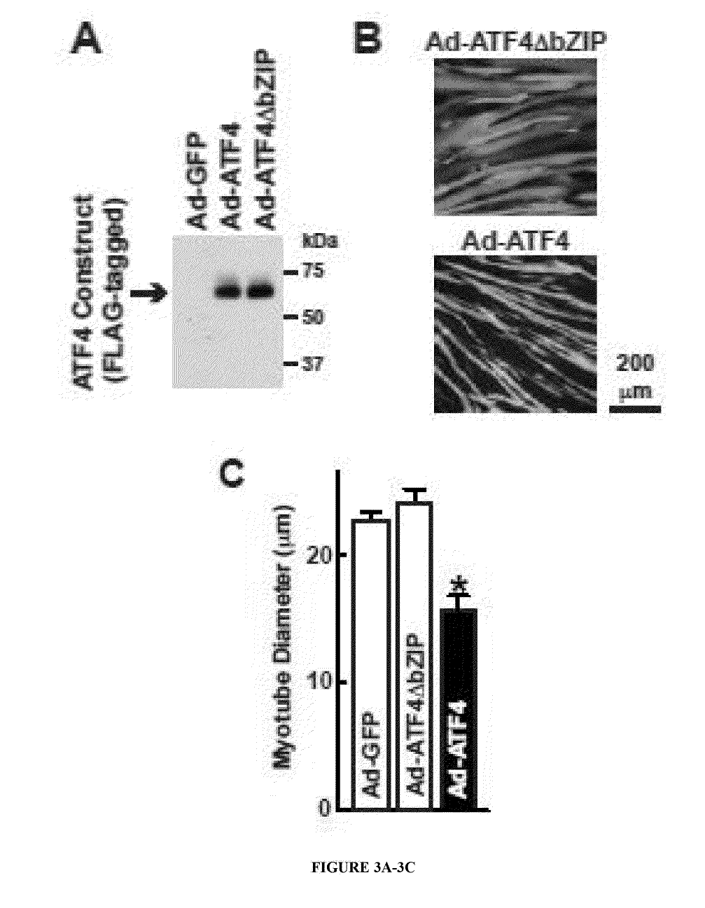

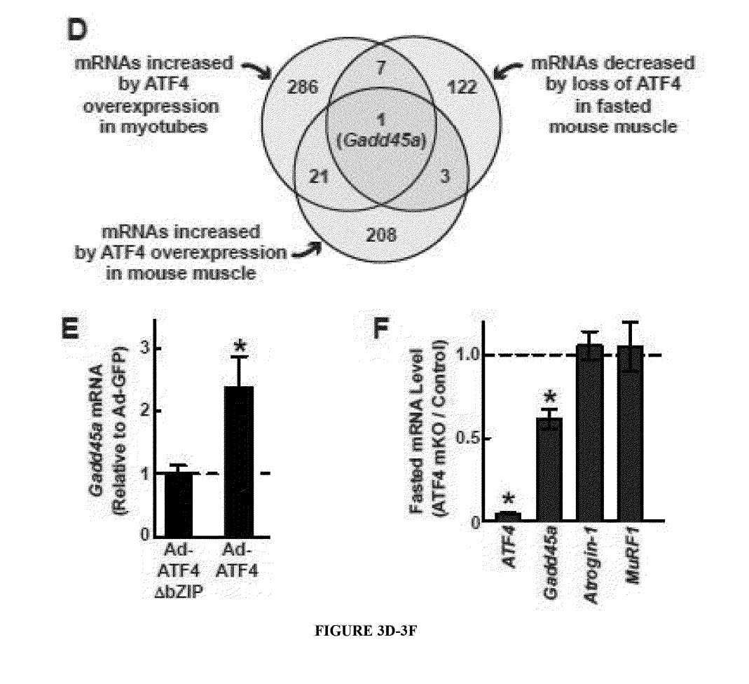

To generate ATF4 mKO mice, the coding region of the mouse ATF4 gene (exons 2 and 3) was flanked with LoxP restriction sites. The floxed ATF4(L) allele was then excised by crossing ATF4(L/L) mice to transgenic mice carrying Cre recombinase under control of the muscle creatine kinase (MCK) promoter (FIG. 1A-1G) (Brüning, J. C., et al. (1998) Mol. Cell 2, 559-569). As expected, the MCK-Cre transgene specifically excised the ATF4(L) allele in skeletal muscle and heart, reducing ATF4 mRNA in skeletal muscle by >95% (FIGS. 1H and 3F). Residual ATF4 mRNA may be from satellite cells and non-muscle cells, which do not express MCK-Cre (34). ATF4 mKO were born at the expected Mendelian frequency and lacked any overt phenotype up to 9 months of age (the longest period of observation). Relative to littermate control mice lacking MCK-Cre, ATF4 mKO mice possessed normal total body, skeletal muscle, heart, and liver weights (FIG. 1J). Histological examination of ATF4 mKO skeletal muscle revealed normal percentages and sizes of type I and type II muscle fibers, and no signs of degeneration, regeneration, or inflammation (FIGS. 1I and 2C and 2E). Thus, skeletal muscle ATF4 expression was not required for skeletal muscle development, and its absence did not induce muscle hypertrophy.

Because RNAi-mediated knockdown of ATF4 reduces atrophy of TA muscle fibers during fasting (Ebert, S. M., et al. (2010) Mol. Endocrinol. 24, 790-799), it was anticipated that ATF4 mKO mice would be resistant to fasting-induced muscle atrophy. After 24 or 48 h of fasting, ATF4 mKO TA muscles and muscle fibers were significantly larger than those of control mice (FIG. 2A-2C). Other skeletal muscles, including fast- and slow-twitch muscles (biceps and soleus, respectively), also exhibited reduced atrophy (FIG. 1J). Interestingly, loss of ATF4 appeared to have a greater protective effect after 24 h of fasting than after 48 h of fasting. This was apparent in the TAs (FIG. 2A) and in the quadriceps and triceps (FIG. 1J). These data indicated that loss of ATF4 reduced fasting-induced atrophy by delaying its progression.

To test whether loss of ATF4 might delay muscle atrophy induced by a different stress: muscle immobilization, one TA was immobilized with a surgical staple (Caron, A. Z., et al. (2009) J. Appl. Physiol. 106, 2049-2059; and Burks, T. N., et al. (2011) Sci. Translat. Med. 3, 82ra37), leaving the contralateral, mobile TA as an internal control. Relative to littermate control TAs, ATF4 mKO TAs underwent less muscle and muscle fiber atrophy during the first 3 days of immobilization (FIG. 2D-2F). However, after 7 days of immobilization, the amount of muscle and muscle fiber atrophy was equivalent between the two genotypes (FIGS. 2D and 2F). Thus, loss of ATF4 delayed immobilization-induced muscle atrophy. Collectively, these results indicate that ATF4 is partially required for early, essential events in immobilization- and fasting-induced skeletal muscle atrophy.

(b) Identification of Gadd45a as a Transcript that is Reduced in ATF4 mKO Muscle and Increased by ATF4 Overexpression in Both Mouse Muscle and Cultured C2C12 Myotubes

Overexpressing ATF4, but not a transcriptionally inactive ATF4 construct (ATF4ΔbZIP), induces skeletal muscle fiber atrophy in mice (Ebert, S. M., et al. (2010) Mol. Endocrinol. 24, 790-799). To develop a complementary in vitro system, C2C12 skeletal myotubes were infected with adenovirus co-expressing ATF4 and GFP (Ad-ATF4). Control myotubes were infected with adenoviruses expressing only GFP (Ad-GFP) or GFP plus ATF4ΔbZIP. Immunoblot analysis confirmed that Ad-ATF4 and Ad-ATF4ΔbZIP generated ATF4 and ATF4ΔbZIP, respectively (FIG. 3A). Similar to its effect in mouse muscle (Ebert, S. M., et al. (2010) Mol. Endocrinol. 24, 790-799), ATF4 overexpression induced myotube atrophy (FIGS. 3B and 3C).

To determine how ATF4 promotes muscle atrophy, genome-wide exon expression arrays were used to search for mRNAs that satisfied three criteria: 1) induced by Ad-ATF4 in myotubes; 2) reduced in muscle from ATF4 mKO mice; and 3) induced by ATF4 overexpression in mouse muscle. Effects of Ad-ATF4 were determined by comparing myotubes infected with Ad-ATF4 and myotubes infected with Ad-ATF4ΔbZIP. Effects of ATF4 mKO were determined by comparing TA muscles from fasted ATF4 mKO mice and TA muscles from ATF4(L/L) littermate controls. Effects of ATF4 overexpression in mouse muscle were determined by comparing C57BL/6 TA muscles that were transfected with plasmid encoding mouse ATF4 empty vector and contralateral TA muscles that were trasfected with empty plasmid, as described previously (Ebert, S. M., et al. (2010) Mol. Endocrinol. 24, 790-799). Using p≦0.01 as the threshold for statistical significance, only one mRNA, Gadd45a, satisfied all three criteria (FIG. 3D and Tables 1 and 2). qPCR analysis confirmed that Ad-ATF4 increased Gadd45a mRNA in C2C12 myotubes (FIG. 3E), and that Gadd45a mRNA was significantly reduced in ATF4 mKO muscle (FIG. 3F). Previous qPCR studies confirmed that ATF4 overexpression increases Gadd45a mRNA in C57BL/6 muscle (Ebert, S. M., et al. (2010) Mol. Endocrinol. 24, 790-799). In contrast, atrogin-1 or MuRF1 mRNAs are not increased by ATF4 overexpression (Ebert, S. M., et al. (2010) Mol. Endocrinol. 24, 790-799), and they were not reduced in ATF4 mKO muscle (FIG. 3F). These data suggested that ATF4 might cause muscle atrophy by inducing Gadd45a.

TABLE 1
C2C12 MYOTUBES MRNAS THAT ARE INCREASED BY AD-ATF4

| | | Log2 Δ |
|---|---|---|
| | | ATF4- |
| gene_assignment | Gene Symbol | ATF4DeltabZIP |
| NM_001032298 // Bglap2 // bone gamma- | Bglap2 | 4.08 |
| carboxyglutamate protein 2 // 3 F1\|3 42.6 c | | |
| NM_153171 // Rgs13 // regulator of G-protein signaling | Rgs13 | 3.86 |
| 13 // 1 F\|1 78.0 cM // 24 | | |
| NM_011315 // Saa3 // serum amyloid A 3 // 7 B4\|7 23.5 cM | Saa3 | 3.74 |
| // 20210 /// ENSMUST000 | | |
| NM_145953 // Cth // cystathionase (cystathionine | Cth | 3.46 |
| gamma-lyase) // 3 H4 // 107869 | | |
| NM_011990 // Slc7a11 // solute carrier family 7 | Slc7a11 | 3.27 |
| (cationic amino acid transporter | | |
| NM_009731 // Akr1b7 // aldo-keto reductase family 1, | Akr1b7 | 3.18 |
| member B7 // 6 B1\|6 14.0 cM | | |
| NM_017396 // Cyp3a41a // cytochrome P450, family 3, | Cyp3a41a | 3.10 |
| subfamily a, polypeptide 41A | | |
| NM_148942 // Serpinb6c // serine (or cysteine) | Serpinb6c | 3.01 |
| peptidase inhibitor, clade B, mem | | |
| NM_145526 // P2rx3 // purinergic receptor P2X, | P2rx3 | 2.81 |
| ligand-gated ion channel, 3 // 2 | | |
| NM_001003405 // Try5 // trypsin 5 // 6 B1 // 103964 /// | Try5 | 2.60 |
| NM_011646 // Try4 // try | | |
| NM_175512 // Dhrs9 // dehydrogenase/reductase (SDR | Dhrs9 | 2.50 |
| family) member 9 // 2 C2 // 2 | | |
| NM_153543 // Aldh1l2 // aldehyde dehydrogenase 1 | Aldh1l2 | 2.44 |
| family, member L2 // 10 C1 // 2 | | |
| NM_030596 // Dsg3 // desmoglein 3 // 18 A2\|18 7.04 cM | Dsg3 | 2.41 |
| // 13512 /// ENSMUST000000 | | |
| NM_027853 // Mettl7b // methyltransferase like 7B // | Mettl7b | 2.29 |
| 10 D3 // 71664 /// ENSMUST0 | | |
| NM_033552 // Slc4a10 // solute carrier family 4, | Slc4a10 | 2.19 |
| sodium bicarbonate cotransporte | | |
| NM_013703 // Vldlr // very low density lipoprotein | Vldlr | 2.12 |
| receptor // 19 C1\|19 20.0 cM | | |
| NM_019549 // Plek // pleckstrin // 11 A2\|11 6.5 cM // | Plek | 2.12 |
| 56193 /// NM_029861 // Cnr | | |
| NM_012044 // Pla2g2e // phospholipase A2, group IIE | Pla2g2e | 2.06 |
| // 4 D3 // 26970 /// ENSMUST | | |
| NM_008470 // Krt16 // keratin 16 // 11 D // 16666 /// | Krt16 | 2.05 |
| ENSMUST00000007280 // Krt1 | | |
| NM_012055 // Asns // asparagine synthetase // 6 A1 // | Asns | 2.04 |
| 27053 /// ENSMUST000000317 | | |
| NM_029001 // Elovl7 // ELOVL family member 7, | Elovl7 | 1.96 |
| elongation of long chain fatty aci | | |
| NM_011643 // Trpc1 // transient receptor potential | Trpc1 | 1.92 |
| cation channel, subfamily C, | | |
| NM_010740 // Cd93 // CD93 antigen // 2 G3\|2 84.0 cM | Cd93 | 1.92 |
| // 17064 /// ENSMUST00000099 | | |
| NM_013463 // Gla // galactosidase, alpha // X E-F1\|X | Gla | 1.87 |
| 53.0 cM // 11605 /// ENSMUS | | |
| NM_205844 // Gfral // GDNF family receptor alpha | Gfral | 1.86 |
| like // 9 D // 404194 /// ENSMU | | |
| NM_177203 // A730037C10Rik // RIKEN cDNA | A730037C10Rik | 1.85 |
| A730037C10 gene // 3 C // 320604 /// EN | | |
| NM_026929 // Chac1 // ChaC, cation transport | Chac1 | 1.85 |
| regulator-like 1 (E. coli) // 2 E5 | | |
| NM_144838 // Sgtb // small glutamine-rich | Sgtb | 1.85 |
| tetratricopeptide repeat (TPR)-contain | | |
| NM_029612 // Slamf9 // SLAM family member 9 // 1 | Slamf9 | 1.84 |
| H3 // 98365 /// ENSMUST00000027 | | |
| NM_175271 // Lpar4 // lysophosphatidic acid receptor | Lpar4 | 1.82 |
| 4 // X D // 78134 /// ENSMU | | |
| NM_026347 // Iah1 // isoamyl acetate-hydrolyzing | Iah1 | 1.81 |
| esterase 1 homolog (S. cerevisi | | |
| NM_016687 // Sfrp4 // secreted frizzled-related protein | Sfrp4 | 1.80 |
| 4 // 13 A2\|13 7.0 cM // | | |
| NM_015774 // Ero1l // ERO1-like (S. cerevisiae) // 14 | Ero1l | 1.79 |
| C-D // 50527 /// ENSMUST00 | | |
| NM_172779 // Ddx26b // DEAD/H (Asp-Glu-Ala- | Ddx26b | 1.79 |
| Asp/His) box polypeptide 26B // X A5 | | |
| NM_207246 // Rasgrp3 // RAS, guanyl releasing | Rasgrp3 | 1.78 |
| protein 3 // 17 E2 // 240168 /// E | | |
| NM_008135 // Slc6a9 // solute carrier family 6 | Slc6a9 | 1.77 |
| (neurotransmitter transporter, gl | | |
| NM_007498 // Atf3 // activating transcription factor 3 // | Atf3 | 1.76 |
| 1 H6\|1 103.2 cM // 119 | | |
| NM_029413 // Morc4 // microrchidia 4 // X F1 // 75746 | Morc4 | 1.73 |
| /// ENSMUST00000033811 // | | |
| NM_018782 // Calcrl // calcitonin receptor-like // 2 D // | Calcrl | 1.73 |
| 54598 /// ENSMUST00000 | | |
| NM_010876 // Ncf1 // neutrophil cytosolic factor 1 // 5 | Ncf1 | 1.71 |
| G2\|5 74.0 cM // 17969 // | | |
| NM_028230 // Shmt2 // serine | Shmt2 | 1.67 |
| hydroxymethyltransferase 2 (mitochondrial) // 10 D3 | | |
| NM_011812 // Fbln5 // fibulin 5 // 12 F1 // 23876 /// | Fbln5 | 1.66 |
| ENSMUST00000021603 // Fbln | | |
| NM_139292 // Reep6 // receptor accessory protein 6 // | Reep6 | 1.64 |
| 10 C1 // 70335 /// ENSMUST | | |
| NM_001093754 // Dennd2d // DENN/MADD domain | Dennd2d | 1.63 |
| containing 2D // 3 F2.3 // 72121 /// | | |
| NM_009452 // Tnfsf4 // tumor necrosis factor (ligand) | Tnfsf4 | 1.62 |
| superfamily, member 4 // 1 | | |
| NM_007515 // Slc7a3 // solute carrier family 7 (cationic | Slc7a3 | 1.62 |
| amino acid transporter, | | |
| NM_022026 // Aqp9 // aquaporin 9 // 9 D // 64008 /// | Aqp9 | 1.60 |
| ENSMUST00000113570 // Aqp9 | | |
| NM_007887 // Dub1 // deubiquitinating enzyme 1 // 7 | Dub1 | 1.60 |
| E3\|7 51.5 cM // 13531 /// U4 | | |
| NM_027236 // Eif1ad // eukaryotic translation initiation | Eif1ad | 1.55 |
| factor 1A domain contai | | |
| NM_198422 // Paqr3 // progestin and adipoQ receptor | Paqr3 | 1.53 |
| family member III // 5 E3 // | | |
| NM_175012 // Grp // gastrin releasing peptide // 18 | Grp | 1.48 |
| E1\|18 40.0 cM // 225642 /// | | |
| NM_025446 // Aig1 // androgen-induced 1 // 10 A2 // | Aig1 | 1.48 |
| 66253 /// ENSMUST00000019942 | | |
| NM_023794 // Etv5 // ets variant gene 5 // 16 B1 // | Etv5 | 1.47 |
| 104156 /// ENSMUST0000007960 | | |
| NM_011404 // Slc7a5 // solute carrier family 7 (cationic | Slc7a5 | 1.45 |
| amino acid transporter, | | |
| NM_021611 // Myl10 // myosin, light chain 10, | Myl10 | 1.45 |
| regulatory // 5 G1 // 59310 /// NM | | |
| NM_181754 // Gpr141 // G protein-coupled receptor | Gpr141 | 1.44 |
| 141 // 13 A2 // 353346 /// ENS | | |
| NM_001111059 // Cd34 // CD34 antigen // 1 H6\|1 | Cd34 | 1.43 |
| 106.6 cM // 12490 /// NM_133654 / | | |
| NM_146041 // Gmds // GDP-mannose 4, 6-dehydratase | Gmds | 1.42 |
| // 13 A3.2 // 218138 /// ENSMU | | |
| NM_001037127 // Musk // muscle, skeletal, receptor | Musk | 1.42 |
| tyrosine kinase // 4 B3\|4 26. | | |
| NM_172262 // Aof1 // amine oxidase, flavin containing | Aof1 | 1.41 |
| 1 // 13 A5 // 218214 /// E | | |
| NM_028035 // Snx10 // sorting nexin 10 // 6 B3 // | Snx10 | 1.40 |
| 71982 /// NM_001127348 // Snx1 | | |
| NM_001161413 // Slc3a2 // solute carrier family 3 | Slc3a2 | 1.39 |
| (activators of dibasic and neu | | |
| NM_145355 // Rnf185 // ring finger protein 185 // 11 | Rnf185 | 1.38 |
| A1 // 193670 /// ENSMUST000 | | |
| NM_133900 // Psph // phosphoserine phosphatase // 5 | Psph | 1.37 |
| G1.3 // 100678 /// ENSMUST00 | | |
| NM_010442 // Hmox1 // heme oxygenase (decycling) 1 | Hmox1 | 1.36 |
| // 8 C1\|8 35.0 cM // 15368 // | | |
| NM_027963 // Wdr16 // WD repeat domain 16 // 11 B3 | Wdr16 | 1.35 |
| // 71860 /// ENSMUST000000212 | | |
| NM_013778 // Akr1c13 // aldo-keto reductase family 1, | Akr1c13 | 1.35 |
| member C13 // 13 A1 // 273 | | |
| NM_009626 // Adh7 // alcohol dehydrogenase 7 (class | Adh7 | 1.34 |
| IV), mu or sigma polypeptide | | |
| NM_008638 // Mthfd2 // methylenetetrahydrofolate | Mthfd2 | 1.33 |
| dehydrogenase (NAD+ dependent), | | |
| NM_009829 // Ccnd2 // cyclin D2 // 6 F3\|6 61.1 cM // | Ccnd2 | 1.32 |
| 12444 /// ENSMUST0000000018 | | |
| NM_008087 // Gas2 // growth arrest specific 2 // 7 B5\|7 | Gas2 | 1.28 |
| 26.8 cM // 14453 /// ENS | | |
| NM_007532 // Bcat1 // branched chain | Bcat1 | 1.28 |
| aminotransferase 1, cytosolic // 6 G3\|6 73. | | |
| NM_010330 // Emb // embigin // 13 D2.3 // 13723 /// | Emb | 1.28 |
| NM_011279 // Rnf7 // ring fi | | |
| NM_009635 // Avil // advillin // 10 D3 // 11567 /// | Avil | 1.26 |
| ENSMUST00000026500 // Avil / | | |
| NM_008681 // Ndrg1 // N-myc downstream regulated | Ndrg1 | 1.26 |
| gene 1 // 15 D2 // 17988 /// EN | | |
| NM_007945 // Eps8 // epidermal growth factor receptor | Eps8 | 1.24 |
| pathway substrate 8 // 6 G | | |
| NM_031159 // Apobec1 // apolipoprotein B mRNA | Apobec1 | 1.23 |
| editing enzyme, catalytic polypept | | |
| NM_020622 // Fam3b // family with sequence | Fam3b | 1.22 |
| similarity 3, member B // 16 C4\|16 71 | | |
| NM_001122768 // Lrrc8d // leucine rich repeat | Lrrc8d | 1.22 |
| containing 8D // 5 E5 // 231549 // | | |
| NM_152801 // Arhgef6 // Rac/Cdc42 guanine | Arhgef6 | 1.22 |
| nucleotide exchange factor (GEF) 6 // | | |
| NM_175093 // Trib3 // tribbles homolog 3 (Drosophila) | Trib3 | 1.22 |
| // 2 G3 // 228775 /// ENSM | | |
| NM_028977 // Lrrc17 // leucine rich repeat containing | Lrrc17 | 1.22 |
| 17 // 5 A3 // 74511 /// NM | | |
| NM_183191 // Plch1 // phospholipase C, eta 1 // 3 E1 // | Plch1 | 1.22 |
| 269437 /// ENSMUST000000 | | |
| AY026045 // 1700016D06Rik // RIKEN cDNA | 1700016D06Rik | 1.21 |
| 1700016D06 gene // 8 A1.1 // 76413 /// B | | |
| NM_053113 // Ear11 // eosinophil-associated, | Ear11 | 1.21 |
| ribonuclease A family, member 11 // | | |
| NM_008132 // Glrp1 // glutamine repeat protein 1 // 1 | Glrp1 | 1.20 |
| D // 14659 /// ENSMUST0000 | | |
| NM_144907 // Sesn2 // sestrin 2 // 4 D2.3 // 230784 /// | Sesn2 | 1.20 |
| ENSMUST00000030724 // Se | | |
| NM_009201 // Slc1a5 // solute carrier family 1 (neutral | Slc1a5 | 1.20 |
| amino acid transporter), | | |
| NM_001122893 // Fyn // Fyn proto-oncogene // 10 | Fyn | 1.20 |
| B1\|10 25.0 cM // 14360 /// NM_00 | | |
| NM_019698 // Aldh18a1 // aldehyde dehydrogenase 18 | Aldh18a1 | 1.19 |
| family, member A1 // 19 C3 // | | |
| NM_177420 // Psat1 // phosphoserine aminotransferase | Psat1 | 1.16 |
| 1 // 19 A\|19 32.5 cM // 107 | | |
| BC024574 // 1110012L19Rik // RIKEN cDNA | 1110012L19Rik | 1.14 |
| 1110012L19 gene // X A7.1 // 68618 /// N | | |
| NM_016745 // Atp2a3 // ATPase, Ca++ transporting, | Atp2a3 | 1.13 |
| ubiquitous // 11 B4 // 53313 / | | |
| NM_178655 // Ank2 // ankyrin 2, brain // 3 G2\|3 62.5 cM | Ank2 | 1.13 |
| // 109676 /// NM_0010341 | | |
| NM_011157 // Srgn // serglycin // 10 B4 // 19073 /// | Srgn | 1.12 |
| ENSMUST00000105446 // Srgn | | |
| NM_013820 // Hk2 // hexokinase 2 // 6 C3\|6 34.5 cM // | Hk2 | 1.11 |
| 15277 /// ENSMUST000000006 | | |
| NM_001122685 // Rhbdd1 // rhomboid domain | Rhbdd1 | 1.11 |
| containing 1 // 1 C5 // 76867 /// NM_0 | | |
| NM_175514 // Fam171b // family with sequence | Fam171b | 1.10 |
| similarity 171, member B // 2 D // | | |
| NM_134086 // Slc38a1 // solute carrier family 38, | Slc38a1 | 1.10 |
| member 1 // 15 F1 // 105727 // | | |
| NM_020013 // Fgf21 // fibroblast growth factor 21 // 7 | Fgf21 | 1.09 |
| B2 // 56636 /// ENSMUST00 | | |
| NM_172715 // Agpat9 // 1-acylglycerol-3-phosphate O- | Agpat9 | 1.09 |
| acyltransferase 9 // 5 E4 // | | |
| NM_001010937 // Gjb6 // gap junction protein, beta 6 // | Gjb6 | 1.07 |
| 14 C3\|14 22.5 cM // 1462 | | |
| NM_025936 // Rars // arginyl-tRNA synthetase // 11 A4 | Rars | 1.07 |
| // 104458 /// ENSMUST00000 | | |
| NM_177906 // Opcml // opioid binding protein/cell | Opcml | 1.07 |
| adhesion molecule-like // 9 A4 | | |
| NM_146489 // Olfr266 // olfactory receptor 266 // --- // | Olfr266 | 1.06 |
| 258482 /// ENSMUST00000 | | |
| ENSMUST00000060610 // Dnahc14 // dynein, | Dnahc14 | 1.06 |
| axonemal, heavy chain 14 // 1 H5 // 240 | | |
| NM_013729 // Mixl1 // Mix1 homeobox-like 1 | Mixl1 | 1.06 |
| (Xenopus laevis) // 1 H4 // 27217 /// | | |
| NM_022331 // Herpud1 // homocysteine-inducible, | Herpud1 | 1.06 |
| endoplasmic reticulum stress-ind | | |
| NM_019733 // Rbpms // RNA binding protein gene | Rbpms | 1.06 |
| with multiple splicing // 8 A4 // | | |
| NM_172308 // Mthfd1l // methylenetetrahydrofolate | Mthfd1l | 1.05 |
| dehydrogenase (NADP+ dependent | | |
| NM_028011 // Tom1l1 // target of myb1-like 1 | Tom1l1 | 1.04 |
| (chicken) // 11 D\|11 53.0 cM // 719 | | |
| NM_011281 // Rorc // RAR-related orphan receptor | Rorc | 1.04 |
| gamma // 3 F2 // 19885 /// ENSM | | |
| NM_009468 // Dpysl3 // dihydropyrimidinase-like 3 // | Dpysl3 | 1.04 |
| 18 B3 // 22240 /// NM_00113 | | |
| NM_028994 // Pck2 // phosphoenolpyruvate | Pck2 | 1.02 |
| carboxykinase 2 (mitochondrial) // 14 C | | |
| NM_145527 // Madd // MAP-kinase activating death | Madd | 1.02 |
| domain // 2 E1 // 228355 /// EN | | |
| NM_178766 // Slc25a40 // solute carrier family 25, | Slc25a40 | 1.00 |
| member 40 // 5 A1 // 319653 / | | |
| NM_001159500 // Esrrb // estrogen related receptor, | Esrrb | 0.99 |
| beta // 12 D2\|12 41.0 cM // | | |
| NM_133829 // Mfsd6 // major facilitator superfamily | Mfsd6 | 0.99 |
| domain containing 6 // 1 C1. | | |
| NM_001039546 // Myo6 // myosin VI // 9 E1\|9 44.0 cM | Myo6 | 0.99 |
| // 17920 /// ENSMUST00000035 | | |
| NM_053093 // Tac4 // tachykinin 4 // 11 D // 93670 /// | Tac4 | 0.98 |
| ENSMUST00000021242 // Tac | | |
| NM_030181 // Vsig1 // V-set and immunoglobulin | Vsig1 | 0.97 |
| domain containing 1 // X F1 // 78 | | |
| NM_008969 // Ptgs1 // prostaglandin-endoperoxide | Ptgs1 | 0.97 |
| synthase 1 // 2 B\|2 29.0 cM // | | |
| NM_173866 // Gpt2 // glutamic pyruvate transaminase | Gpt2 | 0.96 |
| (alanine aminotransferase) 2 | | |
| NM_177909 // Slc9a9 // solute carrier family 9 | Slc9a9 | 0.96 |
| (sodium/hydrogen exchanger), memb | | |
| BC138236 // 4922501L14Rik // RIKEN cDNA | 4922501L14Rik | 0.96 |
| 4922501L14 gene // 3 H4 // 209601 /// EN | | |
| NM_008059 // G0s2 // G0/G1 switch gene 2 // 1 H6\|1 | G0s2 | 0.95 |
| 104.1 cM // 14373 /// ENSMUST | | |
| NM_009255 // Serpine2 // serine (or cysteine) peptidase | Serpine2 | 0.94 |
| inhibitor, clade E, memb | | |
| NM_009884 // Cebpg // CCAAT/enhancer binding | Cebpg | 0.94 |
| protein (C/EBP), gamma // 7 B1 // 1 | | |
| NM_010748 // Lyst // lysosomal trafficking regulator // | Lyst | 0.94 |
| 13 A1\|13 7.0 cM // 17101 | | |
| NM_030203 // Tspyl4 // TSPY-like 4 // 10 B1\|10 22.0 cM | Tspyl4 | 0.94 |
| // 72480 /// ENSMUST00000 | | |
| NM_007918 // Eif4ebp1 // eukaryotic translation | Eif4ebp1 | 0.93 |
| initiation factor 4E binding pro | | |
| NM_029706 // Cpb1 // carboxypeptidase B1 (tissue) // 3 | Cpb1 | 0.92 |
| A2\|3 13.0 cM // 76703 /// | | |
| NM_020259 // Hhip // Hedgehog-interacting protein // | Hhip | 0.92 |
| 8 C3\|8 40.0 cM // 15245 /// | | |
| NM_145512 // Sft2d2 // SFT2 domain containing 2 // 1 | Sft2d2 | 0.91 |
| H2.3 // 108735 /// ENSMUST0 | | |
| NM_026384 // Dgat2 // diacylglycerol O- | Dgat2 | 0.89 |
| acyltransferase 2 // 7 F1 // 67800 /// EN | | |
| NM_176959 // Fbxl7 // F-box and leucine-rich repeat | Fbxl7 | 0.89 |
| protein 7 // 15 B1 // 448987 | | |
| NM_008317 // Hyal1 // hyaluronoglucosaminidase 1 // | Hyal1 | 0.89 |
| 9 F1-F2\|9 60.1 cM // 15586 / | | |
| NM_001161817 // Myo1b // myosin IB // 1 C1.1\|1 24.8 cM | Myo1b | 0.89 |
| // 17912 /// NM_010863 // | | |
| NM_133926 // Camk1 // calcium/calmodulin-dependent | Camk1 | 0.88 |
| protein kinase I // 6 E3\|6 48 | | |
| NM_199468 // Zcchc5 // zinc finger, CCHC domain | Zcchc5 | 0.88 |
| containing 5 // X D // 213436 // | | |
| NM_027409 // Mospd1 // motile sperm domain | Mospd1 | 0.88 |
| containing 1 // X A4 // 70380 /// ENS | | |
| NM_153145 // Abca8a // ATP-binding cassette, sub- | Abca8a | 0.87 |
| family A (ABC1), member 8a // 1 | | |
| NM_009704 // Areg // amphiregulin // 5 E1\|5 51.0 cM // | Areg | 0.87 |
| 11839 /// ENSMUST00000031 | | |
| NM_025400 // Nat9 // N-acetyltransferase 9 (GCN5- | Nat9 | 0.86 |
| related, putative) // 11 E2 // | | |
| NM_028057 // Cyb5r1 // cytochrome b5 reductase 1 // 1 | Cyb5r1 | 0.86 |
| E4 // 72017 /// ENSMUST000 | | |
| NM_013454 // Abca1 // ATP-binding cassette, sub- | Abca1 | 0.86 |
| family A (ABC1), member 1 // 4 A | | |
| NM_007836 // Gadd45a // growth arrest and DNA- | Gadd45a | 0.86 |
| damage-inducible 45 alpha // 6 C1 | | |
| NM_054054 // Brdt // bromodomain, testis-specific // 5 | Brdt | 0.85 |
| E5 // 114642 /// NM_00107 | | |
| NM_023333 // 2210010C04Rik // RIKEN cDNA | 2210010C04Rik | 0.85 |
| 2210010C04 gene // 6 B1 // 67373 /// EN | | |
| NM_011491 // Stc2 // stanniocalcin 2 // 11 A4 // 20856 | Stc2 | 0.84 |
| /// ENSMUST00000020546 // | | |
| NM_029083 // Ddit4 // DNA-damage-inducible | Ddit4 | 0.84 |
| transcript 4 // 10 B3 // 74747 /// EN | | |
| NM_144527 // Ccdc21 // coiled-coil domain containing | Ccdc21 | 0.84 |
| 21 // 4 D3 // 70012 /// ENS | | |
| NM_013851 // Abca8b // ATP-binding cassette, sub- | Abca8b | 0.84 |
| family A (ABC1), member 8b // 1 | | |
| NM_146023 // Evi2b // ecotropic viral integration site | Evi2b | 0.84 |
| 2b // 11 B5 // 216984 /// | | |
| NM_172595 // Arl15 // ADP-ribosylation factor-like 15 | Arl15 | 0.83 |
| // 13 D2.2 // 218639 /// E | | |
| NM_018861 // Slc1a4 // solute carrier family 1 | Slc1a4 | 0.82 |
| (glutamate/neutral amino acid tra | | |
| NM_080288 // Elmo1 // engulfment and cell motility 1, | Elmo1 | 0.82 |
| ced-12 homolog (C. elegans | | |
| NM_028782 // Lonp1 // lon peptidase 1, mitochondrial | Lonp1 | 0.81 |
| // 17 D // 74142 /// ENSMUS | | |
| NM_007757 // Cpox // coproporphyrinogen oxidase // - | Cpox | 0.80 |
| // 12892 /// ENSMUST00000 | | |
| NM_008091 // Gata3 // GATA binding protein 3 // 2 | Gata3 | 0.80 |
| A1\|2 7.0 cM // 14462 /// ENSMU | | |
| NM_020519 // Slurp1 // secreted Ly6/Plaur domain | Slurp1 | 0.80 |
| containing 1 // 15 D3 // 57277 | | |
| NM_026669 // Tmbim6 // transmembrane BAX | Tmbim6 | 0.79 |
| inhibitor motif containing 6 // 15 F3\|1 | | |
| BC004591 // 0610007P14Rik // RIKEN cDNA | 0610007P14Rik | 0.79 |
| 0610007P14 gene // 12 D2 // 58520 /// AF | | |
| BC076612 // 3110043O21Rik // RIKEN cDNA | 3110043O21Rik | 0.79 |
| 3110043O21 gene // 4 A5 // 73205 /// BC0 | | |
| NM_007404 // Adam9 // a disintegrin and | Adam9 | 0.77 |
| metallopeptidase domain 9 (meltrin gamma | | |
| NM_172372 // Wdr45 // WD repeat domain 45 // X | Wdr45 | 0.75 |
| A1.1\|X 1.6 cM // 54636 /// ENSMUS | | |
| NM_029409 // Mff // mitochondrial fission factor // 1 | Mff | 0.75 |
| C5 // 75734 /// ENSMUST000 | | |
| NM_011076 // Abcb1a // ATP-binding cassette, sub- | Abcb1a | 0.75 |
| family B (MDR/TAP), member 1A / | | |
| NM_001113198 // Mitf // microphthalmia-associated | Mitf | 0.75 |
| transcription factor // 6 D3\|6 | | |
| NM_001111316 // Ptprc // protein tyrosine phosphatase, | Ptprc | 0.75 |
| receptor type, C // 1 E4\| | | |
| NM_145616 // Lrrc49 // leucine rich repeat containing | Lrrc49 | 0.74 |
| 49 // 9 B // 102747 /// NM | | |
| NM_008361 // Il1b // interleukin 1 beta // 2 F\|2 73.0 cM | Il1b | 0.74 |
| // 16176 /// ENSMUST000 | | |
| NM_027460 // Slc25a33 // solute carrier family 25, | Slc25a33 | 0.74 |
| member 33 // 4 E1 // 70556 // | | |
| NM_019942 // Sept6 // septin 6 // X A2 // 56526 /// | 38965 | 0.74 |
| ENSMUST00000115241 // Sept6 | | |
| NM_172589 // Lhfpl2 // lipoma HMGIC fusion partner- | Lhfpl2 | 0.73 |
| like 2 // 13 D1 // 218454 /// | | |
| NM_024233 // Rexo2 // REX2, RNA exonuclease 2 | Rexo2 | 0.73 |
| homolog (S. cerevisiae) // 9 A5.3 | | |
| NM_019926 // Mtm1 // X-linked myotubular myopathy | Mtm1 | 0.72 |
| gene 1 // X A7.2\|X 27.8 cM // | | |
| NM_022563 // Ddr2 // discoidin domain receptor | Ddr2 | 0.72 |
| family, member 2 // 1 H1-H5\|1 90. | | |
| NM_177992 // Gmpr2 // guanosine monophosphate | Gmpr2 | 0.71 |
| reductase 2 // 14 C3 // 105446 /// | | |
| NM_011752 // Zfp259 // zinc finger protein 259 // 9 | Zfp259 | 0.71 |
| A5.2 // 22687 /// ENSMUST000 | | |
| NM_027427 // Taf15 // TAF15 RNA polymerase II, | Taf15 | 0.71 |
| TATA box binding protein (TBP)-as | | |
| NM_146573 // Olfr1002 // olfactory receptor 1002 // --- | Olfr1002 | 0.70 |
| // 258566 /// ENSMUST000 | | |
| NM_001013374 // Lman2l // lectin, mannose-binding | Lman2l | 0.70 |
| 2-like // 1 B // 214895 /// EN | | |
| NM_009378 // Thbd // thrombomodulin // 2 G3\|2 84.0 cM | Thbd | 0.70 |
| // 21824 /// ENSMUST000000 | | |
| NM_211358 // Slc35c1 // solute carrier family 35, | Slc35c1 | 0.70 |
| member C1 // 2 E1 // 228368 // | | |
| NM_019829 // Stx5a // syntaxin 5A // 19 A // 56389 /// | Stx5a | 0.69 |
| ENSMUST00000073430 // Stx | | |
| NM_019687 // Slc22a4 // solute carrier family 22 | Slc22a4 | 0.69 |
| (organic cation transporter), m | | |
| NM_021564 // Fetub // fetuin beta // 16 B\|16 14.1 cM // | Fetub | 0.69 |
| 59083 /// NM_001083904 / | | |
| NM_008223 // Serpind1 // serine (or cysteine) peptidase | Serpind1 | 0.69 |
| inhibitor, clade D, memb | | |
| NM_033563 // Klf7 // Kruppel-like factor 7 | Klf7 | 0.69 |
| (ubiquitous) // 1 C1-C3 // 93691 /// | | |
| NM_008801 // Pde6d // phosphodiesterase 6D, cGMP- | Pde6d | 0.69 |
| specific, rod, delta // 1 D // | | |
| NM_198304 // Nup188 // nucleoporin 188 // 2 B // | Nup188 | 0.68 |
| 227699 /// ENSMUST00000064447 / | | |
| NM_028940 // Rlbp1l1 // retinaldehyde binding protein | Rlbp1l1 | 0.68 |
| 1-like 1 // 4 A1 // 74438 | | |
| NM_146416 // Olfr290 // olfactory receptor 290 // --- // | Olfr290 | 0.68 |
| 258411 /// NM_146415 // | | |
| NM_027060 // Btbd9 // BTB (POZ) domain containing | Btbd9 | 0.68 |
| 9 // 17 B1 // 224671 /// NM_17 | | |
| NM_138315 // Mical1 // microtubule associated | Mical1 | 0.68 |
| monoxygenase, calponin and LIM dom | | |
| NM_019738 // Nupr1 // nuclear protein 1 // --- // 56312 | Nupr1 | 0.67 |
| /// ENSMUST00000032961 / | | |
| NM_027495 // Tmem144 // transmembrane protein 144 | Tmem144 | 0.66 |
| // 3 F1 // 70652 /// ENSMUST00 | | |
| NM_008142 // Gnb1 // guanine nucleotide binding | Gnb1 | 0.65 |
| protein (G protein), beta 1 // 4 | | |
| NM_010723 // Lmo4 // LIM domain only 4 // 3 H2\|3 | Lmo4 | 0.65 |
| 73.1 cM // 16911 /// NM_0011617 | | |
| NM_029102 // Glt8d2 // glycosyltransferase 8 domain | Glt8d2 | 0.64 |
| containing 2 // 10 C1 // 747 | | |
| NM_172751 // Arhgef10 // Rho guanine nucleotide | Arhgef10 | 0.64 |
| exchange factor (GEF) 10 // 8 A1 | | |
| NM_001146049 // Htatip2 // HIV-1 tat interactive | Htatip2 | 0.64 |
| protein 2, homolog (human) // 7 | | |
| NM_023190 // Acin1 // apoptotic chromatin | Acin1 | 0.64 |
| condensation inducer 1 // 14 C3 // 562 | | |
| NM_015776 // Mfap5 // microfibrillar associated | Mfap5 | 0.63 |
| protein 5 // 6 F1 // 50530 /// E | | |
| NM_009919 // Cnih // cornichon homolog (Drosophila) | Cnih | 0.63 |
| // 14 C1 // 12793 /// ENSMUS | | |
| NM_009351 // Tep1 // telomerase associated protein 1 | Tep1 | 0.63 |
| // 14 C2-D1\|14 13.5 cM // 2 | | |
| NM_028787 // Slc35f5 // solute carrier family 35, | Slc35f5 | 0.62 |
| member F5 // 1 E3 // 74150 /// | | |
| NM_146703 // Olfr1447 // olfactory receptor 1447 // --- | Olfr1447 | 0.62 |
| // 258698 /// ENSMUST000 | | |
| NM_178686 // Cep120 // centrosomal protein 120 // 18 | Cep120 | 0.61 |
| D1 // 225523 /// ENSMUST000 | | |
| NM_008861 // Pkd2 // polycystic kidney disease 2 // 5 | Pkd2 | 0.61 |
| E5\|5 55.0 cM // 18764 /// | | |
| NM_009396 // Tnfaip2 // tumor necrosis factor, alpha- | Tnfaip2 | 0.59 |
| induced protein 2 // 12 F1\| | | |
| NM_178723 // Zfp385b // zinc finger protein 385B // 2 | Zfp385b | 0.59 |
| C3 // 241494 /// NM_001113 | | |
| NM_023913 // Ern1 // endoplasmic reticulum (ER) to | Ern1 | 0.59 |
| nucleus signalling 1 // 11 E1 | | |
| NM_146090 // Zadh2 // zinc binding alcohol | Zadh2 | 0.58 |
| dehydrogenase, domain containing 2 // | | |
| NM_145220 // Appl2 // adaptor protein, | Appl2 | 0.57 |
| phosphotyrosine interaction, PH domain an | | |
| NM_011636 // Plscr1 // phospholipid scramblase 1 // 9 | Plscr1 | 0.57 |
| E3.3 // 22038 /// ENSMUST0 | | |
| NM_175088 // Mdfic // MyoD family inhibitor domain | Mdfic | 0.56 |
| containing // 6 A1 // 16543 / | | |
| NM_031167 // Il1rn // interleukin 1 receptor antagonist | Il1rn | 0.55 |
| // 2 A 3\|2 10.0 cM // 161 | | |
| NM_175175 // Plekhf2 // pleckstrin homology domain | Plekhf2 | 0.55 |
| containing, family F (with FY | | |
| NM_024207 // Derl1 // Der1-like domain family, | Derl1 | 0.55 |
| member 1 // 15 D2 // 67819 /// EN | | |
| NM_175937 // Cpeb2 // cytoplasmic polyadenylation | Cpeb2 | 0.55 |
| element binding protein 2 // 5 | | |
| NM_012039 // Zw10 // ZW10 homolog (Drosophila), | Zw10 | 0.55 |
| centromere/kinetochore protein / | | |
| NM_177700 // Atmin // ATM interactor // 8 E1 // | Atmin | 0.55 |
| 234776 /// ENSMUST00000109099 // | | |
| NM_008230 // Hdc // histidine decarboxylase // 2 E5-G | Hdc | 0.55 |
| // 15186 /// ENSMUST000000 | | |
| NM_026189 // Eepd1 // | Eepd1 | 0.55 |
| endonuclease/exonuclease/phosphatase family domain | | |
| contain | | |
| NM_010219 // Fkbp4 // FK506 binding protein 4 // 6 F3 | Fkbp4 | 0.54 |
| // 14228 /// ENSMUST000000 | | |
| NM_001012667 // AI316807 // expressed sequence | AI316807 | 0.54 |
| AI316807 // 8 A2 // 102032 /// NM | | |
| NM_171826 // Cldnd1 // claudin domain containing 1 // | Cldnd1 | 0.53 |
| 16 C1.2 // 224250 /// ENSM | | |
| NM_133886 // AU040320 // expressed sequence | AU040320 | 0.53 |
| AU040320 // 4 D2.2 // 100317 /// NM_{—} | | |
| NM_008913 // Ppp3ca // protein phosphatase 3, | Ppp3ca | 0.53 |
| catalytic subunit, alpha isoform / | | |
| NM_025813 // Mfsd1 // major facilitator superfamily | Mfsd1 | 0.52 |
| domain containing 1 // 3 E2 | | |
| NM_175210 // Abca12 // ATP-binding cassette, sub- | Abca12 | 0.52 |
| family A (ABC1), member 12 // 1 | | |
| BC049563 // 1700028P14Rik // RIKEN cDNA | 1700028P14Rik | 0.52 |
| 1700028P14 gene // 19 B // 67483 /// ENS | | |
| NM_029789 // Lass2 // LAG1 homolog, ceramide | Lass2 | 0.51 |
| synthase 2 // 3 F2 // 76893 /// ENS | | |
| NM_007889 // Dvl3 // dishevelled 3, dsh homolog | Dvl3 | 0.51 |
| (Drosophila) // 16 A3\|16 13.7 cM | | |
| NM_010765 // Mapkapk5 // MAP kinase-activated | Mapkapk5 | 0.50 |
| protein kinase 5 // 5 F // 17165 / | | |
| NM_026875 // Ypel3 // yippee-like 3 (Drosophila) // 7 | Ypel3 | 0.50 |
| F3 // 66090 /// NM_025347 | | |
| NM_178775 // Rps6kc1 // ribosomal protein S6 kinase | Rps6kc1 | 0.48 |
| polypeptide 1 // 1 H6 // 320 | | |
| NM_001025250 // Vegfa // vascular endothelial growth | Vegfa | 0.48 |
| factor A // 17 C\|17 24.2 cM | | |
| NM_026732 // Mrpl14 // mitochondrial ribosomal | Mrpl14 | 0.47 |
| protein L14 // 17 B3 // 68463 /// | | |
| NM_021496 // Pvrl3 // poliovirus receptor-related 3 // | Pvrl3 | 0.47 |
| 16 B5 // 58998 /// NM_021 | | |
| NM_011056 // Pde4d // phosphodiesterase 4D, cAMP | Pde4d | 0.47 |
| specific // 13 D2.1-D2.2 // 238 | | |
| NM_024270 // Stard3nl // STARD3 N-terminal like // | Stard3nl | 0.47 |
| 13 A3.1 // 76205 /// ENSMUST0 | | |
| NM_145486 // March2 // membrane-associated ring | 38777 | 0.47 |
| finger (C3HC4) 2 // 17 B1 // 224 | | |
| NM_138681 // Bcas3 // breast carcinoma amplified | Bcas3 | 0.46 |
| sequence 3 // 11 C // 192197 // | | |
| NM_015828 // Gne // glucosamine // 4 B1 // 50798 /// | Gne | 0.44 |
| ENSMUST00000030201 // Gne / | | |
| NM_001081322 // Myo5c // myosin VC // 9 D // | Myo5c | 0.44 |
| 208943 /// ENSMUST00000036555 // My | | |
| NM_033564 // Mpv17l // Mpv17 transgene, kidney | Mpv17l | 0.43 |
| disease mutant-like // 16 A1 // 9 | | |
| NM_026846 // Zfand2b // zinc finger, AN1 type | Zfand2b | 0.43 |
| domain 2B // 1 C3 // 68818 /// NM_{—} | | |
| NM_028121 // Adpgk // ADP-dependent glucokinase // | Adpgk | 0.42 |
| 9 B // 72141 /// ENSMUST00000 | | |
| NM_010885 // Ndufa2 // NADH dehydrogenase | Ndufa2 | 0.42 |
| (ubiquinone) 1 alpha subcomplex, 2 // | | |
| NM_028071 // Cotl1 // coactosin-like 1 (Dictyostelium) | Cotl1 | 0.42 |
| // 8 E1 // 72042 /// ENSM | | |
| NM_144529 // Arhgap17 // Rho GTPase activating | Arhgap17 | 0.41 |
| protein 17 // 7 F3 // 70497 /// N | | |
| NM_172439 // Inpp5j // inositol polyphosphate 5- | Inpp5j | 0.41 |
| phosphatase J // 11 A1 // 170835 | | |
| NM_021554 // Mettl9 // methyltransferase like 9 // 7 F2 | Mettl9 | 0.41 |
| // 59052 /// ENSMUST0000 | | |
| NM_001081228 // Ttc30a2 // tetratricopeptide repeat | Ttc30a2 | 0.41 |
| domain 30A2 // 2 C3 // 62063 | | |
| NM_213614 // Sept5 // septin 5 // 16 A3\|16 11.42 cM // | 38964 | 0.41 |
| 18951 /// NM_001001999 // | | |
| NM_021461 // Mknk1 // MAP kinase-interacting | Mknk1 | 0.41 |
| serine/threonine kinase 1 // 4 D1 / | | |
| NM_020332 // Ank // progressive ankylosis // 15 B1\|15 | Ank | 0.41 |
| 14.4 cM // 11732 /// ENSMU | | |
| NM_177884 // AW146020 // expressed sequence | AW146020 | 0.40 |
| AW146020 // 6 C3 // 330361 /// NM_02 | | |
| NM_008301 // Hspa2 // heat shock protein 2 // 12 C3\|12 | Hspa2 | 0.40 |
| 34.0 cM // 15512 /// NM_0 | | |
| NM_019923 // Itpr2 // inositol 1,4,5-triphosphate | Itpr2 | 0.39 |
| receptor 2 // 6 G3\|6 73.0 cM / | | |
| NM_027154 // Tmbim1 // transmembrane BAX | Tmbim1 | 0.39 |
| inhibitor motif containing 1 // 1 C3 // | | |
| NM_007867 // Dlx4 // distal-less homeobox 4 // 11 | Dlx4 | 0.38 |
| D\|11 55.0 cM // 13394 /// ENSM | | |
| NM_031391 // Gtf2a1 // general transcription factor II | Gtf2a1 | 0.37 |
| A, 1 // 12 E\|12 // 83602 | | |
| NM_001039493 // Plekhm3 // pleckstrin homology | Plekhm3 | 0.37 |
| domain containing, family M, memb | | |
| NM_011304 // Ruvbl2 // RuvB-like protein 2 // 7 B2 // | Ruvbl2 | 0.37 |
| 20174 /// NM_030678 // Gys | | |
| NM_013843 // Zfp53 // zinc finger protein 53 // 17 | Zfp53 | 0.36 |
| A3.2\|17 9.9 cM // 24132 /// N | | |
| NM_008149 // Gpam // glycerol-3-phosphate | Gpam | 0.36 |
| acyltransferase, mitochondrial // 19 D | | |
| NM_008813 // Enpp1 // ectonucleotide | Enpp1 | 0.35 |
| pyrophosphatase/phosphodiesterase 1 // 10 A | | |
| NM_145705 // Tinf2 // Terf1 (TRF1)-interacting | Tinf2 | 0.34 |
| nuclear factor 2 // 14 C3\|14 22.5 | | |
| NM_207670 // Gripap1 // GRIP1 associated protein 1 // | Gripap1 | 0.34 |
| X A1.1\|X 2.0 cM // 54645 / | | |
| NM_172284 // Ddx19b // DEAD (Asp-Glu-Ala-Asp) | Ddx19b | 0.34 |
| box polypeptide 19b // 8 E1 // 234 | | |
| NM_011018 // Sqstm1 // sequestosome 1 // 11 B1.2 // | Sqstm1 | 0.34 |
| 18412 /// ENSMUST00000102774 | | |
| NM_001081009 // Parp8 // poly (ADP-ribose) | Parp8 | 0.33 |
| polymerase family, member 8 // 13 D2. | | |
| NM_001081249 // Vcan // versican // 13 C3\|13 55.0 cM | Vcan | 0.32 |
| // 13003 /// NM_019389 // V | | |
| NM_145390 // Tnpo2 // transportin 2 (importin 3, | Tnpo2 | 0.31 |
| karyopherin beta 2b) // 8 C3 // | | |
| NM_011173 // Pros1 // protein S (alpha) // 16 C1.3 // | Pros1 | 0.31 |
| 19128 /// ENSMUST000000236 | | |
| NM_025575 // Sys1 // SYS1 Golgi-localized integral | Sys1 | 0.31 |
| membrane protein homolog (S. | | |
| NM_146550 // Olfr810 // olfactory receptor 810 // --- // | O1fr810 | 0.30 |
| 258543 /// ENSMUST00000 | | |
| BC043106 // 4933427D14Rik // RIKEN cDNA | 4933427D14Rik | 0.30 |
| 4933427D14 gene // 11 B4 // 74477 /// BC | | |
| NM_025389 // Anapc11 // anaphase promoting | Anapc11 | 0.30 |
| complex subunit 11 // 11 E2 // 66156 | | |
| NM_013842 // Xbp1 // X-box binding protein 1 // 11 | Xbp1 | 0.29 |
| A1\|11 3.0 cM // 22433 /// ENS | | |
| NM_025453 // Tm4sf20 // transmembrane 4 L six | Tm4sf20 | 0.28 |
| family member 20 // 1 C5 // 66261 | | |
| NM_146977 // Olfr1255 // olfactory receptor 1255 // --- | Olfr1255 | 0.28 |
| // 258979 /// ENSMUST000 | | |
| NM_153058 // Mapre2 // microtubule-associated | Mapre2 | 0.27 |
| protein, RP/EB family, member 2 // | | |
| NM_026849 // Mtmr14 // myotubularin related protein | Mtmr14 | 0.27 |
| 14 // 6 E3 // 97287 /// ENSM | | |
| NM_009372 // Tgif1 // TGFB-induced factor homeobox | Tgif1 | 0.26 |
| 1 // 17 E1.3 // 21815 /// ENS | | |
| NM_026321 // Fam174a // family with sequence | Fam174a | 0.26 |
| similarity 174, member A // 1 D // | | |
| NM_001037987 // Edil3 // EGF-like repeats and | Edil3 | 0.26 |
| discoidin I-like domains 3 // --- | | |
| NM_021435 // Slc35b4 // solute carrier family 35, | Slc35b4 | 0.25 |
| member B4 // 6 B1 // 58246 /// | | |
| NM_016846 // Rgl1 // ral guanine nucleotide | Rgl1 | 0.25 |
| dissociation stimulator, -like 1 // 1 | | |
| NM_028223 // Tmem175 // transmembrane protein 175 | Tmem175 | 0.25 |
| // 5 F // 72392 /// NM_199011 | | |
| NM_011120 // Prl7d1 // prolactin family 7, subfamily d, | Prl7d1 | 0.23 |
| member 1 // 13 A3.1 // 1 | | |
| NM_019426 // Atf7ip // activating transcription factor 7 | Atf7ip | 0.23 |
| interacting protein // | | |
| NM_026530 // Mpnd // MPN domain containing // 17 D | Mpnd | 0.23 |
| // 68047 /// ENSMUST000000032 | | |
| NM_009579 // Slc30a1 // solute carrier family 30 (zinc | Slc30a1 | 0.22 |
| transporter), member 1 // | | |
| NM_001162869 // Rab11fip3 // RAB11 family | Rab11fip3 | 0.22 |
| interacting protein 3 (class II) // 17 | | |
| NM_030250 // Nus1 // nuclear undecaprenyl | Nus1 | 0.21 |
| pyrophosphate synthase 1 homolog (S. c | | |
| NM_028835 // Atg7 // autophagy-related 7 (yeast) // 6 | Atg7 | 0.21 |
| E3 // 74244 /// NM_0011150 | | |
| NM_178890 // Abtb2 // ankyrin repeat and BTB (POZ) | Abtb2 | 0.20 |
| domain containing 2 // 2 E2 / | | |
| NM_001080974 // Sri // sorcin // 5 A1-h\|5 1.0 cM // | Sri | 0.19 |
| 109552 /// NM_025618 // Sri | | |
| NM_010915 // Klk1b4 // kallikrein 1-related pepidase | Klk1b4 | 0.18 |
| b4 // 7 B4\|7 23.02 cM // 18 | | |
| NM_009411 // Tpbpa // trophoblast specific protein | Tpbpa | 0.17 |
| alpha // 13 B2\|13 36.0 cM // | | |
| NM_007759 // Crabp2 // cellular retinoic acid binding | Crabp2 | 0.17 |
| protein II // 3F1\|3 // 129 | | |
| NM_011781 // Adam25 // a disintegrin and | Adam25 | 0.17 |
| metallopeptidase domain 25 (testase 2) | | |
| NM_011377 // Sim2 // single-minded homolog 2 | Sim2 | 0.16 |
| (Drosophila) // 16 C3.3-C4\|16 67.57 | | |
| NM_199306 // Wdtc1 // WD and tetratricopeptide | Wdtc1 | 0.15 |
| repeats 1 // 4 D2.3 // 230796 /// | | |
| NM_001145778 // Zkscan3 // zinc finger with KRAB | Zkscan3 | 0.14 |
| and SCAN domains 3 // 13 A3.1 / | | |
| NM_001083927 // Tle3 // transducin-like enhancer of | Tle3 | 0.14 |
| split 3, homolog of Drosophi | | |
| NM_146724 // Olfr512 // olfactory receptor 512 // --- // | Olfr512 | 0.14 |
| 258719 /// NM_146725 // | | |
| NM_008027 // Flot1 // flotillin 1 // 17 B1 // 14251 /// | Flot1 | 0.13 |
| ENSMUST00000001569 // Fl | | |
| NM_175112 // Rae1 // RAE1 RNA export 1 homolog | Rae1 | 0.12 |
| (S. pombe) // 2 H3\|2 103.0 cM // | | |
| NM_175103 // Bola2 // bolA-like 2 (E. coli) // 7 F3 // | Bola2 | 0.11 |
| 66162 /// ENSMUST00000052 | | |

Values are relative to Ad-ATF4ΔbZIP; P ≦ 0.01

TABLE 2
MOUSE TA MUSCLE MRNAS THAT ARE DECREASED IN FASTED ATF4 MKO TA
MUSCLE

| | | Log2 Δ |
|---|---|---|
| | | ATF4 mKO- |
| gene_assignment | Gene Symbol | Control |
| NM_009716 // Atf4 // activating transcription factor 4 // 15 | Atf4 | −1.76 |
| E1\|15 43.3 cM // 11 | | |
| NM_009884 // Cebpg // CCAAT/enhancer binding protein | Cebpg | −0.93 |
| (C/EBP), gamma // 7 B1 // 1 | | |
| NM_013742 // Cars // cysteinyl-tRNA synthetase // 7 F5\|7 | Cars | −0.83 |
| 69.0 cM // 27267 /// EN | | |
| NM_134151 // Yars // tyrosyl-tRNA synthetase // 4 D2.2 // | Yars | −0.81 |
| 107271 /// ENSMUST0000 | | |
| NM_146370 // Olfr47 // olfactory receptor 47 // 6 B2.1 // | Olfr47 | −0.78 |
| 18346 /// ENSMUST00000 | | |
| NM_007593 // Cetn1 // centrin 1 // 18 A2 // 26369 /// | Cetn1 | −0.72 |
| ENSMUST00000062769 // Cetn | | |
| NM_001003913 // Mars // methionine-tRNA synthetase // 10 | Mars | −0.63 |
| D3 // 216443 /// ENSMUS | | |
| NM_172015 // Iars // isoleucine-tRNA synthetase // 13 A5 // | Iars | −0.61 |
| 105148 /// ENSMUST00 | | |
| NM_133800 // Nol12 // nucleolar protein 12 // 15 E1 // | Nol12 | −0.60 |
| 97961 /// ENSMUST00000041 | | |
| NM_207575 // Olfr1480 // olfactory receptor 1480 // 19 A // | Olfr1480 | −0.60 |
| 404339 /// ENSMUST00 | | |
| NM_146217 // Aars // alanyl-tRNA synthetase // 8 E1 // | Aars | −0.57 |
| 234734 /// NM_028274 // E | | |
| NM_180678 // Gars // glycyl-tRNA synthetase // 6 B3\|6 32.5 cM | Gars | −0.52 |
| // 353172 /// ENSM | | |
| NM_146845 // Olfr1098 // olfactory receptor 1098 // --- // | Olfr1098 | −0.52 |
| 258842 /// NM_146843 | | |
| NM_145851 // Cables2 // CDK5 and Abl enzyme substrate 2 | Cables2 | −0.46 |
| // 2 H4 // 252966 /// EN | | |
| NM_134137 // Lars // leucyl-tRNA synthetase // 18 E // | Lars | −0.44 |
| 107045 /// ENSMUST0000009 | | |
| NM_007836 // Gadd45a // growth arrest and DNA-damage- | Gadd45a | −0.43 |
| inducible 45 alpha // 6 C1 | | |
| NM_053113 // Ear11 // eosinophil-associated, ribonuclease | Ear11 | −0.43 |
| A family, member 11 // | | |
| NM_001142950 // Nars // asparaginyl-tRNA synthetase // 18 | Nars | −0.42 |
| E1 // 70223 /// NM_027 | | |
| BC006954 // Epha2 // Eph receptor A2 // 4 D-E\|4 73.2 cM // | Epha2 | −0.42 |
| 13836 | | |
| NM_033074 // Tars // threonyl-tRNA synthetase // 15 A1\|15 | Tars | −0.41 |
| 6.7 cM // 110960 /// E | | |
| NM_030693 // Atf5 // activating transcription factor 5 // 7 B4 | Atf5 | −0.41 |
| // 107503 /// ENS | | |
| NM_008464 // Klra6 // killer cell lectin-like receptor, | Klra6 | −0.40 |
| subfamily A, member 6 // | | |
| NM_183126 // 6030498E09Rik // RIKEN cDNA | 6030498E09Rik | −0.39 |
| 6030498E09 gene // X A3.3 // 77883 /// | | |
| NM_008638 // Mthfd2 // methylenetetrahydrofolate | Mthfd2 | −0.38 |
| dehydrogenase (NAD+ dependent), | | |
| NM_010340 // Gpr50 // G-protein-coupled receptor 50 // X | Gpr50 | −0.38 |
| A7.2\|X 26.0 cM // 14765 | | |
| NM_198251 // Rnf133 // ring finger protein 133 // 6 A3.1 // | Rnf133 | −0.38 |
| 386611 /// ENSMUST00 | | |
| NM_026030 // Eif2s2 // eukaryotic translation initiation | Eif2s2 | −0.38 |
| factor 2, subunit 2 (be | | |
| NM_146817 // Olfr1156 // olfactory receptor 1156 // --- // | Olfr1156 | −0.37 |
| 258814 /// ENSMUST000 | | |
| NM_028622 // Lce1c // late cornified envelope 1C // 3 F1 // | Lce1c | −0.34 |
| 73719 /// ENSMUST000 | | |
| NM_001143689 // H2-gs10 // MHC class I like protein GS10 | H2-gs10 | −0.33 |
| // 17 B1\|17 // 436493 / | | |
| NM_019665 // Arl6 // ADP-ribosylation factor-like 6 // 16 | Arl6 | −0.32 |
| C1.2 // 56297 /// ENSM | | |
| NM_175110 // 5730577I03Rik // RIKEN cDNA 5730577I03 | 5730577I03Rik | −0.31 |
| gene // 9 A3 // 66662 /// EN | | |
| NM_198322 // Zfp273 // zinc finger protein 273 // 13 B3 // | Zfp273 | −0.29 |
| 212569 ///NM_0010011 | | |
| BC049886 // 9130221D24Rik // RIKEN cDNA 9130221D24 | 9130221D24Rik | −0.29 |
| gene // 3 H2 // 77669 /// ENS | | |
| NM_146623 // Olfr357 // olfactory receptor 357 // --- // | Olfr357 | −0.29 |
| 258616 /// ENSMUST00000 | | |
| NM_008487 // Arhgef2 // rho/rac guanine nucleotide | Arhgef2 | −0.29 |
| exchange factor (GEF) 2 // 3 | | |
| NM_009435 // Tssk1 // testis-specific serine kinase 1 // 16 | Tssk1 | −0.28 |
| A3\|16 10.4 cM // 221 | | |
| NM_153512 // Kcng3 // potassium voltage-gated channel, | Kcng3 | −0.28 |
| subfamily G, member 3 // | | |
| NM_025938 // Rpp14 // ribonuclease P 14 subunit (human) // | Rpp14 | −0.27 |
| 14 A1 // 67053 /// EN | | |
| BC099538 // 1700065I17Rik // RIKEN cDNA 1700065I17 | 1700065I17Rik | −0.27 |
| gene // 18 D2 // 67343 /// NM | | |
| NM_020000 // Med8 // mediator of RNA polymerase II | Med8 | −0.27 |
| transcription, subunit 8 homo | | |
| NM_001112731 // C030039L03Rik // RIKEN cDNA | C030039L03Rik | −0.27 |
| C030039L03 gene // 7 A3 // 112415 // | | |
| NM_030013 // Cyp20a1 // cytochrome P450, family 20, | Cyp20a1 | −0.26 |
| subfamily A, polypeptide 1 / | | |
| NM_025323 // 0610009D07Rik // RIKEN cDNA | 0610009D07Rik | −0.25 |
| 0610009D07 gene // 12 A1.1 // 66055 /// | | |
| NM_001136089 // Anxa10 // annexin A10 // 8 B3.1\|8 32.0 cM | Anxa10 | −0.25 |
| // 26359 /// NM_011922 | | |
| NM_020611 // Srd5a3 // steroid 5 alpha-reductase 3 // 5 C3.3 | Srd5a3 | −0.25 |
| // 57357 /// ENSMUS | | |
| NM_009411 // Tpbpa // trophoblast specific protein alpha // | Tpbpa | −0.25 |
| 13 B2\|13 36.0 cM // | | |
| NM_199056 // Ippk // inositol 1,3,4,5,6-pentakisphosphate 2- | Ippk | −0.25 |
| kinase // 13 A5 // 7 | | |
| NM_028057 // Cyb5r1 // cytochrome b5 reductase 1 // 1 E4 | Cyb5r1 | −0.25 |
| // 72017 /// ENSMUST000 | | |
| NM_001039530 // Parp14 // poly (ADP-ribose) polymerase | Parp14 | −0.24 |
| family, member 14 // 16 B | | |
| NM_030558 // Car15 // carbonic anhydrase 15 // 16 A3 // | Car15 | −0.24 |
| 80733 /// ENSMUST0000011 | | |
| NM_172267 // Phyhd1 // phytanoyl-CoA dioxygenase | Phyhd1 | −0.23 |
| domain containing 1 // 2 B // 2 | | |
| NM_173866 // Gpt2 // glutamic pyruvate transaminase | Gpt2 | −0.23 |
| (alanine aminotransferase) 2 | | |
| NM_134138 // Psmg2 // proteasome (prosome, macropain) | Psmg2 | −0.23 |
| assembly chaperone 2 // 18 | | |
| NM_146741 // Olfr1497 // olfactory receptor 1497 // --- // | Olfr1497 | −0.23 |
| 258736 /// ENSMUST000 | | |
| NM_011428 // Snap25 // synaptosomal-associated protein 25 | Snap25 | −0.23 |
| // 2 F3\|2 78.2 cM // 2 | | |
| NM_008188 // Thumpd3 // THUMP domain containing 3 // 6 | Thumpd3 | −0.22 |
| E3\|6 48.7 cM // 14911 /// | | |
| NM_001123372 // Gm3435 // predicted gene 3435 // 17 A2 | Gm3435 | −0.22 |
| // 100041621 /// NM_00112 | | |
| NM_013478 // Azgp1 // alpha-2-glycoprotein 1, zinc // 5 | Azgp1 | −0.22 |
| G2\|5 78.0 cM // 12007 // | | |
| NM_019918 // Vmn2r1 // vomeronasal 2, receptor 1 // 3 E1 | Vmn2r1 | −0.22 |
| // 56544 /// ENSMUST000 | | |
| NM_194055 // Esrp1 // epithelial splicing regulatory protein | Esrp1 | −0.21 |
| 1 // 4 A1 // 207920 | | |
| NM_011731 // Slc6a20b // solute carrier family 6 | Slc6a20b | −0.21 |
| (neurotransmitter transporter), | | |
| NM_027934 // Rnf180 // ring finger protein 180 // 13 D1 // | Rnf180 | −0.21 |
| 71816 /// ENSMUST0000 | | |
| NR_027806 // Pea15b // phosphoprotein enriched in | Pea15b | −0.21 |
| astrocytes 15B // 5 C3.3 // 23 | | |
| NM_029771 // Gper // G protein-coupled estrogen receptor 1 | Gper | −0.20 |
| // 5 G1 // 76854 /// | | |
| NM_010663 // Krt17 // keratin 17 // 11 D\|11 58.7 cM // | Krt17 | −0.20 |
| 16667 /// ENSMUST00000080 | | |
| NM_146200 // Eif3c // eukaryotic translation initiation factor | Eif3c | −0.20 |
| 3, subunit C // - | | |
| NM_146790 // Olfr1238 // olfactory receptor 1238 // --- // | Olfr1238 | −0.20 |
| 258786 /// ENSMUST000 | | |
| NM_001039519 // Gtf2a2 // general transcription factor II A, | Gtf2a2 | −0.19 |
| 2 // 9 D // 235459 | | |
| NM_146035 // Mgat2 // mannoside | Mgat2 | −0.19 |
| acetylglucosaminyltransferase 2 // 12 C2 // 2176 | | |
| NM_145430 // BC017647 // cDNA sequence BC017647 // | BC017647 | −0.19 |
| 11 B5 // 216971 /// ENSMUST00 | | |
| NM_010062 // Dnase2a // deoxyribonuclease II alpha // 8 | Dnase2a | −0.19 |
| C3\|8 38.6 cM // 13423 // | | |
| NM_010323 // Gnrhr // gonadotropin releasing hormone | Gnrhr | −0.18 |
| receptor // 5 E1\|5 44.0 cM | | |
| NM_173395 // Fam132b // family with sequence similarity | Fam132b | −0.18 |
| 132, member B // 1 D // | | |
| NM_148939 // Ly6g5b // lymphocyte antigen 6 complex, | Ly6g5b | −0.18 |
| locus G5B // 17 B1 // 26661 | | |
| NM_172932 // Nlgn3 // neuroligin 3 // X D // 245537 /// | Nlgn3 | −0.17 |
| ENSMUST00000065858 // Nl | | |
| NM_138593 // Larp7 // La ribonucleoprotein domain family, | Larp7 | −0.17 |
| member 7 // 3 G2\|3 62. | | |
| NM_008409 // Itm2a // integral membrane protein 2A // X | Itm2a | −0.17 |
| A2-A3 // 16431 /// ENSMU | | |
| NM_030057 // Trappc6b // trafficking protein particle | Trappc6b | −0.17 |
| complex 6B // 12 C2 // 782 | | |
| NM_008926 // Prkg2 // protein kinase, cGMP-dependent, | Prkg2 | −0.17 |
| type II // 5 E3\|5 53.0 cM | | |
| NM_175460 // Nmnat2 // nicotinamide nucleotide | Nmnat2 | −0.17 |
| adenylyltransferase 2 // 1 G3 // | | |
| BC054747 // Wdr62 // WD repeat domain 62 // 7 B1 // | Wdr62 | −0.17 |
| 233064 /// BC057041 // Wdr62 | | |
| NM_009474 // Uox // urate oxidase // 3 H2\|3 75.0 cM // | Uox | −0.16 |
| 22262 /// ENSMUST00000029 | | |
| NM_145938 // Rpp40 // ribonuclease P 40 subunit (human) // | Rpp40 | −0.16 |
| 13 A3.3 // 208366 /// | | |
| NM_001033123 // Gm14288 // predicted gene 14288 // 2 H4 | Gm14288 | −0.16 |
| // 13999 /// NM_00110180 | | |
| NM_007813 // Cyp2b13 // cytochrome P450, family 2, | Cyp2b13 | −0.16 |
| subfamily b, polypeptide 13 / | | |
| NM_027171 // 2310057J16Rik // RIKEN cDNA 2310057J16 | 2310057J16Rik | −0.16 |
| gene // 8 A1.1 // 69697 /// | | |
| NM_146092 // Taf6l // TAF6-like RNA polymerase II, | Taf6l | −0.16 |
| p300/CBP-associated factor (P | | |
| NM_001161355 // Timd2 // T-cell immunoglobulin and | Timd2 | −0.16 |
| mucin domain containing 2 // | | |
| NM_001081188 // Exosc7 // exosome component 7 // 9 F4 // | Exosc7 | −0.16 |
| 66446 /// ENSMUST000000 | | |
| NM_023598 // Arid5b // AT rich interactive domain 5B | Arid5b | −0.16 |
| (MRF1-like) // 10 B5.1 // 7 | | |
| NM_201255 // Krt9 // keratin 9 // 11 D // 107656 /// | Krt9 | −0.15 |
| ENSMUST00000059707 // Krt9 | | |
| NM_019424 // Hps1 // Hermansky-Pudlak syndrome 1 | Hps1 | −0.15 |
| homolog (human) // 19 C3\|19 42. | | |
| NM_025945 // Polr3d // polymerase (RNA) III (DNA | Polr3d | −0.15 |
| directed) polypeptide D // 14 D | | |
| NM_175020 // BC048599 // cDNA sequence BC048599 // 6 | BC048599 | −0.15 |
| B1 // 232717 /// ENSMUST000 | | |
| NM_030690 // Rai14 // retinoic acid induced 14 // 15 A2 // | Rai14 | −0.15 |
| 75646 /// ENSMUST0000 | | |
| NM_029031 // Shpk // sedoheptulokinase // 11 B4 // 74637 | Shpk | −0.15 |
| /// ENSMUST00000006105 | | |
| NM_026932 // Ebna1bp2 // EBNA1 binding protein 2 // 4 | Ebna1bp2 | −0.14 |
| D2.1 // 69072 /// ENSMUST0 | | |
| NM_175500 // Gpc5 // glypican 5 // --- // 103978 /// | Gpc5 | −0.14 |
| ENSMUST00000022707 // Gpc5 | | |
| NM_029865 // Ocell // occludin/ELL domain containing 1 // | Ocel1 | −0.13 |
| 8 C1 // 77090 /// ENSM | | |
| NM_172587 // Cdc14b // CDC14 cell division cycle 14 | Cdc14b | −0.13 |
| homolog B (S. cerevisiae) // | | |
| NM_011508 // Eif1 // eukaryotic translation initiation factor | Eif1 | −0.13 |
| 1 // 11 D // 20918 | | |
| NM_172590 // Wdr41 // WD repeat domain 41 // 13 D1 // | Wdr41 | −0.12 |
| 218460 /// ENSMUST00000056 | | |
| NM_019488 // Slc2a8 // solute carrier family 2, (facilitated | Slc2a8 | −0.12 |
| glucose transporter | | |
| NM_001142647 // Tmem194b // transmembrane protein | Tmem194b | −0.12 |
| 194B // 1 C1.1 // 227094 /// N | | |
| NM_181402 // Parp11 // poly (ADP-ribose) polymerase | Parp11 | −0.12 |
| family, member 11 // 6 F3 // | | |
| NM_010623 // Kif17 // kinesin family member 17 // 4 D3 // | Kif17 | −0.12 |
| 16559 /// NM_001099631 | | |
| NM_010481 // Hspa9 // heat shock protein 9 // 18 C\|18 15.0 cM | Hspa9 | −0.12 |
| // 15526 /// ENSMU | | |
| NM_175097 // Prickle3 // prickle homolog 3 (Drosophila) // | Prickle3 | −0.11 |
| XA1.1\|X 1.6 cM // 54 | | |
| NM_178367 // Dhx33 // DEAH (Asp-Glu-Ala-His) box | Dhx33 | −0.11 |
| polypeptide 33 // 11 B4 // 2168 | | |
| NM_019781 // Pex14 // peroxisomal biogenesis factor 14 // 4 | Pex14 | −0.11 |
| E2 // 56273 /// ENSM | | |
| NM_198927 // Smgc // submandibular gland protein C // 15 | Smgc | −0.10 |
| E3 // 223809 /// ENSMUS | | |
| NM_013528 // Gfpt1 // glutamine fructose-6-phosphate | Gfpt1 | −0.10 |
| transaminase 1 // 6 D1\|6 35 | | |
| NM_021535 // Smu1 // smu-1 suppressor of mec-8 and unc- | Smu1 | −0.10 |
| 52 homolog (C. elegans) / | | |
| NM_028020 // Cpsf31// cleavage and polyadenylation | Cpsf31 | −0.10 |
| specific factor 3-like // 4 | | |
| NM_025609 // Map3k7ip1 // mitogen-activated protein | Map3k7ip1 | −0.09 |
| kinase kinase kinase 7 inter | | |
| NM_020516 // Slc16a8 // solute carrier family 16 | Slc16a8 | −0.09 |
| (monocarboxylic acid transporte | | |
| NM_007732 // Col17a1 // collagen, type XVII, alpha 1 // 19 | Col17a1 | −0.09 |
| D1\|19 49.0 cM // 1282 | | |
| NM_009840 // Cct8 // chaperonin containing Tcp1, subunit 8 | Cct8 | −0.09 |
| (theta) // 16 C3.3 // | | |
| NM_145436 // Cdc27 // cell division cycle 27 homolog (S. cerevisiae) | Cdc27 | −0.09 |
| // 11 E1\|11 | | |
| NM_133839 // Mmadhc // methylmalonic aciduria | Mmadhc | −0.09 |
| (cobalamin deficiency) cblD type, | | |
| NM_001001445 // Trpv1 // transient receptor potential cation | Trpv1 | −0.09 |
| channel, subfamily | | |
| NM_001146024 // Zfp444 // zinc finger protein 444 // 7 A1 // | Zfp444 | −0.08 |
| 72667 /// NM_028316 | | |
| NM_026409 // Ddx55 // DEAD (Asp-Glu-Ala-Asp) box | Ddx55 | −0.08 |
| polypeptide 55 // 5 F // 67848 | | |
| ENSMUST00000084362 // 9630013D21Rik // RIKEN | 9630013D21Rik | −0.08 |
| cDNA 9630013D21 gene // 4 C7 // 319 | | |
| NM_175112 // Rae1 // RAE1 RNA export 1 homolog (S. pombe) | Rae1 | −0.07 |
| // 2 H3\|2 103.0 cM // | | |
| NM_016775 // Dnajc5 // DnaJ (Hsp40) homolog, subfamily | Dnajc5 | −0.07 |
| C, member 5 // 2 H4\|2 106 | | |
| NM_145224 // Tbx22 // T-box 22 // X D\|X 49.0 cM // | Tbx22 | −0.06 |
| 245572 /// NM_181319 // Tbx22 | | |
| NM_018796 // Eef1b2 // eukaryotic translation elongation | Eef1b2 | −0.06 |
| factor 1 beta 2 // 1 C2 | | |
| NM_031870 // Msh4 // mutS homolog 4 (E. coli) // 3 H3 // | Msh4 | −0.06 |
| 55993 /// ENSMUST000000 | | |
| NM_146577 // Olfr1043 // olfactory receptor 1043 // --- // | Olfr1043 | −0.05 |
| 258570 /// ENSMUST000 | | |
| NM_172669 // Ambra1 // autophagy/beclin 1 regulator 1 // 2 | Ambra1 | −0.04 |
| E1 // 228361 /// NM_0 | | |
| NM_052977 // Adarb2 // adenosine deaminase, RNA- | Adarb2 | −0.03 |
| specific, B2 // 13 A1 // 94191 / | | |

Values are relative to fasted littermate control TA muscle; P ≦ 0.01

(c) Gadd45a is Required for Muscle Fiber a Trophy Induced by Immobilization, Fasting, and Denervation

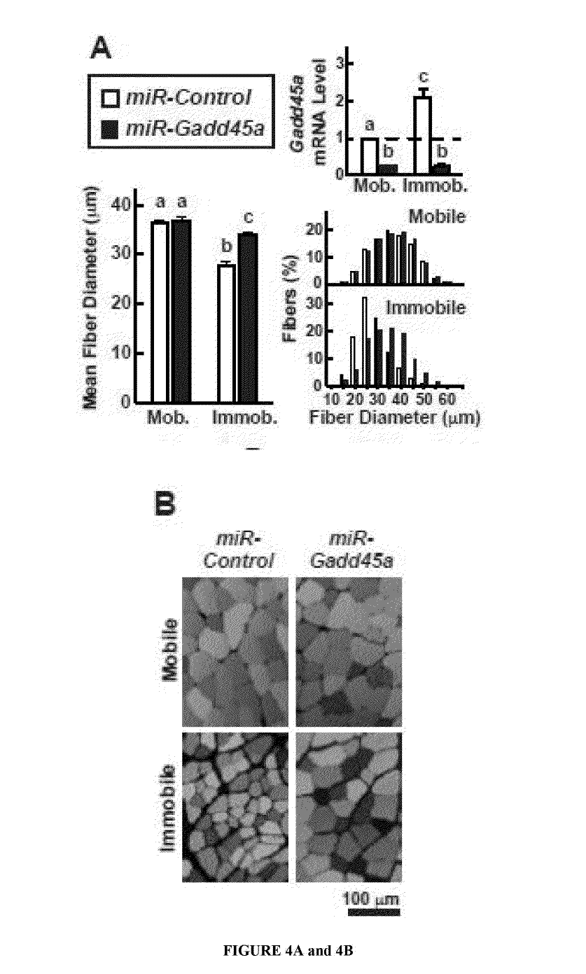

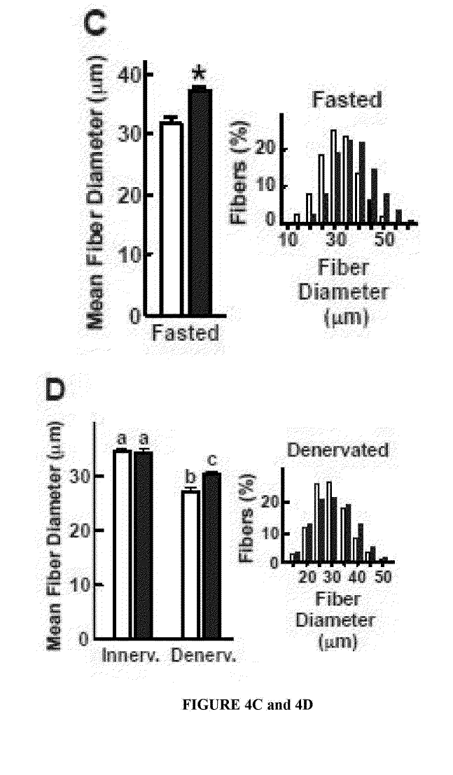

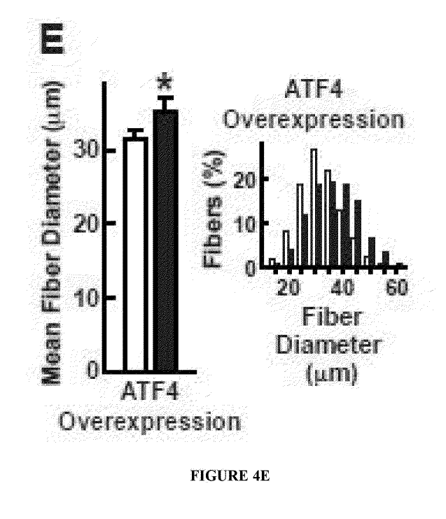

To test the function of Gadd45a, bilateral TAs of C57BL/6 mice were transfected with plasmids encoding artificial miRNAs targeting Gadd45a (miR-Gadd45a). TAs of control mice were transfected with plasmid expressing a non-targeting control miRNA (miR-Control). All plasmids co-expressed EmGFP as a transfection marker. Plasmid transfection was achieved via electroporation, which transfects terminally differentiated muscle fibers, but not satellite or connective tissue cells (Sartori, R., et al. (2009) Am. J. Physiol. Cell Physiol. 296, C1248-C1257). Three days after transfection, unilateral TA immobilization was performed, and 1 week later, bilateral TAs were harvested and compared. In control (mobile) muscles, miR-Gadd45a did not alter muscle fiber size (FIGS. 4A and 4B); thus, reduction of Gadd45a, like loss of ATF4, did not induce fiber hypertrophy. However, in immobilized muscles, miR-Gadd45a prevented the induction of Gadd45a mRNA (FIG. 4A), and reduced muscle fiber atrophy (FIGS. 4A and 4B). Similar results were obtained with a second miR-Gadd45a construct that targeted a different region of the Gadd45a transcript (FIG. 5A). These data indicate that Gadd45a is required for immobilization-induced atrophy.

To determine whether Gadd45a might play a broader role in muscle atrophy, the effects of miR-Gadd45a during fasting and muscle denervation were examined. To investigate fasting, miR-Gadd45a was transfected into one TA, and miR-Control into the contralateral TA. The mice were then subjected to a 24-h fast. miR-Gadd45a significantly impaired fasting-induced muscle fiber atrophy (FIG. 46). Similar results were obtained with a second miR-Gadd45a construct (FIG. 5B).

Like immobilization and fasting, muscle denervation strongly induces atrophy and Gadd45a mRNA (Zeman, R. J., (2009) Pflugers Arch. 458, 525-535). To test the role of Gadd45a in denervated muscle, miR-Control or miR-Gadd45a were transfected bilaterally, then one sciatic nerve was transected to induce atrophy, leaving the contralateral leg as an intrasubject control. One week later, innervated and denervated muscles were compared. Under control conditions, denervation reduced muscle fiber size by 22±3% (FIG. 4D). However, in the presence of miR-Gadd45a, denervation reduced muscle fiber size by only 12±2% (FIG. 4D), indicating a 45% reduction in denervation-mediated atrophy. Interestingly, in all three atrophy models that were examined (immobilization, fasting, and denervation), miR-Gadd45a protected type II but not type I muscle fibers from atrophy (FIG. 5C-5F). The percentages of type I and type II fibers were unchanged (FIG. 5C-5F).

To determine whether Gadd45a is required for atrophy induced by ATF4 overexpression, plasmid encoding ATF4 was co-transfected with miR-Control or miR-Gadd45a. It was found that miR-Gadd45a increased fiber size, indicating reduced ATF4-mediated atrophy (FIG. 4E). Taken together, these data indicate that Gadd45a is required for atrophy induced by immobilization, fasting, denervation, and ATF4 overexpression.

(d) Gadd45a Induces Myotube a Trophy in Vitro and Skeletal Muscle Fiber a Trophy in Vivo

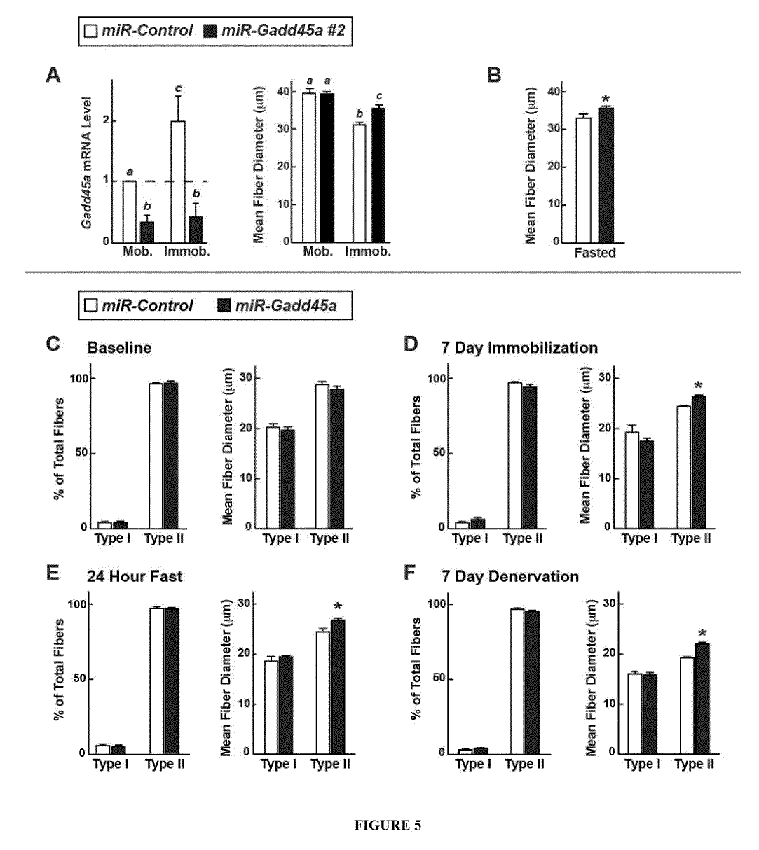

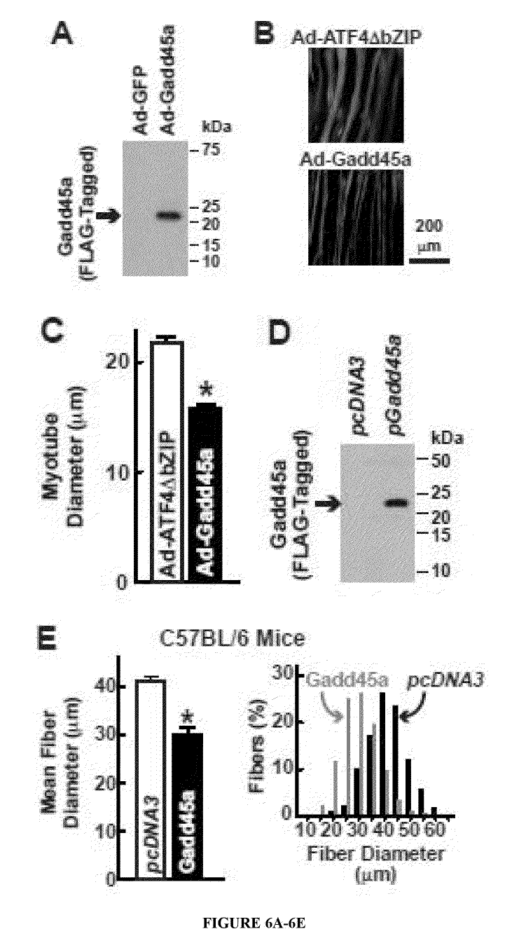

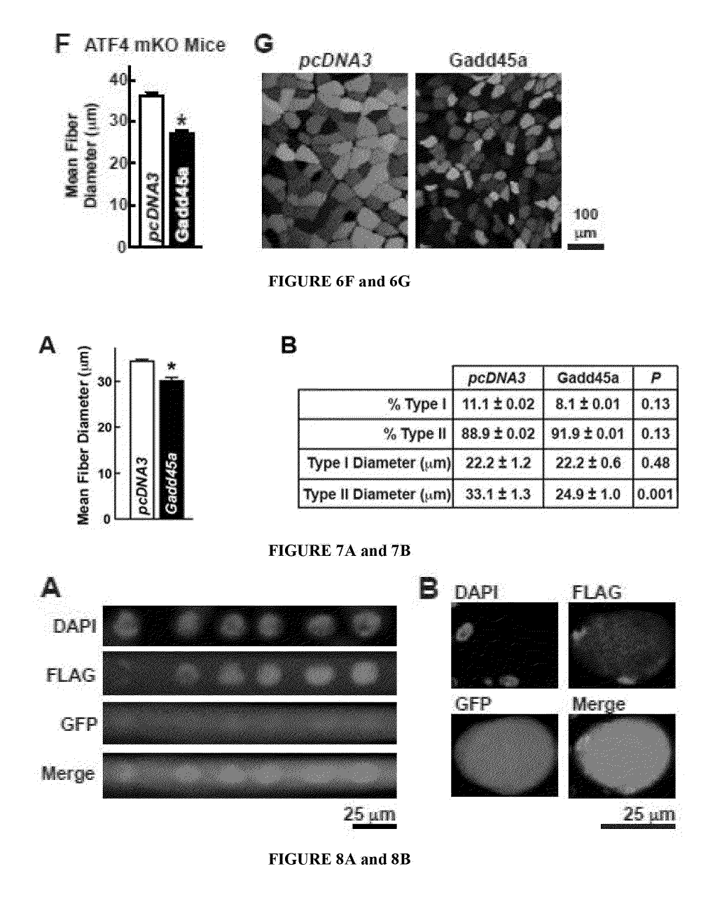

To test whether Gadd45a overexpression induces atrophy, myotubes were transfected with adenovirus co-expressing Gadd45a and GFP (Ad-Gadd45a). Immunoblot analysis confirmed Gadd45a overexpression (FIG. 6A). Like Ad-ATF4, Ad-Gadd45a induced myotube atrophy (FIGS. 6B and 6C).

To determine whether Gadd45a overexpression might induce muscle fiber atrophy in vivo, plasmid encoding Gadd45a was transfected into C57BL/6 TA muscle. The contralateral TA muscle was transfected with empty plasmid vector (pcDNA3). To identify transfected muscle fibers, bilateral TA muscles were co-transfected with plasmid encoding eGFP (pCMV-eGFP), a transfection marker that does not alter muscle fiber size. Immunoblot analysis confirmed Gadd45a over-expression specifically in the TA muscle that was transfected with Gadd45a plasmid (FIG. 6D). Relative to control transfected fibers, muscle fibers transfected with Gadd45a were significantly smaller (FIG. 6E). To test whether Gadd45a-mediated atrophy requires ATF4, Gadd45a plasmid were transfected into ATF4 mKO TA muscles. Gadd45a induced atrophy under both fed conditions (FIGS. 6F and 6G) and fasted conditions (FIG. 7A), indicating that ATF4 is not required for Gadd45a-mediated atrophy. Gadd45a overexpression did not alter the percentages of type I or type II muscle fibers, and it promoted atrophy of type II but not type I fibers (FIG. 7B). Thus, increased Gadd45a expression causes atrophy both in vitro and in vivo.

(e) Gadd45a Enters Myonuclei and Induces Myonuclear Remodeling

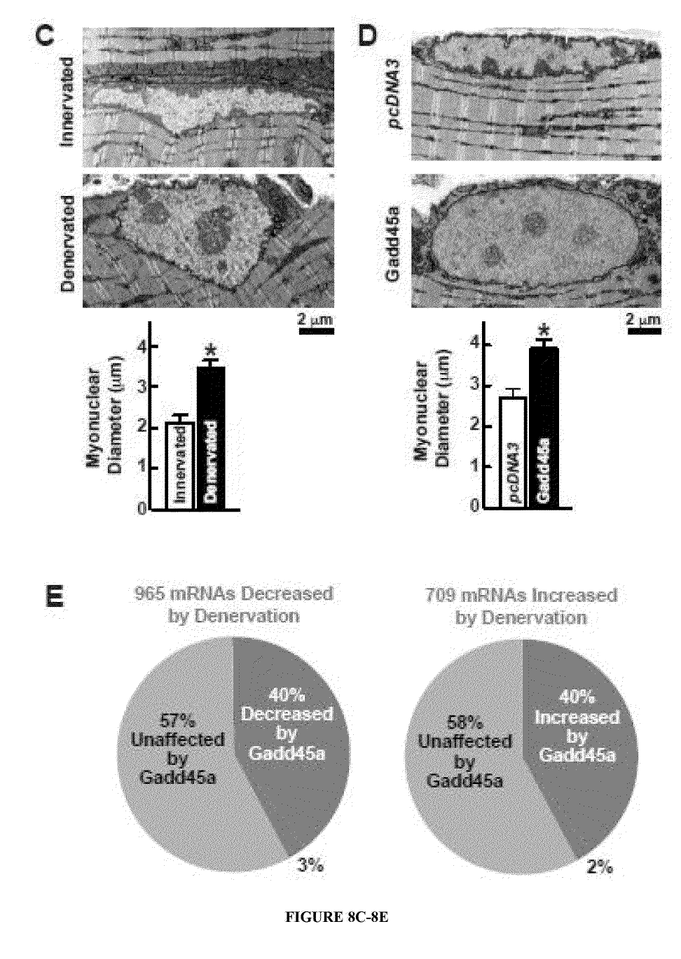

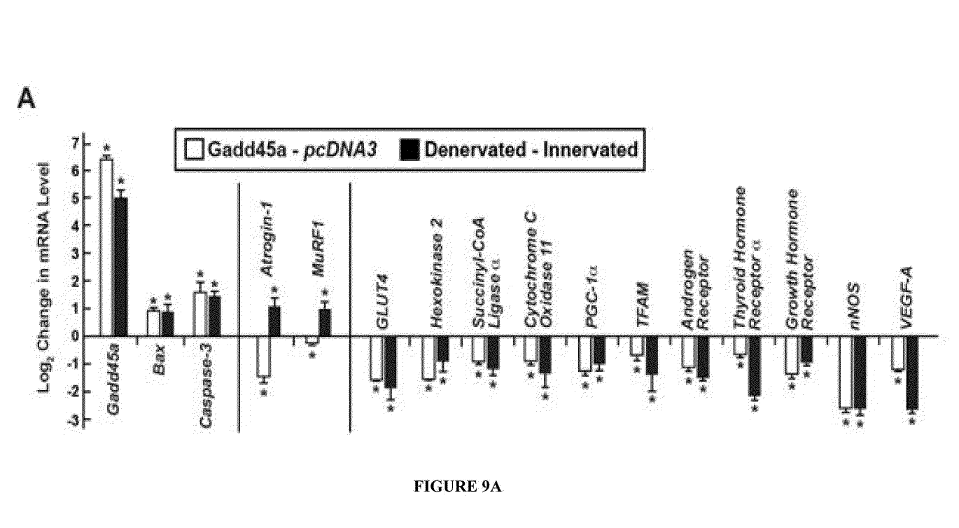

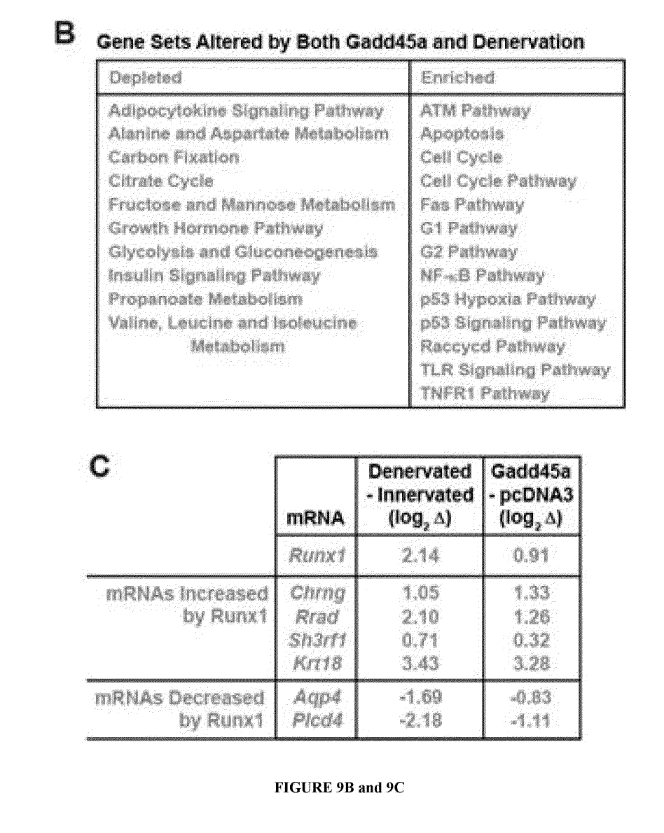

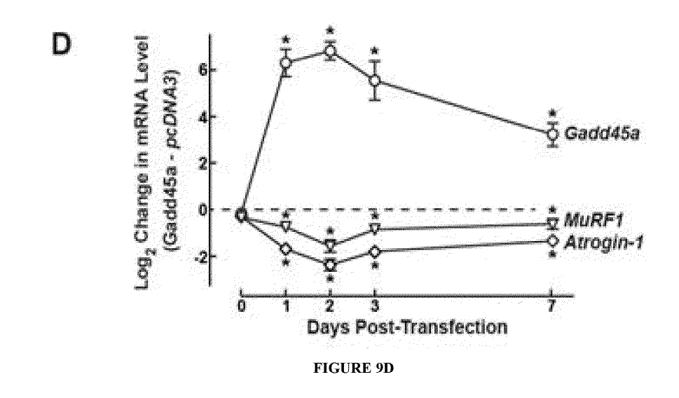

To determine how Gadd45a promotes atrophy, immunohistochemistry was used to localize Gadd45a in myotubes and muscle fibers. Consistent with previous findings in non-muscle cells (Liebermann, D. A., and Hoffman, B. (2008) J. Mol. Signal 3, 15), Gadd45a was predominantly nuclear in myotubes (FIG. 8A) and muscle fibers (FIG. 8B), indicating that Gadd45a promotes muscle atrophy by altering a process within myonuclei. To further investigate this possibility, TEM was used to examine nuclear morphology in muscle fibers that had undergone Gadd45a-mediated atrophy. The positive control was muscle denervation, which increases Gadd45a mRNA to a similar level as Gadd45a overexpression (Table 3 and FIG. 9A). One week of muscle denervation induced the classical ultrastructural changes previously described by Korényi-Both: “the nuclei lose their cigar-like shape and become swollen, rounded and plump, with prominent nucleoli” (Korényi-Both, A. L. (1983) Muscle Pathology in Neuromuscular Disease, C. C. Thomas, Springfield, Ill.) (FIG. 8C). Interestingly, 1 week of Gadd45a overexpression induced similar changes in nuclear morphology (FIG. 8D). These data indicates that Gadd45a plays an important role in the myonuclear remodeling that occurs during muscle atrophy.

(f) Gadd4Sa Generates 40% of the mRNA Expression Changes that Occur During Muscle Denervation

In other cell types, nuclear remodeling is associated with altered gene expression (Easwaran, H. P., and Baylin, S. B. (2010) Cold Spring Harbor Symp. Quant. Biol. 75, 507-515). Thus, the finding that Gadd45a altered myonuclear structure indicated that it might contribute to gene expression changes that occur during muscle atrophy. To test this, exon expression arrays were used to compare effects of denervation and Gadd45a on levels of >16,000 mRNAs. Gadd45a was overexpressed in ATF4 mKO muscle to eliminate any potential contribution from ATF4. Using p≦0.01 as the threshold for statistical significance, it was found that denervation significantly altered levels of 1674 mRNAs, decreasing 965 and increasing 709. Of the 965 mRNAs decreased by denervation, 40% were significantly decreased by Gadd45a, 3% were increased and 57% were unaffected (FIG. 8E). Of the 709 mRNAs increased by denervation, 40% were significantly increased by Gadd45a, 2% were decreased, and 58% were unaffected (FIG. 8E). Altogether, >600 mRNAs were identified whose levels were similarly altered by denervation and Gadd45a overexpression (Table 3). Thus, increased Gadd45a expression generates many, but not all, of the positive and negative mRNA expression changes in denervated muscle.

TABLE 3
MOUSE SKELETAL MUSCLE MRNAS SIMILARLY ALTERED BY 1 WEEK
DENERVATION AND 1 WEEK GADD45A OVEREXPRESSION

| | | | Effect of |
|---|---|---|---|
| | | Effect of | Gadd45a |
| | | Denervation | Log2 Δ |
| | | Log2 Δ | (Gadd45- |
| gene_assignment | Gene Symbol | (Den-Inn) | pcDNA) |
| NM_010664 // Krt18 // keratin 18 // 15 F3\|15 | Krt18 | 3.43 | 3.28 |
| 58.86 cM // 16668 /// ENSMUST000000 | | | |
| NM_007836 // Gadd45a // growth arrest and | Gadd45a | 2.53 | 2.57 |
| DNA-damage-inducible 45 alpha // 6 C1\| | | | |
| NM_001113204 // Ncam1 // neural cell adhesion | Ncam1 | 1.39 | 2.13 |
| molecule 1 // 9 A5.3\|9 28.0 cM // | | | |
| NM_025540 // Sln // sarcolipin // 9\|9 C // 66402 | Sln | 2.66 | 2.12 |
| /// ENSMUST00000048485 // Sln / | | | |
| NM_007389 // Chrna1 // cholinergic receptor, | Chrna1 | 2.89 | 1.91 |
| nicotinic, alpha polypeptide 1 (mus | | | |
| NM_001130174 // Tnnt2 // troponin T2, cardiac | Tnnt2 | 2.05 | 1.90 |
| // 1 E4\|1 60.0 cM // 21956 /// NM_{—} | | | |
| NM_010858 // Myl4 // myosin, light polypeptide | Myl4 | 2.10 | 1.88 |
| 4 // 11 E\|11 65.0 cM // 17896 /// | | | |
| NM_024290 // Tnfrsf23 // tumor necrosis factor | Tnfrsf23 | 3.18 | 1.84 |
| receptor superfamily, member 23 / | | | |
| NM_025273 // Pcbd1 // pterin 4 alpha | Pcbd1 | 0.24 | 1.81 |
| carbinolamine dehydratase/dimerization cofa | | | |
| NM_013468 // Ankrd1 // ankyrin repeat domain | Ankrd1 | 3.34 | 1.76 |
| 1 (cardiac muscle) // 19\|19 C3 // 1 | | | |
| NM_007669 // Cdkn1a // cyclin-dependent | Cdkn1a | 2.67 | 1.71 |
| kinase inhibitor 1A (P21) // 17 A3.3\|17 | | | |
| NM_023680 // Tnfrsf22 // tumor necrosis factor | Tnfrsf22 | 2.04 | 1.62 |
| receptor superfamily, member 22 / | | | |
| NM_001161432 // Eda2r // ectodysplasin A2 | Eda2r | 3.03 | 1.60 |
| receptor // X C3\|X // 245527 /// NM_17 | | | |
| NR_001592 // H19 // H19 fetal liver mRNA // 7 | H19 | 1.09 | 1.50 |
| F5\|7 69.03 cM // 14955 /// NR_0304 | | | |
| NM_008634 // Mtap1b // microtubule-associated | Mtap1b | 1.27 | 1.49 |
| protein 1B // 13 D1\|13 51.0 cM // | | | |
| NM_011670 // Uchl1 // ubiquitin carboxy- | Uchl1 | 1.56 | 1.41 |
| terminal hydrolase L1 // 5 C3.1\|5 36.0 c | | | |
| NM_198161 // Bhlhb9 // basic helix-loop-helix | Bhlhb9 | 1.52 | 1.40 |
| domain containing, class B9 // X F | | | |
| NM_009604 // Chrng // cholinergic receptor, | Chrng | 1.05 | 1.33 |
| nicotinic, gamma polypeptide // 1 D\| | | | |
| NM_177909 // Slc9a9 // solute carrier family 9 | Slc9a9 | 1.15 | 1.27 |
| (sodium/hydrogen exchanger), memb | | | |
| NM_019662 // Rrad // Ras-related associated | Rrad | 2.10 | 1.26 |
| with diabetes // 8 D3\|8 // 56437 /// | | | |
| NM_011175 // Lgmn // legumain // 12 E\|12 // | Lgmn | 0.84 | 1.19 |
| 19141 /// ENSMUST00000021607 // Lgmn | | | |
| NM_001177616 // Arpp21 // cyclic AMP- | Arpp21 | 1.15 | 1.15 |
| regulated phosphoprotein, 21 // 9 F3\|9 59.0 | | | |
| NM_009029 // Rb1 // retinoblastoma 1 // 14 | Rb1 | 2.50 | 1.11 |
| D3\|14 41.0 cM // 19645 /// ENSMUST000 | | | |
| NM_029930 // Fam115a // family with sequence | Fam115a | 0.15 | 1.09 |
| similarity 115, member A // 6 B2\|6 | | | |
| NM_145144 // Aif1l // allograft inflammatory | Aif1l | 1.82 | 1.06 |
| factor 1-like // 2 B\|2 // 108897 // | | | |
| NM_008258 // Hn1 // hematological and | Hn1 | 1.63 | 1.05 |
| neurological expressed sequence 1 // 11 E2 | | | |
| NM_010130 // Emr1 // EGF-like module | Emr1 | 0.43 | 1.05 |
| containing, mucin-like, hormone receptor-li | | | |
| NM_009853 // Cd68 // CD68 antigen // 11 B3\|11 | Cd68 | 1.19 | 1.05 |
| 39.0 cM // 12514 /// ENSMUST000001 | | | |
| NM_172685 // Slc25a24 // solute carrier family | Slc25a24 | 0.55 | 1.04 |
| 25 (mitochondrial carrier, phosph | | | |
| NM_026473 // Tubb6 // tubulin, beta 6 // 18\|18 | Tubb6 | 1.38 | 1.03 |
| E1 // 67951 /// ENSMUST0000000151 | | | |
| NM_021436 // Tmeff1 // transmembrane protein | Tmeff1 | 0.22 | 1.02 |
| with EGF-like and two follistatin-1 | | | |
| NM_010864 // Myo5a // myosin VA // 9 D\|9 | Myo5a | 0.59 | 1.01 |
| 42.0 cM // 17918 /// ENSMUST00000036772 | | | |
| NM_175369 // Ccdc122 // coiled-coil domain | Ccdc122 | 0.66 | 1.01 |
| containing 122 // 14 D3\|14 // 108811 | | | |
| NM_030704 // Hspb8 // heat shock protein 8 // 5 | Hspb8 | 0.95 | 0.99 |
| F\|5 59.0 cM // 80888 /// ENSMUST | | | |
| NM_009779 // C3ar1 // complement component | C3ar1 | 0.66 | 0.94 |
| 3a receptor 1 // 6\|6 F1 // 12267 /// | | | |
| NM_007807 // Cybb // cytochrome b-245, beta | Cybb | 1.02 | 0.93 |
| polypeptide // X A1.1\|X // 13058 /// | | | |
| NM_009370 // Tgfbr1 // transforming growth | Tgfbr1 | 1.44 | 0.92 |
| factor, beta receptor I // 4 B1\|4 19. | | | |
| NM_172868 // Palm2 // paralemmin 2 // 4 B3\|4 // | Palm2 | 1.41 | 0.92 |
| 242481 /// ENSMUST00000102904 // | | | |
| NM_001111023 // Runx1 // runt related | Runx1 | 2.14 | 0.91 |
| transcription factor 1 // 16 C4\|16 62.2 cM | | | |
| NM_001113478 // Frrs1 // ferric-chelate | Frrs1 | 0.39 | 0.91 |
| reductase 1 // 3 G1\|3 // 20321 /// NM_00 | | | |
| NM_181585 // Pik3r3 // phosphatidylinositol 3 | Pik3r3 | 1.38 | 0.91 |
| kinase, regulatory subunit, polype | | | |
| NM_080466 // Kcnn3 // potassium | Kcnn3 | 1.92 | 0.91 |
| intermediate/small conductance calcium- | | | |
| activated | | | |
| NM_001177646 // Sirpa // signal-regulatory | Sirpa | 0.61 | 0.91 |
| protein alpha // 2 F3\|2 73.1 cM // 19 | | | |
| NM_011815 // Fyb // FYN binding protein // 15 | Fyb | 2.31 | 0.90 |
| A1\|15 // 23880 /// ENSMUST00000090 | | | |
| NM_008079 // Galc // galactosylceramidase // 12 | Galc | 0.32 | 0.89 |
| E\|12 48.0 cM // 14420 /// NM_001 | | | |
| NM_001127259 // Trp63 // transformation | Trp63 | 1.08 | 0.88 |
| related protein 63 // 16 B1\|16 19.3 cM / | | | |
| NM_009600 // Macf1 // microtubule-actin | Macf1 | 0.92 | 0.88 |
| crosslinking factor 1 // 4 D2.2\|4 57.4 c | | | |
| NM_007793 // Cstb // cystatin B // 10 C1\|10 42.0 cM | Cstb | 0.95 | 0.87 |
| 13014 /// ENSMUST00000005 | | | |
| NM_001081396 // Wdr67 // WD repeat domain | Wdr67 | 1.89 | 0.87 |
| 67 // 15 D1\|15 // 210544 /// NM_001167 | | | |
| NM_181348 // Prune2 // prune homolog 2 | Prune2 | 1.98 | 0.87 |
| (Drosophila) // 19 B\|19 // 353211 /// ENS | | | |
| NM_153507 // Cpne2 // copine II // 8 C5\|8 // | Cpne2 | 2.55 | 0.85 |
| 234577 /// ENSMUST00000048653 // Cp | | | |
| NM_013454 // Abca1 // ATP-binding cassette, | Abca1 | 1.03 | 0.84 |
| sub-family A (ABC1), member 1 // 4 A | | | |
| NM_029522 // Gpsm2 // G-protein signalling | Gpsm2 | 0.31 | 0.84 |
| modulator 2 (AGS3-like, C. elegans) / | | | |
| NM_011882 // Rnasel // ribonuclease L (2′,5′- | Rnasel | 0.45 | 0.83 |
| oligoisoadenylate synthetase-depen | | | |
| NM_012054 // Aoah // acyloxyacyl hydrolase // | Aoah | 0.34 | 0.82 |
| 13 A2\|13 // 27052 /// ENSMUST00000 | | | |
| NM_030261 // Sesn3 // sestrin 3 // 9\|9 A3 // | Sesn3 | 0.70 | 0.81 |
| 75747 /// ENSMUST00000034507 // Ses | | | |
| NM_146114 // Dclre1c // DNA cross-link repair | Dclre1c | 0.83 | 0.80 |
| 1C, PSO2 homolog (S. cerevisiae) / | | | |
| NM_015735 // Ddb1 // damage specific DNA | Ddb1 | 0.90 | 0.80 |
| binding protein 1 // 19 centromere\|19 5 | | | |
| NM_011878 // Tiam2 // T-cell lymphoma | Tiam2 | 1.19 | 0.80 |
| invasion and metastasis 2 // 17 A1\|17 4.0 | | | |
| NM_001082414 // Sh3d19 // SH3 domain | Sh3d19 | 2.18 | 0.79 |
| protein D19 // 3 F1\|3 // 27059 /// ENSMUST0 | | | |
| NM_153197 // Clec4a3 // C-type lectin domain | Clec4a3 | 0.56 | 0.79 |
| family 4, member a3 // 6 F2\|6 // 73 | | | |
| NM_009810 // Casp3 // caspase 3 // 8 B1.1\|8 | Casp3 | 0.99 | 0.78 |
| 26.0 cM // 12367 /// ENSMUST00000093 | | | |
| NM_011712 // Wbp5 // WW domain binding | Wbp5 | 0.30 | 0.77 |
| protein 5 // X F1\|X // 22381 /// ENSMUST0 | | | |
| NM_145494 // Me2 // malic enzyme 2, NAD(+)- | Me2 | 0.87 | 0.76 |
| dependent, mitochondrial // 18 E2\|18 | | | |
| NM_011673 // Ugcg // UDP-glucose ceramide | Ugcg | 2.35 | 0.76 |
| glucosyltransferase // 4 B3\|4 32.0 cM | | | |
| NM_011607 // Tnc // tenascin C // 4 C1\|4 32.2 cM | Tnc | 0.47 | 0.75 |
| // 21923 /// ENSMUST00000107379 | | | |
| NM_009601 // Chrnb1 // cholinergic receptor, | Chrnb1 | 1.24 | 0.75 |
| nicotinic, beta polypeptide 1 (musc | | | |
| NM_017372 // Lyz2 // lysozyme 2 // 10 D2\|10 | Lyz2 | 1.13 | 0.75 |
| 66.0 cM // 17105 /// NM_013590 // Ly | | | |
| NM_053257 // Rpl31 // ribosomal protein L31 // | Rpl31 | 0.54 | 0.75 |
| 1 B\|1 // 114641 /// NM_018775 // | | | |
| NM_011655 // Tubb5 // tubulin, beta 5 // 17 | Tubb5 | 0.86 | 0.72 |
| B1\|17 // 22154 /// ENSMUST0000000156 | | | |
| NM_013813 // Epb4.1l3 // erythrocyte protein | Epb4.1l3 | 0.27 | 0.72 |
| band 4.1-like 3 // 17 E1.3\|17 42.5 | | | |
| NM_023056 // Tmem176b // transmembrane | Tmem176b | 0.71 | 0.71 |
| protein 176B // 6 B2.3\|6 19.0 cM // 65963 | | | |
| NM_001170433 // Ppfibp1 // PTPRF interacting | Ppfibp1 | 0.91 | 0.71 |
| protein, binding protein 1 (liprin | | | |
| NM_011149 // Ppib // peptidylprolyl isomerase | Ppib | 0.20 | 0.71 |
| B // 9 C\|9 // 19035 /// NM_0010256 | | | |
| NM_001001650 // Prss48 // protease, serine, 48 | Prss48 | 1.77 | 0.70 |
| // 3 F1\|3 // 368202 /// ENSMUST00 | | | |
| NM_011662 // Tyrobp // TYRO protein tyrosine | Tyrobp | 1.56 | 0.70 |
| kinase binding protein // 7 B\|7 10. | | | |
| NR_030673 // D930016D06Rik // RIKEN | D930016D06Rik | 1.18 | 0.70 |
| cDNA D930016D06 gene // 5 E5\|5 // 100662 /// | | | |
| NM_009061 // Rgs2 // regulator of G-protein | Rgs2 | 0.88 | 0.69 |
| signaling 2 // 1 F\|1 78.0 cM // 1973 | | | |
| NM_001113460 // Tec // tec protein tyrosine | Tec | 1.70 | 0.69 |
| kinase // 5 C3.2\|5 41.0 cM // 21682 | | | |
| NM_175116 // Lpar6 // lysophosphatidic acid | Lpar6 | 0.69 | 0.69 |
| receptor 6 // 14 D3\|14 // 67168 /// | | | |
| NM_145628 // Usp11 // ubiquitin specific | Usp11 | 0.60 | 0.69 |
| peptidase 11 // X A1.3\|X // 236733 /// | | | |
| NM_023065 // Ifi30 // interferon gamma | Ifi30 | 0.62 | 0.68 |
| inducible protein 30 // 8 B3.3\|8 // 65972 | | | |
| NM_001001184 // Ccdc111 // coiled-coil | Ccdc111 | 0.36 | 0.68 |
| domain containing 111 // 8 B1.1\|8 // 4080 | | | |
| NM_010496 // Id2 // inhibitor of DNA binding 2 | Id2 | 1.51 | 0.68 |
| // 12 B\|12 7.0 cM // 15902 /// EN | | | |
| NM_029787 // Cyb5r3 // cytochrome b5 | Cyb5r3 | 0.72 | 0.68 |
| reductase 3 // 15 E\|15 45.2 cM // 109754 // | | | |
| NM_028696 // Obfc2a // | Obfc2a | 1.98 | 0.68 |
| oligonucleotide/oligosaccharide-binding fold | | | |
| containing 2 | | | |
| NM_176930 // Nrcam // neuron-glia-CAM- | Nrcam | 0.72 | 0.68 |
| related cell adhesion molecule // 12 B3\|12 | | | |
| NM_013471 // Anxa4 // annexin A4 // 6 D1\|6 | Anxa4 | 1.06 | 0.67 |
| 38.0 cM // 11746 /// ENSMUST000001136 | | | |
| NM_013556 // Hprt // hypoxanthine guanine | Hprt | 1.32 | 0.66 |
| phosphoribosyl transferase // X A6\|X 1 | | | |
| NM_178045 // Rassf4 // Ras association | Rassf4 | 0.60 | 0.66 |
| (RalGDS/AF-6) domain family member 4 // 6 | | | |
| NM_009163 // Sgpl1 // sphingosine phosphate | Sgpl1 | 0.57 | 0.66 |
| lyase 1 // 10 B4\|10 32.0 cM // 20397 | | | |
| NM_029926 // Irak4 // interleukin-1 receptor- | Irak4 | 0.44 | 0.66 |
| associated kinase 4 // 15 F1\|15 // | | | |
| NM_022325 // Ctsz // cathepsin Z // 2 H4\|2 | Ctsz | 0.45 | 0.65 |
| 103.5 cM // 64138 /// ENSMUST00000016 | | | |
| NM_011353 // Serf1 // small EDRK-rich factor 1 | Serf1 | 1.40 | 0.64 |
| // 13 D1\|13 53.0 cM // 20365 /// | | | |
| NM_177686 // Clec12a // C-type lectin domain | Clec12a | 0.82 | 0.64 |
| family 12, member a // 6 F3\|6 // 23 | | | |
| NM_021297 // Tlr4 // toll-like receptor 4 // 4 | Tlr4 | 1.68 | 0.63 |
| C1\|4 33.0 cM // 21898 /// ENSMUST | | | |
| NM_198018 // Abr // active BCR-related gene // | Abr | 0.32 | 0.63 |
| 11 B5\|11 45.0 cM // 109934 /// NM | | | |
| NM_008575 // Mdm4 // transformed mouse 3T3 | Mdm4 | 1.69 | 0.62 |
| cell double minute 4 // 1 G-G\|1 70.0 | | | |
| NM_175489 // Osbpl8 // oxysterol binding | Osbpl8 | 0.89 | 0.62 |
| protein-like 8 // 10 D1\|10 // 237542 // | | | |
| NM_001145953 // Lgals3 // lectin, galactose | Lgals3 | 1.23 | 0.61 |
| binding, soluble 3 // 14 C1\|14 // 16 | | | |
| NM_011484 // Stam // signal transducing | Stam | 0.59 | 0.60 |
| adaptor molecule (SH3 domain and ITAM mo | | | |
| NM_199035 // Alg8 // asparagine-linked | Alg8 | 0.45 | 0.60 |
| glycosylation 8 homolog (yeast, alpha-1,3 | | | |
| NM_027556 // Cep192 // centrosomal protein | Cep192 | 0.91 | 0.60 |
| 192 // 18\|18 E1 // 70799 /// ENSMUST0 | | | |
| NM_001109757 // Atp7a // ATPase, Cu++ | Atp7a | 0.27 | 0.60 |
| transporting, alpha polypeptide // X D\|X 4 | | | |
| NM_018796 // Eef1b2 // eukaryotic translation | Eef1b2 | 0.37 | 0.59 |
| elongation factor 1 beta 2 // 1 C2 | | | |
| NM_007564 // Zfp3611 // zinc finger protein 36, | Zfp36l1 | 1.06 | 0.59 |
| C3H type-like 1 // 12 C3\|12 // 1 | | | |
| NM_027968 // Fbxo30 // F-box protein 30 // | Fbxo30 | 1.50 | 0.59 |
| 10\|10 A2 // 71865 /// NM_001168297 // | | | |
| NM_021281 // Ctss // cathepsin S // 3 F2.1\|3 | Ctss | 1.55 | 0.58 |
| 42.7 cM // 13040 /// ENSMUST0000001 | | | |
| NM_001038602 // Marveld2 // MARVEL | Marveld2 | 0.72 | 0.57 |
| (membrane-associating) domain containing 2 // | | | |
| NM_018811 // Abhd2 // abhydrolase domain | Abhd2 | 0.45 | 0.57 |
| containing 2 // 7\|7 D2 // 54608 /// ENS | | | |
| NM_001081274 // Pgd // phosphogluconate | Pgd | 0.78 | 0.57 |
| dehydrogenase // 4 E2\|4 77.6 cM // 11020 | | | |
| NM_010786 // Mdm2 // transformed mouse 3T3 | Mdm2 | 1.01 | 0.56 |
| cell double minute 2 // 10 C1-C3\|10 6 | | | |
| NM_008046 // Fst // follistatin // 13 D2.2\|13 // | Fst | 0.73 | 0.56 |
| 14313 /// ENSMUST00000022287 // | | | |
| NM_011604 // Tlr6 // toll-like receptor 6 // 5 | Tlr6 | 0.60 | 0.56 |
| C3.1\|5 37.0 cM // 21899 /// NM_03 | | | |
| NM_008696 // Map4k4 // mitogen-activated | Map4k4 | 0.56 | 0.55 |
| protein kinase kinase kinase kinase 4 / | | | |
| NM_027230 // Zmynd8 // zinc finger, MYND- | Zmynd8 | 0.60 | 0.55 |
| type containing 8 // 2 H3\|2 // 228880 / | | | |
| NM_001005341 // Ypel2 // yippee-like 2 | Ypel2 | 1.33 | 0.55 |
| (Drosophila) // 11 C\|11 // 77864 /// ENSM | | | |
| NM_011072 // Pfn1 // profilin 1 // 11 B4\|11 42.0 cM | Pfn1 | 0.43 | 0.55 |
| // 18643 /// ENSMUST00000018 | | | |
| NM_001042719 // Ddhd1 // DDHD domain | Ddhd1 | 0.56 | 0.55 |
| containing 1 // 14 C1\|14 // 114874 /// NM_1 | | | |
| NM_025846 // Rras2 // related RAS viral (r-ras) | Rras2 | 1.20 | 0.55 |
| oncogene homolog 2 // 7 F2\|7 // | | | |
| NM_012017 // Zfp346 // zinc finger protein 346 | Zfp346 | 1.29 | 0.54 |
| // 13 B2\|13 // 26919 /// ENSMUST0 | | | |
| NM_027439 // Atp6ap2 // ATPase, H+ | Atp6ap2 | 1.03 | 0.54 |
| transporting, lysosomal accessory protein 2 / | | | |
| NM_008625 // Mrc1 // mannose receptor, C type | Mrc1 | 0.68 | 0.53 |
| 1 // 2 A2\|2 5.0 cM // 17533 /// EN | | | |
| NM_024225 // Snx5 // sorting nexin 5 // 2 H1\|2 | Snx5 | 0.68 | 0.52 |
| 80.0 cM // 69178 /// NR_030762 // | | | |
| NM_010315 // Gng2 // guanine nucleotide | Gng2 | 0.59 | 0.51 |
| binding protein (G protein), gamma 2 // | | | |
| NM_019653 // Wsb1 // WD repeat and SOCS | Wsb1 | 0.64 | 0.51 |
| box-containing 1 // 11 B5\|11 // 78889 // | | | |
| NM_029734 // Wdyhv1 // WDYHV motif | Wdyhv1 | 0.59 | 0.51 |
| containing 1 // 15 D2\|15 // 76773 /// ENSMUST | | | |
| NM_010378 // H2-Aa // histocompatibility 2, | H2-Aa | 1.45 | 0.51 |
| class II antigen A, alpha // 17 B1\|1 | | | |
| NM_008657 // Myf6 // myogenic factor 6 // 10 | Myf6 | 1.88 | 0.50 |
| D1\|10 59.0 cM // 17878 /// ENSMUST0 | | | |
| NR_028367 // Nip7 // nuclear import 7 homolog | Nip7 | 0.69 | 0.50 |
| (S. cerevisiae) // 8\|8 D2 // 66164 | | | |
| NM_007733 // Col19a1 // collagen, type XIX, | Col19a1 | 2.01 | 0.50 |
| alpha 1 // 1\|1 A3 // 12823 /// NM_14 | | | |
| NM_020483 // Sap30bp // SAP30 binding | Sap30bp | 0.57 | 0.50 |
| protein // 11 E2\|11 // 57230 /// ENSMUST00 | | | |
| NM_018868 // Nop58 // NOP58 | Nop58 | 1.02 | 0.50 |
| ribonucleoprotein homolog (yeast) // 1 C1.3\|1 // | | | |
| 559 | | | |
| NM_016798 // Pdlim3 // PDZ and LIM domain 3 | Pdlim3 | 1.03 | 0.49 |
| // 8 B1.1\|8 // 53318 /// ENSMUST0000 | | | |
| NM_144731 // Galnt7 // UDP-N-acetyl-alpha-D- | Galnt7 | 1.83 | 0.49 |
| galactosamine:polypeptide N-acetylg | | | |
| NM_008487 // Arhgef2 // rho/rac guanine | Arhgef2 | 0.52 | 0.49 |
| nucleotide exchange factor (GEF) 2 // 3 | | | |
| NM_029999 // Lbh // limb-bud and heart // 17 | Lbh | 0.94 | 0.48 |
| E2\|17 // 77889 /// BC052470 // Lbh | | | |
| NM_133826 // Atp6v1h // ATPase, H+ | Atp6v1h | 0.81 | 0.48 |
| transporting, lysosomal V1 subunit H // 1 A1\| | | | |
| NM_177353 // Slc9a7 // solute carrier family 9 | Slc9a7 | 0.98 | 0.48 |
| (sodium/hydrogen exchanger), memb | | | |
| NM_010248 // Gab2 // growth factor receptor | Gab2 | 0.29 | 0.47 |
| bound protein 2-associated protein 2 | | | |
| NM_001146049 // Htatip2 // HIV-1 tat | Htatip2 | 1.00 | 0.47 |
| interactive protein 2, homolog (human) // 7 | | | |
| NM_009983 // Ctsd // cathepsin D // 7 F5\|7 // | Ctsd | 0.78 | 0.47 |
| 13033 /// ENSMUST00000151120 // Ct | | | |
| NM_026162 // Plxdc2 // plexin domain | Plxdc2 | 1.18 | 0.47 |
| containing 2 // 2 A2-A3\|2 // 67448 /// ENSM | | | |
| NM_007656 // Cd82 // CD82 antigen // 2 E1\|2 | Cd82 | 1.27 | 0.47 |
| 49.6 cM // 12521 /// NM_001136055 // | | | |
| NM_133953 // Sf3b3 // splicing factor 3b, | Sf3b3 | 0.31 | 0.46 |
| subunit 3 // 8 E1\|8 53.5 cM // 101943 | | | |
| NM_009663 // Alox5ap // arachidonate 5- | Alox5ap | 0.69 | 0.46 |
| lipoxygenase activating protein // 5 G3\|5 | | | |
| NM_008188 // Thumpd3 // THUMP domain | Thumpd3 | 0.83 | 0.46 |
| containing 3 // 6 E3\|6 48.7 cM // 14911 /// | | | |
| NM_007527 // Bax // BCL2-associated X protein | Bax | 0.69 | 0.46 |
| // 7 B5\|7 23.0 cM // 12028 /// ENS | | | |
| NM_019830 // Prmt1 // protein arginine N- | Prmt1 | 1.02 | 0.46 |
| methyltransferase 1 // 7 B4\|7 23.1 cM / | | | |
| NM_009667 // Ampd3 // adenosine | Ampd3 | 1.61 | 0.45 |
| monophosphate deaminase 3 // 7 E2-E3\|7 52.0 cM / | | | |
| NM_207671 // Zfp318 // zinc finger protein 318 | Zfp318 | 1.15 | 0.45 |
| // 17\|17 C // 57908 /// NM_021346 | | | |
| NM_172410 // Nup93 // nucleoporin 93 // 8 C5\|8 | Nup93 | 0.59 | 0.44 |
| // 71805 /// ENSMUST00000079961 / | | | |
| NM_145979 // Chd4 // chromodomain helicase | Chd4 | 0.51 | 0.44 |
| DNA binding protein 4 // 6 F3\|6 58.4 | | | |
| NM_144920 // Plekha5 // pleckstrin homology | Plekha5 | 0.76 | 0.44 |
| domain containing, family A member 5 | | | |
| NM_019824 // Arpc3 // actin related protein 2/3 | Arpc3 | 0.56 | 0.44 |
| complex, subunit 3 // 5 F\|5 // 5 | | | |
| NM_001005868 // Erbb2ip // Erbb2 interacting | Erbb2ip | 0.94 | 0.44 |
| protein // 13 D1\|13 // 59079 /// NM | | | |
| NM_011276 // Rlim // ring finger protein, LIM | Rlim | 0.51 | 0.43 |
| domain interacting // X D\|X 45.0 c | | | |
| NM_172600 // 6720456H20Rik // RIKEN | 6720456H20Rik | 0.22 | 0.43 |
| cDNA 6720456H20 gene // 14 C1\|14 // 218989 / | | | |
| NM_021608 // Dctn5 // dynactin 5 // 7\|7 F3 // | Dctn5 | 0.41 | 0.43 |
| 59288 /// ENSMUST00000033156 // Dc | | | |
| NM_026122 // Hmgn3 // high mobility group | Hmgn3 | 0.34 | 0.43 |
| nucleosomal binding domain 3 // 9\|9 E3 | | | |
| NM_011945 // Map3k1 // mitogen-activated | Map3k1 | 0.30 | 0.43 |
| protein kinase kinase kinase 1 // 13 D2 | | | |
| NM_011933 // Decr2 // 2-4-dienoyl-Coenzyme | Decr2 | 0.47 | 0.42 |
| A reductase 2, peroxisomal // 17 B1\|1 | | | |
| NM_133722 // Fam108c // family with sequence | Fam108c | 0.88 | 0.42 |
| similarity 108, member C // 7 D3\|7 | | | |
| NM_029447 // Nln // neurolysin | Nln | 0.56 | 0.42 |
| (metallopeptidase M3 family) // 13 D1\|13 // | | | |
| 75805 | | | |
| NM_008621 // Mpp1// membrane protein, | Mpp1 | 0.36 | 0.41 |
| palmitoylated // X A7.3\|X 30.48 cM // 175 | | | |
| NM_008567 // Mcm6 // minichromosome | Mcm6 | 0.51 | 0.41 |
| maintenance deficient 6 (MIS5 homolog, S. po | | | |
| NM_153808 // Smc5 // structural maintenance of | Smc5 | 0.59 | 0.41 |
| chromosomes 5 // 19 B\|19 // 22602 | | | |
| NM_008018 // Sh3pxd2a // SH3 and PX | Sh3pxd2a | 0.42 | 0.41 |
| domains 2A // 19\|19 D2 // 14218 /// NM_00116 | | | |
| NM_008143 // Gnb2l1 // guanine nucleotide | Gnb2l1 | 0.41 | 0.41 |
| binding protein (G protein), beta poly | | | |
| NM_018753 // Ywhab // tyrosine 3- | Ywhab | 0.56 | 0.41 |
| monooxygenase/tryptophan 5-monooxygenase | | | |
| activa | | | |
| NM_024207 // Derl1 // Der1-like domain family, | Derl1 | 0.92 | 0.40 |
| member 1 // 15\|15 D2 // 67819 /// | | | |
| NM_013660 // Sema4d // sema domain, | Sema4d | 0.26 | 0.40 |
| immunoglobulin domain (Ig), transmembrane do | | | |
| NM_172768 // Gramd1b // GRAM domain | Gramd1b | 0.68 | 0.40 |
| containing 1B // 9\|9 B // 235283 /// ENSMUST | | | |
| NM_019835 // B4galt5 // UDP-Gal:betaGlcNAc | B4galt5 | 0.28 | 0.39 |
| beta 1,4-galactosyltransferase, polyp | | | |
| NM_029878 // Tbcd // tubulin-specific | Tbcd | 0.27 | 0.39 |
| chaperone d // 11 E2\|11 // 108903 /// ENSM | | | |
| NM_008448 // Kif5b // kinesin family member | Kif5b | 0.84 | 0.39 |
| 5B // 18 A2-B1\|18 1.0 cM // 16573 // | | | |
| NM_026770 // Cgref1 // cell growth regulator | Cgref1 | 0.73 | 0.38 |
| with EF hand domain 1 // 5 B1\|5 // | | | |
| NM_011156 // Prep // prolyl endopeptidase // 10 | Prep | 0.62 | 0.38 |
| B2-B3110 28.5 cM // 19072 /// EN | | | |
| NM_172716 // Pcgf3 // polycomb group ring | Pcgf3 | 1.09 | 0.38 |
| finger 3 // 5 F\|5 // 69587 /// NM_0010 | | | |
| NM_177461 // Micall1 // microtubule associated | Micall1 | 0.67 | 0.38 |
| monoxygenase, calponin and LIM do | | | |
| NM_009012 // Rad50 // RAD50 homolog (S. cerevisiae) | Rad50 | 0.48 | 0.38 |
| // 11 A5-B1\|11 28.9 cM // 19 | | | |
| NM_001162906 // 2410089E03Rik // RIKEN | 2410089E03Rik | 0.32 | 0.37 |
| cDNA 2410089E03 gene // 15 A1\|15 // 73692 | | | |
| NM_144863 // Wdr36 // WD repeat domain 36 // | Wdr36 | 0.52 | 0.37 |
| 18 B1\|18 // 225348 /// NM_001110015 | | | |
| NM_025735 // Map1lc3a // microtubule- | Map1lc3a | 0.83 | 0.37 |
| associated protein 1 light chain 3 alpha // | | | |
| NM_018775 // Tbc1d8 // TBC1 domain family, | Tbc1d8 | 0.40 | 0.37 |
| member 8 // 1 B\|1 // 54610 /// NM_053 | | | |
| NM_009837 // Cct4 // chaperonin containing | Cct4 | 0.52 | 0.37 |
| Tcp1, subunit 4 (delta) // 11 A3.2\|11 | | | |
| NM_011843 // Esyt1 // extended synaptotagmin- | Esyt1 | 0.51 | 0.37 |
| like protein 1 // 10 D3\|10 // 23943 | | | |
| NM_008378 // Impact // imprinted and ancient // | Impact | 0.96 | 0.36 |
| 18\|18 A2-B2 // 16210 /// ENSMUST | | | |
| NM_008410 // Itm2b // integral membrane | Itm2b | 0.59 | 0.36 |
| protein 2B // 14 D3\|14 32.5 cM // 16432 | | | |
| NM_009546 // Trim25 // tripartite motif- | Trim25 | 0.30 | 0.36 |
| containing 25 // 11 C\|11 // 217069 /// E | | | |
| NM_010516 // Cyr61 // cysteine rich protein 61 | Cyr61 | 0.82 | 0.36 |
| // 3 H2\|3 72.9 cM // 16007 /// EN | | | |
| NM_001025156 // Ccdc93 // coiled-coil domain | Ccdc93 | 0.24 | 0.35 |
| containing 93 // 1\|1 E2 // 70829 // | | | |
| NM_007842 // Dhx9 // DEAH (Asp-Glu-Ala- | Dhx9 | 0.67 | 0.35 |
| His) box polypeptide 9 // 1 G3\|1 77.0 cM | | | |
| NM_054089 // Tgs1 // trimethylguanosine | Tgs1 | 0.41 | 0.35 |
| synthase homolog (S. cerevisiae) // 4 A1 | | | |
| NM_010771 // Matr3 // matrin 3 // 18 C\|18 15.0 cM | Matr3 | 0.44 | 0.35 |
| // 17184 /// NR_002905 // Snor | | | |
| NM_018737 // Ctps2 // cytidine 5′-triphosphate | Ctps2 | 1.17 | 0.35 |
| synthase 2 // X\|X F5 // 55936 /// | | | |
| NM_001123372 // Gm3435 // predicted gene | Gm3435 | 0.32 | 0.35 |
| 3435 // 17 A2\|17 // 100041621 /// NM_00 | | | |
| NM_007700 // Chuk // conserved helix-loop- | Chuk | 0.59 | 0.35 |
| helix ubiquitous kinase // 19 C3\|19 45 | | | |
| NM_008060 // Ganab // alpha glucosidase 2 | Ganab | 0.13 | 0.34 |
| alpha neutral subunit // 19 A\|19 // 14 | | | |
| NM_010831 // Sik1 // salt inducible kinase 1 // | Sik1 | 1.31 | 0.34 |
| 17 B1\|17 18.18 cM // 17691 /// E | | | |
| NM_026325 // Tmem179b // transmembrane | Tmem179b | 0.32 | 0.34 |
| protein 179B // 19 A\|19 // 67706 /// NR_0 | | | |
| NM_175174 // Klhl5 // kelch-like 5 (Drosophila) | Klhl5 | 0.88 | 0.33 |
| // 5 C3.1\|5 // 71778 /// ENSMUST | | | |
| NM_001025375 // Wdr61 // WD repeat domain | Wdr61 | 0.58 | 0.33 |
| 61 // 9\|9 C // 66317 /// NM_023191 // | | | |
| NM_020043 // Igdcc4 // immunoglobulin | Igdcc4 | 0.22 | 0.33 |
| superfamily, DCC subclass, member 4 // 9 C | | | |
| NM_010761 // Ccndbp1 // cyclin D-type | Ccndbp1 | 0.56 | 0.33 |
| binding-protein 1 // 2 E5\|2 68.6 cM // 171 | | | |
| NM_057172 // Fubp1 // far upstream element | Fubp1 | 0.57 | 0.33 |
| (FUSE) binding protein 1 // 3 H3\|3 70 | | | |
| NM_007869 // Dnajc1 // DnaJ (Hsp40) homolog, | Dnajc1 | 0.53 | 0.33 |
| subfamily C, member 1 // 2\|2 A2 // | | | |
| NM_019710 // Smc1a // structural maintenance | Smc1a | 0.42 | 0.33 |
| of chromosomes 1A // X F3\|X // 2406 | | | |
| NM_021506 // Sh3rf1 // SH3 domain containing | Sh3rf1 | 0.71 | 0.32 |
| ring finger 1 // 8 B3.1\|8 // 59009 | | | |
| NM_001113486 // Sept9 // septin 9 // 11 E2\|11 | 9-Sep | 0.37 | 0.32 |
| 79.2 cM // 53860 /// NM_001113487 | | | |
| NM_145502 // Erlin1 // ER lipid raft associated | Erlin1 | 0.67 | 0.32 |
| 1 // 19 C3\|19 // 226144 /// NM_0 | | | |
| NM_007874 // Reep5 // receptor accessory | Reep5 | 0.80 | 0.32 |
| protein 5 // 18 B1\|18 // 13476 /// ENSM | | | |
| NM_146224 // Zfp280d // zinc finger protein | Zfp280d | 0.83 | 0.31 |
| 280D // 9 D\|9 // 235469 /// ENSMUST0 | | | |
| NM_008301 // Hspa2 // heat shock protein 2 // | Hspa2 | 0.36 | 0.31 |
| 12 C3\|12 34.0 cM // 15512 /// NM_0 | | | |
| NM_029767 // Rps9 // ribosomal protein S9 // 7 | Rps9 | 0.29 | 0.31 |
| A1\|7 // 76846 /// ENSMUST00000108 | | | |
| NM_080708 // Bmp2k // BMP2 inducible kinase | Bmp2k | 0.51 | 0.31 |
| // 5 E3\|5 // 140780 /// ENSMUST00000 | | | |
| NM_028053 // Tmem38b // transmembrane | Tmem38b | 0.55 | 0.31 |
| protein 38B // 4 B2\|4 28.5 cM // 52076 /// | | | |
| NM_011539 // Tbxas1 // thromboxane A | Tbxas1 | 0.23 | 0.31 |
| synthase 1, platelet // 6 F1-pter\|6 20.5 cM | | | |
| NM_023799 // Mgea5 // meningioma expressed | Mgea5 | 0.68 | 0.31 |
| antigen 5 (hyaluronidase) // 19 D1\|19 | | | |
| NM_008300 // Hspa4 // heat shock protein 4 // | Hspa4 | 0.49 | 0.30 |
| 11 B1.3\|11 29.0 cM // 15525 /// EN | | | |
| NM_007471 // App // amyloid beta (A4) | App | 0.72 | 0.30 |
| precursor protein // 16 C3-qter\|16 56.0 cM | | | |
| NM_012052 // Rps3 // ribosomal protein S3 // 7 | Rps3 | 0.28 | 0.29 |
| F1\|7 49.6 cM // 27050 /// ENSMUST | | | |
| NM_009703 // Araf // v-raf murine sarcoma | Araf | 0.42 | 0.29 |
| 3611 viral oncogene homolog // X A2-A3 | | | |
| NM_024182 // Riok3 // RIO kinase 3 (yeast) // | Riok3 | 1.45 | 0.29 |
| 18 A2\|18 3.0 cM // 66878 /// NM_02 | | | |
| NM_011159 // Prkdc // protein kinase, DNA | Prkdc | 0.52 | 0.29 |
| activated, catalytic polypeptide // 16 | | | |
| NM_008881 // Plxna1 // plexin A1 // 6 E2\|6 // | Plxna1 | 0.13 | 0.28 |
| 18844 /// ENSMUST00000049845 // P1 | | | |
| NM_011185 // Psmb1 // proteasome (prosome, | Psmb1 | 0.86 | 0.28 |
| macropain) subunit, beta type 1 // 17 | | | |
| NM_007958 // Smarcad1 // SWI/SNF-related, | Smarcad1 | 0.35 | 0.28 |
| matrix-associated actin-dependent regu | | | |
| NM_198111 // Akap6 // A kinase (PRKA) | Akap6 | 0.76 | 0.28 |
| anchor protein 6 // 12 C1\|12 // 238161 /// | | | |
| NM_173001 // Kdm3a // lysine (K)-specific | Kdm3a | 0.69 | 0.28 |
| demethylase 3A // 6 C1\|6 // 104263 /// | | | |
| NM_001164885 // Lpin2 // lipin 2 // 17 E1.3\|17 | Lpin2 | 1.65 | 0.28 |
| // 64898 /// NM_022882 // Lpin2 / | | | |
| NM_027432 // Wdr77 // WD repeat domain 77 // | Wdr77 | 0.33 | 0.28 |
| 3 F2.2\|3 // 70465 /// ENSMUST000000 | | | |
| NM_013700 // Usp5 // ubiquitin specific | Usp5 | 0.36 | 0.28 |
| peptidase 5 (isopeptidase T) // 6 F2\|6 6 | | | |
| NM_010561 // Ilf3 // interleukin enhancer | Ilf3 | 0.40 | 0.28 |
| binding factor 3 // 9 A4-A5\|9 4.0 cM / | | | |
| NM_001001488 // Atp8b1 // ATPase, class I, | Atp8b1 | 0.32 | 0.27 |
| type 8B, member 1 // 18 E1\|18 // 5467 | | | |
| NM_009273 // Srp14 // signal recognition | Srp14 | 0.55 | 0.27 |
| particle 14 // 2 E5\|2 // 20813 /// ENSM | | | |
| NM_153405 // Rbm45 // RNA binding motif | Rbm45 | 0.39 | 0.27 |
| protein 45 // 2 C3\|2 // 241490 /// AY152 | | | |
| NM_026899 // Ssu72 // Ssu72 RNA polymerase | Ssu72 | 0.87 | 0.27 |
| II CTD phosphatase homolog (yeast) // | | | |
| NM_009818 // Ctnna1 // catenin (cadherin | Ctnna1 | 0.61 | 0.26 |
| associated protein), alpha 1 // 18 B1\|1 | | | |
| NM_178625 // Tmem209 // transmembrane | Tmem209 | 0.39 | 0.26 |
| protein 209 // 6 A3.3\|6 // 72649 /// ENSMU | | | |
| NM_009984 // Cts1 // cathepsin L // 13 B3\|13 | Cts1 | 0.96 | 0.26 |
| 30.0 cM // 13039 /// ENSMUST0000002 | | | |
| NM_153423 // Wasf2 // WAS protein family, | Wasf2 | 0.36 | 0.26 |
| member 2 // 4 D2.3\|4 65.7 cM // 242687 | | | |
| NM_080554 // Psmd5 // proteasome (prosome, | Psmd5 | 0.61 | 0.26 |
| macropain) 26S subunit, non-ATPase, 5 | | | |
| NM_021521 // Med12 // mediator of RNA | Med12 | 0.34 | 0.26 |
| polymerase II transcription, subunit 12 ho | | | |
| NM_178363 // Ylpm1 // YLP motif containing 1 | Ylpm1 | 0.32 | 0.25 |
| // 12 D3\|12 // 56531 /// AB033168 / | | | |
| NM_018744 // Sema6a // sema domain, | Sema6a | 0.56 | 0.25 |
| transmembrane domain (TM), and cytoplasmic d | | | |
| NM_054102 // Ivns1abp // influenza virus NS1A | Ivns1abp | 0.76 | 0.25 |
| binding protein // 1 G2\|1 // 11719 | | | |
| NM_007597 // Canx // calnexin // 11 B1.3\|11 | Canx | 0.51 | 0.25 |
| 30.0 cM // 12330 /// NM_001110499 // | | | |
| NM_019776 // Snd1 // staphylococcal nuclease | Snd1 | 0.80 | 0.25 |
| and tudor domain containing 1 // 6 | | | |
| NM_153547 // Gnl3 // guanine nucleotide | Gnl3 | 0.82 | 0.24 |
| binding protein-like 3 (nucleolar) // 14 | | | |
| NM_177852 // Dido1 // death inducer-obliterator | Dido1 | 0.21 | 0.23 |
| 1 // 2 H4\|2 // 23856 /// NM_1755 | | | |
| NM_015827 // Copb2 // coatomer protein | Copb2 | 0.24 | 0.22 |
| complex, subunit beta 2 (beta prime) // 9 | | | |
| NM_145552 // Gnl2 // guanine nucleotide | Gnl2 | 0.73 | 0.22 |
| binding protein-like 2 (nucleolar) // 4 | | | |
| NM_031179 // Sf3b1 // splicing factor 3b, | Sf3b1 | 0.26 | 0.22 |
| subunit 1 // 1 C1.2\|1 28.9 cM // 81898 | | | |
| NM_009089 // Polr2a // polymerase (RNA) II | Polr2a | 0.21 | 0.21 |
| (DNA directed) polypeptide A // 11 B1 | | | |
| NM_026171 // Nvl // nuclear VCP-like // 1 H4\|1 | Nvl | 0.39 | 0.21 |
| // 67459 /// ENSMUST00000027797 / | | | |
| NM_019426 // Atf7ip // activating transcription | Atf7ip | 0.22 | 0.21 |
| factor 7 interacting protein // | | | |
| NM_011317 // Khdrbs1 // KH domain | Khdrbs1 | 0.33 | 0.21 |
| containing, RNA binding, signal transduction a | | | |
| BC003288 // D10Wsu52e // DNA segment, Chr | D10Wsu52e | 0.15 | 0.21 |
| 10, Wayne State University 52, express | | | |
| NM_172438 // Thoc5 // THO complex 5 // 11 | Thoc5 | 0.75 | 0.21 |
| A1\|11 // 107829 /// ENSMUST00000038237 | | | |
| NM_001145978 // Parp4 // poly (ADP-ribose) | Parp4 | 0.63 | 0.20 |
| polymerase family, member 4 //14 C3\| | | | |
| NM_015747 // Slc20a1 // solute carrier family | Slc20a1 | 0.51 | 0.20 |
| 20, member 1 // 2 F1\|2 73.0 cM // | | | |
| NM_026274 // Rspry1 // ring finger and SPRY | Rspry1 | 0.60 | 0.20 |
| domain containing 1 // 8 C5\|8 // 676 | | | |
| NM_019749 // Gabarap // gamma-aminobutyric | Gabarap | 0.40 | 0.19 |
| acid receptor associated protein // 1 | | | |
| NM_198606 // Dcaf13 // DDB1 and CUL4 | Dcaf13 | 0.65 | 0.19 |
| associated factor 13 // 15 B3.1\|15 // 22349 | | | |
| NM_008784 // Igbp1 // immunoglobulin | Igbp1 | 0.40 | 0.19 |
| (CD79A) binding protein 1 // X C3\|X // 1851 | | | |
| NM_013676 // Supt5h // suppressor of Ty 5 | Supt5h | 0.47 | 0.18 |
| homolog (S. cerevisiae) // 7 A3\|7 10.0 | | | |
| NM_027423 // Polr3b // polymerase (RNA) III | Polr3b | 0.30 | 0.18 |
| (DNA directed) polypeptide B // 10 C | | | |
| NM_008302 // Hsp90ab1 // heat shock protein | Hsp90ab1 | 0.53 | 0.18 |
| 90 alpha (cytosolic), class B member | | | |
| NM_016805 // Hnrnpu // heterogeneous nuclear | Hnrnpu | 0.30 | 0.18 |
| ribonucleoprotein U // 1 H4\|1 // 51 | | | |
| NM_178601 // Imp4 // IMP4, U3 small nucleolar | Imp4 | 0.24 | 0.18 |
| ribonucleoprotein, homolog (yeast) | | | |
| NM_001164118 // Serpinb6a // serine (or | Serpinb6a | 0.45 | 0.18 |
| cysteine) peptidase inhibitor, clade B, | | | |
| NM_054078 // Baz2a // bromodomain adjacent | Baz2a | 0.15 | 0.18 |
| to zinc finger domain, 2A // 10 D3\|10 | | | |
| NM_007907 // Eef2 // eukaryotic translation | Eef2 | 0.56 | 0.17 |
| elongation factor 2 // 10 C1\|10 // 1 | | | |
| NM_024479 // Wbscr27 // Williams Beuren | Wbscr27 | 0.50 | 0.17 |
| syndrome chromosome region 27 (human) // | | | |
| NM_011631 // Hsp90b1 // heat shock protein 90, | Hsp90b1 | 0.33 | 0.17 |
| beta (Grp94), member 1 // 10 C1\|1 | | | |
| NM_146043 // Spin1 // spindlin 1 // 13 A5\|13 | Spin1 | 0.52 | 0.16 |
| 31.0 cM // 20729 /// NM_011462 // S | | | |
| NM_026081 // Gprasp1 // G protein-coupled | Gprasp1 | 0.41 | 0.14 |
| receptor associated sorting protein 1 | | | |
| NM_025445 // Arfgap3 // ADP-ribosylation | Arfgap3 | 0.43 | 0.14 |
| factor GTPase activating protein 3 // 1 | | | |
| NM_172695 // Plaa // phospholipase A2, | Plaa | 0.60 | 0.12 |
| activating protein // 4 C5\|4 44.5 cM // 1 | | | |
| NM_146112 // Gigyf2 // GRB10 interacting | Gigyf2 | 0.29 | 0.11 |
| GYF protein 2 // 1 D\|1 // 227331 /// NM_{—} | | | |
| NM_009394 // Tnnc2 // troponin C2, fast // 2 | Tnnc2 | −0.22 | −0.07 |
| H3\|2 // 21925 /// ENSMUST0000010309 | | | |
| NM_177215 // Ocrl // oculocerebrorenal | Ocrl | −1.01 | −0.07 |
| syndrome of Lowe // X A4\|X // 320634 /// | | | |
| NM_144512 // Slc6a13 // solute carrier family 6 | Slc6a13 | −0.18 | −0.09 |
| (neurotransmitter transporter, G | | | |
| NM_030225 // Dlst // dihydrolipoamide S- | Dlst | −0.62 | −0.11 |
| succinyltransferase (E2 component of 2-o | | | |
| NM_177845 // Pla2g4e // phospholipase A2, | Pla2g4e | −0.57 | −0.11 |
| group IVE // 2 E5\|2 // 329502 /// ENSM | | | |
| NM_001164223 // Rpa1 // replication protein A1 | Rpa1 | −0.25 | −0.11 |
| // 11 B5\|11 44.0 cM // 68275 /// | | | |
| NM_007933 // Eno3 // enolase 3, beta muscle // | Eno3 | −2.54 | −0.12 |
| 11 B4\|11 42.0 cM // 13808 /// NM_{—} | | | |
| NM_001014423 // Abi3bp // ABI gene family, | Abi3bp | −0.69 | −0.12 |
| member 3 (NESH) binding protein // 16 | | | |
| NM_028032 // Ppp2r2a // protein phosphatase 2 | Ppp2r2a | −0.82 | −0.12 |
| (formerly 2A), regulatory subunit | | | |
| NM_019804 // B4galt4 // UDP-Gal:betaGlcNAc | B4galt4 | −0.36 | −0.13 |
| beta 1,4-galactosyltransferase, polyp | | | |
| NM_001080797 // G3bp2 // GTPase activating | G3bp2 | −0.54 | −0.13 |
| protein (SH3 domain) binding protein | | | |
| NM_009384 // Tiam1 // T-cell lymphoma | Tiam1 | −0.62 | −0.13 |
| invasion and metastasis 1 // 16 C3-4\|16 61 | | | |
| NM_026125 // Fam132a // family with sequence | Fam132a | −0.45 | −0.15 |
| similarity 132, member A // 4 E2\|4 | | | |
| NM_173029 // Adcy10 // adenylate cyclase 10 // | Adcy10 | −0.18 | −0.15 |
| 1 H2.3\|1 // 271639 /// ENSMUST000 | | | |
| NM_172672 // Ganc // glucosidase, alpha; | Ganc | −1.20 | −0.17 |
| neutral C // 2\|2 F1 // 76051 /// NM_007 | | | |
| NM_019837 // Nudt3 // nudix (nucleotide | Nudt3 | −0.29 | −0.17 |
| diphosphate linked moiety X)-type motif | | | |
| NM_026614 // Ndufa5 // NADH dehydrogenase | Ndufa5 | −0.75 | −0.17 |
| (ubiquinone) 1 alpha subcomplex, 5 // | | | |
| NM_009405 // Tnni2 // troponin I, skeletal, fast | Tnni2 | −0.23 | −0.17 |
| 2 // 7 F5\|7 70.0 cM // 21953 // | | | |
| NM_023220 // 2010106G01Rik // RIKEN | 2010106G01Rik | −0.26 | −0.18 |
| cDNA 2010106G01 gene // 2\|2 F3 // 66552 /// | | | |
| NM_008787 // Pcnt // pericentrin (kendrin) // 10 | Pcnt | −0.34 | −0.18 |
| C1\|10 // 18541 /// ENSMUST00000 | | | |
| NM_153589 // Ano2 // anoctamin 2 // 6 F3\|6 // | Ano2 | −0.14 | −0.18 |
| 243634 /// ENSMUST00000160496 // A | | | |
| NM_007602 // Capn5 // calpain 5 // 7 E2\|7 // | Capn5 | −0.15 | −0.18 |
| 12337 /// ENSMUST00000040971 // Cap | | | |
| NM_020260 // Arhgap31 // Rho GTPase | Arhgap31 | −0.34 | −0.18 |
| activating protein 31 // 16\|16 B4 // 12549 / | | | |
| NM_027123 // Fastkd3 // FAST kinase domains | Fastkd3 | −0.34 | −0.19 |
| 3 // 13 B3\|13 // 69577 /// ENSMUST00 | | | |
| NM_010437 // Hivep2 // human | Hivep2 | −0.28 | −0.19 |
| immunodeficiency virus type I enhancer binding | | | |
| prot | | | |
| NM_001040396 // 2810407C02Rik // RIKEN | 2810407C02Rik | −0.30 | −0.19 |
| cDNA 2810407C02 gene // 3 D\|3 // 69227 // | | | |
| NM_011351 // Sema6c // sema domain, | Sema6c | −0.77 | −0.20 |
| transmembrane domain (TM), and cytoplasmic d | | | |
| NM_015804 // Atp11a // ATPase, class VI, type | Atp11a | −0.42 | −0.21 |
| 11A // 8\|8 A2 // 50770 /// ENSMUST | | | |
| NM_016973 // St6galnac6 // ST6 (alpha-N- | St6galnac6 | −0.42 | −0.21 |
| acetyl-neuraminyl-2,3-beta-galactosyl-1, | | | |
| NM_011948 // Map3k4 // mitogen-activated | Map3k4 | −0.36 | −0.21 |
| protein kinase kinase kinase 4 // 17 A1 | | | |
| NM_027457 // 5730437N04Rik // RIKEN | 5730437N04Rik | −0.45 | −0.22 |
| cDNA 5730437N04 gene // 17 A1\|17 // 70544 // | | | |
| NM_181039 // Lphn1 // latrophilin 1 // 8 C3\|8 // | Lphn1 | −0.29 | −0.22 |
| 330814 /// ENSMUST00000141158 / | | | |
| NM_020007 // Mbnl1 // muscleblind-like 1 | Mbnl1 | −0.39 | −0.22 |
| (Drosophila) // 3 E1\|3 // 56758 /// ENS | | | |
| NR_033188 // 4931409K22Rik // RIKEN cDNA | 4931409K22Rik | −0.12 | −0.22 |
| 4931409K22 gene // 5 A3\|5 // 231045 /// | | | |
| NM_001012330 // Zfp238 // zinc finger protein | Zfp238 | −0.37 | −0.22 |
| 238 // 1\|1 H3 // 30928 /// NM_0139 | | | |
| NM_001102455 // Aplp2 // amyloid beta (A4) | Aplp2 | −1.11 | −0.22 |
| precursor-like protein 2 // 9 A2-B\|9 | | | |
| NM_207682 // Kif1b // kinesin family member | Kif1b | −1.00 | −0.23 |
| 1B // 4 E\|4 70.9 cM // 16561 /// NM_{—} | | | |
| NM_145466 // A2ld1 // AIG2-like domain 1 // | A2ld1 | −0.31 | −0.23 |
| 14 E5\|14 // 223267 /// BC006662 // A | | | |
| NM_018814 // Pcnx // pecanex homolog | Pcnx | −0.67 | −0.23 |
| (Drosophila) // 12 D1\|12 // 54604 /// ENSMU | | | |
| NM_001160127 // Smyd1 // SET and MYND | Smyd1 | −1.29 | −0.23 |
| domain containing 1 // 6 C1\|6 30.5 cM // 1 | | | |
| NM_009817 // Cast // calpastatin // 13 C1\|13 // | Cast | −0.32 | −0.23 |
| 12380 /// ENSMUST00000065629 // | | | |
| NM_145463 // Shisa2 // shisa homolog 2 | Shisa2 | −0.83 | −0.23 |
| (Xenopus laevis) // 14 D1\|14 // 219134 // | | | |
| NM_177470 // Acaa2 // acetyl-Coenzyme A | Acaa2 | −1.48 | −0.23 |
| acyltransferase 2 (mitochondrial 3-oxoac | | | |
| NM_021559 // Zfp191 // zinc finger protein 191 | Zfp191 | −0.49 | −0.24 |
| // 18\|18 B1 // 59057 /// ENSMUST0 | | | |
| NM_175118 // Dusp28 // dual specificity | Dusp28 | −0.25 | −0.24 |
| phosphatase 28 // 1 D\|1 // 67446 /// ENS | | | |
| NM_026695 // Etfb // electron transferring | Etfb | −0.49 | −0.25 |
| flavoprotein, beta polypeptide // 7 B | | | |
| NR_024093 // U05342 // sequence U05342 // 19 | U05342 | −0.66 | −0.25 |
| A\|19 // 664779 /// NM_053009 // Zfp | | | |
| NM_007505 // Atp5a1 // ATP synthase, H+ | Atp5a1 | −0.73 | −0.25 |
| transporting, mitochondrial F1 complex, | | | |
| NM_133987 // Slc6a8 // solute carrier family 6 | Slc6a8 | −0.33 | −0.25 |
| (neurotransmitter transporter, cr | | | |
| NM_007862 // Dlg1 // discs, large homolog 1 | Dlg1 | −0.65 | −0.26 |
| (Drosophila) // 16 B2\|16 21.2 cM // | | | |
| NM_172925 // Klhl31 // kelch-like 31 | Klhl31 | −1.26 | −0.26 |
| (Drosophila) // 9 E1\|9 // 244923 /// ENSMUS | | | |
| NM_001110783 // Ank1 // ankyrin 1, erythroid // | Ank1 | −0.57 | −0.26 |
| 8 A2\|8 9.5 cM // 11733 /// NM_03 | | | |
| NM_009551 // Zfand5 // zinc finger, AN1-type | Zfand5 | −0.09 | −0.27 |
| domain 5 // 19 B\|19 15.0 cM // 2268 | | | |
| NM_133668 // Slc25a3 // solute carrier family | Slc25a3 | −0.67 | −0.27 |
| 25 (mitochondrial carrier, phospha | | | |
| NM_032543 // Rnf123 // ring finger protein 123 | Rnf123 | −0.51 | −0.27 |
| // 9 F2\|9 // 84585 /// ENSMUST000 | | | |
| NM_023644 // Mccc1 // methylcrotonoyl- | Mccc1 | −0.52 | −0.28 |
| Coenzyme A carboxylase 1 (alpha) // 3 B\|3 | | | |
| NM_015802 // Dlc1 // deleted in liver cancer 1 // | Dlc1 | −0.37 | −0.28 |
| 8 A4-B2\|8 21.0 cM // 50768 /// | | | |
| NM_023055 // Slc9a3r2 // solute carrier family 9 | Slc9a3r2 | −0.24 | −0.28 |
| (sodium/hydrogen exchanger), me | | | |
| NM_001037170 // Tomm40l // translocase of | Tomm40l | −0.56 | −0.28 |
| outer mitochondrial membrane 40 homolo | | | |
| NM_001164172 // Map2k7 // mitogen-activated | Map2k7 | −0.32 | −0.29 |
| protein kinase kinase 7 // 8 A1.1\|8 | | | |
| NM_023908 // Slco3a1 // solute carrier organic | Slco3a1 | −0.49 | −0.29 |
| anion transporter family, member | | | |
| NM_172274 // Cc2d2a // coiled-coil and C2 | Cc2d2a | −0.51 | −0.29 |
| domain containing 2A // 5 B3\|5 // 2312 | | | |
| NM_025983 // Atp5e // ATP synthase, H+ | Atp5e | −0.59 | −0.29 |
| transporting, mitochondrial F1 complex, e | | | |
| NM_026794 // Deb1 // differentially expressed | Deb1 | −0.29 | −0.29 |
| in B16F10 1 // 9 F4\|9 // 26901 /// | | | |
| NM_009831 // Ccng1 // cyclin G1 // 11\|11 B1.1 | Ccng1 | −0.57 | −0.29 |
| // 12450 /// ENSMUST00000020576 // | | | |
| NM_010209 // Fh1 // fumarate hydratase 1 // 1 | Fh1 | −0.59 | −0.30 |
| H4\|1 // 14194 /// ENSMUST000000278 | | | |
| NM_178700 // Grsf1 // G-rich RNA sequence | Grsf1 | −0.60 | −0.30 |
| binding factor 1 // 5 E1\|5 48.0 cM // | | | |
| NM_019677 // Plcb1 // phospholipase C, beta 1 | Plcb1 | −0.21 | −0.30 |
| // 2 F3\|2 76.7 cM // 18795 /// NM_{—} | | | |
| NM_025710 // Uqcrfs1 // ubiquinol-cytochrome | Uqcrfs1 | −0.45 | −0.30 |
| c reductase, Rieske iron-sulfur pol | | | |
| NM_175114 // Trak1 // trafficking protein, | Trak1 | −0.64 | −0.30 |
| kinesin binding 1 // 9 F4\|9 71.0 cM / | | | |
| NM_172146 // Ppat // phosphoribosyl | Ppat | −0.74 | −0.30 |
| pyrophosphate amidotransferase // 5 C3.3\|5 / | | | |
| NM_008323 // Idh3g // isocitrate dehydrogenase | Idh3g | −0.64 | −0.30 |
| 3 (NAD+), gamma // X A7.3-B\|X 29. | | | |
| NM_145614 // Dlat // dihydrolipoamide S- | Dlat | −0.85 | −0.30 |
| acetyltransferase (E2 component of pyruv | | | |
| NM_009176 // St3gal3 // ST3 beta-galactoside | St3gal3 | −0.46 | −0.31 |
| alpha-2,3-sialyltransferase 3 // 4\| | | | |
| NM_026734 // Tmem126b // transmembrane | Tmem126b | −0.59 | −0.31 |
| protein 126B // 7\|7 E1 // 68472 /// ENSMU | | | |
| NM_054077 // Prelp // proline arginine-rich end | Prelp | −0.30 | −0.31 |
| leucine-rich repeat // 1 E4\|1 74 | | | |
| NM_133798 // Exd2 // exonuclease 3′-5′ domain | Exd2 | −0.93 | −0.31 |
| containing 2 // 12 C3\|12 // 97827 | | | |
| NM_023172 // Ndufb9 // NADH dehydrogenase | Ndufb9 | −0.75 | −0.31 |
| (ubiquinone) 1 beta subcomplex, 9 // 1 | | | |
| NM_138667 // Tab2 // TGF-beta activated | Tab2 | −0.70 | −0.31 |
| kinase 1/MAP3K7 binding protein 2 // 10 | | | |
| NM_001081242 // Tln2 // talin 2 // 9 C\|9 // | Tln2 | −0.50 | −0.32 |
| 70549 /// NR_029576 // Mir190 // mic | | | |
| NM_172903 // Man2a2 // mannosidase 2, alpha | Man2a2 | −0.35 | −0.32 |
| 2 // 7 D2\|7 // 140481 /// ENSMUST000 | | | |
| NM_031176 // Tnxb // tenascin XB // 17 B1\|17 | Tnxb | −0.15 | −0.32 |
| 18.74 cM // 81877 /// ENSMUST000000 | | | |
| NM_023281 // Sdha // succinate dehydrogenase | Sdha | −0.91 | −0.32 |
| complex, subunit A, flavoprotein (F | | | |
| NM_001161765 // Fmo5 // flavin containing | Fmo5 | −0.28 | −0.32 |
| monooxygenase 5 // 3 F2.2\|3 // 14263 / | | | |
| NM_175347 // Srl // sarcalumenin // 16 A1\|16 // | Srl | −1.23 | −0.32 |
| 106393 /// ENSMUST00000023161 // | | | |
| NM_019718 // Arl3 // ADP-ribosylation factor- | Arl3 | −1.02 | −0.33 |
| like 3 // 19\|19 D1 // 56350 /// ENS | | | |
| NM_008885 // Pmp22 // peripheral myelin | Pmp22 | −1.63 | −0.33 |
| protein 22 // 11 B3\|11 34.45 cM // 18858 | | | |
| NM_177860 // Gm5105 // predicted gene 5105 // | Gm5105 | −0.95 | −0.33 |
| 3 G3\|3 // 329763 /// ENSMUST000000 | | | |
| NM_173397 // Fitm2 // fat storage-inducing | Fitm2 | −0.62 | −0.33 |
| transmembrane protein 2 // 2 H3\|2 // | | | |
| NM_026448 // Klhl7 // kelch-like 7 (Drosophila) | Klhl7 | −0.29 | −0.33 |
| // 5 A3\|5 12.0 cM // 52323 /// N | | | |
| NM_172426 // Slc24a2 // solute carrier family | Slc24a2 | −0.39 | −0.34 |
| 24 (sodium/potassium/calcium excha | | | |
| NM_001136056 // Cntfr // ciliary neurotrophic | Cntfr | −0.28 | −0.34 |
| factor receptor // 4 A5\|4 19.9 cM | | | |
| NM_181417 // Csrp2bp // cysteine and glycine- | Csrp2bp | −0.60 | −0.34 |
| rich protein 2 binding protein // 2 | | | |
| NM_146193 // Btbd1 // BTB (POZ) domain | Btbd1 | −0.37 | −0.34 |
| containing 1 // 7 D1\|7 // 83962 /// ENSMU | | | |
| NM_145999 // Rhot2 // ras homolog gene | Rhot2 | −0.64 | −0.34 |
| family, member T2 // 17 A3.3\|17 11.6 cM / | | | |
| NM_013881 // Ulk2 // Unc-51 like kinase 2 (C. elegans) | Ulk2 | −0.54 | −0.34 |
| // 11 B2\|11 // 29869 /// | | | |
| NM_178060 // Thra // thyroid hormone receptor | Thra | −0.71 | −0.34 |
| alpha // 11 D-E\|11 57.0 cM // 2183 | | | |
| NM_001172146 // Aimp2 // aminoacyl tRNA | Aimp2 | −1.85 | −0.34 |
| synthetase complex-interacting multifunc | | | |
| NM_130892 // Rtn4ip1 // reticulon 4 interacting | Rtn4ip1 | −1.07 | −0.35 |
| protein 1 // 10 B2\|10 29.0 cM // | | | |
| NM_134117 // Pkdcc // protein kinase domain | Pkdcc | −0.54 | −0.35 |
| containing, cytoplasmic // 17 E4\|17 | | | |
| NM_010898 // Nf2 // neurofibromatosis 2 // 11 | Nf2 | −0.29 | −0.35 |
| A1\|11 0.25 cM // 18016 /// ENSMUST | | | |
| NM_001114124 // Dab2ip // disabled homolog 2 | Dab2ip | −0.33 | −0.35 |
| (Drosophila) interacting protein // | | | |
| NM_021508 // Myoz1 // myozenin 1 // 14 A3\|14 | Myoz1 | −1.81 | −0.35 |
| // 59011 /// ENSMUST00000090469 // | | | |
| NM_009124 // Atxn1 // ataxin 1 // 13 A5\|13 28.0 cM | Atxn1 | −0.19 | −0.35 |
| // 20238 /// ENSMUST000000916 | | | |
| NM_008664 // Myom2 // myomesin 2 // 8 A1.1\|8 | Myom2 | −1.62 | −0.36 |
| // 17930 /// ENSMUST00000033842 // | | | |
| NM_133825 // D1Ertd622e // DNA segment, | D1Ertd622e | −0.78 | −0.36 |
| Chr 1, ERATO Doi 622, expressed // 1 D\|1 | | | |
| NM_008910 // Ppm1a // protein phosphatase 1A, | Ppm1a | −0.55 | −0.36 |
| magnesium dependent, alpha isoform | | | |
| NM_001025250 // Vegfa // vascular endothelial | Vegfa | −0.87 | −0.36 |
| growth factor A // 17 C\|17 24.2 cM | | | |
| NM_172710 // Sel1l3 // sel-1 suppressor of lin- | Sel1l3 | −1.03 | −0.36 |
| 12-like 3 (C. elegans) // 5 C1\|5 | | | |
| NM_009694 // Apobec2 // apolipoprotein B | Apobec2 | −2.13 | −0.36 |
| mRNA editing enzyme, catalytic polypept | | | |
| NM_177407 // Camk2a // calcium/calmodulin- | Camk2a | −1.37 | −0.37 |
| dependent protein kinase II alpha // 1 | | | |
| NM_025287 // Spop // speckle-type POZ protein | Spop | −0.79 | −0.37 |
| // 11 D\|11 55.6 cM // 20747 /// EN | | | |
| NM_021515 // Ak1 // adenylate kinase 1 // 2 B\|2 | Ak1 | −1.56 | −0.37 |
| 21.6 cM // 11636 /// ENSMUST0000 | | | |
| NM_010158 // Khdrbs3 // KH domain | Khdrbs3 | −0.89 | −0.37 |
| containing, RNA binding, signal transduction a | | | |
| NM_199008 // Cox11 // COX11 homolog, | Cox11 | −0.55 | −0.37 |
| cytochrome c oxidase assembly protein (yeas | | | |
| NM_011918 // Ldb3 // LIM domain binding 3 // | Ldb3 | −0.80 | −0.37 |
| 14 B\|14 // 24131 /// NM_001039072 / | | | |
| NM_133666 // Ndufv1 // NADH dehydrogenase | Ndufv1 | −0.90 | −0.37 |
| (ubiquinone) flavoprotein 1 // 19 A\|19 | | | |
| NM_009868 // Cdh5 // cadherin 5 // 8 D3\|8 51.0 cM | Cdh5 | −0.44 | −0.38 |
| // 12562 /// ENSMUST0000003433 | | | |
| NM_212433 // Fbxo3 // F-box protein 3 // 2 E2\|2 | Fbxo3 | −0.73 | −0.38 |
| // 57443 /// NM_020593 // Fbxo3 | | | |
| NM_080444 // Asb10 // ankyrin repeat and | Asb10 | −0.43 | −0.38 |
| SOCS box-containing 10 // 5 A3\|5 // 117 | | | |
| NM_144784 // Acat1 // acetyl-Coenzyme A | Acat1 | −0.39 | −0.39 |
| acetyltransferase 1 // 9 C-D19 30.0 cM / | | | |
| NM_009943 // Cox6a2 // cytochrome c oxidase, | Cox6a2 | −0.59 | −0.39 |
| subunit VI a, polypeptide 2 // 7 F3 | | | |
| NM_001013013 // Dhrs7c // | Dhrs7c | −3.28 | −0.39 |
| dehydrogenase/reductase (SDR family) member | | | |
| 7C // 11 B | | | |
| NM_144821 // AI317395 // expressed sequence | AI317395 | −0.41 | −0.39 |
| AI317395 // 10 B1\|10 // 215929 /// N | | | |
| NM_008063 // Slc37a4 // solute carrier family | Slc37a4 | −1.57 | −0.39 |
| 37 (glucose-6-phosphate transporte | | | |
| NM_026703 // Ndufa8 // NADH dehydrogenase | Ndufa8 | −0.69 | −0.39 |
| (ubiquinone) 1 alpha subcomplex, 8 // | | | |
| NM_172707 // Ppp1cb // protein phosphatase 1, | Ppp1cb | −0.41 | −0.39 |
| catalytic subunit, beta isoform // | | | |
| NM_007450 // Slc25a4 // solute carrier family | Slc25a4 | −0.93 | −0.39 |
| 25 (mitochondrial carrier, adenine | | | |
| NM_001033208 // Gcom1 // GRINL1A complex | Gcom1 | −0.57 | −0.40 |
| locus // 9 D\|9 // 102371 /// NM_178602 | | | |
| NM_178675 // Slc35f1 // solute carrier family | Slc35f1 | −0.64 | −0.40 |
| 35, member F1 // 10 B3\|10 // 21508 | | | |
| NM_019879 // Suclg1 // succinate-CoA ligase, | Suclg1 | −0.80 | −0.40 |
| GDP-forming, alpha subunit // 6\|6 C | | | |
| NM_008212 // Hadh // hydroxyacyl-Coenzyme | Hadh | −0.98 | −0.40 |
| A dehydrogenase // 3 G3\|3 // 15107 /// | | | |
| NM_018870 // Pgam2 // phosphoglycerate | Pgam2 | −1.55 | −0.40 |
| mutase 2 // 11 A1\|11 // 56012 /// ENSMUST | | | |
| NM_052992 // Fxyd1 // FXYD domain- | Fxyd1 | −0.65 | −0.40 |
| containing ion transport regulator 1 // 7\|7 B1 | | | |
| NM_001160038 // Ndufs1 // NADH | Ndufs1 | −1.12 | −0.41 |
| dehydrogenase (ubiquinone) Fe—S protein 1 // 1 | | | |
| C2 | | | |
| NM_024211 // Slc25a11 // solute carrier family | Slc25a11 | −0.94 | −0.41 |
| 25 (mitochondrial carrier oxoglut | | | |
| NM_008618 // Mdh1 // malate dehydrogenase 1, | Mdh1 | −1.25 | −0.41 |
| NAD (soluble) // 11 A3.1\|11 12.0 cM | | | |
| NM_025631 // Bpil1 // bactericidal/permeability- | Bpil1 | −0.14 | −0.41 |
| increasing protein-like 1 // 2 H | | | |
| NM_026172 // Decr1 // 2,4-dienoyl CoA | Decr1 | −0.64 | −0.41 |
| reductase 1, mitochondrial // 4 A2\|4 // 67 | | | |
| NM_011101 // Prkca // protein kinase C, alpha // | Prkca | −0.86 | −0.41 |
| 11 E1\|11 68.0 cM // 18750 /// E | | | |
| NM_027748 // Taf3 // TAF3 RNA polymerase | Taf3 | −0.67 | −0.41 |
| II, TATA box binding protein (TBP)-asso | | | |
| NM_026530 // Mpnd // MPN domain containing | Mpnd | −0.47 | −0.41 |
| // 17\|17 D // 68047 /// ENSMUST000001 | | | |
| NM_177784 // Klhl23 // kelch-like 23 | Klhl23 | −1.15 | −0.41 |
| (Drosophila) // 2 C2\|2 // 277396 /// ENSMUS | | | |
| NM_144870 // Ndufs8 // NADH dehydrogenase | Ndufs8 | −0.95 | −0.41 |
| (ubiquinone) Fe—S protein 8 // 19 A\|19 | | | |
| NM_025460 // Tmem126a // transmembrane | Tmem126a | −0.63 | −0.42 |
| protein 126A // 7\|7 E1 // 66271 /// ENSMU | | | |
| NM_153064 // Ndufs2 // NADH dehydrogenase | Ndufs2 | −1.02 | −0.42 |
| (ubiquinone) Fe—S protein 2 // 1 H3\|1 | | | |
| NM_023784 // Yipf7 // Yip1 domain family, | Yipf7 | −1.32 | −0.42 |
| member 7 // 5\|5 D // 75581 /// ENSMUST | | | |
| NM_026061 // Ndufb8 // NADH dehydrogenase | Ndufb8 | −0.97 | −0.42 |
| (ubiquinone) 1 beta subcomplex 8 // 19 | | | |
| NM_009653 // Alas2 // aminolevulinic acid | Alas2 | −0.66 | −0.42 |
| synthase 2, erythroid // X F3\|X 63.0 c | | | |
| NM_008095 // Gbas // glioblastoma amplified | Gbas | −0.87 | −0.42 |
| sequence // 5 G1.3\|5 // 14467 /// EN | | | |
| NM_009948 // Cpt1b // carnitine | Cpt1b | −1.30 | −0.42 |
| palmitoyltransferase 1b, muscle // 15 E3\|15 52.6 | | | |
| NM_177694 // Ano5 // anoctamin 5 // 7 B5\|7 // | Ano5 | −3.39 | −0.43 |
| 233246 /// ENSMUST00000043944 // A | | | |
| NM_001111059 // Cd34 // CD34 antigen // 1 | Cd34 | −0.42 | −0.43 |
| H6\|1 106.6 cM // 12490 /// NM_133654 / | | | |
| NM_008617 // Mdh2 // malate dehydrogenase 2, | Mdh2 | −0.96 | −0.43 |
| NAD (mitochondrial) // 5 G2\|5 78.0 | | | |
| NM_025407 // Uqcrc1 // ubiquinol-cytochrome c | Uqcrc1 | −0.81 | −0.43 |
| reductase core protein 1 // 9 F2\|9 | | | |
| NM_007996 // Fdx1 // ferredoxin 1 // 9\|9 B // | Fdx1 | −0.69 | −0.43 |
| 14148 /// ENSMUST00000034552 // Fd | | | |
| NM_054087 // Slc19a2 // solute carrier family | Slc19a2 | −0.32 | −0.44 |
| 19 (thiamine transporter), member | | | |
| NM_175535 // Arhgap20 // Rho GTPase | Arhgap20 | −1.25 | −0.44 |
| activating protein 20 // 9 A5.3\|9 // 244867 | | | |
| NM_019874 // Dnajb5 // DnaJ (Hsp40) | Dnajb5 | −1.08 | −0.44 |
| homolog, subfamily B, member 5 // 4\|4 B1 // | | | |
| NM_008377 // Lrig1 // leucine-rich repeats and | Lrig1 | −0.82 | −0.44 |
| immunoglobulin-like domains 1 // | | | |
| NM_175650 // Atp13a5 // ATPase type 13A5 // | Atp13a5 | −0.43 | −0.44 |
| 16 B2\|16 // 268878 /// ENSMUST000000 | | | |
| NM_010636 // Klf12 // Kruppel-like factor 12 // | Klf12 | −0.75 | −0.44 |
| 14 E2.2\|14 47.5 cM // 16597 /// | | | |
| NM_010612 // Kdr // kinase insert domain | Kdr | −0.77 | −0.44 |
| protein receptor // 5 C3.3\|5 42.0 cM // | | | |
| NM_030678 // Gys1 // glycogen synthase 1, | Gys1 | −1.18 | −0.44 |
| muscle // 7 B4\|7 23.0 cM // 14936 /// | | | |
| NM_013598 // Kitl // kit ligand // 10 D1\|10 57.0 cM | Kitl | −0.47 | −0.45 |
| // 17311 /// ENSMUST00000105 | | | |
| NR_027897 // 0610012G03Rik // RIKEN cDNA | 0610012G03Rik | −0.50 | −0.45 |
| 0610012G03 gene // 16 B2\|16 // 106264 / | | | |
| NM_178726 // Ppm11 // protein phosphatase 1 | Ppm11 | −1.09 | −0.45 |
| (formerly 2C)-like // 3 E1\|3 // 2420 | | | |
| NM_021896 // Gucy1a3 // guanylate cyclase 1, | Gucy1a3 | −0.23 | −0.45 |
| soluble, alpha 3 // 3 E3\|3 // 60596 | | | |
| NM_010141 // Epha7 // Eph receptor A7 // 4 | Epha7 | −0.61 | −0.45 |
| A4\|4 1.9 cM // 13841 /// NM_001122889 | | | |
| NM_013917 // Pttg1 // pituitary tumor- | Pttg1 | −0.38 | −0.45 |
| transforming gene 1 // 11 A5\|11 // 30939 / | | | |
| NM_012029 // Ecsit // ECSIT homolog | Ecsit | −0.54 | −0.45 |
| (Drosophila) // 9\|9 A4 // 26940 /// ENSMUST0 | | | |
| NM_021398 // Slc43a3 // solute carrier family | Slc43a3 | −0.28 | −0.45 |
| 43, member 3 // 2 E1\|2 // 58207 // | | | |
| NM_011697 // Vegfb // vascular endothelial | Vegfb | −0.43 | −0.46 |
| growth factor B // 19\|19 B // 22340 / | | | |
| NM_007931 // Endog // endonuclease G // 2 B\|2 | Endog | −0.57 | −0.46 |
| // 13804 /// NM_172660 // D2Wsu81e | | | |
| NM_173866 // Gpt2 // glutamic pyruvate | Gpt2 | −1.19 | −0.46 |
| transaminase (alanine aminotransferase) 2 | | | |
| NM_008831 // Phb // prohibitin // 11 D\|11 55.6 cM | Phb | −1.01 | −0.46 |
| // 18673 /// NR_028263 // B130 | | | |
| NM_001127363 // Inpp5a // inositol | Inpp5a | −0.51 | −0.46 |
| polyphosphate-5-phosphatase A // 7 F4\|7 // 21 | | | |
| NM_008914 // Ppp3cb // protein phosphatase 3, | Ppp3cb | −0.65 | −0.46 |
| catalytic subunit, beta isoform // | | | |
| NM_027295 // Rab28 // RAB28, member RAS | Rab28 | −0.87 | −0.47 |
| oncogene family // 5\|5 B2 // 100972 /// | | | |
| NM_026514 // Cdc42ep3 // CDC42 effector | Cdc42ep3 | −1.23 | −0.47 |
| protein (Rho GTPase binding) 3 // 17 E3\| | | | |
| NM_008393 // Irx3 // Iroquois related homeobox | Irx3 | −0.82 | −0.47 |
| 3 (Drosophila) // 8 C5\|8 42.1 cM | | | |
| NM_172734 // Stk381// serine/threonine kinase | Stk38l | −0.71 | −0.47 |
| 38 like // 6 G3\|6 // 232533 /// E | | | |
| NM_020604 // Jph1 // junctophilin 1 // 1\|1 A4 // | Jph1 | −1.11 | −0.47 |
| 57339 /// ENSMUST00000038382 // | | | |
| NM_008551 // Mapkapk2 // MAP kinase- | Mapkapk2 | −1.43 | −0.47 |
| activated protein kinase 2 // 1 E4\|1 // 1716 | | | |
| NM_172752 // Sorbs2 // sorbin and SH3 domain | Sorbs2 | −1.64 | −0.47 |
| containing 2 // 8 B1.1\|8 // 234214 | | | |
| NM_178681 // Dgkb // diacylglycerol kinase, | Dgkb | −0.27 | −0.47 |
| beta // 12 A3\|12 // 217480 /// ENSMU | | | |
| NM_001038999 // Atp8a1 // ATPase, | Atp8a1 | −1.17 | −0.47 |
| aminophospholipid transporter (APLT), class I, | | | |
| NM_177271 // Samd5 // sterile alpha motif | Samd5 | −0.33 | −0.48 |
| domain containing 5 // 10 A1\|10 // 320 | | | |
| NM_007917 // Eif4e // eukaryotic translation | Eif4e | −1.62 | −0.48 |
| initiation factor 4E //3 G3-H1\|3 2 | | | |
| NM_022322 // Tnmd // tenomodulin // X E3\|X // | Tnmd | −0.80 | −0.48 |
| 64103 /// ENSMUST00000033602 // Tn | | | |
| NM_009261 // Strbp // spermatid perinuclear | Strbp | −0.95 | −0.48 |
| RNA binding protein // 2 B\|2 // 2074 | | | |
| NM_001080818 // Cdc14a // CDC14 cell | Cdc14a | −0.69 | −0.48 |
| division cycle 14 homolog A (S. cerevisiae) | | | |
| NM_172904 // Fsd2 // fibronectin type III and | Fsd2 | −2.43 | −0.48 |
| SPRY domain containing 2 // 7 D3\|7 | | | |
| NM_145076 // Trim24 // tripartite motif- | Trim24 | −0.85 | −0.48 |
| containing 24 // 6 B1\|6 // 21848 /// ENS | | | |
| NM_010347 // Aes // amino-terminal enhancer | Aes | −1.02 | −0.48 |
| of split // 10 C1\|10 43.0 cM // 1479 | | | |
| NM_010284 // Ghr // growth hormone receptor // | Ghr | −0.40 | −0.49 |
| 15 A1\|15 4.6 cM // 14600 /// NM_0 | | | |
| NM_013584 // Lifr // leukemia inhibitory factor | Lifr | −0.94 | −0.49 |
| receptor // 15 A1\|15 4.6 cM // 1 | | | |
| NM_025352 // Uqcrq // ubiquinol-cytochrome c | Uqcrq | −0.65 | −0.49 |
| reductase, complex III subunit VII | | | |
| NM_025558 // Cyb5b // cytochrome b5 type B // | Cyb5b | −0.91 | −0.49 |
| 8\|8 D2 // 66427 /// ENSMUST0000003 | | | |
| NM_009739 // Bckdk // branched chain ketoacid | Bckdk | −0.54 | −0.50 |
| dehydrogenase kinase // 7 F3\|7 // | | | |
| NM_023844 // Jam2 // junction adhesion | Jam2 | −0.59 | −0.50 |
| molecule 2 // 16 C3.3\|16 // 67374 /// ENS | | | |
| NM_007906 // Eef1a2 // eukaryotic translation | Eef1a2 | −0.63 | −0.50 |
| elongation factor 1 alpha 2 // 2 H | | | |
| NM_011076 // Abcb1a // ATP-binding cassette, | Abcb1a | −0.23 | −0.50 |
| sub-family B (MDR/TAP), member 1A / | | | |
| NM_026272 // Narf // nuclear prelamin A | Narf | −0.76 | −0.50 |
| recognition factor // 11 E2\|11 // 67608 | | | |
| NM_053123 // Smarca1 // SWI/SNF related, | Smarca1 | −1.01 | −0.50 |
| matrix associated, actin dependent regu | | | |
| NM_010164 // Eya1 // eyes absent 1 homolog | Eya1 | −0.91 | −0.50 |
| (Drosophila) // 1 A3\|1 10.4 cM // 140 | | | |
| NM_199448 // Fez2 // fasciculation and | Fez2 | −1.20 | −0.50 |
| elongation protein zeta 2 (zygin II) // 1 | | | |
| NM_027924 // Pdgfd // platelet-derived growth | Pdgfd | −0.24 | −0.50 |
| factor, D polypeptide // 9 A1\|9 // | | | |
| NM_024255 // Hsdl2 // hydroxysteroid | Hsdl2 | −0.62 | −0.51 |
| dehydrogenase like 2 // 4\|4 C1 // 72479 /// | | | |
| NM_133741 // Snrk // SNF related kinase // 9 | Snrk | −0.62 | −0.51 |
| F4\|9 // 20623 /// NM_001164572 // S | | | |
| NM_013864 // Ndrg2 // N-myc downstream | Ndrg2 | −0.73 | −0.51 |
| regulated gene 2 // 14 C2\|14 // 29811 /// | | | |
| NM_172925 // Klhl31 // kelch-like 31 | Klhl31 | −1.58 | −0.51 |
| (Drosophila) // 9 E1\|9 // 244923 /// ENSMUS | | | |
| NM_080858 // Asb12 // ankyrin repeat and | Asb12 | −2.49 | −0.51 |
| SOCS box-containing 12 // X\|X C1 // 703 | | | |
| NM_172310 // Tarsl2 // threonyl-tRNA | Tarsl2 | −1.06 | −0.51 |
| synthetase-like 2 // 7 C\|7 // 272396 /// EN | | | |
| NM_007710 // Ckm // creatine kinase, muscle // | Ckm | −1.00 | −0.51 |
| 7 A3\|7 4.5 cM // 12715 /// ENSMUS | | | |
| NM_198013 // Cuedc1 // CUE domain | Cuedc1 | −0.67 | −0.52 |
| containing 1 // 11 C\|11 // 103841 /// NM_00117 | | | |
| NM_001005863 // Mtus1 // mitochondrial tumor | Mtus1 | −1.21 | −0.52 |
| suppressor 1 // 8 A4\|8 // 102103 // | | | |
| NM_198308 // Pdpr // pyruvate dehydrogenase | Pdpr | −0.94 | −0.52 |
| phosphatase regulatory subunit // 8 | | | |
| NM_023374 // Sdhb // succinate dehydrogenase | Sdhb | −1.23 | −0.52 |
| complex, subunit B, iron sulfur (Ip | | | |
| NM_008425 // Kcnj2 // potassium inwardly- | Kcnj2 | −0.65 | −0.52 |
| rectifying channel, subfamily J, member | | | |
| NM_017366 // Acadvl // acyl-Coenzyme A | Acadvl | −0.58 | −0.52 |
| dehydrogenase, very long chain // 11 B2-B | | | |
| NM_172406 // Trak2 // trafficking protein, | Trak2 | −0.86 | −0.53 |
| kinesin binding 2 // 1 C1.3\|1 // 7082 | | | |
| NM_025336 // Chchd3 // coiled-coil-helix- | Chchd3 | −0.91 | −0.53 |
| coiled-coil-helix domain containing 3 / | | | |
| NM_016978 // Oat // ornithine aminotransferase | Oat | −0.63 | −0.54 |
| // 7 F3\|7 63.0 cM // 18242 /// EN | | | |
| NM_011144 // Ppara // peroxisome proliferator | Ppara | −0.54 | −0.54 |
| activated receptor alpha // 15 E2\| | | | |
| NM_027093 // 2310003L22Rik // RIKEN cDNA | 2310003L22Rik | −1.14 | −0.54 |
| 2310003L22 gene // 2\|2 G3 // 69487 /// | | | |
| NM_027667 // Arhgap19 // Rho GTPase | Arhgap19 | −1.38 | −0.54 |
| activating protein 19 // 19\|19 D1 // 71085 / | | | |
| NM_024469 // Bhlhe41 // basic helix-loop-helix | Bhlhe41 | −1.18 | −0.55 |
| family, member e41 // 6 G2-G3\|6 7 | | | |
| NM_020295 // Lmbr1 // limb region 1 // 5 B1\|5 | Lmbr1 | −0.94 | −0.55 |
| 15.8 cM // 56873 /// ENSMUST000000 | | | |
| NM_144853 // Cyyr1 // cysteine and tyrosine- | Cyyr1 | −0.62 | −0.55 |
| rich protein 1 // 16 C3.3\|16 56.2 cM | | | |
| NM_008623 // Mpz // myelin protein zero // 1 | Mpz | −2.41 | −0.55 |
| H3\|1 92.4 cM // 17528 /// ENSMUST00 | | | |
| NM_175207 // Ankrd9 // ankyrin repeat domain | Ankrd9 | −0.20 | −0.56 |
| 9 // 12 F1\|12 // 74251 /// ENSMUST0 | | | |
| NM_001002004 // 2610507B11Rik // RIKEN | 2610507B11Rik | −1.04 | −0.56 |
| cDNA 2610507B11 gene // 11 B5\|11 44.93 cM | | | |
| NM_027126 // Hfe2 // hemochromatosis type 2 | Hfe2 | −0.67 | −0.56 |
| (juvenile) (human homolog) // 3 F2.1 | | | |
| NM_172656 // Stradb // STE20-related kinase | Stradb | −0.73 | −0.57 |
| adaptor beta // 1 C1.3\|1 34.0 cM // | | | |
| NM_025790 // Acot13 // acyl-CoA thioesterase | Acot13 | −0.94 | −0.57 |
| 13 // 13\|13 A3.2 // 66834 /// ENSMU | | | |
| NM_080450 // Gjc3 // gap junction protein, | Gjc3 | −0.60 | −0.57 |
| gamma 3 // 5 G2\|5 // 118446 /// ENSMU | | | |
| NM_026452 // Coq9 // coenzyme Q9 homolog | Coq9 | −0.68 | −0.57 |
| (yeast) // 8 C5-D1\|8 // 67914 /// ENSMU | | | |
| NM_177003 // 9630033F20Rik // RIKEN cDNA | 9630033F20Rik | −0.67 | −0.57 |
| 9630033F20 gene // 6 F3\|6 // 319801 /// | | | |
| NM_011838 // Lynx1 // Ly6/neurotoxin 1 // 15 | Lynx1 | −1.56 | −0.57 |
| D3\|15 // 23936 /// ENSMUST000000232 | | | |
| NM_020332 // Ank // progressive ankylosis // 15 | Ank | −0.54 | −0.57 |
| B1\|15 14.4 cM // 11732 /// ENSMU | | | |
| NM_175413 // Lrrc39 // leucine rich repeat | Lrrc39 | −1.73 | −0.58 |
| containing 39 // 3\|3 G2 // 109245 /// | | | |
| NM_011505 // Stxbp4 // syntaxin binding | Stxbp4 | −1.07 | −0.58 |
| protein 4 // 11\|11 C // 20913 /// ENSMUS | | | |
| NM_029573 // Idh3a // isocitrate dehydrogenase | Idh3a | −1.87 | −0.58 |
| 3 (NAD+) alpha // 9 A5.3\|9 // 678 | | | |
| NM_009063 // Rgs5 // regulator of G-protein | Rgs5 | −0.77 | −0.58 |
| signaling 5 // 1 H2\|1 86.5 cM // 197 | | | |
| NM_175329 // Chchd10 // coiled-coil-helix- | Chchd10 | −0.87 | −0.58 |
| coiled-coil-helix domain containing 10 | | | |
| NM_147176 // Homer1 // homer homolog 1 | Homer1 | −1.54 | −0.58 |
| (Drosophila) // 13 C3\|13 // 26556 /// NM_{—} | | | |
| NM_153803 // Glb1l2 // galactosidase, beta 1- | Glb1l2 | −0.64 | −0.59 |
| like 2 // 9 A4\|9 // 244757 /// ENSM | | | |
| NM_025508 // Gmpr // guanosine | Gmpr | −1.15 | −0.59 |
| monophosphate reductase // 13 A5\|13 // 66355 /// | | | |
| NM_028091 // Osgepl1 // O-sialoglycoprotein | Osgepl1 | −0.57 | −0.59 |
| endopeptidase-like 1 // 1 C1.1\|1 0.0 | | | |
| NM_009721 // Atp1b1 // ATPase, Na+/K+ | Atp1b1 | −0.57 | −0.59 |
| transporting, beta 1 polypeptide // 1 H2.2 | | | |
| NM_008810 // Pdha1 // pyruvate dehydrogenase | Pdha1 | −0.94 | −0.59 |
| E1 alpha 1 // X F3-F4\|X 66.5 cM // | | | |
| NM_153794 // 4933403F05Rik // RIKEN cDNA | 4933403F05Rik | −0.70 | −0.59 |
| 4933403F05 gene // 18 E2\|18 // 108654 / | | | |
| NM_010267 // Gdap1 // ganglioside-induced | Gdap1 | −1.80 | −0.60 |
| differentiation-associated-protein 1 / | | | |
| NM_008509 // Lpl // lipoprotein lipase // 8 | Lpl | −1.46 | −0.60 |
| B3.3\|8 33.0 cM // 16956 /// ENSMUST0 | | | |
| NM_177787 // Slc15a5 // solute carrier family | Slc15a5 | −1.13 | −0.61 |
| 15, member 5 // 6 G1\|6 // 277898 / | | | |
| NM_145619 // Parp3 // poly (ADP-ribose) | Parp3 | −0.68 | −0.61 |
| polymerase family, member 3 // 9 F1\|9 // | | | |
| NM_008862 // Pkia // protein kinase inhibitor, | Pkia | −0.83 | −0.61 |
| alpha // 3 A1\|3 // 18767 /// ENSM | | | |
| NM_011273 // Xpr1 // xenotropic and polytropic | Xpr1 | −0.83 | −0.62 |
| retrovirus receptor 1 // 1 G3\|1 8 | | | |
| NM_018760 // Slc4a4 // solute carrier family 4 | Slc4a4 | −1.41 | −0.62 |
| (anion exchanger), member 4 // 5\| | | | |
| NM_007751 // Cox8b // cytochrome c oxidase, | Cox8b | −0.76 | −0.62 |
| subunit VIIIb // 7 F5\|7 68.8 cM // 1 | | | |
| NM_010271 // Gpd1 // glycerol-3-phosphate | Gpd1 | −2.22 | −0.62 |
| dehydrogenase 1 (soluble) // 15 F1-F3\| | | | |
| NM_011694 // Vdac1 // voltage-dependent anion | Vdac1 | −0.67 | −0.62 |
| channel 1 // 11 B1.3\|11 29.0 cM // | | | |
| NM_183028 // Pcmtd1 // protein-L-isoaspartate | Pcmtd1 | −0.58 | −0.62 |
| (D-aspartate) O-methyltransferase | | | |
| NM_001024504 // Dcun1d2 // DCN1, defective | Dcun1d2 | −0.51 | −0.62 |
| in cullin neddylation 1, domain conta | | | |
| NM_008209 // Mr1 // major histocompatibility | Mr1 | −0.45 | −0.63 |
| complex, class I-related // 1\|1 H1 | | | |
| NR_033306 // Ndufs6 // NADH dehydrogenase | Ndufs6 | −0.50 | −0.63 |
| (ubiquinone) Fe—S protein 6 // 13 D2\|1 | | | |
| NM_001038653 // Slc16a3 // solute carrier | Slc16a3 | −1.98 | −0.63 |
| family 16 (monocarboxylic acid transpo | | | |
| NM_024291 // Ky // kyphoscoliosis peptidase // | Ky | −1.80 | −0.63 |
| 9 F1\|9 56.0 cM // 16716 /// ENSMU | | | |
| NM_207246 // Rasgrp3 // RAS, guanyl releasing | Rasgrp3 | −1.71 | −0.63 |
| protein 3 // 17 E2\|17 // 240168 // | | | |
| NM_178934 // Slc2a12 // solute carrier family 2 | Slc2a12 | −0.76 | −0.63 |
| (facilitated glucose transporter | | | |
| NM_019510 // Trpc3 // transient receptor | Trpc3 | −0.35 | −0.64 |
| potential cation channel, subfamily C, | | | |
| NM_007729 // Col11a1 // collagen, type XI, | Col11a1 | −0.47 | −0.64 |
| alpha 1 // 3 F3\|3 53.1 cM // 12814 // | | | |
| NM_027981 // 2310002L09Rik // RIKEN cDNA | 2310002L09Rik | −1.39 | −0.64 |
| 2310002L09 gene // 4 C3\|4 // 71886 /// | | | |
| NM_080465 // Kcnn2 // potassium | Kcnn2 | −0.37 | −0.64 |
| intermediate/small conductance calcium- | | | |
| activated | | | |
| NM_018784 // St3gal6 // ST3 beta-galactoside | St3gal6 | −1.55 | −0.64 |
| alpha-2,3-sialyltransferase 6 // 16 | | | |
| NM_001145820 // Gpd2 // glycerol phosphate | Gpd2 | −1.19 | −0.64 |
| dehydrogenase 2, mitochondrial // 2 C | | | |
| NM_007382 // Acadm // acyl-Coenzyme A | Acadm | −1.62 | −0.65 |
| dehydrogenase, medium chain // 3 H3\|3 73.6 | | | |
| NM_029928 // Ptprb // protein tyrosine | Ptprb | −0.61 | −0.65 |
| phosphatase, receptor type, B // 10 D2\|10 | | | |
| NM_025348 // Ndufa3 // NADH dehydrogenase | Ndufa3 | −0.54 | −0.65 |
| (ubiquinone) 1 alpha subcomplex, 3 // | | | |
| NM_178597 // Camk2g // calcium/calmodulin- | Camk2g | −0.59 | −0.66 |
| dependent protein kinase II gamma // 1 | | | |
| NM_008358 // Il15ra // interleukin 15 receptor, | Il15ra | −0.25 | −0.66 |
| alpha chain // 2 A1\|2 6.4 cM // | | | |
| NM_133199 // Scn4a // sodium channel, voltage- | Scn4a | −0.36 | −0.66 |
| gated, type IV, alpha // 11 E1\|11 | | | |
| NM_009261 // Strbp // spermatid perinuclear | Strbp | −1.05 | −0.68 |
| RNA binding protein // 2 B\|2 // 2074 | | | |
| NR_027710 // Ppargc1a // peroxisome | Ppargc1a | −1.57 | −0.68 |
| proliferative activated receptor, gamma, coa | | | |
| NM_029475 // Adal // adenosine deaminase-like | Adal | −0.64 | −0.68 |
| // 2\|2 F1 // 75894 /// ENSMUST0000 | | | |
| NM_024200 // Mfn1 // mitofusin 1 // 3 B\|3 13.0 cM | Mfn1 | −0.98 | −0.68 |
| // 67414 /// ENSMUST0000009125 | | | |
| NM_008413 // Jak2 // Janus kinase 2 // 19 C1\|19 | Jak2 | −1.26 | −0.68 |
| 24.0 cM // 16452 /// NM_00104817 | | | |
| NM_007643 // Cd36 // CD36 antigen // 5 A3\|5 | Cd36 | −0.67 | −0.69 |
| 2.0 cM // 12491 /// NM_001159555 // | | | |
| NM_053078 // D0H4S114 // DNA segment, | D0H4S114 | −2.48 | −0.69 |
| human D4S114 // 18 B1\|18 // 27528 /// NM_0 | | | |
| NM_146189 // Mybpc2 // myosin binding | Mybpc2 | −1.06 | −0.70 |
| protein C, fast-type // 7 B4\|7 // 233199 / | | | |
| NM_145434 // Nr1d1 // nuclear receptor | Nr1d1 | −0.32 | −0.70 |
| subfamily 1, group D, member 1 // 11 D\|11 | | | |
| NM_013491 // Clcn1 // chloride channel 1 // 6 | Clcn1 | −1.39 | −0.70 |
| B2.1\|6 22.5 cM // 12723 /// ENSMUS | | | |
| NM_023196 // Pla2g12a // phospholipase A2, | Pla2g12a | −0.56 | −0.71 |
| group XIIA // 3 H1\|3 // 66350 /// NM_{—} | | | |
| NM_010137 // Epas1 // endothelial PAS domain | Epas1 | −0.69 | −0.71 |
| protein 1 // 17 E4\|17 // 13819 /// | | | |
| NM_009832 // Ccnk // cyclin K // 12 F1\|12 52.0 cM | Ccnk | −0.80 | −0.72 |
| // 12454 /// ENSMUST0000010105 | | | |
| NM_174847 // C2cd2 // C2 calcium-dependent | C2cd2 | −0.47 | −0.72 |
| domain containing 2 // 16 C4\|16 // 20 | | | |
| NM_009122 // Satb1 // special AT-rich sequence | Satb1 | −0.62 | −0.73 |
| binding protein 1 // 17 C\|17 // 2 | | | |
| NM_172621 // Clic5 // chloride intracellular | Clic5 | −1.22 | −0.74 |
| channel 5 // 17\|17 C // 224796 /// | | | |
| NM_011224 // Pygm // muscle glycogen | Pygm | −1.35 | −0.74 |
| phosphorylase // 19 A\|19 2.0 cM // 19309 // | | | |
| NM_172436 // Slc25a12 // solute carrier family | Slc25a12 | −1.36 | −0.74 |
| 25 (mitochondrial carrier, Aralar | | | |
| NM_172992 // Phtf2 // putative homeodomain | Phtf2 | −1.70 | −0.75 |
| transcription factor 2 // 5 A3\|5 // 6 | | | |
| NM_182997 // Prkab2 // protein kinase, AMP- | Prkab2 | −0.52 | −0.76 |
| activated, beta 2 non-catalytic subun | | | |
| NM_133201 // Mfn2 // mitofusin 2 // 4 E2\|4 // | Mfn2 | −1.08 | −0.76 |
| 170731 /// ENSMUST00000030884 // M | | | |
| NM_001141927 // Pln // phospholamban // 10 | Pln | −0.57 | −0.76 |
| B3\|10 // 18821 /// NM_023129 // Pln / | | | |
| NM_026189 // Eepd1 // | Eepd1 | −0.78 | −0.77 |
| endonuclease/exonuclease/phosphatase family | | | |
| domain contain | | | |
| NM_001166388 // Ptp4a3 // protein tyrosine | Ptp4a3 | −0.90 | −0.77 |
| phosphatase 4a3 // 15\|15 E1 // 19245 | | | |
| NM_001163487 // Pfkm // phosphofructokinase, | Pfkm | −1.52 | −0.77 |
| muscle // 15 F1\|15 // 18642 /// NM_{—} | | | |
| NM_001013833 // Prkg1 // protein kinase, | Prkg1 | −0.70 | −0.78 |
| cGMP-dependent, type I // 19\|19 C2 // 1 | | | |
| NM_025363 // 1110001J03Rik // RIKEN cDNA | 1110001J03Rik | −1.51 | −0.78 |
| 1110001J03 gene // 6 B1\|6 // 66117 /// | | | |
| NM_172563 // Hlf // hepatic leukemia factor // | Hlf | −0.89 | −0.78 |
| 11 C-D\|11 52.0 cM // 217082 /// E | | | |
| NM_030598 // Rcan2 // regulator of calcineurin | Rcan2 | −0.83 | −0.78 |
| 2 // 17 C\|17 22.0 cM // 53901 /// | | | |
| NM_010726 // Phyh // phytanoyl-CoA | Phyh | −0.95 | −0.78 |
| hydroxylase // 2 A1\|2 // 16922 /// ENSMUST000 | | | |
| NM_025599 // 2610528E23Rik // RIKEN cDNA | 2610528E23Rik | −1.62 | −0.79 |
| 2610528E23 gene // 16 C1.1\|16 // 66497 | | | |
| NM_008055 // Fzd4 // frizzled homolog 4 | Fzd4 | −0.47 | −0.79 |
| (Drosophila) // 7 E1\|7 44.5 cM // 14366 | | | |
| NM_080639 // Timp4 // tissue inhibitor of | Timp4 | −0.73 | −0.80 |
| metalloproteinase 4 // 6 E3\|6 46.0 cM | | | |
| NM_025569 // Mgst3 // microsomal glutathione | Mgst3 | −2.65 | −0.80 |
| S-transferase 3 // 1\|1 H2 // 66447 | | | |
| NM_010228 // Flt1 // FMS-like tyrosine kinase 1 | Flt1 | −0.58 | −0.80 |
| // 5 G\|5 82.0 cM // 14254 /// EN | | | |
| NM_026950 // Ociad2 // OCIA domain | Ociad2 | −2.06 | −0.81 |
| containing 2 // 5\|5 D // 433904 /// ENSMUST00 | | | |
| NM_001082975 // Sdr39u1 // short chain | Sdr39u1 | −0.74 | −0.81 |
| dehydrogenase/reductase family 39U, membe | | | |
| NM_016872 // Vamp5 // vesicle-associated | Vamp5 | −0.65 | −0.81 |
| membrane protein 5 // 6 C1\|6 // 53620 / | | | |
| NM_001009935 // Txnip // thioredoxin | Txnip | −1.55 | −0.82 |
| interacting protein // 3 F2.2\|3 47.1 cM // | | | |
| NM_007563 // Bpgm // 2,3-bisphosphoglycerate | Bpgm | −2.04 | −0.82 |
| mutase // 6 B1\|6 // 12183 /// ENSMU | | | |
| NM_008993 // Pxmp2 // peroxisomal membrane | Pxmp2 | −0.86 | −0.83 |
| protein 2 // 5 F\|5 59.0 cM // 19301 / | | | |
| NM_011943 // Map2k6 // mitogen-activated | Map2k6 | −2.52 | −0.83 |
| protein kinase kinase 6 // 11\|11 E1 // | | | |
| NM_010324 // Got1 // glutamate oxaloacetate | Got1 | −1.46 | −0.83 |
| transaminase 1, soluble // 19 C3\|19 | | | |
| NM_009700 // Aqp4 // aquaporin 4 // 18 A1\|18 | Aqp4 | −1.69 | −0.83 |
| 6.0 cM // 11829 /// ENSMUST00000079 | | | |
| NM_145823 // Pitpnc1 // phosphatidylinositol | Pitpnc1 | −1.28 | −0.83 |
| transfer protein, cytoplasmic 1 // | | | |
| NM_181407 // Me3 // malic enzyme 3, | Me3 | −0.97 | −0.84 |
| NADP(+)-dependent, mitochondrial // 7 E1\|7 / | | | |
| NM_138628 // Txlnb // taxilin beta // 10 A2\|10 // | Txlnb | −1.28 | −0.84 |
| 378431 /// NM_009881 // Cdyl / | | | |
| NM_013476 // Ar // androgen receptor // X C3\|X | Ar | −0.84 | −0.84 |
| 36.0 cM // 11835 /// ENSMUST00000 | | | |
| NM_010596 // Kcna7 // potassium voltage-gated | Kcna7 | −1.45 | −0.85 |
| channel, shaker-related subfamily, | | | |
| NM_198414 // Paqr9 // progestin and adipoQ | Paqr9 | −1.01 | −0.85 |
| receptor family member IX // 9 E3.3\|9 | | | |
| NM_001081322 // Myo5c // myosin VC // 9 D\|9 | Myo5c | −1.12 | −0.85 |
| // 208943 /// ENSMUST00000036555 // | | | |
| NM_007940 // Ephx2 // epoxide hydrolase 2, | Ephx2 | −0.84 | −0.85 |
| cytoplasmic // 14 D\|14 32.5 cM // 138 | | | |
| NM_028283 // Uaca // uveal autoantigen with | Uaca | −0.53 | −0.86 |
| coiled-coil domains and ankyrin repe | | | |
| NM_017379 // Tuba8 // tubulin, alpha 8 // 6 F1\|6 | Tuba8 | −1.41 | −0.86 |
| // 53857 /// ENSMUST00000032233 | | | |
| NM_007592 // Car8 // carbonic anhydrase 8 // 4 | Car8 | −0.97 | −0.86 |
| A1\|4 7.7 cM // 12319 /// BC010773 | | | |
| NM_027430 // Brp44 // brain protein 44 // 1\|1 | Brp44 | −1.44 | −0.86 |
| H2 // 70456 /// ENSMUST00000027853 | | | |
| NM_019686 // Cib2 // calcium and integrin | Cib2 | −1.30 | −0.86 |
| binding family member 2 // 9 A5.3\|9 // | | | |
| NM_153103 // Kif1c // kinesin family member | Kif1c | −0.41 | −0.88 |
| 1C // 11 B3\|11 // 16562 /// ENSMUST0 | | | |
| NM_001005423 // Mreg // melanoregulin // 1 | Mreg | −0.43 | −0.89 |
| C3\|1 // 381269 /// ENSMUST00000048860 | | | |
| NM_026626 // Efcab2 // EF-hand calcium | Efcab2 | −0.87 | −0.89 |
| binding domain 2 // 1\|1 H3 // 68226 /// E | | | |
| NM_009255 // Serpine2 // serine (or cysteine) | Serpine2 | −1.45 | −0.90 |
| peptidase inhibitor, clade E, memb | | | |
| NM_009396 // Tnfaip2 // tumor necrosis factor, | Tnfaip2 | −0.63 | −0.91 |
| alpha-induced protein 2 // 12 F1\| | | | |
| NM_009204 // Slc2a4 // solute carrier family 2 | Slc2a4 | −1.52 | −0.91 |
| (facilitated glucose transporter) | | | |
| BC144873 // A930018M24Rik // RIKEN cDNA | A930018M24Rik | −0.99 | −0.92 |
| A930018M24 gene // 14 C1\|14 // 328399 | | | |
| NM_053200 // Ces1d // carboxylesterase 1D // 8 | Ces1d | −0.85 | −0.92 |
| C5\|8 // 104158 /// NM_144930 // C | | | |
| NM_175212 // Tmem65 // transmembrane | Tmem65 | −1.55 | −0.93 |
| protein 65 // 15 D1\|15 // 74868 /// ENSMUST | | | |
| NM_015763 // Lpin1 // lipin 1 // 12 A1.1\|12 9.0 cM | Lpin1 | −1.19 | −0.93 |
| // 14245 /// NM_001130412 // | | | |
| NM_001039543 // Mlf1 // myeloid leukemia | Mlf1 | −2.89 | −0.93 |
| factor 1 // 3 E1\|3 31.0 cM // 17349 /// | | | |
| NM_008009 // Fgfbp1 // fibroblast growth factor | Fgfbp1 | −1.55 | −0.95 |
| binding protein 1 // 5 B3\|5 // 1 | | | |
| NM_134079 // Adk // adenosine kinase // 14\|14 | Adk | −1.15 | −0.95 |
| A2-B // 11534 /// ENSMUST000000453 | | | |
| NM_001033767 // Gm4951 // predicted gene | Gm4951 | −0.46 | −0.96 |
| 4951 // 18 D3\|18 // 240327 /// NM_00114 | | | |
| NM_173752 // 1110067D22Rik // RIKEN | 1110067D22Rik | −1.48 | −0.98 |
| cDNA 1110067D22 gene // 11 A3.1\|11 11.0 cM / | | | |
| NM_011994 // Abcd2 // ATP-binding cassette, | Abcd2 | −1.71 | −0.99 |
| sub-family D (ALD), member 2 // 15 E | | | |
| NM_008830 // Abcb4 // ATP-binding cassette, | Abcb4 | −1.71 | −0.99 |
| sub-family B (MDR/TAP), member 4 // | | | |
| NM_177665 // Gm4861 // predicted gene 4861 // | Gm4861 | −0.79 | −1.00 |
| 3 G3\|3 // 229862 /// ENSMUST000000 | | | |
| NM_080847 // Asb15 // ankyrin repeat and | Asb15 | −2.09 | −1.00 |
| SOCS box-containing 15 // 6 A3.1\|6 // 7 | | | |
| NM_177647 // Cdnf // cerebral dopamine | Cdnf | −0.59 | −1.01 |
| neurotrophic factor // 2 A1\|2 // 227526 / | | | |
| NM_177225 // Samd12 // sterile alpha motif | Samd12 | −0.70 | −1.01 |
| domain containing 12 // 15 C-D1\|15 // | | | |
| NM_008712 // Nos1 // nitric oxide synthase 1, | Nos1 | −0.69 | −1.01 |
| neuronal // 5 F\|5 65.0 cM // 18125 | | | |
| NM_007421 // Adssl1 // adenylosuccinate | Adssl1 | −1.14 | −1.01 |
| synthetase like 1 // 12 F1\|12 // 11565 / | | | |
| NM_011542 // Tcea3 // transcription elongation | Tcea3 | −1.54 | −1.02 |
| factor A (SII), 3 // 4 D3\|4 // 21 | | | |
| NM_145525 // Osbpl6 // oxysterol binding | Osbpl6 | −0.89 | −1.02 |
| protein-like 6 // 2 C3\|2 // 99031 /// E | | | |
| NM_019410 // Pfn2 // profilin 2 // 3 D\|3 29.3 cM | Pfn2 | −1.13 | −1.03 |
| // 18645 /// ENSMUST00000066882 | | | |
| NM_172778 // Maob // monoamine oxidase B // | Maob | −0.86 | −1.03 |
| X A1.2\|X 5.2 cM // 109731 /// ENSMUS | | | |
| NM_011079 // Phkg1 // phosphorylase kinase | Phkg1 | −1.67 | −1.06 |
| gamma 1 // 5 G1.3\|5 72.0 cM // 18682 | | | |
| NM_013820 // Hk2 // hexokinase 2 // 6 C3\|6 | Hk2 | −0.87 | −1.08 |
| 34.5 cM // 15277 /// ENSMUST000000006 | | | |
| NM_148937 // Plcd4 // phospholipase C, delta 4 | Plcd4 | −2.18 | −1.11 |
| // 1 C3\|1 39.2 cM // 18802 /// NM | | | |
| NM_021391 // Ppp1r1a // protein phosphatase 1, | Ppp1r1a | −1.08 | −1.12 |
| regulatory (inhibitor) subunit 1A | | | |
| NM_008832 // Phka1 // phosphorylase kinase | Phka1 | −1.87 | −1.15 |
| alpha 1 // X D\|X 39.0 cM // 18679 /// | | | |
| NM_181588 // Cmbl // | Cmbl | −3.61 | −1.17 |
| carboxymethylenebutenolidase-like | | | |
| (Pseudomonas) // 15 B2\|15 | | | |
| NM_198112 // Ostn // osteocrin // 16 B2\|16 // | Ostn | −3.31 | −1.18 |
| 239790 /// ENSMUST00000066852 // O | | | |
| NM_001163945 // Rpl3l// ribosomal protein L3- | Rpl3l | −2.62 | −1.20 |
| like // 17 A3.3\|17 // 66211 /// NM | | | |
| NM_026853 // Asb11 // ankyrin repeat and | Asb11 | −1.57 | −1.21 |
| SOCS box-containing 11 // X F5\|X // 688 | | | |
| NM_001034859 // Gm4841 // predicted gene | Gm4841 | −1.92 | −1.23 |
| 4841 // 18 D3\|18 // 225594 /// NM_02179 | | | |
| NM_001177833 // Smox // spermine oxidase // 2 | Smox | −3.47 | −1.23 |
| F1\|2 // 228608 /// NM_145533 // Sm | | | |
| NM_028802 // Gpcpd1 // glycerophosphocholine | Gpcpd1 | −1.23 | −1.24 |
| phosphodiesterase GDE1 homolog (S. | | | |
| NM_001170748 // Asb14 // ankyrin repeat and | Asb14 | −1.12 | −1.25 |
| SOCS box-containing 14 // 14 A3\|14 / | | | |
| NM_007994 // Fbp2 // fructose bisphosphatase 2 | Fbp2 | −3.01 | −1.25 |
| // 13 B3\|13 // 14120 /// ENSMUST0 | | | |
| NM_013467 // Aldh1a1 // aldehyde | Aldh1a1 | −1.32 | −1.32 |
| dehydrogenase family 1, subfamily A1 // 19 B\|19 | | | |
| NM_183283 // 2310010M20Rik // RIKEN | 2310010M20Rik | −2.48 | −1.36 |
| cDNA 2310010M20 gene // 16 B2\|16 // 69576 // | | | |
| NM_001013390 // Scn4b // sodium channel, type | Scn4b | −1.18 | −1.39 |
| IV, beta // 9 A5.2\|9 // 399548 /// | | | |
| NM_001177753 // Pfkfb3 // 6-phosphofructo-2- | Pfkfb3 | −1.64 | −1.48 |
| kinase/fructose-2,6-biphosphatase 3 | | | |

(g) Gadd45a Represses Anti-Atrophy Genes and Induces Pro-Atrophy Genes

Gadd45a altered levels of many mRNAs whose roles in muscle atrophy are unknown (Table 3). However, some patterns could be discerned. For example, both denervation and Gadd45a repressed many interconnected mRNAs involved in anabolic signaling and protein synthesis (growth hormone receptor (Ghr), JAK2 kinase (Jak2), androgen receptor (Ar) and Eif4e), mitochondrial biogenesis (PGC-1α (Ppargc1a), thyroid hormone receptor α (Thra), PPARα (Ppara), mitofusin 1 and 2 (Mfn1 and Mfn2), calcium/calmodulin-dependent protein kinase II α and γ (Camk2a and Camk2g) and protein phosphatase 3 (Ppp3cb)), angiogenesis, vascular flow and oxygen delivery (nNOS (Nos1), Vegfa, Vegfb), glucose uptake (GLUT4 (Slc2a4), GLUT12 (Slc2a12)), glucose utilization (Hexokinase 2 (Hk2), Pfkm, Pgam2, Eno3, Pdhal and Dlat), fatty acid oxidation (Cpt1b, Acaa2, Acadm, Acadvl, Hadh and Decr1), citric acid cycle (Idh3g, Idh3a, Dlst, Suclg1, Sdha, Sdhb, Fh1, Mdh2 and Mdh1), oxidative phosphorylation (Ndufs6, Ndufa3, Ndufa8, Ndufa5, Ndufb9, Ndufv1, Ndufs8, Ndufb8, Ndufs2, Ndufs1, Uqcrfs1, Uqcrq, Uqcrc1, Cox6a2, Cox8b, Cox11, Atp5e and Atp5a1) and creatine phosphorylation (Ckm) (Table 3). qPCR was used to validate 11 representative changes, including repression of mRNAs encoding PGC-1α, the growth hormone receptor, androgen receptor, GLUT4, hexokinase-2, VEGF-A, nNOS, and thyroid hormone receptor-α (FIG. 9A). Gene set enrichment analysis also indicated that Gadd45a repressed growth and energy-yielding pathways; 10 gene sets were significantly depleted by both denervation and Gadd45a, including growth hormone signaling, insulin signaling, glycolysis, and the citrate cycle (FIG. 9B). Because anabolic signaling and PGC-1α prevent skeletal muscle atrophy (Sandri, M., et al. (2006) Proc. Natl. Acad. Sci. U.S.A. 103, 16260-16265; Wenz, T., et al. (2009) Proc. Natl. Acad. Sci. U.S.A. 106, 20405-20410; Laure, L., et al. (2009) FEBS J. 276, 669-684; Kunkel, S. D., (2011) Cell Metab. 13, 627-638; Sartori, R., (2009) Am. J. Physiol. Cell Physiol. 296, C1248-C1257; Bodine, S. C., et al. (2001) Nat. Cell Biol. 3, 1014-1019; and Schiaffino, S., and Mammucari, C. (2011) Skelet. Muscle 1, 4), these data indicated that Gadd45a reprograms myonuclei in a manner that removes barriers to muscle atrophy.

In addition to reducing mRNAs that maintain muscle, Gadd45a induced genes that promote atrophy. For example, both denervation and Gadd45a increased several mRNAs involved in lysosome-mediated proteolysis (LC3a (Mapl1c3a), Gabarap, Ctsd, Ctsl, Ctss, Ctsz, Atp6ap2 and Atp6vIh) and caspase-mediated proteolysis (Bax and caspase 3 (Casp3)) (Table 3). Lysosome- and caspase-mediated proteolysis are also essential for muscle atrophy (Mammucari, C., et al. (2007) Cell Metab. 6, 458-47115-17; Zhao, J., et al. (2007) Cell Metab. 6, 472-483; and Plant, P. J., et al. (2009) J. Appl. Physiol. 107, 224-234). Some representative changes were confirmed by using qPCR (FIG. 9A). Gene set enrichment analysis also supported the notion that Gadd45a increased pro-atrophy mRNAs. Thirteen gene sets were significantly enriched by both denervation and Gadd45a (FIG. 9B). These included several stress-signaling pathways that promote muscle atrophy (e.g. NF-kB, p53, TLR, and TNFR1 pathways (Cai, D., et al. (2004) Cell 119, 285-298; Peterson, et al. (2011) Curr. Top. Dev. Biol. 96, 85-119; Schwarzkopf, M., (2006) Genes Dev. 20, 3440-3452)), indicating that Gadd45a mediates or resembles these pro-atrophy pathways.

Interestingly, both denervation and Gadd45a increased Runx1 mRNA and six known Runx1 targets (FIG. 9C). These mRNAs limit muscle damage in denervated, atrophying muscle (Wang, X., et al. (2005) Genes Dev. 19, 1715-1722).

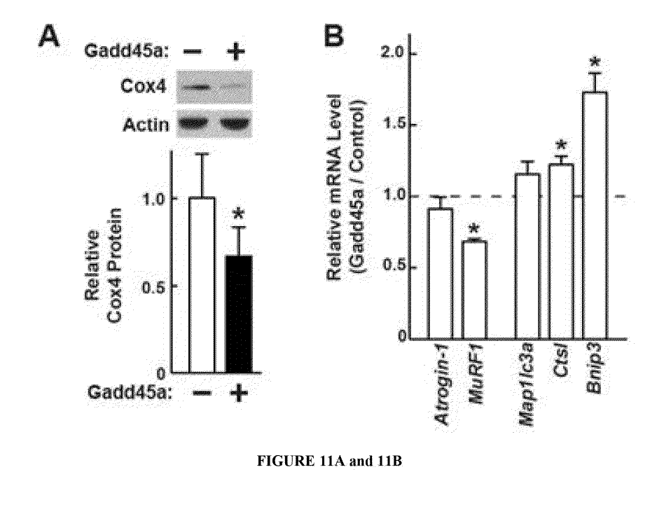

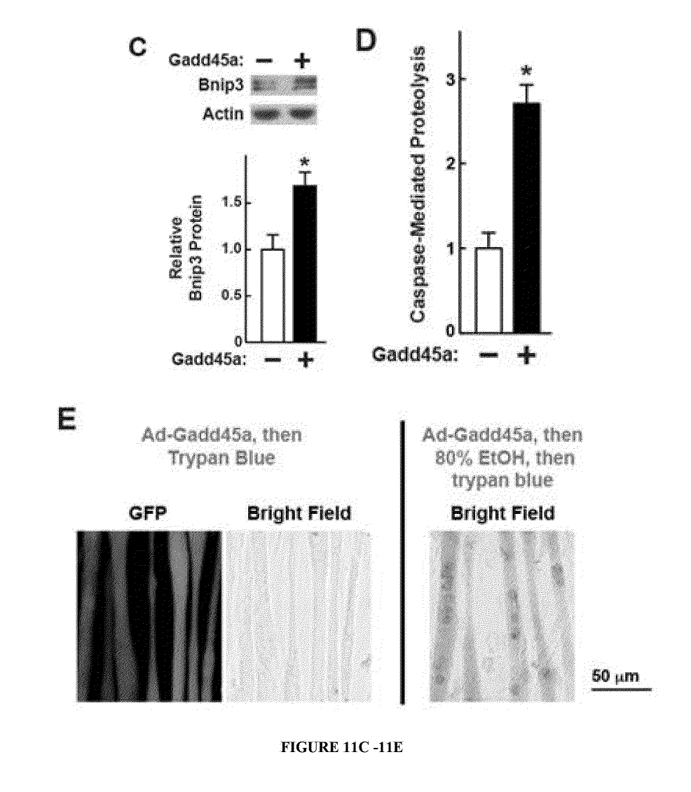

Because atrogin-1 and MuRF1 also mediate a component of proteolysis during muscle atrophy (Bodine, S. C., et al. (2001) Science 294, 1704-17089; Sandri, M., et al. (2004). Cell 117, 399-412; Stitt, T. N., et al. (2004) Mol. Cell 14, 395-403; Moresi, V., et al. (2010) Cell 143, 35-45; and Acharyya, S., et al. (2004) J. Clin. Investig. 114, 370-378), the effect of Gadd45a on atrogin-1 and MuRF1 mRNAs was examined. Although denervation increased atrogin-1 or MuRF1 mRNAs (FIG. 9A), Gadd45a did not increase these mRNAs at early or late time points in skeletal muscle (FIG. 9D) or myotubes (FIG. 11B). Thus, atrogin-1 and MuRF1 are among the 58% of denervation-induced mRNAs that are not under the control of Gadd45a. Taken together, these data indicate that Gadd45a orchestrates a multitude of nuclear changes that are predicted to promote skeletal muscle atrophy.

(h) Gadd45a Reduces PGC-14 Expression, Akt Activity, and Protein Synthesis, and Stimulates Autophagy and Caspase-Mediated Proteolysis

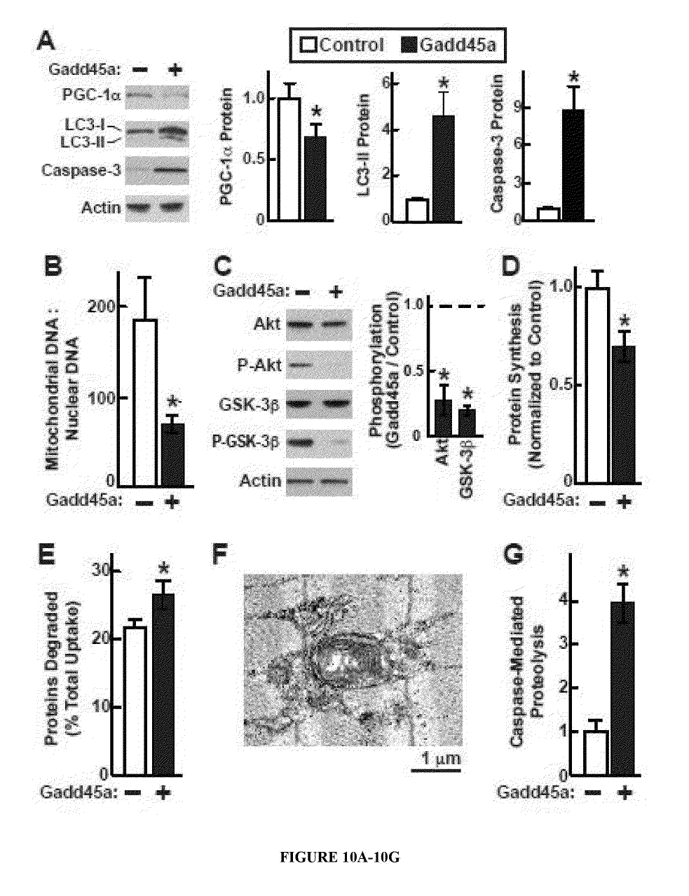

It was further investigated how the effects of Gadd45a on myonuclei and skeletal muscle mRNA expression might impact downstream proteins and cellular processes known to be involved in muscle atrophy. PGC-1α inhibits muscle atrophy (Sandri, M., et al. (2006) Proc. Natl. Acad. Sci. U.S.A. 103, 16260-16265; and Wenz, T., et al. (2009) Proc. Natl. Acad. Sci. U.S.A. 106, 20405-20410 19), promotes mitochondrial biogenesis (Uldry, M., et al. (2006) Cell Metab. 3, 333-341), and induces Slc2a4 (GLUT4) transcription (Michael, L. F., et al. (2001) Proc. Natl. Acad. Sci. U.S.A. 98, 3820-3825). Because Gadd45a reduced Ppargc1a (PGC-1α) and Slc2a4 (GLUT4) mRNAs, as well as a number of mRNAs encoding mitochondrial proteins (Table 3 and FIG. 9A), it was hypothesized that Gadd45a might reduce PGC-1α protein and thus the amount of mitochondria in skeletal muscle. Indeed, in skeletal muscle, Gadd45a reduced PGC-1α protein (FIG. 10A), mitochondrial DNA (FIG. 10B), and the mitochondrial protein COX4 (FIG. 11A).

Gadd45a also reduced mRNAs involved in anabolic signaling and protein synthesis (Table 3 and FIG. 9A). Therefore, the effects of Gadd45a on Akt kinase activity were examined. Akt inhibits muscle atrophy and increases protein synthesis by multiple mechanisms, including phosphorylation (inhibition) of its target GSK-3β (Kunkel, S. D., et al. (2011) Cell Metab. 13, 627-638; Sartori, R., et al. (2009) Am. J. Physiol. Cell Physiol. 296, C1248-C1257; Bodine, S. C., et al. (2001) Nat. Cell Biol. 3, 1014-1019; Schiaffino, S., and Mammucari, C. (2011) Skelet. Muscle 1, 4; Frame, S., and Cohen, P. (2001) Biochem. J. 359, 1-16; Verhees, K. J., et al. (2011) Am. J. Physiol. Cell Physiol. 301, C995-C1007). It was found that Gadd45a reduced Akt and GSK-3β phosphorylation in cultured myotubes (FIG. 10C). Consistent with reduced Akt activity and increased GSK-3β activity, Gadd45a also reduced protein synthesis (FIG. 10D).

In contrast to its effect on anabolic mRNAs, Gadd45a increased mRNAs involved in autophagy (including Mapl1c3a, which encodes LC3) and the caspase pathway (including Casp3, which encodes caspase-3) (Table 3 and FIG. 9A). This suggested that Gadd45a might also increase proteolysis, which was confirmed in myotubes (FIG. 10E). In addition, atrophic muscle fibers overexpressing Gadd45a contained increased total and lipidated LC3 (FIG. 10A), as well as autophagosomes (FIG. 10F), indicating increased autophagy. Similarly, Gadd45a increased two key autophagy mRNAs (Bnip3 and Ctsl), as well as Bnip3 protein, in myotubes (supplemental FIGS. 11B and 11C). Furthermore, atrophic muscle fibers overexpressing Gadd45a contained increased caspase-3 protein (FIG. 10A). As a result, caspase-mediated proteolysis was also increased (FIG. 10G). Similarly, Gadd45a increased caspase-mediated proteolysis in cultured myotubes (FIG. 11D), without causing myotube death (FIG. 11E).

Thus, by altering skeletal muscle mRNA expression, Gadd45a reduced two proteins that inhibit muscle atrophy (PGC-1α and activated Akt), reduced mitochondria, increased three proteins that promote muscle atrophy (activated GSK-3β, lipidated LC3, and caspase-3), inhibited a critical anabolic process (protein synthesis), and induced two key proteolytic systems (autophagy and caspase-mediated proteolysis). These data support the notion that Gadd45a causes muscle atrophy by reprogramming skeletal muscle gene expression.

(3) Discussion of FIGS. 1-11

The pathogenesis of skeletal muscle atrophy is complex. Previous studies demonstrated important roles for reduced PGC-1α expression (Sandri, M., et al. (2006) Proc. Natl. Acad. Sci. U.S.A. 103, 16260-16265), reduced Akt signaling (Bodine, S. C., et al. (2001) Nat. Cell Biol. 3, 1014-1019), increased ATF4 expression (Ebert, S. M., et al. (2010) Mol. Endocrinol. 24, 790-799), increased GSK-3β signaling (Verhees, K. J., et al. (2011) Am. J. Physiol. Cell Physiol. 301, C995-C1007), increased caspase-3 activity (Plant, P. J., et al. (2009) J. Appl. Physiol. 107, 224-234), and increased autophagy (Mammucari, C., et al. (2007) Cell Metab. 6, 458-471; Zhao, J., et al. (2007) Cell Metab. 6, 472-483). In addition, microarray studies showed that atrophying muscles contain very high levels of Gadd45a mRNA (Welle, S., et al. (2004) Exp. Gerontol. 39, 369-377; Welle, S., et al. (2003) Physiol. Genomics 14, 149-159; Edwards, M. G., et al. (2007) BMC Genomics 8, 80; Stevenson, E. J., et al. (2003) J. Physiol. 551, 33-48; Gonzalez de Aguilar, et al. (2008) Physiol. Genomics 32, 207-218; Ebert, S. M., et al. (2010) Mol. Endocrinol. 24, 790-799). The data shown in FIGS. 1-11 elucidate a pathway that connects these previous findings.

In healthy muscle, ATF4 and Gadd45a levels are relatively low. However, acute stresses such as fasting and muscle disuse stimulate ATF4 expression (Sacheck, J. M., et al. (2007) FASEB J. 21, 140-155), which contributes to the induction of Gadd45a expression. Gadd45a translocates to the nucleus, where it alters myonuclear morphology and induces widespread changes in skeletal muscle mRNA expression. mRNAs involved in anabolic signaling, protein synthesis, glucose uptake, glycolysis, oxygen delivery, mitochondrial biogenesis, citric acid cycle, and oxidative phosphorylation are repressed. Conversely, mRNAs involved in autophagy and caspase-mediated proteolysis are induced. By reprogramming skeletal muscle gene expression, Gadd45a stimulates multiple interconnected atrophy mechanisms in the cytosol. On one hand, Gadd45a reduces barriers to atrophy, including PGC-1α expression, Akt activity, protein synthesis, and mitochondria. On the other hand, Gadd45a increases mediators of atrophy, including activated GSK-3β, activated caspase-3, and autophagy. Thus, Gadd45a coordinates a comprehensive program for skeletal muscle atrophy.

Because ATF4 and Gadd45a are not highly expressed under basal conditions, interventions that specifically target these proteins do not cause muscle fiber hypertrophy. However, the pathway emerges during stress, and thus reducing ATF4 or Gadd45a diminishes stress-induced muscle atrophy. Moreover, forced expression of ATF4 or Gadd45a induces atrophy in the absence of upstream stress. These data indicate a critical role in muscle atrophy, and suggest ATF4 and Gadd45a as potential therapeutic targets.

The ATF4/Gadd45a pathway is part of a larger signaling network with other important components. Loss of ATF4 only partially reduced Gadd45a expression, and it delayed but did not prevent muscle atrophy. This indicates that ATF4 plays an important early role in muscle atrophy, but other atrophy mechanisms compensate for the loss of ATF4 during prolonged stress. It also indicates the existence of other regulators upstream of Gadd45a. Potential candidates include FoxO transcription factors and p53, which are known to induce Gadd45a transcription in other settings (Tran, H., et al. (2002) Science 296, 530-534; Kastan, M. B., (1992) Cell 71, 587-597, 50). In addition, the microarray studies that pointed to Gadd45a as a key ATF4 target gene do not rule out the possibility that other important ATF4 target genes might also exist. Finally, Gadd45a generated >600 mRNA expression changes that occur during muscle denervation; however, this accounts for only 40% of the total changes in denervated muscles. This indicates the existence of other regulators that act in parallel to Gadd45a. Important mRNAs that are not controlled by Gadd45a include atrogin-1 and MuRF1. Because Akt activity and PGC-1α repress atrogin-1 and MuRF1 transcription (Sandri, M., et al. (2004) Cell 117, 399-412; Stitt, T. N., et al. (2004) Mol. Cell 14, 395-403; Sandri, M., et al. (2006) Proc. Natl. Acad. Sci. U.S.A. 103, 16260-16265), and because Gadd45a decreased Akt activity and PGC-1α, it was surprising that Gadd45a did not increase atrogin-1 and MuRF1 mRNAs. This can reflect a requirement for other transcription factors that induce atrogin-1 and MuRF1, such as FoxO transcription factors (Sandri, M., et al. (2004) Cell 117, 399-412; Stitt, T. N., et al. (2004) Mol. Cell 14, 395-403), NF-kB (Cai, D., et al. (2004) Cell 119, 285-298; Peterson, J. M., et al. (2011) Curr. Top. Dev. Biol. 96, 85-119), and myogenin (Moresi, V., et al. (2010) Cell 143, 35-45). Alternatively, atrogin-1 and MuRF1 transcription could lie upstream of Gadd45a. Furthermore, the current data do not rule out a role for atrogin-1 and MuRF1 proteins in Gadd45a-mediated atrophy. It is also important to note that Gadd45a regulates hundreds of mRNAs whose roles in muscle atrophy are not yet known.

Importantly, Gadd45a did not induce myonuclear pyknosis even though it increased caspase activity. Similarly, Gadd45a overexpression increased caspase activity in cultured myotubes without causing cell death. These findings are consistent with previous studies of denervated muscle: caspase-3 activity is required during the first 2 weeks of denervation-induced muscle atrophy (Plant, P. J., et al. (2009) J. Appl. Physiol. 107, 224-234), however, apoptotic loss of myonuclei does not occur in this time frame (Gundersen, K., and Bruusgaard, J. C. (2008) J. Physiol. 586, 2675-2681). It is also interesting that Gadd45a reduced mitochondria, but did not decrease the amount or size of type I fibers, which are particularly rich in mitochondria. RNAi targeting Gadd45a reduced atrophy in type II but not type I fibers. Selective effects on type II fibers may reflect previous findings that type II fibers are more prone to atrophy during fasting and denervation (Dedkov, E. I., et al. (2003) J. Gerontol. 58, 984-991; Li, J. B., and Goldberg, A. L. (1976) Am. J. Physiol. 231, 441-44856).

In summary, the data shown in FIGS. 1-11 elucidate a stress-induced pathway with a critical role in the signaling network that drives skeletal muscle atrophy. The centerpiece of this pathway is Gadd45a, a nuclear protein that stimulates myonuclear remodeling and widespread changes in skeletal muscle gene expression. By reprogramming muscle gene expression, Gadd45a reduces multiple barriers to atrophy and stimulates multiple atrophy mechanisms.

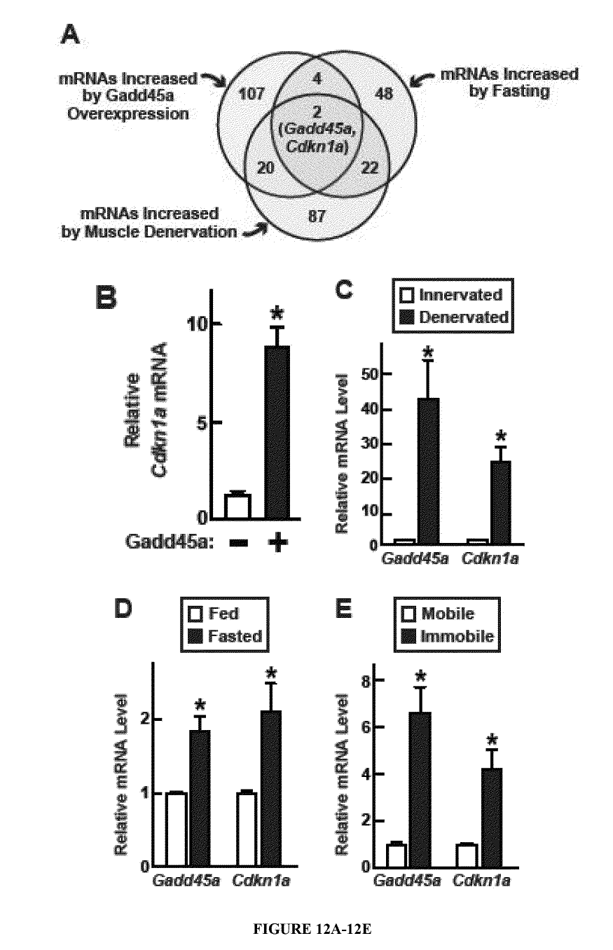

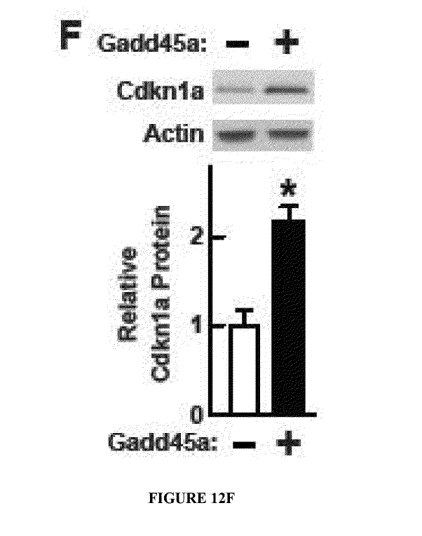

(4) Introduction to FIGS. 12-18

FIGS. 1-11 demonstrated that a small nuclear protein, Gadd45a, is an important molecular mediator of skeletal muscle atrophy (Banduseela, V. C., et al. (2009) Physiol. Genomics 39, 141-159). Skeletal muscle Gadd45a expression is low under basal conditions, but rises during muscle disuse, fasting, illness and aging secondary to stress-induced transcription of the Gadd45a gene (Banduseela, V. C., et al. (2009) Physiol. Genomics 39, 141-159; Welle, S., et al. (2004) Exp. Gerontol. 39, 369-377; Welle, S., et al. (2003) Physiol. Genomics 14, 149-159; Edwards, M. G., et al. (2007) BMC Genomics 8, 80; Stevenson, E. J., et al. (2003) J. Physiol. 551, 33-48; Gonzalez de Aguilar, J. L., et al. (2008) Physiol. Genomics 32, 207-218; Bodine, S. C., et al. (2001) Science 294, 1704-1708; Sandri, M., et al. (2004) Cell 117, 399-412; Stitt, T. N., et al. (2004) Mol. Cell 14, 395-403; Moresi, V., et al. (2010) Cell 143, 35-45). This increase in Gadd45a expression is sufficient to induce atrophy of mouse skeletal muscle fibers in vivo, and atrophy of cultured skeletal myotubes in vitro (Banduseela, V. C., et al. (2009) Physiol. Genomics 39, 141-159). It is also partially required for atrophy of skeletal muscle fibers during fasting, muscle denervation and muscle immobilization (Banduseela, V. C., et al. (2009) Physiol. Genomics 39, 141-159).

The molecular mechanism by which Gadd45a promotes skeletal muscle atrophy is not yet known. Following its induction by stress, Gadd45a localizes to skeletal myonuclei (Banduseela, V. C., et al. (2009) Physiol. Genomics 39, 141-159). There, through an unknown mechanism, Gadd45a alters myonuclear structure and reprograms skeletal muscle gene expression in a manner that generates many of the cellular changes that are known to contribute to skeletal muscle atrophy, including reduced anabolic signaling, decreased mitochondria, reduced protein synthesis and increased proteolysis (Banduseela, V. C., et al. (2009) Physiol. Genomics 39, 141-159). An intriguing possibility is that Gadd45a might alter myonuclear structure and gene expression by an epigenetic mechanism, such as DNA demethylation. Interestingly, Gadd45a is an important component of DNA demethylase complexes in other cell types (Cai, D., et al. (2004) Cell 119, 285-298; Acharyya, S., et al. (2004) J. Clin. Investig. 114, 370-378; Mammucari, C., (2007) Cell Metab. 6, 458-471; Zhao, J., et al. (2007) Cell Metab. 6, 472-483), and dynamic changes in skeletal muscle DNA methylation are known to accompany exercise and type 2 diabetes (Plant, P. J., et al. (2009) J. Appl. Physiol. 107, 224-234; Sandri, M., et al. (2006) Proc. Natl. Acad. Sci. U.S.A. 103, 16260-16265).

The following study tested the hypothesis that Gadd45a might cause skeletal muscle atrophy by stimulating DNA demethylation. Using mouse skeletal muscle and cultured skeletal myotubes, it was discovered that Gadd45a interacts with a specific region in the Cdkn1a gene promoter and stimulates its demethylation, leading to Cdkn1a gene activation. As a result, Cdkn1a mRNA increases, leading to an increased level of Cdkn1a protein, which is also known as p21^{WAF1/Cip1}. Interestingly, increased Cdkn1a expression accounts for many of the effects of Gadd45a on skeletal muscle gene expression, mitochondria, anabolic signaling, protein synthesis and protein degradation. Consistent with these effects, Cdkn1a expression induces atrophy of mouse muscle fibers and cultured myotubes. These data explain how Gadd45a causes muscle atrophy and elucidate important roles for DNA demethylation and Cdkn1a.

(a) Gadd45a Induces Cdkn1a mRNA During Skeletal Muscle Atrophy

Since demethylation frequently leads to gene activation (Niehrs, C., and Schafer, A. (2012) Trends in cell biology 22, 220-227; Bird, A. (2002) Genes & development 16, 6-21), it was reasoned that a Gadd45a demethylation target would likely yield high levels of mRNA in the presence of Gadd45a. Thus, to identify potential Gadd45a gene targets, previously published exon expression data were used (Ebert, S. M., et al. (2012) The Journal of biological chemistry 287, 27290-27301; Ebert, S. M., et al. (2010) Molecular Endocrinology 24, 790-799) to search for mouse tibialis anterior (TA) muscle mRNAs whose levels were increased at least 2-fold by three distinct atrophy stimuli: muscle denervation, fasting and Gadd45a overexpression. Only two mRNAs met these criteria: Gadd45a, as expected, and Cdkn1a (FIG. 12A). qPCR analysis confirmed that Gadd45a overexpression, denervation and fasting increased Cdkn1a mRNA in mouse skeletal muscle (FIG. 12B-12D). Similarly, muscle immobilization, another form of disuse atrophy that requires Gadd45a induction (Ebert, S. M., et al. (2012) The Journal of biological chemistry 287, 27290-27301), increased both Gadd45a and Cdkn1a mRNAs in mouse skeletal muscle (FIG. 12E). Consistent with its capacity to increase Cdkn1a mRNA, Gadd45a overexpression also increased Cdkn1a protein (also known as p21^{WAF1/Cip1}) in mouse skeletal muscle (FIG. 12F). Taken together, these data indicated that Gadd45a increases Cdkn1a mRNA during skeletal muscle atrophy, and identified the Cdkn1a gene as a potential target of Gadd45a-mediated demethylation.

(b) Gadd45a Demethylates and Activates the Cdkn1a Gene Promoter

To test the hypothesis that Gadd45a might alter Cdkn1a gene methylation, a previously described in vitro model of muscle atrophy was used: Gadd45a overexpression in fully differentiated C2C12 skeletal myotubes (Ebert, S. M., et al. (2012) The Journal of biological chemistry 287, 27290-27301). To overexpress Gadd45a, myotubes were infected with Ad-Gadd45a. Control myotubes were infected with Ad-ATF4ΔbZIP. Cdkn1a gene methylation was analyzed with methylated DNA immunoprecipitation (MeDIP)-chip.

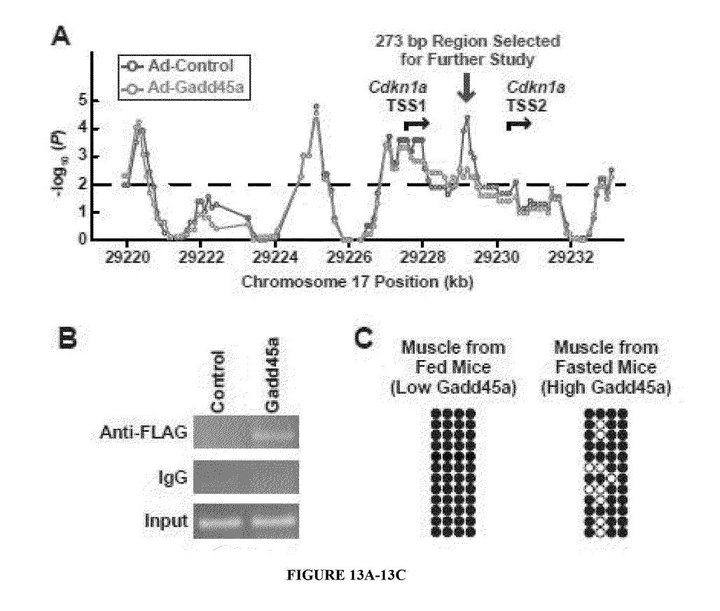

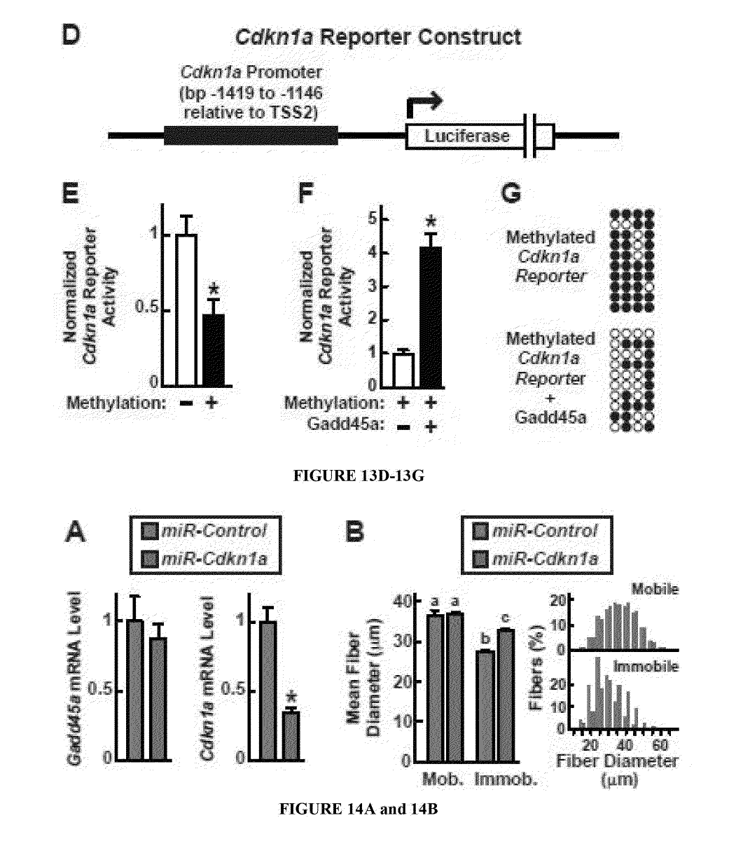

The Cdkn1a gene possesses two transcription start sites (TSS 1 and TSS2) that generate transcripts with variable 5′ untranslated regions but identical coding sequences. In non-atrophied control myotubes, four methylation peaks were present: two peaks upstream of TSS1, one peak overlying TSS1, and one peak between TSS1 and TSS2 (FIG. 13A). However, in atrophied myotubes overexpressing Gadd45a, the methylation peak between TSS1 and TSS2 was absent (FIG. 13A). Within this peak, a region containing four CpG dinucleotides that is located between 1419 and 1146 by upstream of Cdkn1a TSS2 was targeted. Using chromatin immunoprecipitation, it was found that Gadd45a interacted with this portion of the Cdkn1a promoter (FIG. 13B). Taken together, these data indicated that Gadd45a interacts with and stimulates demethylation of a specific portion of the Cdkn1a promoter region in skeletal myotubes.

To determine if the same portion of the Cdkn1a promoter is demethylated during skeletal muscle atrophy in vivo, muscles from fed (non-atrophied) and fasted (atrophied) mice were obtained. Bisulfite sequencing was used to evaluate the methylation status of the four CpG dinucleotides. In skeletal muscles from fed mice, the Cdkn1a promoter region was fully methylated (FIG. 13C). However, in muscles from fasted mice, methylation was reduced (FIG. 13C). Thus, fasting, which induces Gadd45a and Cdkn1a expression (FIG. 12D), stimulated Cdkn1a promoter demethylation in mouse skeletal muscle.

To further investigate this portion of the Cdkn1a promoter, it was inserted into a luciferase reporter plasmid to generate a Cdkn1a reporter construct (FIG. 13D). To test whether methylation reduces Cdkn1a reporter activity, the Cdkn1a reporter plasmid was methylated in vitro, and then transfected mouse skeletal muscles with either the unmethylated reporter (left TA) or the methylated reporter (right TA). In vitro methylation reduced in vivo luciferase activity by ≈50% (FIG. 13E). Next tested was whether Gadd45a might activate the methylated Cdkn1a reporter by co-transfecting the methylated reporter with either empty plasmid (left TA) or Gadd45a plasmid (right TA). Gadd45a increased luciferase expression from the methylated reporter ≈4-fold (FIG. 13F). To determine if Gadd45a-mediated activation of the methylated Cdkn1a reporter was associated with Cdkn1a promoter demethylation, reporter plasmid was extracted from transfected mouse muscles and performed bisulfite sequencing, which showed that Gadd45a significantly reduced methylation (FIG. 13G). Collectively, these data indicate that during skeletal muscle atrophy, Gadd45a interacts with the Cdkn1a promoter and stimulates its demethylation, leading to Cdkn1a gene activation and increased Cdkn1a expression.

(c) Cdkn1a is Required for Skeletal Muscle Fiber Atrophy During Immobilization, Fasting, Denervation and Gadd45a Overexpression

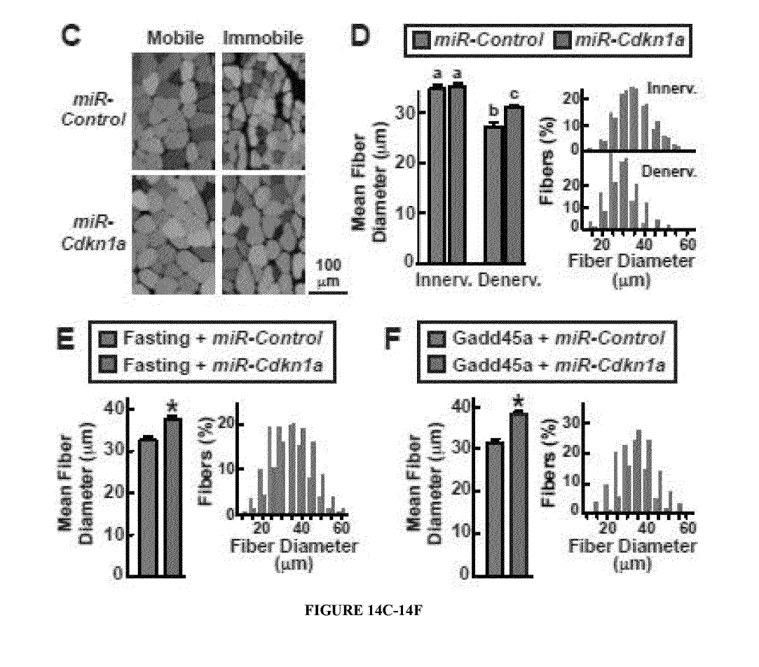

To determine if increased Cdkn1a expression is required for muscle fiber atrophy, bilateral TAs of mice was transfected with plasmid encoding an artificial miRNA that targets Cdkn1a (miR-Cdkn1a). TAs of control mice were transfected with plasmid encoding a nontargeting control miRNA (miR-Control). Both plasmids co-expressed EmGFP as a transfection marker. Plasmid transfection was achieved via electroporation. Three days after transfection, unilateral TA immobilization was performed. One week later, bilateral TAs were harvested and compared. As expected, miR-Cdkn1a significantly reduced Cdkn1a mRNA but not Gadd45a mRNA in immobilized muscles (FIG. 14A). In addition, miR-Cdkn1a reduced muscle fiber atrophy in immobilized muscles (FIGS. 14B and 14C). miR-Cdkn1a did not alter muscle fiber size in the absence of immobilization (FIGS. 14B and 14C). This is consistent with the finding that Cdkn1a mRNA is low under basal conditions (FIG. 12C-12E). Similar results were seen with a second miR-Cdkn1a construct that targeted different regions of the Cdkn1a transcript (FIGS. 15A and 15B).

Since Cdkn1a expression is also increased by muscle denervation and fasting (FIGS. 12B and 12D), the effects of miR-Cdkn1a in denervated and fasted muscles were tested. To test the role of Cdkn1a in muscle denervation, bilateral mouse TAs were transfected with either miR-Control or miR-Cdkn1a. One sciatic nerve was then transfected to induce unilateral muscle atrophy. The contralateral TA muscle remained innervated and served as an intrasubject control. One week later, innervated and denervated muscles were compared. miR-Cdkn1a significantly reduced atrophy of denervated muscle fibers (FIG. 14D). To test the role of Cdkn1a expression during fasting, miR-Cdkn1a was transfected into one TA, and miR-Control into the contralateral TA. The mice were then subjected to a 24 h fast. In fasted mice, miR-Cdkn1a significantly reduced muscle fiber atrophy (FIG. 14E). Similar results were obtained with a second miR-Cdkn1a construct (FIG. 15C).

Because Gadd45a induces Cdkn1a via promoter demethylation (FIG. 13A-13G), it was hypothesized that Cdkn1a is required for Gadd45a-mediated atrophy. To test this, plasmid encoding Gadd45a (both TAs) was co-transfected with miR-Control (left TA) or miR-Cdkn1a (right TA). In the presence of Gadd45a overexpression, miR-Cdkn1a significantly increased skeletal muscle fiber size, indicating reduced atrophy (FIG. 14F). Thus, Cdkn1a is required for skeletal muscle fiber atrophy during immobilization, denervation, fasting and Gadd45a overexpression.

(d) Increased Cdkn1a Expression is Sufficient to Induce Skeletal Muscle Atrophy

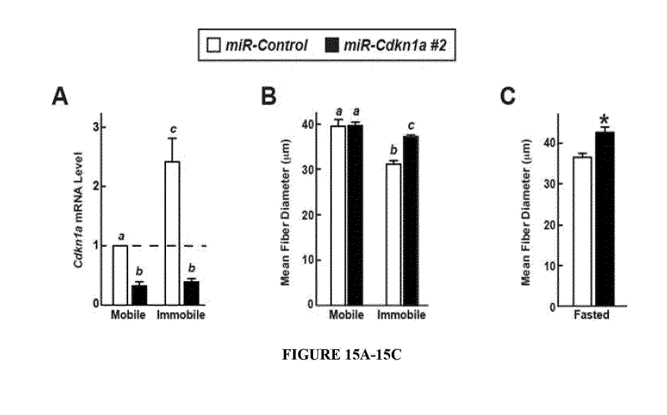

To determine if increased Cdkn1a expression promotes skeletal muscle fiber atrophy, plasmid encoding mouse Cdkn1a was transfected into mouse TA muscle. The contralateral TA muscle was transfected with empty plasmid vector (pcDNA3) and served as an intrasubject control. Bilateral TA muscles were co-transfected with plasmid encoding eGFP (pCMV-eGFP), which served as a transfection marker. Immunoblot analysis confirmed that Cdkn1a plasmid transfection increased Cdkn1a protein (FIG. 16A). Relative to control transfected fibers, muscle fibers transfected with Cdkn1a were significantly smaller, indicating skeletal muscle fiber atrophy (FIGS. 16B and 16C). Consistent with atrophy, Cdkn1a also decreased the maximum isometric tetanic force generated by skeletal muscles ex vivo (from 509±8 mN to 428±28 mN; P=0.02), as well as the specific tetanic force generated by these muscles (FIG. 16D). Thus, increased Cdkn1a expression is sufficient to induce atrophy of skeletal muscle fibers in vivo.

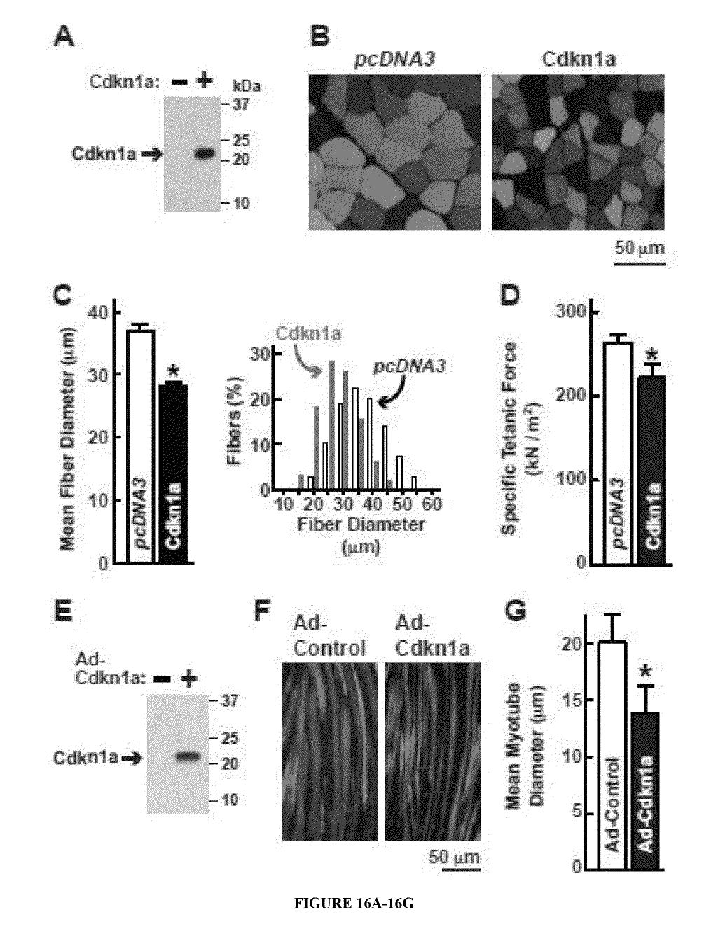

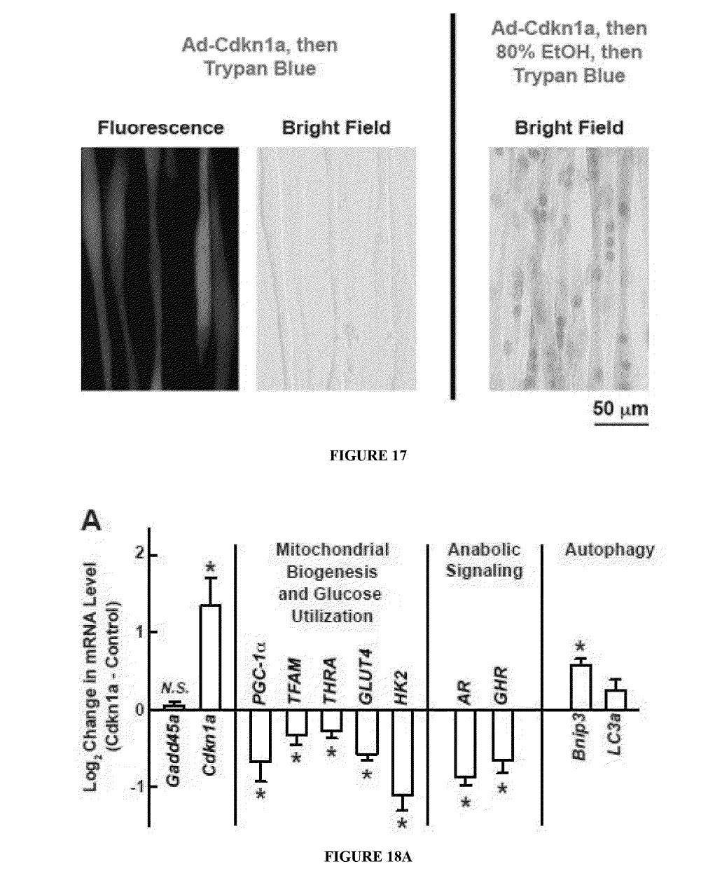

As a complementary system, the effect of adenovirus co-expressing GFP and Cdkn1a (Ad-Cdkn1a) in differentiated skeletal myotubes was tested. In myotubes, Ad-Cdkn1a increased Cdkn1a protein (FIG. 16E) and induced atrophy (FIGS. 16F and 16G), but did not cause myotube death (FIG. 17). Thus, increased Cdkn1a expression is sufficient to cause atrophy of skeletal muscle cells both in vivo and in vitro. These effects of Cdkn1a resembled the effects of Gadd45a (Ebert, S. M., et al. (2012) The Journal of biological chemistry 287, 27290-27301), further supporting the notion that Gadd45a causes atrophy by increasing Cdkn1a.

(e) Cdkn1a Reduces Barriers to Muscle Atrophy (PGC-1a Expression, Mitochondria, Akt Activity and Protein Synthesis) and Stimulates Protein Breakdown

Since Cdkn1a induced skeletal muscle atrophy, it was hypothesized that Cdkn1a might account for some of the downstream changes that Gadd45a generates in skeletal muscle, including alterations in muscle gene expression that increase protein breakdown and diminish PGC-1α, mitochondria, anabolic signaling and protein synthesis (Table 3 and FIG. 9-11). Importantly, PGC-1α, mitochondria, anabolic signaling and protein synthesis maintain healthy muscle and protect against atrophy (Kunkel, S. D., et al. (2011) Cell metabolism 13, 627-638; Sandri, M., et al. (2006) Proceedings of the National Academy of Sciences of the United States of America 103, 16260-16265; Wenz, T., et al. (2009) Proceedings of the National Academy of Sciences of the United States of America 106, 20405-20410; Bodine, S. C., et al. (2001) Nature cell biology 3, 1014-1019; Schiaffino, S., and Mammucari, C. (2011) Skelet Muscle 1, 4; Fry, C. S., and Rasmussen, B. B. (2011) Current aging science 4, 260-268; Powers, S. K., et al. (2012) Am J Physiol Endocrinol Metab 303, E31-39), whereas protein breakdown is a critical pro-atrophy process (Zhao, J., (2007) Cell metabolism 6, 472-48330; Mammucari, C., et al. (2007) Cell metabolism 6, 458-471).

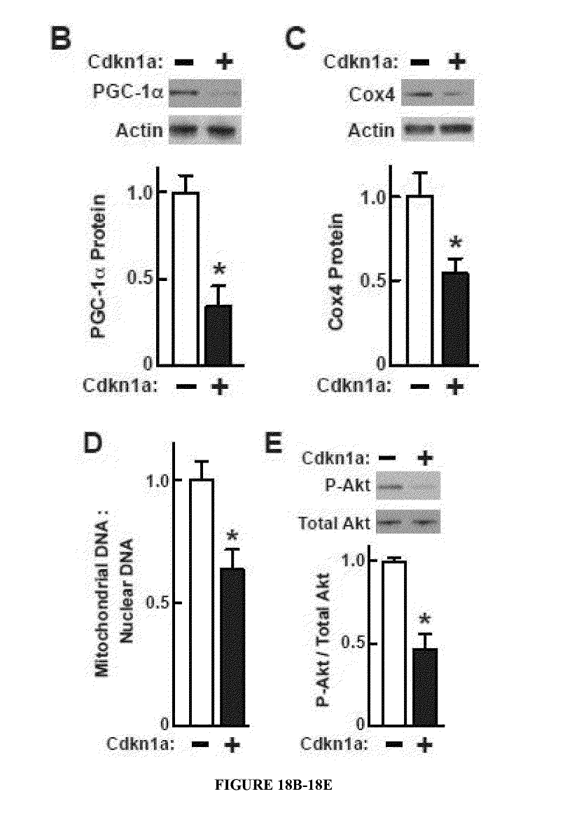

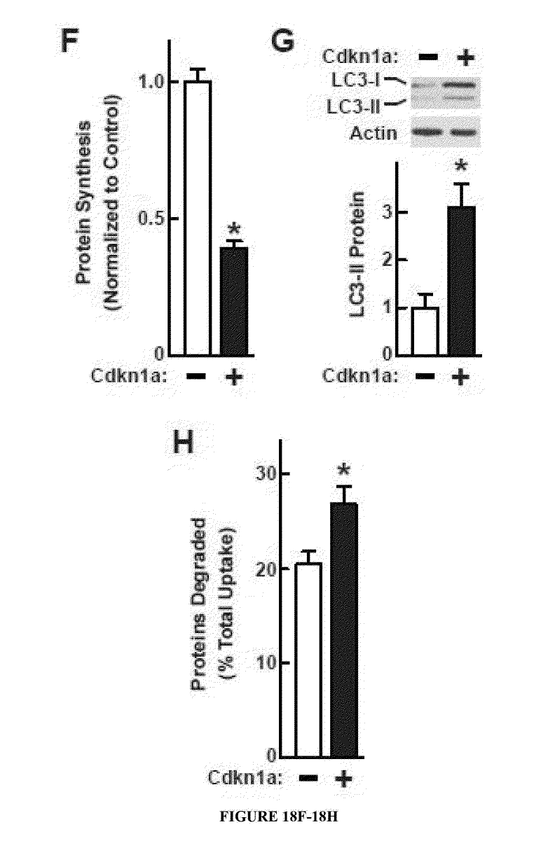

It was previously established that Gadd45a decreased mRNAs involved in mitochondrial biogenesis and glucose utilization, including PGC-1α, TEAM, THRA (thyroid hormone receptor alpha), GLUT4 and HK2 (hexokinase-2) (Ebert, S. M., et al. (2012) The Journal of biological chemistry 287, 27290-27301). Similarly, Cdkn1a overexpression significantly reduced these mRNAs in mouse skeletal muscle (FIG. 18A). Consistent with these changes, Cdkn1a also reduced PGC-1α protein, the mitochondrial protein Cox4, and mitochondrial DNA in mouse skeletal muscle (FIG. 18B-18D). Also like Gadd45a (Ebert, S. M., et al. (2012) The Journal of biological chemistry 287, 27290-27301), Cdkn1a decreased mRNAs involved in anabolic signaling, including AR (androgen receptor) and GHR (growth hormone receptor) (FIG. 18A). Consistent with these changes, Cdkn1a reduced phosphorylation (activity) of a key anabolic mediator, the protein kinase Akt in cultured skeletal myotubes (FIG. 18E). As expected, this was accompanied by decreased protein synthesis (FIG. 18F).

In addition to repressing genes and cellular processes that protect skeletal muscle fibers from atrophy, Gadd45a stimulates protein degradation, which promotes atrophy (Ebert, S. M., et al. (2012) The Journal of biological chemistry 287, 27290-27301). Similarly, Cdkn1a significantly induced a key autophagy mRNA (Bnip3) in mouse skeletal muscle (FIG. 18A). Cdkn1a also tended to increase another key autophagy mRNA (LC3a), and Cdkn1a significantly increased total and lipidated LC3 protein in mouse muscle (FIGS. 18A and 18G). Consistent with these effects, Cdkn1a increased protein degradation in skeletal myotubes (FIG. 18H).

Collectively, these data indicate that Cdkn1a causes skeletal muscle atrophy by altering muscle gene expression in a manner that reduces barriers to atrophy (PGC-1α, mitochondria, Akt activity, protein synthesis) and stimulates protein breakdown. In addition, increased Cdkn1a expression appears to account for many of the downstream effects of Gadd45a on skeletal muscle gene expression, mitochondria, cellular signaling, protein metabolism and muscle fiber size.

(5) Discussion of FIGS. 12-18

The current study elucidates a new molecular mechanism of skeletal muscle atrophy. The data in FIGS. 1-11 identified Gadd45a as an important stress-induced factor that alters skeletal muscle gene expression to increase proteolysis and decrease anabolic signaling, protein synthesis and mitochondria, leading to muscle atrophy (Ebert, S. M., et al. (2012) The Journal of biological chemistry 287, 27290-27301). However, the molecular target of Gadd45a remained unknown. The data in FIGS. 12-18 demonstrate that Gadd45a causes atrophy by interacting with the Cdkn1a promoter and stimulating its demethylation. This increases the level of Cdkn1a mRNA and then Cdkn1a (p21^{WAF1/Cip1}) protein, which is sufficient to induce many of the downstream effects of Gadd45a, including altered muscle gene expression, decreased anabolic signaling, reduced protein synthesis and decreased mitochondria, increased proteolysis and ultimately, muscle atrophy. Taken together, these results identify the Cdkn1a promoter as a key molecular target of Gadd45a, and uncover new and important roles for DNA demethylation and Cdkn1a in the pathogenesis of skeletal muscle atrophy.

Interestingly, previous studies in humans, mice, rats and pigs demonstrated that Cdkn1a mRNA is among the most highly induced transcripts in atrophying skeletal muscle (Ebert, S. M., et al. (2012) The Journal of biological chemistry 287, 27290-27301; Ebert, S. M., et al. (2010) Molecular Endocrinology 24, 790-799; Banduseela, V. C., et al. (2009) Physiological genomics 39, 141-159; Llano-Diez, M., et al. (2011) BMC genomics 12, 602; Welle, S., et al. (2004) Experimental gerontology 39, 369-377; Welle, S., et al. (2003) Physiological genomics 14, 149-159; Edwards, M. G., et al. (2007) BMC genomics 8, 80; Stevenson, E. J., et al. (2003) The Journal of physiology 551, 33-48; Gonzalez de Aguilar, J. L., et al. (2008) Physiological genomics 32, 207-218; Laure, L., (2009) The FEBS journal 276, 669-684; Bodine, S. C., et al. (2001) Science (New York, N. Y 294, 1704-1708). In addition, a prior study showed that Cdkn1a protein was markedly induced in skeletal muscle following muscle denervation (Ishido, M., et al. (2004) American journal of physiology 287, C484-493). However, the consequence of increased Cdkn1a expression in atrophying skeletal muscle was not known. The current study demonstrates that increased Cdkn1a expression is required for skeletal muscle fiber atrophy during three very different types of stress: muscle immobilization, muscle denervation and fasting. Moreover, forced expression of Cdkn1a in skeletal muscle fibers or cultured skeletal myotubes is sufficient to generate atrophy in the absence of upstream stress. These new data explain the previous observations that Cdkn1a is highly induced in atrophic muscle and identify Cdkn1a as a pivotal molecular mediator of skeletal muscle atrophy.

Although Cdkn1a increases protein degradation, it was found that Cdkn1a does not increase atrogin-1/MAFbx or MuRF1 mRNAs in skeletal muscle or myotubes (not shown). This is consistent with previous findings that Gadd45a does not increase atrogin-1/MAFbx or MuRF1 mRNAs in skeletal muscle or myotubes (FIGS. 9D and 11B). Atrogin-1/MAFbx and MuRF1 mRNAs encode E3 ubiquitin ligases that contribute to proteolysis and play essential roles in skeletal muscle atrophy, but are not sufficient to induce atrophy (Bodine, S. C., et al. (2001) Science (New York, N.Y 294, 1704-1708; Sandri, M., et al. (2004) Cell 117, 399-412; Moresi, V., et al. (2010) Cell 143, 35-45; Stitt, T. N., et al. (2004) Mol Cell 14, 395-403). Since Gadd45a and Cdkn1a are sufficient to induce proteolysis of total cellular protein and muscle fiber atrophy, but not sufficient to induce atrogin-1/MAFbx or MuRF1 mRNAs, this indicates that atrogin-1 and MuRF1 could function upstream of Gadd45a and Cdkn1a during the pathogenesis of skeletal muscle atrophy. An alternative possibility is that atrogin-1 and MuRF1 mediate a parallel pathway to skeletal muscle proteolysis.

In summary, the data in FIGS. 12-18 provide a molecular explanation for how Gadd45a causes skeletal muscle atrophy, and elucidate a role for active DNA demethylation and Cdkn1a in the pathogenesis of skeletal muscle atrophy.

The data in FIGS. 1-18 have therapeutic implications. First, they discover Gadd45a, Cdkn1a and DNA demethylation as therapeutic targets in skeletal muscle atrophy. Second, these data discover that, during muscle atrophy, Cdkn1a is responsible for repressing mRNAs involved in anabolic signaling, including mRNAs encoding the androgen receptor and growth hormone receptor.

xi) Ursolic Acid Reduces Gadd45a and Cdkn1a Expression in Skeletal Muscle

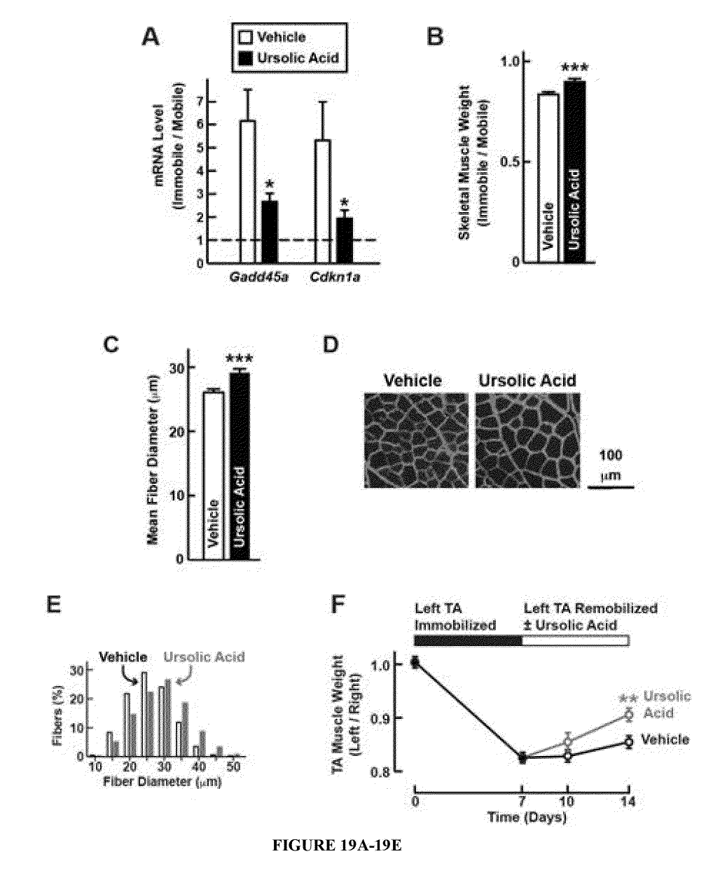

The effect of ursolic acid on Gadd45a and Cdkn1a expression was investigated. Beginning on day 0, C57BL/6 mice were given intraperitoneal injections of ursolic acid (200 mg/kg) or an equal volume of vehicle (corn oil) twice a day. On day 2, the left tibialis anterior (TA) muscle was immobilized using an Autosuture Royal 35W skin stapler (Tyco Healthcare, Point Claire, Q C, Canada) as described previously (Caron et al., 2009). On day 5, bilateral TA muscles were harvested and mRNA was isolated for qPCR analysis. In each mouse, mRNA levels in the left (immobile) TA were normalized to mRNA levels in the right (mobile) TA, which were set at one and indicated by the dashed line. As shown in FIG. 19A, ursolic acid prevented induction of Gadd45a and Cdkn1a mRNAs during immobilization-induced skeletal muscle atrophy. (Data are means±SEM from 10 mice per condition; *p<0.05).

The effect of ursolic acid on immobilization-induced skeletal muscle atrophy was investigated. 6-8 wk old male C57BL/6 mice were obtained from the National Cancer Institute. Beginning on day 0, mice were given i.p. injections of ursolic acid (200 mg/kg) or an equal volume of vehicle (corn oil) twice a day. On day 2, the left tibialis anterior (TA) muscle of each mouse was immobilized using an Autosuture Royal 35W skin stapler (Tyco Healthcare, Point Claire, Q C, Canada) to induce skeletal muscle atrophy as described previously (Caron et al., 2009). During immobilization, vehicle or ursolic acid continued to be administered via i.p. injection twice daily, and the right TA remained mobile and served as an intrasubject control. On day 8, bilateral TA muscles were harvested and weighed. In each mouse, the left (immobile) TA weight was normalized to the right (mobile) TA weight. As shown in FIG. 19B, ursolic acid reduced the loss of muscle mass in immobilized muscles. Moreover, ursolic acid increased the size of skeletal muscle fibers in immobilized muscles (FIGS. 19C-E), indicating reduced muscle atrophy. Data in FIG. 19B are means±SEM from 19 mice per condition; ***P<0.001 by unpaired t-test. Data in FIG. 19C are mean fiber diameters ±SEM from 10 immobilized TA muscles per condition; ***P<0.001 by unpaired t-test. Data in FIG. 19D are representative cross-sections of muscle fibers immunostained with anti-laminin antibody. Data in FIG. 19E are fiber size distributions of >3000 fibers from 10 immobilized TA muscles per condition. These data indicate that ursolic acid prevents immobilization-induced skeletal muscle atrophy.

To determine whether ursolic acid might enhance recovery from skeletal muscle atrophy, mouse TA muscles were immobilized for 7 days to induce atrophy (FIG. 19F). Muscles were then remobilized by removing the staple from the left TA muscle. Treatment with vehicle or ursolic acid (200 mg/kg) was then initiated. Both vehicle and ursolic acid were given via i.p. injection twice daily. Ursolic acid significantly enhanced the recovery of skeletal muscle mass (FIG. 19F). Data in FIG. 19F are means±SEM from 8 mice per condition; **P<0.01 by unpaired t-test.

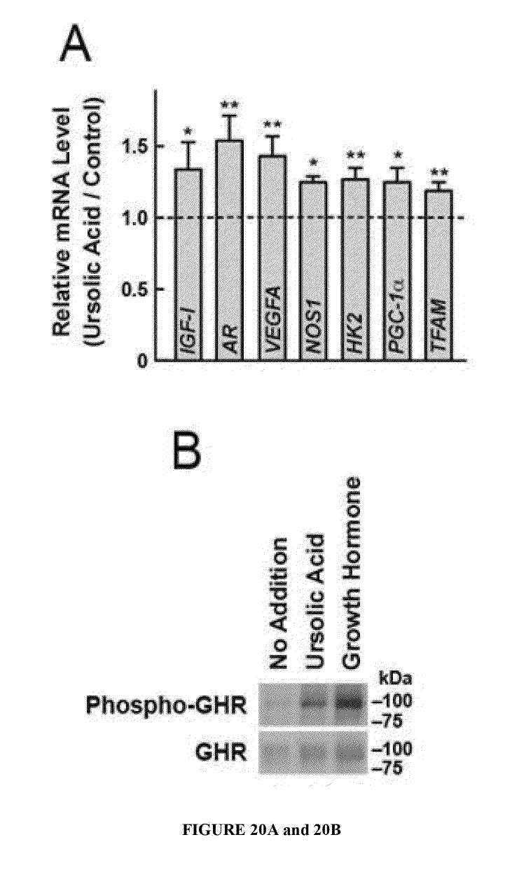

The finding that ursolic acid reduced Gadd45a and Cdkn1a expression (FIG. 19A) suggested that ursolic acid might reverse downstream molecular effects of Gadd45a and Cdkn1a. To test this, C57BL/6 mice were fed diets lacking or containing 0.14% ursolic acid for 6 weeks before quadriceps muscles were harvested for qPCR analysis. FIG. 20A shows that ursolic acid increased mRNAs involved in anabolic signaling (IGF-I and androgen receptor (Ar)), mitochondrial biogenesis (PGC-1α(Ppargc1a) and Tfam), angiogenesis, vascular flow and oxygen delivery (Nos1 and Vegfa) and glucose utilization (Hk2). These effects of ursolic acid are consistent with ursolic acid's capacity to decrease Gadd45a and Cdkn1a expression.

The effect of ursolic acid on the growth hormone receptor GHR) was then investigated. Cultured C2C12 myoblasts were serum-starved for 6 hours, and then incubated for 2 minutes in the absence or presence of ursolic acid (10 μM) and/or recombinant human growth hormone (100 ng/ml), as indicated. Total cellular protein extracts were subjected to immunoprecipitation with anti-GHR antibody, followed by SDS-PAGE and immunoblot analysis with anti-phospho-tyrosine or anti-GHR antibodies to assess phospho-GHR and total GHR, respectively. FIG. 20B shows that ursolic acid increased GHR phosphorylation, indicating that ursolic acid activates GHR.

xii) The Treatment of Muscle Atrophy

Ursolic acid is administered to an animal with muscle atrophy in a dose ranging from 0.1-10 g per day. Testosterone is administered as a topical gel (2.5-81 mg per day), as a topical patch (2.5-7.5 mg per day), as a topical solution (30-120 mg per day), or as an intramuscular injection (50-400 mg testosterone enanthate or testosterone cypionate, given every 1-4 weeks).

Ursolic acid is administered to an animal with muscle atrophy in a dose ranging from 0.1-10 g per day. Growth hormone is administered as a subcutaneously injection (in a dose ranging from 0.04 mg to 8 mg per day).

All of the compositions and/or methods disclosed and claimed herein can be made and executed without undue experimentation in light of the present disclosure. It will be apparent to those skilled in the art that various modifications and variations can be made in the present invention without departing from the scope or spirit of the invention.

More specifically, certain agents which are both chemically and physiologically related can be substituted for the agents described herein while the same or similar results can be achieved. All such similar substitutes and modifications apparent to those skilled in the art are deemed to be within the spirit, scope and concept of the invention as defined by the appended claims.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### G. REFERENCES

Abbas T, et al. (2009). p21 in cancer: intricate networks and multiple activities. Nat Rev Cancer 9, 400-414.
Acharyya, S., Ladner, K. J., Nelsen, L. L., Damrauer, J., Reiser, P. J., Swoap, S., and Guttridge, D. C. (2004) Cancer cachexia is regulated by selective targeting of skeletal muscle gene products. J. Clin. Investig. 114, 370-378
Amthor, H., Macharia, R., Navarrete, R., Schuelke, M., Brown, S. C., Otto, A., Voit, T., Muntoni, F., Vrbóva, G., Partridge, T., Zammit, P., Bunger, L., and Patel, K. (2007) Lack of myostatin results in excessive muscle growth but impaired force generation. Proc. Natl. Acad. Sci. U.S.A. 104, 1835-1840
Anderson, R. D., Haskell, R. E., Xia, H., Roessler, B. J., and Davidson, B. L. (2000) A simple method for the rapid generation of recombinant adenovirus vectors. Gene Ther. 7, 1034-1038
Banduseela, V. C., Ochala, J., Chen, Y. W., Göransson, H., Norman, H., Radell, P., Eriksson, L. I., Hoffman, E. P., and Larsson, L. (2009) Gene expression and muscle fiber function in a porcine ICU model. Physiol. Genomics 39, 141-159
Barres, R., Osler, M. E., Yan, J., Rune, A., Fritz, T., Caidahl, K., Krook, A., and Zierath, J. R. (2009) Cell metabolism 10, 189-198
Barres, R., Yan, J., Egan, B., Treebak, J. T., Rasmussen, M., Fritz, T., Caidahl, K., Krook, A., O'Gorman, D. J., and Zierath, J. R. (2012) Cell metabolism 15, 405-411
Barreto G, et al. (2007). Gadd45a promotes epigenetic gene activation by repair-mediated DNA demethylation. Nature 445, 671-675.
Benson, E. K., Zhao, B., Sassoon, D. A., Lee, S. W., and Aaronson, S. A. (2009) Cell cycle (Georgetown, Tex. 8, 2002-2004
Bird, A. (2002) Genes & development 16, 6-21
Bodine, S. C., Latres, E., Baumhueter, S., Lai, V. K., Nunez, L., Clarke, B. A., Poueymirou, W. T., Panaro, F. J., Na, E., Dharmarajan, K., Pan, Z. Q., Valenzuela, D. M., DeChiara, T. M., Stitt, T. N., Yancopoulos, G. D., and Glass, D. J. (2001) Identification of ubiquitin ligases required for skeletal muscle atrophy. Science 294, 1704-1708
Bodine, S. C., Stitt, T. N., Gonzalez, M., Kline, W. 0., Stover, G. L., Bauerlein, R., Zlotchenko, E., Scrimgeour, A., Lawrence, J. C., Glass, D. J., and Yancopoulos, G. D. (2001) Akt/mTOR pathway is a crucial regulator of skeletal muscle hypertrophy and can prevent muscle atrophy in vivo. Nat. Cell Biol. 3, 1014-1019
Brüning, J. C., Michael, M. D., Winnay, J. N., Hayashi, T., Hörsch, D., Accili, D., Goodyear, L. J., and Kahn, C. R. (1998) Amuscle-specific insulin receptor knockout exhibits features of the metabolic syndrome of NIDDM without altering glucose tolerance. Mol. Cell 2, 559-569
Burks, T. N., Andres-Mateos, E., Marx, R., Mejias, R., Van Erp, C., Simmers, J. L., Walston, J. D., Ward, C. W., and Cohn, R. D. (2011) Losartan restores skeletal muscle remodeling and protects against disuse atrophy in sarcopenia. Sci. Translat. Med. 3, 82ra37
Cai, D., Frantz, J. D., Tawa, N. E., Jr., Melendez, P. A., Oh, B. C., Lidov, H. G., Hasselgren, P. 0., Frontera, W. R., Lee, J., Glass, D. J., and Shoelson, S. E. (2004) IKKβ/NF-kB activation causes severe muscle wasting in mice. Cell 119, 285-298
Caron, A. Z., Drouin, G., Desrosiers, J., Trensz, F., and Grenier, G. (2009) A novel hindlimb immobilization procedure for studying skeletal muscle atrophy and recovery in mouse. J. Appl. Physiol. 106, 2049-2059
Chedin, F. (2011) Progress in molecular biology and translational science 101, 255-285
Chen, H., Vermulst, M., Wang, Y. E., Chomyn, A., Prolla, T. A., McCaffery, J. M., and Chan, D. C. (2010) Mitochondrial fusion is required for mtDNA stability in skeletal muscle and tolerance of mtDNA mutations. Cell 141, 280-289
Cohn, R. D., Henry, M. D., Michele, D. E., Barresi, R., Saito, F., Moore, S. A., Flanagan, J. D., Skwarchuk, M. W., Robbins, M. E., Mendell, J. R., Williamson, R. A., and Campbell, K. P. (2002) Disruption of DAG1 in differentiated skeletal muscle reveals a role for dystroglycan in muscle regeneration. Cell 110, 639-648
Cortellino, S., Xu, J., Sannai, M., Moore, R., Caretti, E., Cigliano, A., Le Coz, M., Devarajan, K., Wessels, A., Soprano, D., Abramowitz, L. K., Bartolomei, M. S., Rambow, F., Bassi, M. R., Bruno, T., Fanciulli, M., Renner, C., Klein-Szanto, A. J., Matsumoto, Y., Kobi, D., Davidson, I., Alberti, C., Lame, L., and Bellacosa, A. (2011) Thymine DNA glycosylase is essential for active DNA demethylation by linked deamination-base excision repair. Cell 146, 67-79
Dedkov, E. I., Borisov, A. B., and Carlson, B. M. (2003) Dynamics of post-denervation atrophy of young and old skeletal muscles. Differential responses of fiber types and muscle types. J. Gerontol. 58, 984-991
Deng Y, et al. (2000). Peg3/Pw1 promotes p53-mediated apoptosis by inducing Bax translocation from cytosol to mitochondria. Proceedings of the National Academy of Sciences of the United States of America 97, 12050-12055.
Dubowitz, V., Lane, R., and Sewry, C. A. (2007) Muscle Biopsy: A Practical Approach, 3rd ed., Saunders Elsevier, Philadelphia, Pa.
Easwaran, H. P., and Baylin, S. B. (2010) Role of nuclear architecture in epigenetic alterations in cancer. Cold Spring Harbor Symp. Quant. Biol. 75, 507-515
Ebert, S. M., Dyle, M. C., Kunkel, S. D., Bullard, S. A., Bongers, K. S., Fox, D. K., Dierdorff, J. M., Foster, E. D., and Adams, C. M. (2012) The Journal of biological chemistry 287, 27290-27301
Ebert, S. M., Monteys, A. M., Fox, D. K., Bongers, K. S., Shields, B. E., Malmberg, S. E., Davidson, B. L., Suneja, M., and Adams, C. M. (2010) The transcription factor ATF4 promotes skeletal myofiber atrophy during fasting. Mol. Endocrinol. 24, 790-799
Edwards, M. G., Anderson, R. M., Yuan, M., Kendziorski, C. M., Weindruch, R., and Prolla, T. A. (2007) Gene expression profiling of aging reveals activation of a p53-mediated transcriptional program. BMC Genomics 8, 80
el-Deiry, W. S., Tokino, T., Waldman, T., Oliner, J. D., Velculescu, V. E., Burrell, M., Hill, D. E., Healy, E., Rees, J. L., Hamilton, S. R., and et al. (1995) Cancer research 55, 2910-2919
Fearon, K. C., Glass, D. J., and Guttridge, D. C. (2012) Cell metabolism 16, 153-166
Frame, S., and Cohen, P. (2001) GSK3 takes center stage more than 20 years after its discovery. Biochem. J. 359, 1-16
Fry, C. S., and Rasmussen, B. B. (2011) Current aging science 4, 260-268
Glass, D., and Roubenoff, R. (2010) Ann N Y Acad Sci 1211, 25-36
Gomes, M. D., Lecker, S. H., Jagoe, R. T., Navon, A., and Goldberg, A. L. (2001) Atrogin-1, a muscle-specific F-box protein highly expressed during muscle atrophy. Proc. Natl. Acad. Sci. U.S.A. 98, 14440-14445
Gonzalez de Aguilar, J. L., Niederhauser-Wiederkehr, C., Halter, B., De Tapia, M., Di Scala, F., Demougin, P., Dupuis, L., Primig, M., Meininger, V., and Loeffler, J. P. (2008) Gene profiling of skeletal muscle in an amyotrophic lateral sclerosis mouse model. Physiol. Genomics 32, 207-218
Gundersen, K., and Bruusgaard, J. C. (2008) Nuclear domains during muscle atrophy. Nuclei lost or paradigm lost? J. Physiol. 586, 2675-2681
Harding, H. P., Zhang, Y., Zeng, H., Novoa, I., Lu, P. D., Calfon, M., Sadri, N., Yun, C., Popko, B., Paules, R., Stojdl, D. F., Bell, J. C., Hettmann, T., Leiden, J. M., and Ron, D. (2003) An integrated stress response regulates amino acid metabolism and resistance to oxidative stress. Mol. Cell 11, 619-633
Ishido, M., Kami, K., and Masuhara, M. (2004) American journal of physiology 287, C484-493
Jiang H, et al. (2007). The eukaryotic initiation factor-2 kinase pathway facilitates differential GADD45a expression in response to environmental stress. The Journal of biological chemistry 282, 3755-3765.
Jung Y, et al. (2010). Examination of the expanding pathways for the regulation of p21 expression and activity. Cell Signal 22, 1003-1012.
Kamei, Y., Miura, S., Suzuki, M., Kai, Y., Mizukami, J., Taniguchi, T., Mochida, K., Hata, T., Matsuda, J., Aburatani, H., Nishino, I., and Ezaki, 0. (2004) The Journal of biological chemistry 279, 41114-41123
Kaneko-Ishino T, et al. (1995). Pegl/Mest imprinted gene on chromosome 6 identified by cDNA subtraction hybridization. Nature genetics 11, 52-59.
Kastan, M. B., Zhan, Q., el-Deiry, W. S., Carrier, F., Jacks, T., Walsh, W. V., Plunkett, B. S., Vogelstein, B., and Fornace, A. J., Jr. (1992) A mammalian cell cycle checkpoint pathway utilizing p53 and GADD45 is defective in ataxia-telangiectasia. Cell 71, 587-597
Korényi-Both, A. L. (1983) Muscle Pathology in Neuromuscular Disease, C. C. Thomas, Springfield, Ill.
Kunkel, S. D., Suneja, M., Ebert, S. M., Bongers, K. S., Fox, D. K., Malmberg, S. E., Alipour, F., Shields, R. K., and Adams, C. M. (2011) mRNA expression signatures of human skeletal muscle atrophy identify a natural compound that increases muscle mass. Cell Metab. 13, 627-638
Kutner, M. H., Nachtsheim, C., and Neter, J. (2004) Applied Linear Regression Models, 4th ed., McGraw-Hill/Irwin, Boston
Lal, A., and Gorospe, M. (2006) Cell cycle (Georgetown, Tex. 5, 1422-1425
Laure, L., Suel, L., Roudaut, C., Bourg, N., Ouali, A., Bartoli, M., Richard, I., and Danièle, N. (2009) Cardiac ankyrin repeat protein is a marker of skeletal muscle pathological remodeling. FEBS J. 276, 669-684
Lecker, S. H., Jagoe, R. T., Gilbert, A., Gomes, M., Baracos, V., Bailey, J., Price, S. R., Mitch, W. E., and Goldberg, A. L. (2004) Multiple types of skeletal muscle atrophy involve a common program of changes in gene expression. FASEB J. 18, 39-51
Le May, N., Mota-Fernandes, D., Velez-Cruz, R., Iltis, I., Biard, D., and Egly, J. M. (2010) Mol Cell 38, 54-66
Li, J. B., and Goldberg, A. L. (1976) Effects of food deprivation on protein synthesis and degradation in rat skeletal muscles. Am. J. Physiol. 231, 441-448
Liebermann, D. A., and Hoffman, B. (2008) Gadd45 in stress signaling. J. Mol. Signal 3, 15
Lin H, et al. (2010) Skp2 targeting suppresses tumorigenesis by Arf-p53-independent cellular senescence. Nature 464, 374-379.
Llano-Diez, M., Gustafson, A. M., Olsson, C., Goransson, H., and Larsson, L. (2011) BMC genomics 12, 602
Malmberg, S. E., and Adams, C. M. (2008) Insulin signaling and the general amino acid control response. Two distinct pathways to amino acid synthesis and uptake. J. Biol. Chem. 283, 19229-19234
Mammucari, C., Milan, G., Romanello, V., Masiero, E., Rudolf, R., Del Piccolo, P., Burden, S. J., Di Lisi, R., Sandri, C., Zhao, J., Goldberg, A. L., Schiaffino, S., and Sandri, M. (2007) FoxO3 controls autophagy in skeletal muscle in vivo. Cell Metab. 6, 458-471
Masuoka H, et al. (2002). Targeted disruption of the activating transcription factor 4 gene results in severe fetal anemia in mice. Blood 99, 736-745.
Mendez, J., Keys, A. (1960) Metabolism: clinical and experimental 9, 184-189
Michael, L. F., Wu, Z., Cheatham, R. B., Puigserver, P., Adelmant, G., Lehman, J. J., Kelly, D. P., and Spiegelman, B. M. (2001) Restoration of insulin-sensitive glucose transporter (GLUT4) gene expression in muscle cells by the transcriptional coactivator PGC-1. Proc. Natl. Acad. Sci. U.S.A. 98, 3820-3825
Moresi, V., Williams, A. H., Meadows, E., Flynn, J. M., Potthoff, M. J., McAnally, J., Shelton, J. M., Backs, J., Klein, W. H., Richardson, J. A., Bassel-Duby, R., and Olson, E. N. (2010) Myogenin and class II HDACs control neurogenic muscle atrophy by inducing E3 ubiquitin ligases. Cell 143, 35-45
Niehrs, C., and Schafer, A. (2012) Trends Cell Biol 22, 220-227
Palus S, et al., (2011). Ghrelin and Its Analogues, BIM-28131 and BIM-28125, Improve Body Weight and Regulate the Expression of MuRF-1 and MAFbx in a Rat Heart Failure Model. PLoS One, 6(11): e26865.
Peterson, J. M., Bakkar, N., and Guttridge, D. C. (2011) NF-kB signaling in skeletal muscle health and disease. Curr. Top. Dev. Biol. 96, 85-119
Plant, P. J., Bain, J. R., Correa, J. E., Woo, M., and Batt, J. (2009) Absence of caspase-3 protects against denervation-induced skeletal muscle atrophy. J. Appl. Physiol. 107, 224-234
Porter, D. C., Farmaki, E., Altilia, S., Schools, G. P., West, D. K., Chen, M., Chang, B. D., Puzyrev, A. T., Lim, C. U., Rokow-Kittell, R., Friedhoff, L. T., Papavassiliou, A. G., Kalurupalle, S., Hurteau, G., Shi, J., Baran, P. S., Gyorffy, B., Wentland, M. P., Broude, E. V., Kiaris, H., and Roninson, I. B. (2012) Proceedings of the National Academy of Sciences of the United States of America 109, 13799-13804
Powers, S. K., Wiggs, M. P., Duarte, J. A., Zergeroglu, A. M., and Demirel, H. A. (2012) Am J Physiol Endocrinol Metab 303, E31-39
Reinhardt, H. C., Hasskamp, P., Schmedding, I., Morandell, S., van Vugt, M. A., Wang, X., Linding, R., Ong, S. E., Weaver, D., Can, S. A., and Yaffe, M. B. (2010) Mol Cell 40, 34-49
Sacheck, J. M., Hyatt, J. P., Raffaello, A., Jagoe, R. T., Roy, R. R., Edgerton, V. R., Lecker, S. H., and Goldberg, A. L. (2007) Rapid disuse and denervation atrophy involve transcriptional changes similar to those of muscle wasting during systemic diseases. FASEB J. 21, 140-155
Sandri M. (2008). Signaling in muscle atrophy and hypertrophy. Physiology (Bethesda) 23, 160-170.
Sandri, M., Lin, J., Handschin, C., Yang, W., Arany, Z. P., Lecker, S. H., Goldberg, A. L., and Spiegelman, B. M. (2006) PGC-1α protects skeletal muscle from atrophy by suppressing FoxO3 action and atrophy-specific gene transcription. Proc. Natl. Acad. Sci. U.S.A. 103, 16260-16265
Sandri, M., Sandri, C., Gilbert, A., Skurk, C., Calabria, E., Picard, A., Walsh, K., Schiaffino, S., Lecker, S. H., and Goldberg, A. L. (2004) FOXO transcription factors induce the atrophy-related ubiquitin ligase atrogin-1 and cause skeletal muscle atrophy. Cell 117, 399-412
Sartori, R., Milan, G., Patron, M., Mammucari, C., Blaauw, B., Abraham, R., and Sandri, M. (2009) Smad2 and -3 transcription factors control muscle mass in adulthood. Am. J. Physiol. Cell Physiol. 296, C1248-C1257
Sen, G. L., Reuter, J. A., Webster, D. E., Zhu, L., and Khavari, P. A. (2010) Nature 463, 563-567
Schiaffino, S., and Mammucari, C. (2011) Regulation of skeletal muscle growth by the IGF1-Akt/PKB pathway. Insights from genetic models. Skelet. Muscle 1, 4
Schmitz, K. M., Schmitt, N., Hoffmann-Rohrer, U., Schafer, A., Grummt, I., and Mayer, C. (2009) Mol Cell 33, 344-353
Schwarzkopf, M., Coletti, D., Sassoon, D., and Marazzi, G. (2006) Muscle cachexia is regulated by a p53-PW1/Peg3-dependent pathway. Genes Dev. 20, 3440-3452
Scime, A., Grenier, G., Huh, M. S., Gillespie, M. A., Bevilacqua, L., Harper, M. E., and Rudnicki, M. A. (2005) Cell metabolism 2, 283-295
Stevenson, E. J., Giresi, P. G., Koncarevic, A., and Kandarian, S. C. (2003) Global analysis of gene expression patterns during disuse atrophy in rat skeletal muscle. J. Physiol. 551, 33-48
Stitt, T. N., Drujan, D., Clarke, B. A., Panaro, F., Timofeyva, Y., Kline, W. 0., Gonzalez, M., Yancopoulos, G. D., and Glass, D. J. (2004) The IGF-1/PI3K/Akt pathway prevents expression of muscle atrophy-induced ubiquitin ligases by inhibiting FOXO transcription factors. Mol. Cell 14, 395-403
Subramanian A, et al. (2005). Gene set enrichment analysis: a knowledge-based approach for interpreting genome-wide expression profiles. Proceedings of the National Academy of Sciences of the United States of America 102, 15545-15550.
Sytnikova, Y. A., Kubarenko, A. V., Schafer, A., Weber, A. N., and Niehrs, C. (2011) Gadd45a is an RNA-binding protein and is localized in nuclear speckles. PloS One 6, e14500
Tian, J., Huang, H., Hoffman, B., Liebermann, D. A., Ledda-Columbano, G. M., Columbano, A., and Locker, J. (2011) Gadd45β is an inducible coactivator of transcription that facilitates rapid liver growth in mice. J. Clin. Investig. 121, 4491-4502
Tran, H., Brunet, A., Grenier, J. M., Datta, S. R., Fornace, A. J., Jr., Di-Stefano, P. S., Chiang, L. W., and Greenberg, M. E. (2002) DNA repair pathway stimulated by the forkhead transcription factor FOXO3a through the Gadd45 protein. Science 296, 530-534
Uldry, M., Yang, W., St-Pierre, J., Lin, J., Seale, P., and Spiegelman, B. M. (2006) Complementary action of the PGC-1 coactivators in mitochondrial biogenesis and brown fat differentiation. Cell Metab. 3, 333-341
Verhees, K. J., Schols, A. M., Kelders, M. C., Op den Kamp, C. M., van der Velden, J. L., and Langen, R. C. (2011) Glycogen synthase kinase-3β is required for the induction of skeletal muscle atrophy. Am. J. Physiol. Cell Physiol. 301, C995-C1007
Volk, K. A., Husted, R. F., Sigmund, R. D., and Stokes, J. B. (2005) The Journal of biological chemistry 280, 18348-18354
Wang, X., Blagden, C., Fan, J., Nowak, S. J., Taniuchi, I., Littman, D. R., and Burden, S. J. (2005) Runx1 prevents wasting, myofibrillar disorganization, and autophagy of skeletal muscle. Genes Dev. 19, 1715-1722
Weber M, et al. (2005). Chromosome-wide and promoter-specific analyses identify sites of differential DNA methylation in normal and transformed human cells. Nature genetics 37, 853-862.
Welle, S., Brooks, A. I., Delehanty, J. M., Needler, N., and Thornton, C. A. (2003) Gene expression profile of aging in human muscle. Physiol. Genomics 14, 149-159
Welle, S., Brooks, A. I., Delehanty, J. M., Needler, N., Bhatt, K., Shah, B., and Thornton, C. A. (2004) Skeletal muscle gene expression profiles in 20-29-year-old and 65-71-year-old women. Exp. Gerontol. 39, 369-377
Wenz, T., Rossi, S. G., Rotundo, R. L., Spiegelman, B. M., and Moraes, C. T. (2009) Increased muscle PGC-1α expression protects from sarcopenia and metabolic disease during aging. Proc. Natl. Acad. Sci. U.S.A. 106, 20405-20410
Yi, Y. W., Kim, D., Jung, N., Hong, S. S., Lee, H. S., and Bae, I. (2000) Gadd45 family proteins are coactivators of nuclear hormone receptors. Biochem. Biophys. Res. Commun. 272, 193-198
Zeman, R. J., Zhao, J., Zhang, Y., Zhao, W., Wen, X., Wu, Y., Pan, J., Bauman, W. A., and Cardozo, C. (2009) Differential skeletal muscle gene expression after upper or lower motor neuron transection. Pflugers Arch. 458, 525-535
Zhan, Q. (2005) Mutation research 569, 133-143
Zhang, Z., Winborn, C. S., Marquez de Prado, B., and Russo, A. F. (2007) Sensitization of calcitonin gene-related peptide receptors by receptor activity-modifying protein-1 in the trigeminal ganglion. J. Neurosci. 27, 2693-2703
Zhao H, et al. (2000). The central region of Gadd45 is required for its interaction with p21/WAF1. Experimental cell research 258, 92-100.
Zhao, J., Brault, J. J., Schild, A., Cao, P., Sandri, M., Schiaffino, S., Lecker, S. H.,
and Goldberg, A. L. (2007) FoxO3 coordinately activates protein degradation by the autophagic/lysosomal and proteasomal pathways in atrophying muscle cells. Cell Metab. 6, 472-483

1. A composition for treating or inhibiting the progression of skeletal muscle atrophy, the composition comprising:
(a) a Gadd45a and/or Cdkn1a inhibitor; and
(b) an androgen and/or growth hormone receptor elevator or an androgen and/or growth hormone receptor activator.

2. The composition of claim 1, wherein the inhibitor is ursolic acid.

3. (canceled)

4. (canceled)

5. The composition of claim 1, wherein the inhibitor is a compound of the formula: 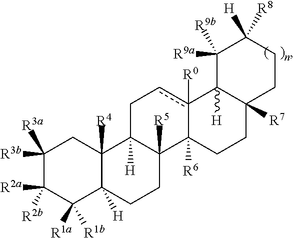
wherein each [symbol] is an optional covalent bond, and R⁰ is optionally present;
wherein n is 0 or 1;
wherein R⁰, when present, is hydrogen;
wherein R^{1a} is selected from C1-C6 alkyl and —C(O)ZR¹⁰; wherein R^{1b} is selected from C1-C6 alkyl; or wherein R^{1a} and R^{1b} are covalently bonded and, along with the intermediate carbon, together comprise optionally substituted C3-C5 cycloalkyl or optionally substituted C2-C5 heterocycloalkyl;
wherein R^{2a} and R^{2b} are independently selected from hydrogen and —OR¹¹, provided that at least one of R^{2a} and R^{2b} is —OR¹¹; or wherein R^{2a} and R^{2b} together comprise ═O;
wherein each of R^{3a} and R^{3b} is independently selected from hydrogen, hydroxyl, C1-C6 alkyl, and C1-C6 alkoxyl, provided that R^{3a} and R^{3b} are not simultaneously hydroxyl; or
wherein R^{3a} and R^{3b} are covalently bonded and, along with the intermediate carbon, together comprise optionally substituted C3-C5 cycloalkyl or optionally substituted C2-C5 heterocycloalkyl;
wherein each of R⁴, R⁵, and R⁶ is independently selected from C1-C6 alkyl;
wherein R⁷ is selected from C1-C6 alkyl, —CH₂OR¹², and —C(O)ZR¹²;
wherein R⁸ is selected from hydrogen and C1-C6 alkyl;
wherein each of R^{9a} and R^{9b} is independently selected from hydrogen and C1-C6 alkyl, provided that R^{9a} and R^{9b} are not simultaneously hydrogen; or wherein R^{9a} and R^{9b} are covalently bonded and, along with the intermediate carbon, together comprise optionally substituted C3-C5 cycloalkyl or optionally substituted C2-C5 heterocycloalkyl;
wherein R¹⁰ is selected from hydrogen and C1-C6 alkyl;
wherein each R¹¹ is independently selected from hydrogen, C1-C6 alkyl, C1-C5 heteroalkyl, C3-C6 cycloalkyl, C4-C6 heterocycloalkyl, phenyl, heteroaryl, and —C(O)R¹⁴; wherein R¹¹, where permitted, is substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl;
wherein R¹² is selected from hydrogen and optionally substituted organic residue having from 1 to 20 carbons;
wherein Z is selected from —O— and —NR¹³—;
wherein R¹³ is selected from hydrogen and C1-C4 alkyl; or, wherein Z is N, R¹² and R¹³ are covalently bonded and —NR¹²R¹³ comprises a moiety of the formula: 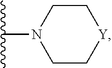
wherein Y is selected from —O—, —S—, —SO—, —SO₂—, —NH—, —NCH₃—; and
wherein R¹⁴ is C1-C6 alkyl and substituted with 0-2 groups selected from cyano, acyl, fluoro, chloro, bromo, iodo, methyl, ethyl, propyl, butyl, pentyl, hexyl, hydroxyl, acetoxyl, methoxyl, ethoxyl, propoxyl, and butoxyl;
or a pharmaceutically acceptable salt, hydrate, solvate, or polymorph thereof.

6-8. (canceled)

9. A method for treating or inhibiting the progression of muscle atrophy in an animal, the method comprising administering to the animal an effective amount of a composition comprising a Gadd45a and/or Cdkn1a inhibitor and an androgen and/or growth hormone elevator or receptor activator.

10. (canceled)

11. A method for activating growth hormone receptor in a mammal, the method comprising administering a composition comprising ursolic acid or an ursolic acid derivative.

12. A method according to claim 9 for treating or inhibiting the progression of muscle atrophy in an animal, the method comprising administering to the animal an effective amount of an androgen and/or growth hormone elevator subsequent to the animal having received a Gadd45a and/or Cdkn1a inhibitor.

13. A method according to claim 9 for treating or inhibiting the progression of muscle atrophy in an animal, the method comprising administering to the animal an effective amount of a Gadd45a and/or Cdkn1a inhibitor subsequent to the animal having received an androgen and/or growth hormone elevator.

14. A method according to claim 9 for treating or inhibiting the progression of muscle atrophy in an animal, the method comprising administering to the animal an effective amount of an androgen and/or growth hormone receptor activator subsequent to the animal having received a Gadd45a and/or Cdkn1a inhibitor.

15. A method according to claim 9 for treating or inhibiting the progression of muscle atrophy in an animal, the method comprising administering to the animal an effective amount of a Gadd45a and/or Cdkn1a inhibitor subsequent to the animal having received an androgen and/or growth hormone receptor activator.

16. A method for facilitating muscle hypertrophy, the method comprising the steps of:
inhibiting expression of Gadd45a and/or Cdkn1a; and
increasing activity of androgen and/or growth hormone receptor.

17. (canceled)

18. (canceled)

19. A composition according to claim 1 comprising:
(a) an androgen and/or growth hormone receptor activator or an androgen and/or growth hormone elevator,
(b) a Gadd45a and/or Cdkn1a inhibitor, and
(c) a pharmaceutically acceptable carrier.

20. (canceled)

21. A composition according to claim 1 wherein said androgen and/or growth hormone receptor elevator or androgen and/or growth hormone receptor activator is chosen from testosterone, dihydrotestosterone, androstenedione, ghrelin, BIM-28125, BIM-28131, aminoglutethimide, testolactone, anastrozole, letrozole, exemestane, vorozole, formestane, fadrozole, 4-hydroxyandrostenedione, 1,4,6-androstatrien-3,17-dione, 4-androstene-3,6,17-trione, growth hormone, and a SARM.

22. A composition according to claim 21, wherein said Gadd45a and/or Cdkn1a inhibitor is ursolic acid.


SEQUENCE LISTING (8 sequences)

SEQ ID NO: 1 - DNA, 29 bp, Artificial Sequence
  Description of Artificial Sequence/Note = Synthetic Construct
gcagacgttcctgggttagatacaataac

SEQ ID NO: 2 - DNA, 25 bp, Artificial Sequence
  Description of Artificial Sequence/Note = Synthetic Construct
gccactgtttactatcaccccagcc

SEQ ID NO: 3 - DNA, 20 bp, Artificial Sequence
  Description of Artificial Sequence/Note = Synthetic Construct
cttctgctgggtgtgatggc

SEQ ID NO: 4 - DNA, 21 bp, Artificial Sequence
  Description of Artificial Sequence/Note = Synthetic Construct
cccaagatccagacagtccac

SEQ ID NO: 5 - DNA, 23 bp, Artificial Sequence
  Description of Artificial Sequence/Note = Synthetic Construct
tgtagttttaattttaagtaagg

SEQ ID NO: 6 - DNA, 25 bp, Artificial Sequence
  Description of Artificial Sequence/Note = Synthetic Construct
cactaaaataacattaataaaaaac

SEQ ID NO: 7 - DNA, 23 bp, Artificial Sequence
  Description of Artificial Sequence/Note = Synthetic Construct
aggtattatttttgttgggtgtg

SEQ ID NO: 8 - DNA, 22 bp, Artificial Sequence
  Description of Artificial Sequence/Note = Synthetic Construct
caaaatccaaacaatccactaa